(19) United States
(12) Patent Application Publication
(10) Pub. No.: US 2006/0073496 A1
(43) Pub. Date: Apr. 6, 2006

(54) Methods of identifying patients at risk of developing encephalitis following immunotherapy for Alzheimer's disease

(76) Inventors: Margot O'Toole, Newtonville, MA (US); Andrew J. Dorner, Lexington, MA (US); Derek B. Janszen, Royersford, PA (US); Donna K. Slonim, North Andover, MA (US); William M. Mounts, Andover, MA (US); Padmalatha S. Reddy, Lexington, MA (US); Andrew A. Hill, Cambridge, MA (US)

Correspondence Address: FITZPATRICK CELLA (WYETH), 30 ROCKEFELLER PLAZA, NEW YORK, NY, 10112-3800, US

(21) Appl. No.: 11/186,236

(22) Filed: Jul. 20, 2005

Related U.S. Application Data
(60) Provisional application No. 60/589,877, filed on Jul. 20, 2004.
(60) Provisional application No. 60/672,716, filed on Apr. 18, 2005.

(51) Int. Cl. C12Q 1/68; G06F 19/00

(52) U.S. Cl. 435/6; 705/3

(57) ABSTRACT

The present invention generally relates to a method for an improved treatment for Alzheimer's disease (AD) using immunotherapy, e.g., immunotherapy targeting β amyloid (Aβ), e.g., immunotherapy based on AN1792. In one embodiment, the method allows for predicting an adverse clinical response, and therefore allows for an improved safety profile of AN1792. In another embodiment, the method allows for predicting a favorable clinical response, and therefore allows for an improved efficacy profile of AN1792. The methods of the present invention may be combined to predict a favorable clinical response and the lack of an adverse clinical response.

This application claims the benefit of U.S. Provisional Application Ser. No. 60/589,877, filed Jul. 20, 2004, and U.S. Provisional Application Ser. No. 60/672,716, filed Apr. 18, 2005, both of which are incorporated herein by reference in their entireties.

### BACKGROUND OF THE INVENTION

1. Field of the Invention

The present invention generally relates to methods for an improved treatment for Alzheimer's disease. The methods employ pharmacogenomic information to develop an immunotherapy targeted against Aβ peptide, e.g., an immunotherapy based on AN1792, that exhibits a reduction in adverse clinical responses and/or an increased incidence of favorable clinical responses to such immunotherapy resulting in its improved safety and efficacy.

2. Related Background Art

Alzheimer's disease (AD) is a progressive degenerative disease of the brain primarily associated with aging. Clinical presentation of AD is characterized by loss of memory, cognition, reasoning, judgment, and orientation. As the disease progresses, motor, sensory, and linguistic abilities are also affected until there is global impairment of multiple cognitive functions. These cognitive losses may occur gradually, but typically lead to severe impairment and eventual death in the range of four to twelve years.

Alzheimer's disease is characterized by major pathologic observations in the brain: neurofibrillary tangles, the accumulation of β-amyloid (or neuritic) plaques (comprised predominantly of an aggregate of a peptide fragment known as Aβ), and by increased rates of neuronal atrophy. Individuals with AD exhibit characteristic β-amyloid deposits in the brain (β-amyloid plaques), cerebral blood vessels (β-amyloid angiopathy), and neurofibrillary tangles. Neurofibrillary tangles occur not only in AD but also in other dementia-inducing disorders. On autopsy, large numbers of these lesions are generally found in areas of the human brain important for memory and cognition.

Smaller numbers of these lesions in a more restricted anatomical distribution are found in the brains of most elderly humans who do not have clinical AD. Amyloidogenic plaques and vascular amyloid angiopathy also characterize the brains of individuals with trisomy 21 (Down syndrome), hereditary cerebral hemorrhage with amyloidosis of the Dutch-type (HCHWA-D), and other neurodegenerative disorders. β-amyloid is a defining feature of AD, and is now believed to be a causative precursor or factor in the development of disease. Deposition of Aβ in areas of the brain responsible for cognitive activities is a major factor in the development of AD. β-amyloid plaques predominantly are composed of amyloid β peptide (Aβ, also sometimes designated β-A/4). Aβ peptide is derived by proteolysis of the amyloid precursor protein (APP). Several proteases called secretases are involved in the processing of APP.

Cleavage of APP at the N-terminus of the Aβ peptide by β-secretase and at the C-terminus by one or more γ-secretases constitutes the α-amyloidogenic pathway, i.e., the pathway by which Aβ is formed. Cleavage of APP by α-secretase produces α-sAPP, a secreted form of APP that does not result in β-amyloid plaque formation. This alternate pathway precludes the formation of Aβ peptide. A description of the proteolytic processing fragments of APP is found, for example, in U.S. Pat. Nos. 5,441,870; 5,721,130; and 5,942,400.

Several lines of evidence indicate that progressive cerebral deposition of β-amyloid peptide (Aβ) plays an influential role in the pathogenesis of AD and can precede cognitive symptoms by years or decades (see, e.g., Selkoe (1991) Neuron 6 (4):487-98). Release of Aβ from neuronal cells grown in culture and the presence of Aβ in cerebrospinal fluid of both normal individuals and AD patients has been demonstrated (see, e.g., Seubert et al. (1992) Nature 359:325-27).

At present there is no effective treatment for preventing, slowing, arresting, and/or reversing the progression of AD. Therefore, there is an urgent need for pharmaceutical agents capable of preventing, slowing, arresting and/or, reversing the progression of AD.

One problem with finding a treatment for AD is that, in general, there is great heterogeneity in the way that humans respond to medications. Currently, empirical methods are typically used to find the appropriate drug therapy for an individual patient. However, such empirical strategies run the risk that a patient will receive a drug that is ineffective, thus delaying effective therapy, or that a patient may develop an adverse clinical response or side effect to the drug. When the subset of patients at risk of the development of an adverse clinical response cannot be identified prior to the administration of a given drug, the development of that drug may be terminated; thus, the possibility of benefiting from therapy involving that drug may be denied to those patients who are not susceptible to an adverse clinical response to that drug.

One such adverse drug reaction was seen with AN1792, a peptide immunogen consisting of Aβ1-42, the section of amyloid recognized as a major component of AD-related plaques (Iwatsubo et al. (1994) Neuron 13:45-53). Administration of AN1792 is an experimental therapeutic strategy against AD based on the theory that administration of β-amyloid might activate the immune system to raise its own protective anti-amyloid antibodies that “recognize” and attack the β-amyloid plaques that are a hallmark of AD brain abnormality (Schenk et al (2000) Arch. Neurol. 57:934-36).

In 1999, the first preclinical animal studies with AN1792 were reported (see Schenk et al. (1999) Nature 400:173-77). Studies in transgenic mouse models of cognitive impairment and amyloid plaque-associated CNS pathology demonstrated that immunization with AN1792 resulted in improved cognitive function and inhibited the development of AD-like amyloid plaques, neuritic dystrophy, and gliosis in mice (Games et al. (1995) Nature 373:523-27; Schenk et al. (1999) Nature 400:173-77; Morgan et al. (2000) Nature 408:982-85; Janus et al. (2000) Nature 408:979-82; DeMattos et al. (2001) Proc. Natl. Acad. Sci. USA 98:8850-55; McLaurin et al. (2002) Nat. Med. 8:1263-69). The mice treated with AN1792, and not those treated with placebo, had improved performance in memory tests. Based on these preclinical results, both the U.S. Food and Drug Administration and the U.K. Medicines Control Agency permitted Phase I human trials of AN1792 to assess its safety and tolerability in people with mild to moderate AD.

The U.K. trial enrolled about 80 patients and the U.S. trial enrolled about 24 patients with mild to moderate AD for the Phase I trials. Results from the Phase I trials were announced in 2000 and indicated that AN1792 was well tolerated in human recipients and that a portion of the participants developed amyloid antibodies, as was seen in the preclinical animal studies (Klocinski and Karlawish (2002) University of Pennsylvania Memory Disorders Clinic News Letter, 1 (4):5-8; Bayer et al (2005) Neurology 64:94-101). Based on these outcomes, in late 2001, a small Phase Ia double-blind, placebo-controlled, multi-centered trial began in the United States and Europe enrolling 372 patients with mild to moderate AD to evaluate safety, tolerability and pilot-efficacy of AN1792 administered with QS-21 adjuvant (Fox et al. (2005) Neurology 64:1563-72; Gilman et al. (2005) Neurology 64:1553-62; Orgogozo et al. (2003) Neurology 61:46-54). For the Phase Ia trials, 300 patients were randomly selected to receive six immunizations of AN1792 and 72 patients were randomly selected to receive placebo (Gilman, supra). Four of the participants developed signs of meningoencephalitis at an early phase of the clinical trial, and the trial was suspended. Soon after the suspension, 14 more patients developed signs of meningoencephalitis; the Safety Monitoring Committee concluded that dosing with the immunotherapeutic AN1792 should be discontinued. At the time the treatments were discontinued, the maximum number of immunization received was three (by 24 patients), with the majority of patients receiving two immunizations (274 patients) and two patients receiving one immunization. Ultimately, meningoencephalitis was reported in 18 of 300 immunized patients (Orgogozo (2003) supra). All 18 patients had received AN1792, whereas no patient in the placebo group developed encephalitis (Orgogozo (2003) supra).

Trial researchers continued to follow all participants, i.e., cognitive function, memory and executive function, and anti-AN1792 antibody, CSF tau, and CSF Aβ1-42 levels were assessed to the conclusion of the original follow-up period. Antibody responders were compared to placebo controls. Two sets of measurements, levels of CSF tau and a battery of neuropsychological tests, gave results favoring patients with a positive IgG titer (Gilman, supra). However, the exact cause of the brain inflammation, i.e., meningoencephalitis, in some subjects is not yet known. The follow-up studies showed that the participants who suffered from meningoencephalitis developed antibodies to β-amyloid but that there did not appear to be any correlation between antibody levels and the risk of developing brain inflammation. An autopsy of one participant who died of causes unrelated to treatment showed signs of brain inflammation. Interestingly, significant areas of the brain lacked the β-amyloid plaques targeted by the immunotherapeutic, a phenomenon not seen in the brains of patients diagnosed with AD. Whole-trial analysis remains ongoing (Gilman, supra).

In order for AN1792 to be considered a possible therapy for AD, it is desirable to understand how the immune system responds to AN1792 such that the complications associated with the therapy, e.g., inflammation leading to, e.g., meningoencephalitis, may be reduced. Pharmacogenomics may allow the identification of predictive biomarkers of responsiveness to the immunotherapeutic, e.g., for the identification of patients, prior to therapy, who are most likely to develop a favorable clinical response, e.g., a protective immune response, (e.g., an antibody response) and/or least likely to develop an adverse clinical response, e.g., inflammation that may result in, e.g., encephalitis (e.g., meningoencephalitis).

Pharmacogenomics seeks to investigate and identify genomic factors that contribute to drug response variation(s) among individuals with seemingly equivalent disease symptoms. Recent advances in the sequencing of the human genome have enabled researchers to more efficiently and effectively link certain genomic variations to particular diseases. Pharmacogenomics has the potential to revolutionize treatment strategies and to aid in the development of clinical in vitro diagnostics, which would be far superior to empirical treatment. Increasing knowledge about the interactions between genes and drug treatment should create a proportionate demand for rapid and reliable pretreatment diagnostic tests to ensure the safest and most effective treatment possible.

By utilizing the tools of pharmacogenomics, the present invention overcomes the inadequacies of AN1792 immunotherapy by providing an effective method for optimizing both the efficacy and safety of AN1792. The present invention draws correlations between gene expression patterns and clinical responses to a treatment for AD (particularly administration of AN1792), provides methods for predicting clinical and pathological responses, and provides methods for using this information to improve the clinical response profile of AN1792 and to develop a therapeutic product for patients preselected for optimal safety and efficacy (e.g., a “genomically guided” therapeutic product).

### SUMMARY OF THE INVENTION

The present invention is directed to a method of using pharmacogenomic information to predict a clinical response in an AD patient to a treatment for AD. In one embodiment of the invention, the treatment is an immunotherapeutic, e.g., an active immunotherapeutic. In particular, the present invention is directed to active immunotherapy targeting Aβ peptide, e.g., an immunotherapy based on AN1792.

Accordingly, the invention provides methods of compiling pharmacogenomic information to associate a unique gene expression pattern of a patient sample with a particular clinical response to a treatment for AD. Generally, the methods for compiling pharmacogenomic information to associate a unique gene expression pattern of a patient sample with a particular clinical response to a treatment for AD comprise the steps of procuring at least one patient sample from a patient of a first population of patients and at least one patient sample from a patient of a second population of patients (wherein the first population consists of one or more patients who developed the particular clinical response to the treatment for AD and wherein the second population consists of one or more patients who did not develop the particular response to the treatment for AD); acquiring a gene expression pattern from each procured patient sample; and determining whether at least most of the patient samples procured from the first population have a unique gene expression pattern not found in at least most of the patient samples procured from the second population; wherein a determination that at least most of the patient samples procured from the first population have a unique gene expression pattern not found in at least most of the patient samples procured from the second population results in associating the unique gene expression pattern with the particular clinical response to the treatment for AD. In one embodiment of the invention, the particular clinical response is one that is neither favorable nor adverse (e.g., antibody nonresponsiveness). In some embodiments, the particular clinical response is either a favorable clinical response or an adverse clinical response. In other embodiments, the particular clinical response is both a favorable and adverse clinical response. For example, the particular clinical response may be inflammation, and said inflammation may encompass development of both an IgG response and encephalitis.

The invention thus provides a method for compiling pharmacogenomic information to associate a unique gene expression pattern of a patient sample with a favorable clinical response to a treatment for AD comprising the steps of procuring at least one patient sample from a patient of a first population of patients and at least one patient sample from a patient of a second population of patients (wherein the first population consists of one or more patients who developed the favorable clinical response to the treatment for AD and wherein the second population consists of one or more patients who did not develop the favorable response to the treatment for AD); acquiring a gene expression pattern from each procured patient sample; and determining whether at least most of the patient samples procured from the first population have a unique gene expression pattern not found in at least most of the patient samples procured from the second population; wherein a determination that at least most of the patient samples procured from the first population have a unique gene expression pattern not found in at least most of the patient samples procured from the second population results in associating the unique gene expression pattern with the favorable clinical response to the treatment for AD. In one embodiment of the invention, the second population consists of one or more patients who did not develop the favorable clinical response to the treatment and also developed an adverse clinical response. In another embodiment of the invention, the method further comprises excluding patients who also developed an adverse clinical response to the treatment for AD from the first population of patients.

The present invention also provides a method of compiling pharmacogenomic information to associate a unique gene expression pattern of a patient sample with an adverse clinical response to a treatment for AD comprising the steps of procuring at least one patient sample from a patient of a first population of patients and at least one patient sample from a patient of a second population of patients (wherein the first population consists of one or more patients who developed the adverse clinical response to the treatment for AD and wherein the second population consists of one or more patients who did not develop the adverse response to the treatment for AD); acquiring a gene expression pattern from each procured patient sample; and determining whether at least most of the patient samples procured from the first population have a unique gene expression pattern not found in at least most of the patient samples procured from the second population, wherein a determination that at least most of the patient samples procured from the first population have a unique gene expression pattern not found in at least most of the patient samples procured from the second population results in associating the unique gene expression pattern with the adverse clinical response to the treatment for AD. In one embodiment of the invention, the second population consists of one or more patients who did not develop the adverse clinical response to the treatment and also developed a favorable clinical response. In another embodiment of the invention, the method further comprises excluding patients who also developed a favorable response to the treatment for AD from the first population of patients. In some embodiments, selected genes or groups of genes are excluded before acquiring a gene expression pattern to improve the accuracy of statistical findings, e.g., genes identified as significant covariates.

In some methods of compiling pharmacogenomic information, samples are placed under a certain culture condition(s) prior to acquisition of gene expression patterns. In some embodiments, the clinical response that is neither favorable nor adverse is low to undetectable antibody production. In some embodiments, the favorable clinical response is a protective immune response. In some embodiments, the favorable clinical response is an antibody response, e.g., an IgG response. In some embodiments, the adverse clinical response is an inflammatory response. In some embodiments, the inflammatory response leads to encephalitis, e.g., meningoencephalitis. In some embodiments of the methods of compiling pharmacogenomic information, the patient samples are peripheral blood mononuclear cells. In some embodiments, the gene expression pattern is selected from the group consisting of protein expression patterns and RNA expression patterns.

In some embodiments of the invention, the methods of compiling pharmacogenomic information are used to associate a unique gene expression pattern of a patient sample with a particular clinical response to administration of AN1792. Accordingly, the invention also provides methods of compiling pharmacogenomic information to associate a unique gene expression pattern of a patient sample taken from a patient treated with AN1792 with a clinical response to the administration of AN1792. In one embodiment of the invention, gene expression patterns are acquired from unstimulated samples. In another embodiment of the invention, gene expression patterns are acquired from stimulated (e.g., cultured) samples.

The invention also provides a method for determining a unique gene expression pattern for predicting whether a candidate AD patient is likely to develop an IgG response to administration of AN1792 comprising referring to nucleic acid samples from a patient population previously exposed to AN1792, wherein the patient population includes IgG responders and IgG nonresponders, and wherein IgG expression is developed in response to administration of AN1792; and comparing the nucleic acid samples of the IgG responders with the nucleic acid samples of the IgG nonresponders to determine the unique gene expression pattern associated with IgG responders. Also provided is a method for determining a unique gene expression pattern for predicting whether a candidate AD patient is likely to not develop an IgG response to administration of AN1792 comprising referring to nucleic acid samples from a patient population previously exposed to AN1792, wherein the patient population includes IgG responders and IgG nonresponders, and wherein IgG expression is developed in response to administration of AN1792; and comparing the nucleic acid samples of the IgG nonresponders with the nucleic acid samples of the IgG responders to determine the unique gene expression pattern associated with the IgG nonresponders. The invention also provides a method for determining a unique gene expression pattern for predicting whether a candidate AD patient is likely to develop inflammation in response to administration of AN1792 comprising referring to nucleic acid samples from a patient population previously exposed to AN1792, wherein the patient population includes inflammation developers and inflammation nondevelopers, and wherein inflammation is developed in response to administration of AN1792; and comparing the nucleic acid samples of the inflammation developers with the nucleic acid samples of the inflammation nondevelopers to determine the unique gene expression pattern associated with inflammation developers.

The invention also provides a method for determining a unique gene expression pattern for predicting whether a candidate AD patient is likely to develop an IgG response to administration of AN1792 comprising acquiring gene expression patterns from a patient population previously exposed to AN1792, wherein the patient population includes IgG responders and IgG nonresponders, and wherein IgG expression is developed in response to administration of AN1792; and comparing the gene expression patterns of the IgG responders to the gene expression patterns of the IgG nonresponders to determine the unique gene expression pattern associated with the IgG responders. The invention also provides a method for determining a unique gene expression pattern for predicting whether a candidate AD patient is likely to not develop an IgG response to administration of AN1792 comprising acquiring gene expression patterns from a patient population previously exposed to AN1792, wherein the patient population includes IgG nonresponders and IgG responders, and wherein IgG expression is developed in response to administration of AN1792; and comparing the gene expression patterns of the IgG nonresponders to the gene expression patterns of the IgG responders to determine the unique gene expression pattern associated with the IgG nonresponders. Additionally, the invention provides a method for determining a unique gene expression pattern for predicting whether a candidate AD patient is likely to develop inflammation in response to administration of AN1792 comprising acquiring gene expression patterns from a patient population previously exposed to AN1792, wherein the patient population includes inflammation developers and inflammation nondevelopers, and wherein inflammation is developed in response to administration of AN1792; and comparing the gene expression patterns of the inflammation developers to the gene expression patterns of the inflammation nondevelopers to determine the unique gene expression pattern associated with the inflammation developers.

The invention also provides a method for determining a unique gene expression pattern for predicting whether a candidate AD patient is likely to develop an IgG response to administration of AN1792 comprising procuring blood samples from a patient population previously exposed to AN1792, wherein the patient population includes IgG responders and IgG nonresponders, and wherein IgG expression is developed in response to administration of AN1792; purifying total RNA from the blood samples, thereby producing RNA samples; assaying RNA expression levels from the RNA samples to obtain gene expression patterns for the IgG responders and the IgG nonresponders; and comparing the gene expression patterns of the IgG responders to the gene expression patterns of the IgG nonresponders to determine the unique gene expression pattern associated with the IgG responders. Also provided is a method for determining a unique gene expression pattern for predicting whether a candidate AD patient is likely to not develop an IgG response to administration of AN1792 comprising procuring blood samples from a patient population previously exposed to AN1792, wherein the patient population includes IgG nonresponders and IgG responders, and wherein IgG expression is developed in response to administration of AN1792; purifying total RNA from the blood samples, thereby producing RNA samples; assaying RNA expression levels from the RNA samples to obtain gene expression patterns for the IgG nonresponders and the IgG responders; and comparing the gene expression patterns of the IgG nonresponders to the gene expression patterns of the IgG responders to determine the unique gene expression pattern associated with the IgG nonresponders. The invention also provides a method for determining a unique gene expression pattern for predicting whether a candidate AD patient is likely to develop inflammation in response to administration of AN1792 comprising procuring blood samples from a patient population previously exposed to AN1792, wherein the patient population includes inflammation developers and inflammation nondevelopers, and wherein inflammation is developed in response to administration of AN1792; purifying total RNA from the blood samples, thereby producing RNA samples; assaying RNA expression levels from the RNA samples to obtain gene expression patterns for the inflammation developers and the inflammation nondevelopers; and comparing the gene expression patterns of the inflammation developers to the gene expression patterns of the inflammation nondevelopers to determine the unique gene expression pattern associated with the inflammation developers.

In some embodiments of methods of determining a unique gene expression pattern, the gene expression pattern is selected from the group consisting of protein gene expression patterns and RNA gene expression patterns. In other embodiments of methods of determining a unique gene expression pattern, the methods further comprise assaying total RNA expression levels from an RNA sample obtained from the patient population to acquire the gene expression pattern. Other embodiments of methods of determining a unique gene expression pattern further comprise assaying total protein expression levels from a protein sample obtained from the patient population to acquire the gene expression pattern.

The invention also provides unique gene expression patterns that are associated with a particular response to a treatment for AD. In some embodiments, a gene expression pattern of the invention is a protein gene expression pattern. In other embodiments, a gene expression pattern of the invention is an RNA gene expression pattern. In some embodiments, the unique gene expression pattern comprises the expression level of one gene that may be considered individually. In other embodiments, the invention provides a unique gene expression pattern that comprises expression levels of a panel of genes, wherein the expression levels are or will be measured, e.g., by the measurement of gene products (e.g., RNA, proteins, etc.). In one embodiment, a panel of the invention may comprise 2-5, 5-15, 15-35, 35-50, 50-100, or more than 100 genes. In one embodiment, a panel may comprise 15-20 genes. In another embodiment, a panel may comprise two genes.

The invention also provides kits, e.g., a kit comprising one or more polynucleotides, each capable of hybridizing under stringent conditions to an RNA transcript, or the complement thereof, of a gene differentially expressed in a unique gene expression pattern of the invention; and/or one or more antibodies, each capable of binding to a polypeptide encoded by a gene differentially expressed in a unique gene expression pattern of the invention. In some embodiments, a gene differentially expressed in a unique gene expression pattern of the invention is a gene differentially expressed in PBMCs of AD patients likely to develop a particular clinical response when treated with AN1792 as compared to PBMCs of AD patients likely not to develop the particular clinical response when treated with AN1792. For example, in some embodiments, the particular clinical response may be an antibody response (e.g., an IgG response). In other embodiments, the particular clinical response is inflammation, e.g., encephalitis (e.g., meningoencephalitis). In some embodiments, the polynucleotides and/or antibodies of a kit of the invention are coupled to a solid support.

In one embodiment, a panel or kit of the invention comprises genes selected from one of Tables 10-12, 18, and 24-37. In another embodiment, a panel or kit of the invention comprises a combination of genes selected from those listed in Tables 10-12, 18, and 24-37. In a further embodiment, a panel or kit of the invention comprises genes listed in Table 36. In another embodiment, a panel or kit of the invention comprises a pair of genes, e.g., any of the pairs of genes listed in Table 37.

It is an object of the invention to use unique gene expression patterns associated with particular clinical responses to predict the clinical response of a candidate patient to a treatment for AD. Thus the invention also provides methods of predicting whether a candidate patient who has not been previously exposed to a treatment for AD will develop a particular clinical response to a treatment for AD, the methods generally comprising the steps of associating at least one unique gene expression pattern of a patient sample with a particular clinical response to the treatment for AD; procuring a test sample from the candidate patient who has not been previously exposed to the treatment for AD; and determining that the test sample procured from the candidate patient who has not been previously exposed to the treatment for AD has a test gene expression pattern that is substantially similar to the at least one gene expression pattern associated with the particular clinical response to the treatment for AD (i.e., the at least one reference gene expression pattern), wherein if it is determined that the test sample has a test gene expression pattern that is substantially similar to the at least one gene expression pattern associated with the particular clinical response, it may be predicted that the candidate patient will develop the particular clinical response. In one embodiment, the particular clinical response is neither favorable nor adverse. In another embodiment, the particular clinical response is either a favorable or adverse clinical response. In an additional embodiment, the particular clinical response is both a favorable and adverse clinical response.

In a specific embodiment, the methods of the present invention include obtaining and/or determining a first population of patients that develops a particular clinical response (wherein the particular clinical response is, e.g., the development of an inflammatory response, particularly encephalitis, and/or the development of an IgG response, but may be any other particular clinical response, such as decrease in plaque formation, to a treatment for AD (e.g., an immunotherapeutic-based treatment for AD, e.g., AN1792)), and a second population of patients that does not develop the particular clinical response. The method of the present invention further comprises examining the gene expression patterns of the first population to discover whether there are any specific gene expression patterns associated with the particular clinical response. Phenotypic characteristics may further define genomic populations and result in further improved response profiles of treatments for AD, e.g., immunotherapeutics, including but not limited to AN1792; for example, in some treatments, females may have a greater degree of adverse clinical responses than males. The method then comprises associating a unique gene expression pattern with the particular clinical response(s), wherein the unique gene expression pattern defines a population having, e.g., an improved therapeutic response profile to a treatment. The gene expression pattern predicts patients, for example, who may develop inflammation and/or who may have or develop a certain level of IgG response.

In a further aspect of the invention, there is provided a system comprising a computer readable memory which stores at least one reference gene expression pattern of one or more genes wherein each of the one or more genes is differentially expressed in patient samples procured from AD patients who are likely to develop a particular clinical response to a therapy for AD, e.g., AN1792 treatment, compared to patient samples procured from AD patients who are not likely to develop the particular clinical response to the therapy for AD; a program capable of comparing a test gene expression pattern to the reference gene expression pattern and a processor capable of executing the program are also provided in the system.

When such computer readable memory and program exist, i.e., where there already exists reference gene expression patterns (e.g., wherein the reference gene expression pattern is associated with a particular response to the treatment for AD by any method of compiling pharmacogenomic information), the methods of predicting a clinical response of a candidate patient comprise the steps of procuring a test sample from the candidate patient not previously exposed to the treatment for AD, and determining whether the test sample from the candidate patient has a test gene expression pattern that is substantially similar to a reference gene expression pattern that has been previously associated with a particular clinical response, wherein if it is determined that the test sample has a test gene expression pattern that is substantially similar to a reference gene expression pattern that has been previously associated with the particular clinical response, it may be predicted that the candidate patient will develop the particular clinical response. In some embodiments, the particular clinical response is neither a favorable nor an adverse clinical response. In other embodiments, the particular clinical response is a favorable or an adverse clinical response.

In particular, the invention provides methods for predicting whether an AD patient is likely to benefit from treatment for AD comprising the steps of collecting a blood sample from the patient; isolating blood cells from the sample; purifying total RNA from the cells, thereby producing an RNA sample; assaying RNA expression levels from the RNA sample to obtain a gene expression pattern; and comparing the gene expression pattern of the patient with the gene expression pattern of patients who benefited from the treatment, whereby a substantial similarity between the gene expression patterns indicates the patient is likely to benefit from the treatment for AD. For example, the invention provides a method for predicting whether an AD patient is likely to develop an immune response to an immunotherapy treatment for AD comprising collecting a blood sample from the patient; isolating blood cells from the sample; purifying total RNA from the cells, thereby producing an RNA sample; assaying RNA expression levels from the RNA sample to obtain a gene expression pattern; and comparing the gene expression pattern of the patient with the gene expression pattern of patients who developed an immune response to the immunotherapy, whereby a substantial similarity between the gene expression patterns indicates the patient is likely to develop an immune response to the immunotherapy treatment for AD. In some embodiments of the invention, the particular immune response is neither a favorable nor an adverse clinical response, e.g., the clinical response may be undetectable to low IgG production. In other embodiments, the clinical response is both favorable and adverse. In another embodiment, the clinical response is an immune response, e.g., an IgG response. In other embodiments, the clinical response is the development of inflammation, e.g., meningoencephalitis.

Additionally, the invention provides a method for predicting whether an AD patient is likely to develop an adverse reaction in response to a treatment for AD comprising collecting a blood sample from the patient; isolating blood cells from the sample; purifying total RNA from the cells, thereby producing an RNA sample; assaying RNA expression levels from the RNA sample to obtain a gene expression pattern; and comparing the gene expression pattern of the patient with the gene expression pattern of patients who developed an adverse reaction in response to the treatment, whereby a substantial similarity between the gene expression patterns indicates the patient is likely to develop an adverse reaction in response to the treatment for AD. For example, the invention provides a method for predicting whether an AD patient is likely to develop an adverse reaction in response to an immunotherapy treatment for AD comprising collecting a blood sample from the patient; isolating blood cells from the sample; purifying total RNA from the cells, thereby producing an RNA sample; assaying RNA expression levels from the RNA sample to obtain a gene expression pattern; and comparing the gene expression pattern of the patient with the gene expression pattern of patients who developed an adverse reaction in response to the immunotherapy, whereby a substantial similarity between the gene expression patterns indicates the patient is likely to develop an adverse reaction in response to the immunotherapy treatment for AD.

In some embodiments, a candidate patient's clinical response to AN1792 is predicted. Therefore the present invention relates to a method of predicting whether a candidate patient will develop a particular clinical response when administered AN1792 comprising the steps of compiling pharmacogenomic information to associate at least one unique gene expression pattern of a preimmunization patient sample procured from a patient who has been treated with AN1792 with a particular clinical response, procuring a test sample from the candidate patient, and determining whether the test sample has a test gene expression pattern that is substantially similar to the at least one unique gene expression pattern, wherein if the test sample has a test gene expression pattern that is substantially similar to the at least one gene expression pattern associated with the particular clinical response, it may be predicted that the candidate patient will develop the particular clinical response. In some embodiments, the step of determining is performed with unstimulated patient samples. In other embodiments, the step of determining is performed with in vitro cultured patient samples. In one embodiment, the particular clinical response is neither favorable nor adverse, e.g., nonresponsiveness. In another embodiment, the particular clinical response to AN1792 is a favorable clinical response, e.g., a protective immune response, e.g., an IgG antibody response. In another embodiment, the particular clinical response to AN1792 is an adverse clinical response, e.g., an inflammatory response, e.g., encephalitis. Thus, the invention also provides methods of identifying an AD patient who is likely not to develop an IgG response when treated with AN1792, comprising the steps of providing at least one test patient sample of a candidate AD patient; and comparing a test gene expression pattern of one or more genes to at least one reference gene expression pattern, wherein each of the one or more genes of the reference gene expression pattern is differentially expressed in patient samples procured from AD patients who are likely not to develop an IgG response when treated with AN1792 as compared to patient samples procured from AD patients who are likely to develop an IgG response when treated with AN1792. The invention also provides a method of identifying an AD patient who is likely to develop inflammation when treated with AN1792, comprising the steps of providing at least one test patient sample of a candidate AD patient; and comparing a test gene expression pattern of one or more genes in the at least one test patient sample to at least one reference gene expression pattern from an AD patient treated with AN1792, wherein each of the one or more genes is differentially expressed in patient samples procured from patients who are likely to develop inflammation when treated with AN1792 as compared to in patient samples procured from patients who are not likely to develop inflammation when treated with AN1792. In the methods of identifying an AD patient unlikely or likely to develop a particular clinical response when treated with AN1792, the patient sample may comprise enriched PBMCs. In some embodiments, the patient sample is a whole blood sample. In some embodiments, the gene expression pattern is determined using quantitative RT-PCR or an immunoassay.

In some embodiments, the clinical response of a candidate patient to treatment with AN1792 may be predicted, and/or AD patients may be identified using gene expression patterns, kits, and systems of the invention. In some embodiments, a gene expression pattern described in Table 10-12, 18, or 24-37 is used.

Also provided by the invention is a method for increasing the chances that an AD patient develops a favorable clinical response to a therapeutic administration of a treatment for AD, such as AN1792, by determining, prior to treatment, whether the patient has a unique gene expression pattern associated with the development of a favorable clinical response to the treatment.

Accordingly, the present invention provides a method for predicting whether a candidate AD patient is likely to develop a favorable clinical response, particularly a favorable immune response (e.g., an antibody response), to administration of a treatment for AD, particularly AN1792, comprising determining whether the candidate AD patient has a unique gene expression pattern associated with development of a favorable immune response, particularly the development of IgG antibodies, to the treatment. In some embodiments, the method further comprises referring to an AD patient population previously exposed to AN1792, wherein the patient population includes IgG responders and IgG nonresponders, and the unique gene expression is associated with a favorable immune response (e.g., IgG responders). In some embodiments, the presence of the unique gene expression pattern associated with a favorable immune response in the candidate AD patient predicts that the patient is likely to develop an IgG response to the administration of AN1792.

In some embodiments of the invention, the gene expression pattern of IgG responders is acquired from unstimulated patient samples and includes a moderate to high level of expression of at least one of the genes listed in Table 24 as having higher average expression in IgG responders (i.e., the odds ratio is greater than 1), and/or a low level of at least one of the genes listed in Table 24 as having lower average expression in IgG responders (i.e., the odds ration is less than 1). In other embodiments, the gene expression pattern of IgG responders is acquired from in vitro stimulated patient samples and includes a moderate to high level of expression of at least one of the genes listed in Table 18 as having higher average expression in IgG responders, and/or a low level of at least one of the genes listed in Table 18 as having lower average expression in IgG responders

Also provided by the invention is a method for reducing the risk that an AD patient develops meningoencephalitis, or another form of inflammation, or another adverse clinical response to the therapeutic administration of a treatment for AD, including but not limited to AN1792, by determining, prior to treatment, whether the patient has a unique gene expression pattern associated with the development of an adverse clinical response, e.g., an inflammatory response, including but not limited to the development of encephalitis (e.g., meningoencephalitis), to the treatment.

Accordingly, the present invention provides a method for predicting whether a candidate AD patient is likely to develop an adverse clinical response, e.g., an inflammatory response, particularly encephalitis, to administration of a treatment for AD, particularly AN1792, comprising determining whether the candidate AD patient has a unique gene expression pattern associated with development of an adverse clinical response, e.g., an inflammatory response, particularly encephalitis, to the treatment. In one embodiment, the method further comprises referring to an AD patient population previously exposed to AN1792, wherein the patient population includes inflammation developers and inflammation nondevelopers, and the unique gene expression pattern is associated with inflammation developers. In some embodiments, the presence of the unique gene expression pattern associated with inflammation developers in the candidate AD patient predicts that the patient is likely to develop inflammation in response to administration of AN1792.

In another embodiment, the gene expression pattern associated with an adverse clinical response is procured from an unstimulated sample and includes a moderate to high level of expression at least one of the genes listed in Tables 32-36 as having a higher average expression in encephalitis developers and/or a low level of expression of at least one of the genes listed in Tables 32-36 as having lower average expression in encephalitis developers (i.e., higher-odds ratio>1; lower-odds ratio<1). In other embodiments, the gene expression pattern associated with an adverse clinical response is procured from an in vitro stimulated sample and includes a moderate to high level of expression at least one of the genes listed in Tables 10 and 11 as having a higher or increased expression in meningoencephalitis (inflammation) developers and/or a low level of expression of at least one of the genes listed in Tables 10 and 12 as having lower expression in meningoencephalitis (inflammation) developers.

Another aspect of the invention relates to a method comprising the steps of providing at least one peripheral blood sample of an AD patient; and comparing a unique gene expression pattern of one or more genes in the at least one peripheral blood sample to at least one reference gene expression pattern of the one or more genes from an AD patient(s) treated with AN1792. Each of the genes is differentially expressed in peripheral blood mononuclear cells (PBMCs) of AD patients who, e.g., developed encephalitis, or did not develop an IgG response, or both, in response to AN1792 treatment as compared to AD patients who, e.g., did not develop encephalitis, or did develop an IgG response, or both, respectively, in response to AN1792 treatment. The method may be used to predict whether an AD patient is likely to develop an IgG response to AN1792, is likely not to develop an IgG response to AN1792, or is likely or not likely to develop inflammation in response to AN1792. In some embodiments, the step of providing at least one peripheral blood sample of an AD patient comprises the steps of collecting a blood sample form the patient; isolating blood cells from the sample; culturing the cells in the absence of AN1792; purifying total RNA fro the cells, thereby producing an RNA sample; and assaying RNA expression levels from the RNA sample to obtain a gene expression pattern. In other embodiments, assaying RNA expression levels from the RNA sample to obtain a gene expression pattern, wherein the expression levels comprise expression levels of one or more genes listed in, e.g., Tables 10-12 with a predictive strength ≧0.95, predicts that the AD patient is likely to develop inflammation. In another embodiment, assaying RNA expression levels from the RNA sample to obtain a gene expression pattern, wherein the expression levels comprise expression levels of one or more genes listed in, e.g., Table 18 with a predictive strength ≧0.95, predicts that the AD patient is likely not to develop an IgG response.

The invention is also directed to a method for using pharmacogenomics and/or other assays that measure gene expression levels to develop an improved, genomically guided AN1792 therapeutic product or therapy for treating AD having improved efficacy and/or safety profiles. The methods of the present invention are based on the utilization of gene expression patterns in a patient(s) with mild to moderate AD and the therapeutic response profiles to AN1792 in the patient(s).

Thus, the present invention provides methods for improving a response profile of a treatment for AD by increasing the chances that an AD patient develops a favorable and/or nonadverse clinical response to the treatment for AD, comprising the steps of determining that the AD patient, e.g., has a unique gene expression pattern associated with a favorable clinical response to the treatment for AD and/or does not have a unique gene expression pattern associated with an adverse clinical response, and administering the treatment for AD to the AD patient. The present invention also provides methods for improving a response profile of a treatment for AD by decreasing the chances that an AD patient develops an adverse clinical response to the treatment for AD, comprising determining that the AD patient has a unique gene expression pattern associated with an adverse clinical response to the treatment for AD, and not administering the treatment for AD to the AD patient.

The present invention also seeks to improve a response profile of a treatment for AD by regulating the expression levels of one or more genes of a patient sample procured from a candidate patient to be substantially similar to the expression levels of the same one or more genes that are involved in a unique gene expression pattern associated with a favorable clinical response (or associated with the lack of an adverse clinical response). In one embodiment of the invention, regulation of such expression levels is effected by the use of agents, e.g., inhibitory polynucleotides. Administration of such a therapeutic regulatory agent may regulate the aberrant expression of at least one gene that is part of a unique gene expression pattern, and therefore may be used to increase the chances for a favorable clinical response and/or decrease the risk of an adverse clinical response to a treatment for AD. Accordingly, the present invention also provides methods of improving the efficacy of a clinical trial of a treatment for AD, the methods generally comprising the steps of collecting blood samples from candidate patients; isolating blood cells from the samples; purifying total RNA from the cells, thereby producing an RNA sample; assaying RNA expression levels from the RNA samples to obtain gene expression patterns; and comparing the gene expression patterns of the candidate patients with the gene expression patterns of individuals who developed a particular clinical response to the treatment. In some embodiments, candidate patients with a substantially similar gene expression pattern to the gene expression pattern of individuals who developed a favorable clinical response to the treatment are included in the clinical trial of the treatment for AD. In other embodiments, candidate patients with a substantially similar gene expression pattern to the gene expression pattern of individuals who did not respond to the treatment are not included in the clinical trial of the treatment for AD. In another embodiment, candidate patients with a substantially similar gene expression pattern to the gene expression pattern of individuals who developed an adverse clinical response to the treatment are not included in the clinical trial of the treatment for AD; the method of this embodiment may also be used to improve the safety of a clinical trial of a treatment for AD.

Additionally, the present invention is directed to a method for treating AD comprising determining that an AD patient has a unique gene expression pattern previously determined to be associated with the development of a favorable clinical response, e.g., a favorable immune response, e.g., IgG antibodies, to a treatment for AD, including but not limited to AN1792, and administering the treatment for AD to the AD patient. The present invention is also directed to a method for treating AD comprising determining that an AD patient does not have a unique gene expression pattern previously determined to be associated with the development of an adverse clinical response, e.g., inflammation, to administration of, e.g., AN1792, and administering a treatment for AD to the AD patient. In one embodiment, the inflammation is encephalitis and the treatment is AN1792. In another embodiment, the invention provides a method for treating AD comprising determining that an AD patient does not have a unique gene expression pattern previously determined to be associated with the lack of a development of a favorable clinical response and administering the treatment, e.g., AN1792, to the AD patient. In another method of treating embodied in the invention, an AD patient who has a gene expression pattern associated with the lack of a development of a favorable clinical response, e.g., a gene expression pattern associated with a poor IgG response, is administered the treatment in combination with an agent that enhances a favorable clinical response.

The present invention is also directed to a new genomically guided AN1792 for treating AD that is developed using the methods of the present invention, and methods for developing such genomically guided AN1792. The genomically guided AN1792 includes AN1792 having an improved therapeutic response profile for an individual or a group of individuals belonging to a genomically defined population selected from a nongenomically defined population having AD, wherein the genomically defined population is preidentified as having or not having a particular gene expression pattern(s), and wherein the particular gene expression pattern(s) is associated with an improved response to AN1792. The compositions of the present invention are administered to at least one individual of the genomically defined population and are capable of treating AD in the genomically defined population more effectively or safely than treating a nongenomically defined population of individuals having AD. The genomically defined population would typically be identified as part of the indication by information printed on the label or packaging of the genomically guided therapeutic product or composition, e.g., genomically guided AN1792, but any means of communicating the relevant information is contemplated. A skilled artisan will recognize that a genomically guided version of another therapy for Alzheimer's disease (i.e., a therapy other than AN1792) can be developed by using the methods of the present invention, and is also contemplated as part of the present invention.

In some embodiments, a unique gene expression pattern of the invention comprises different expression levels in inflammation developers, as compared to inflammation nondevelopers, of one or more genes selected from the group consisting of TPR, NKTR, XTP2, SRPK2, THOC2, PSME3, DAB2, SCAP2, furin, and CD54. In other embodiments, the one or more genes are selected from the group consisting of ASRGL1, TPR, and SRPK2. In another embodiment, a unique gene expression pattern comprises high expression levels of at least one gene selected from the group consisting of FCGRT and granulin and/or low expression levels of at least one gene selected from the group consisting of IARS and MCM3.

### BRIEF DESCRIPTION OF THE DRAWINGS

FIG. 1 is a schematic diagram summarizing the design of the pharmacogenomics study of the present invention.

FIG. 2 shows the efficiency of removal of neutrophils by CPT fractionation.

FIG. 3 provides an overview of the samples generated and the samples selected for pharmacogenomic analysis.

FIG. 4 shows the gene expression frequency pattern for TPR.

FIG. 5 shows the gene expression frequency pattern for NKTR.

FIG. 6 shows the gene expression frequency pattern for XTP2.

FIG. 7 shows the gene expression frequency pattern for SRPK2.

FIG. 8 shows the gene expression frequency pattern for THOC2.

FIG. 9 shows the gene expression frequency pattern for PSME3.

FIG. 10 shows the gene expression frequency pattern for DAB2.

FIG. 11 shows the gene expression frequency pattern for SCAP2.

FIG. 12 shows the gene expression frequency pattern for furin.

FIG. 13 shows the gene expression frequency pattern for ICAM1 (CD54).

FIG. 14 shows the gene expression levels of IARS.

FIG. 15 shows the gene expression levels of FCGRT.

FIG. 16 shows the gene expression levels of granulin.

FIG. 17 shows the gene expression levels of MCM3.

FIG. 18 shows the disposition of patients from whom samples were analyzed in Example 2. The asterisk (*) represents that in 14 of 167 cases, pharmacogenomic data are not available for patients who consented to participate in the study. In 6 of these 14 cases, shipping time exceeded specifications. In the remaining 8 cases, yield of RNA or amplification product (IVT) was insufficient for chip hybridization

FIG. 19 shows the ratio of monocytes to lymphocytes for each of the 123 immunized patients.

FIG. 20 shows a classification by GeneCluster of the five encephalitis patients and a representative 30 (of 118) nonencephalitis patients using the optimal classifier set of 8 genes selected by GeneCluster.

FIG. 21 shows the gene expression frequencies of 123 immunized patients (X encephalitis developers and ●=nonencephalitis developers): a) frequencies of AKAP13 and NPukP68, the top ranked pairwise combination identified by logistic models for classification of encephalitis patients; and b) frequencies of STAT1 and TPR, the top ranked pairwise combination containing STAT1 (third ranked pairwise combination overall) identified by logistic models for classification of encephalitis patients. Solid lines indicate decision boundaries where encephalitis and nonencephalitis classes were equiprobable (i.e., log odds ratio=0) in the logistic models.

FIG. 22 shows the gene expression frequencies in 123 immunized patients (X=encephalitis developers and ●=nonencephalitis developers) for 18 other pairs of genes. The number of the graph indicates the pair's rank among pairwise combinations of genes identified by logistic models for classification of encephalitis patients (as shown in Table 37): (2) 213064_at (NPukP68) and 211962_{—}_at (ZFP36L1); (4) 212152_x_at (ARID1A) and 209969_s_at (STAT1); (5) 213064_at (NPukP68) and 221753_at (SSH1); (6) 211960_s_at (RAB7) and 209969_s_at (STAT1); (7) 213064_at (NPukP68) and 202469_s_at (CPSF6); (8) 213064_at (NPukP68) and 21010_x_at (HNRPH3); (9) 208657_s_at (MSF) and 209969_s_at (STAT1); (10) 213064_at (NPukP68) and 205281_s_at (PIGA); (11) 221753_at (SSH1) and 209969_s_at (STAT1); (12) 211960_s_at (RAB7) and 213064_at (NPukP68); (13) 202270_at (GBP1) and 215823_x_at (PABPC1); (14) 209969_s_at (STAT1) and 201394_s_at (RBM5); (15) 203159_at (GLS) and 209969_sat (STAT1); (16) 202256_at (CD2BP2) and 209969_s_at (STAT1); (17) 209484_s_at (DC8) and 202256_at (CD2BP2); (18) 214911_s_at (BRD2) and 209969_s_at (STAT1); (19) 205988_at (CD84) and 209969_s_at (STAT1); and (20) 200626_s_at (MATR3) and 213064_at (NPukP68). Solid lines indicate decision boundaries where encephalitis and nonencephalitis classes were equiprobable (i.e., log odds ratio=0) in the logistic models.

### DETAILED DESCRIPTION OF THE INVENTION

In order that the present invention may be more readily understood, certain terms are first defined. Additional definitions are set forth throughout the detailed description.

The term “adjuvant” refers to one or more biological immunomodulators that enhance antigen-specific immune responses.

The term “ApoE4” refers to apolipoprotein E, allele 4.

The term “cell saturation ratio” refers to the number of saturated features divided by the total number of features on the array.

The term “chip sensitivity” refers to the concentration level, in ppm, at which there is a 70% probability of obtaining a Present call, as calculated using Microarray Suite 5.0 (MAS 5.0; Affymetrix, Inc., Santa Clara, Calif.).

The term “cRNA” refers to complementary RNA.

The term “defect on visual inspection” refers to patterns in chip fluorescence visible after the chip has been run that reveal scratches, uneven staining, or other defects.

The term “EPIKS” refers to the Wyeth Expression Profiling Information and Knowledge System, an Oracle database (Oracle Corporation, Redwood Shores, Calif.) containing probe intensities and Absent/Present calls for each gene.

The term “final dataset” refers to the raw dataset which has been processed, and from which chips and genes not meeting various criteria have been filtered.

The term “FDR” refers to false discovery rate, an estimate of the percentage of genes that are false positive in a set of statistically significant genes.

The term “GEDS” refers to a graphical user interface that allows users to manually provide sample descriptions to EPIKS.

The term “GeneChip®” refers to an Affymetrix high-density array (Affymetrix, Inc., Santa Clara, Calif.) containing oligonucleotides of defined sequences that probe the cRNA derived from a target sample.

The term “GeneCluster” refers to an academic software application from the Whitehead Institute for Biomedical Research (Cambridge, Mass.) that chooses marker genes based on a signal-to-noise metric, and evaluates them by their ability to predict a given response parameter using a weighted voting algorithm.

The term “gene frequency” refers to a quantitative representation of the amount of gene present in a target sample, expressed as ppm.

The term “GLP” refers to Good Laboratory Practice.

The term “IVT” refers to in vitro transcription (used to generate the probe for hybridization to a gene chip).

The term “mitogen” refers to a compound with the property of inducing mitosis in culture.

The term “number of outliers across the array” refers to the capability of Affymetrix MAS 5.0 to detect outlier features. The MAS 5.0 manual indicates “outliers are probe cells that are obscured or nonuniform in intensity.” High numbers of outliers can indicate a poorly placed grid or a poorly aligned scanner. The MAS 5.0 software determines this number.

The term “PBMC” refers to peripheral blood mononuclear cells.

The term “PHA” refers to phytohemagglutinin, a T cell mitogen.

The term “ppm” refers to parts per million.

The term “probeset” refers to the oligonucleotides tiled on the gene chip representing a particular gene.

The term “QC” refers to quality control.

The term “QCP probability average difference” refers to the signal value for which there is a 70% probability of a Present call, as determined by the MAS 5.0 software.

The term “QCP probability frequency” refers to the QCP probability average difference expressed in ppm units.

The term “raw dataset” refers to the original gene expression and chip QC data, as stored on EPIKS.

The term “raw Q” refers to a measure of the noise level of the array. It is the degree of pixel-to-pixel variation among the probe cells used to calculate the background. Raw Q is an Affymetrix QC metric, which is determined by the MAS 5.0 software.

The term “scale factor” refers to the value required to obtain a trimmed mean intensity indicated by the target value. For all data in this study, the target value was set to a value of 100 and the scale factor was determined by dividing the trimmed mean of all probesets by the target value.

The term “U133A” refers to the commercial Affymetrix GeneChip® (Affymetrix, Inc., Santa Clara, Calif.) used in this study, which has been tiled with approximately 22,000 human probesets.

Generally, the present invention provides methods for predicting a clinical response of an AD patient to a treatment for AD to increase the chances for a favorable clinical response and/or reduce the risk of an adverse clinical response in an AD patient to a treatment for AD. The methods provided herein employ pharmacogenomic information to determine gene expression patterns associated with particular clinical responses. In one embodiment, the treatment is an immunotherapeutic, such as an active immunotherapeutic. The immunotherapeutic or immunotherapeutic agent is sometimes also termed an immunogen or immunogenic agent (see, e.g., WO 99/27944, to Schenk, incorporated by reference herein in its entirety). In another embodiment, the immunotherapeutic targets Aβ peptide. An example of such an immunotherapeutic is AN1792. In one embodiment of the invention, a favorable clinical response is the development of a protective immune response; in some embodiments, the protective immune response involves protective antibodies, e.g., IgG antibodies. In another embodiment, an adverse clinical response is the development of inflammation, e.g., encephalitis, e.g., meningoencephalitis. Methods for associating a gene expression pattern with a particular clinical response

Accordingly, the invention provides methods of compiling pharmacogenomic information to associate a unique gene expression pattern of a patient sample with a particular clinical response to a treatment for AD. Generally, the methods for compiling pharmacogenomic information to associate a unique gene expression pattern of a patient sample with a particular clinical response to a treatment for AD comprise the following steps: (1) procuring at least one patient sample from a patient of a first population of patients and at least one patient sample from a patient of a second population of patients, wherein the first population consists of one or more patients who developed the particular clinical response to the treatment for AD and wherein the second population consists of one or more patients who did not develop the particular response to the treatment for AD; (2) acquiring a gene expression pattern from each procured patient sample; and (3) determining whether at least most of the patient samples procured from the first population have a unique gene expression pattern not found in at least most of the patient samples procured from the second population, wherein a determination that at least most of the patient samples procured from the first population have a unique gene expression pattern not found in at least most of the patient samples procured from the second population results in associating the unique gene expression pattern with the particular clinical response to the treatment for AD. In one embodiment of the invention, the particular clinical response is one that is neither favorable nor adverse (e.g., antibody nonresponsiveness). In some embodiments, the particular clinical response is either a favorable clinical response or an adverse clinical response. In other embodiments, the particular clinical response is both a favorable and adverse clinical response.

For example, the invention also provides a method for compiling pharmacogenomic information to associate a unique gene expression pattern of a patient sample with a favorable clinical response to a treatment for AD comprising the following steps: (1) procuring at least one patient sample from a patient of a first population of patients and at least one patient sample from a patient of a second population of patients, wherein the first population consists of one or more patients who developed the favorable clinical response to the treatment for AD and wherein the second population consists of one or more patients who did not develop the favorable response to the treatment for AD; (2) acquiring a gene expression pattern from each procured patient sample; and (3) determining whether at least most of the patient samples procured from the first population have a unique gene expression pattern not found in at least most of the patient samples procured from the second population, wherein a determination that at least most of the patient samples procured from the first population have a unique gene expression pattern not found in at least most of the patient samples procured from the second population results in associating the unique gene expression pattern with the favorable clinical response to the treatment for AD.

In one embodiment of the invention, the second population consists of one or more patients who did not develop the favorable clinical response to the treatment and also developed an adverse clinical response. In another embodiment of the invention, the method further comprises excluding patients who also developed an adverse clinical response to the treatment for AD from the first population of patients.

The present invention also provides a method of compiling pharmacogenomic information to associate a unique gene expression pattern of a patient sample with an adverse clinical response to a treatment for AD comprising the following steps: (1) procuring at least one patient sample from a patient of a first population of patients and at least one patient sample from a patient of a second population of patients, wherein the first population consists of one or more patients who developed the adverse clinical response to the treatment for AD and wherein the second population consists of one or more patients who did not develop the adverse response to the treatment for AD; (2) acquiring a gene expression pattern from each procured patient sample; and (3) determining whether at least most of the patient samples procured from the first population have a unique gene expression pattern not found in at least most of the patient samples procured from the second population, wherein a determination that at least most of the patient samples procured from the first population have a unique gene expression pattern not found in at least most of the patient samples procured from the second population results in associating the unique gene expression pattern with the adverse clinical response to the treatment for AD. In one embodiment of the invention, the second population consists of one or more patients who did not develop the adverse clinical response to the treatment and also developed a favorable adverse clinical response. In another embodiment of the invention, the method further comprises excluding patients who also developed a favorable clinical response from the first population of patients.

Although the inventors were able to associate unique gene expression patterns to either favorable or adverse clinical responses to the AD treatment comprising administration of AN1792, a skilled artisan will recognize that the methods of compiling pharmacogenomic information provided herein may be used to associate unique gene expression profiles with either, neither, or both favorable or adverse clinical responses to any treatment for AD, e.g., including, but not limited to, immunotherapies, i.e., active or passive immunotherapies. In one embodiment, the treatment for AD comprises administration of AN1792.

A skilled artisan will recognize that a unique gene expression pattern may be defined as the pattern created by the differential, i.e., increased or decreased, expression level(s) of one or more genes in at least most patient samples from one population compared to expression level(s) of the same one or more genes in at least most patient samples from a second population. As used herein, an increased or decreased expression level relates to any statistically significant increase or decrease. Additionally, one of skill in the art will recognize that a unique gene expression pattern may consist of (1) the upregulation of one or more genes, (2) the downregulation of one or more genes, or (3) the upregulation of one or more genes and the downregulation of one or more other genes. Finally, a skilled artisan will recognize that a gene expression pattern may be considered unique when it can be used to differentiate the clinical response(s) of at least most of one patient population from the clinical response(s) of at least most of a second patient population, i.e., when it is associated with either a favorable or adverse clinical response, with both a favorable and adverse clinical response, or with neither favorable nor an adverse clinical response.

Methods of procuring a patient sample and what would constitute an appropriate patient sample are well known in the art. Additionally in the provided methods of compiling pharmacogenomic information, a patient sample may be taken before, during, or after the patient is treated with a treatment for AD, as long as the patient sample may be correlated with the final clinical response developed by the patient from which the sample was procured. In one embodiment of the invention, the patient sample is a PBMC fraction. In another embodiment, the patient sample is procured prior to the patient being treated with a treatment for AD. In another embodiment of the invention, the sample may be further processed, e.g., stimulated (e.g., placed under a certain in vitro culture condition), prior to the acquisition of its gene expression pattern, and the gene expression pattern of the sample cultured under a certain culture condition may be associated with either a favorable or adverse clinical response to a treatment for AD. For example, a sample may be placed under culture conditions that mimic the treatment for AD, e.g., incubated with an immunotherapeutic that is administered as a treatment for AD. A skilled artisan will be able to determine appropriate culture conditions, e.g., media, temperature, atmosphere, etc., for this type of analysis, and will know to include appropriate control conditions, e.g., the absence of the immunotherapeutic, the presence of a cell activator, etc.

To determine whether a gene expression pattern is unique, i.e., may be associated with a particular clinical response to a treatment for AD, a comparison must be made between gene expression patterns of samples procured from patients who developed a particular clinical response to a treatment for AD and gene expression patterns of samples procured from patients who did not develop the particular clinical response to the same treatment for AD. Consequently, patient samples must be procured from at least one patient of a first patient population consisting of one or more patients who developed the particular clinical response and from at least one patient of a second patient population consisting of one or more patients who did not develop the particular clinical response, such that a comparison of the gene expression patterns of the two populations may be made. Additionally, the patient populations must comprise patients who have been treated with the treatment for AD or will be treated with the treatment for AD (e.g., if the patient sample is taken before the treatment for begins) so that the patients will have a clinical response to the treatment. A skilled artisan will recognize that the association of a unique gene expression pattern with a favorable or adverse clinical response will be stronger if more AD patients are within the patient populations. Additionally, a skilled artisan will recognize that, in addition to patients who did not develop a favorable and/or adverse clinical response to the treatment for AD, samples may be procured from patients who developed a clinical response to a treatment for AD that is neither favorable nor adverse, AD patients who were given a placebo, and/or patients who do not have AD, e.g., healthy patients, etc. A skilled artisan will recognize that the phrase “AD patient” may also refer to candidates for AD therapy, e.g., individuals not presently diagnosed with AD, for example, patients only at risk of developing AD, or patients (e.g., elderly patients) presently in good health. Gene expression patterns from such patients may serve to corroborate the association of a unique gene expression pattern with a particular clinical response, as controls, etc. For example, where the favorable and adverse clinical responses are at opposite ends of the spectrum of one response, or where the clinical response may be graduated (e.g., an immune response) the gene expression pattern of a sample procured from an AD patient who developed a clinical response that is neither favorable nor adverse may prove to be one that is in between, or intermediate compared to, the expression levels(s) of the gene(s) involved in the a unique gene expression pattern associated with a favorable clinical response and the expression levels(s) of the gene(s) involved in a unique gene expression pattern associated with an adverse clinical response.

Since an object of the invention is to provide methods by which a unique gene expression pattern may be associated with either a favorable or an adverse clinical response, the clinical responses of each patient from whom a sample was procured should be monitored and recorded. A skilled artisan will recognize that, generally, a favorable clinical response to a treatment for AD may include the prevention, slowing down, arrest, and/or reversal of the development of AD, and may include the biological responses that promote the prevention, slowing down, arrest, and/or reversal of the development of AD (e.g., a protective immune response, e.g., an antibody response). A skilled artisan will also recognize that an adverse clinical response (1) is more than the natural progression of AD despite of the treatment for AD, (2) generally involves responses to the treatment for AD, and (3) is harmful to the patient. In other words, an adverse clinical response may be considered a harmful side effect of the treatment for AD and may include the biological responses that cause the side effects. For example, an adverse clinical response to a treatment for AD may be encephalitis, e.g., meningoencephalitis, and/or the inflammatory response that leads to encephalitis. Thus, in some instances, it may be that what constitutes a favorable clinical response only can be determined after the patient population has been treated and a favorable clinical response(s) is observed. Similarly, in some instances, it may be that what constitutes an adverse clinical response only can be determined after the patient population has been treated and an adverse clinical response(s) is observed. In this situation, it becomes clear why procurement of a patient sample prior to treating the patient with a treatment for AD is preferable. Thus, the methods provided herein may be used to associate a unique gene expression pattern with a favorable clinical response, e.g., a protective immune response, to a treatment for AD. In one embodiment, the favorable clinical response is an antibody response. In a more specific embodiment, the favorable clinical response is an IgG antibody response. The methods provided herein may also be used to associate a unique gene expression pattern with an adverse clinical response. In one embodiment, the adverse clinical response is inflammation, e.g., encephalitis, e.g., meningoencephalitis.

A skilled artisan will recognize the well-known methods for acquiring a gene expression pattern from a patient sample, e.g., methods of using preexisting gene expression patterns of a patient sample (e.g., those that may be stored in a database), and methods for detecting gene products (e.g., mRNA, proteins, etc.) such as, but not limited to, RT-PCR, in situ hybridization, slot-blotting, nuclease protection assays, Southern blot analysis, Northern blot analysis, microarray analysis, ELISA, RIA, FACS, dot blot analysis, Western blot analysis, immunohistochemistry, etc. In one embodiment of the invention, the patient sample is a PBMC fraction. In another embodiment, gene expression patterns are measured using RNA isolated from a patient sample. In another embodiment, a gene expression pattern is acquired by methods of microarray hybridization and microarray data analyses. In another embodiment, gene expression patterns are measured using protein isolated from a patient sample.

In the methods of compiling pharmacogenomic information that will determine an association between a unique gene expression pattern of a patient sample with a particular clinical response to a treatment for AD, all that is required for the association is that at least most of the patient samples procured from patients that developed a particular clinical response have a unique gene expression pattern that is not found in at least most of the patient samples procured from patients who did not develop the particular response. At least most encompasses at least 51%. In one embodiment, at least most means at least 75%. In another embodiment, at least most means at least 80%. Additionally, a skilled artisan will recognize that cross-validation studies of the association between a gene expression and a clinical response will serve to corroborate the association.

A skilled artisan will recognize that the step of excluding patients from a first population of patients may encompass, but is not limited to, the following: 1) excluding patient samples procured from patients prior to the step of acquiring a gene expression pattern from each procured patient sample, and/or 2) excluding from the unique gene expression pattern genes that are part of a gene expression pattern associated with another clinical response. For example, as described below, treatment with AN1792 led to some patients developing only the favorable IgG response and some patients developing both the favorable IgG response and encephalitis. Thus, a unique gene expression pattern may be associated with a favorable clinical response by excluding patient samples, procured from patients who also developed an adverse clinical response, prior to acquiring a gene expression pattern from each procured sample, and/or by excluding from the unique gene expression pattern to be associated with the favorable clinical response one or more genes that may also be associated with an adverse clinical response. Similarly, a unique gene expression pattern may be associated with an adverse clinical response by excluding patient samples, procured from patients who also developed a favorable clinical response, prior to acquiring a gene expression pattern from each procured sample, and/or excluding from the gene expression pattern genes to be associated with the adverse clinical response one or more genes that may also be associated with a favorable clinical response.

As noted above, AN1792 is considered a promising treatment for AD. However, although a subset of patients developed a favorable clinical response to AN1792 that correlated with a protective immune response, e.g., the development of antibodies, a smaller subset of AD patients developed an adverse clinical response, e.g., inflammation leading to encephalitis, and the immunotherapeutic dosing was discontinued. The information obtained during the clinical trials and the availability of samples from patients who participated in the study has allowed for the pharmacogenomic studies disclosed herein. In other words, the methods of compiling pharmacogenomic information as provided herein were used to associate at least one gene expression pattern of a sample procured from an AD patient treated with AN1792 with a favorable or adverse clinical response to AN1792.

In one embodiment, blood samples were taken from participants in the AN1792 phase II clinical trial (see Examples 1 and 2). For each sample, the peripheral blood mononuclear cell (PBMC) fraction was purified by CPT (cell preparation tube) fractionation. However, the PBMCs may be purified by flotation or density barrier, or any other means known in the art. After the PBMCs have been purified from the total cell population, which increases the percentage of neutrophils in the remaining cell population, some of the PBMCs were cultured, e.g., with AN1792 (see Example 1). However a skilled artisan will recognize that samples may be cultured by any means known in the art, and also that gene expression patterns may be acquired from unstimulated samples (see, e.g., Example 2). After culture, the nonadherent cultured cells were harvested and removed from the culture media by centrifugation and the RNA was purified by conventional means, specifically by QIAshredders and Qiagen RNeasy mini-kits (Qiagen Inc., Valencia, Calif.); the same purification steps were used for unstimulated cells. Any method known in the art for purifying RNA may be used. The purified RNA was then amplified by in vitro translation amplification with biotinylated nucleotides, to make biotinylated cRNA. The biotinylated cRNA was then hybridized to known sequences to determine which sequences are present or absent in the RNA sample. For example, the amplified, biotinylated cRNA was hybridized to the Affymetrix human U133A oligonucleotide GeneChip, which interrogates the RNA levels of over 22,000 sequences. The GeneChip was then washed to remove unhybridized cRNA, stained with streptavidin, and scanned to produce array images that were processed with the Affymetrix MicroArray Suite (MAS 5.0) software and was further processed to create probeset summary values. Probe intensities were summarized for each message using the Affymetrix Signal algorithm and the Affymetrix Absolute Detection metric (Absent, Present, or Marginal) for each probeset. Normalization, filtering, and identification and reporting of outlier samples were then performed. The data was then statistically analyzed using, e.g., analysis of variance (ANOVA) and signal-to-noise metrics to determine a unique gene expression patterns of cultured or unstimulated patient samples associated with encephalitis, IgG responsiveness, and/or IgG nonresponsiveness, as noted in Example 1. Other well-known combinations of computer programs, databases, and/or statistical algorithms, including, but not limited to, Affymetrix programs (e.g., MAS 5.0, SAS, etc.), the EPIKS database, determination of Pearson correlation coefficients (r²), analysis of covariance (ANCOVA), analysis of variance (ANOVA), Benjamini and Hochberg's False Discovery Rate (FDR) procedure, logistic regression, Ingenuity pathways analysis, GeneCluster analysis, etc., may be used to associate gene expression patterns with particular clinical outcomes (see also, e.g., Example 2). The skilled artisan will recognize that other means may be used to analyze the data from the hybridizations and acquire a gene expression profile from a procured sample.

Accordingly, the invention also provides methods of compiling pharmacogenomic information to associate a unique gene expression pattern of a patient sample taken from a patient treated with AN1792 with a clinical response to the administration of AN1792. In one embodiment of the invention, gene expression patterns are acquired from unstimulated samples. In another embodiment, samples are placed under a certain culture condition prior to acquisition of gene expression patterns. In one embodiment, the favorable clinical response is a protective immune response. In another embodiment, the favorable clinical response is an antibody response, e.g., an IgG response. In another embodiment, the adverse clinical response is an inflammatory response. In one embodiment, the inflammatory response leads to encephalitis, e.g., meningoencephalitis. A skilled artisan will recognize that the term “inflammation,” or “inflammatory response” refers to an innate immune response that results in an adverse clinical response when used regarding or in the context of discussing encephalitis (or other adverse inflammatory side effects, e.g., vasculitis, cellulitis, nephritis, etc.) and/or results in absence of a favorable response. A skilled artisan also will recognize that, as described above, a favorable or adverse clinical response to AN1792 may be chosen from a variety of responses, including but not limited to the prevention, slowing down, arrest and/or reversal of the development of AD (e.g., a protective immune response) or an adverse drug response (e.g., an inflammatory response).

Applying the methods of compiling pharmacogenomic information as provided herein to at least one patient of a first patient population consisting of one or more patients who developed a particular clinical response and at least one patient of a second patient population consisting of one ore more patients who did not develop the particular clinical response to AN1792, several unique gene expression patterns were obtained that may be associated with a particular clinical response to AN1792, e.g., IgG responders, IgG partial responders, IgG nonresponders, encephalitis developers, and/or encephalitis nondevelopers.

In practicing the methods of compiling pharmacogenomic information, the inventors were able to associate gene expression patterns of cultured patient samples, e.g., patient samples incubated with AN1792, with a particular response (e.g., encephalitis developers, IgG nonresponders) to AN1792. The genes of expression patterns of stimulated samples that may be associated with either a favorable or adverse clinical response to AN1792 are listed in Tables 10-12 and 18. Additionally, the inventors were able to associate unique gene expression patterns of unstimulated samples with a particular clinical response to AN1792 (e.g., IgG responders and/or encephalitis developers). The gene expression patterns of unstimulated samples that may be associated with either a favorable or adverse clinical response to AN1792 are listed in Tables 24-37.

The genes listed in Table 10 (and discussed in Example 1) are associated with the development of encephalitis and are either upregulated or downregulated in cultured patient samples procured from encephalitis developers, i.e., encephalitis developers may have increased or decreased levels of these genes as compared to encephalitis nondevelopers.

In one embodiment, increased gene expression levels of one or more of the genes listed in Table 11 (and discussed in Example 1) in a cultured patient sample are associated with the development of encephalitis.

In another embodiment, decreased gene expression levels of one or more of the genes listed in Table 12 (and discussed in Example 1) in a cultured patient sample are associated with the development of encephalitis.

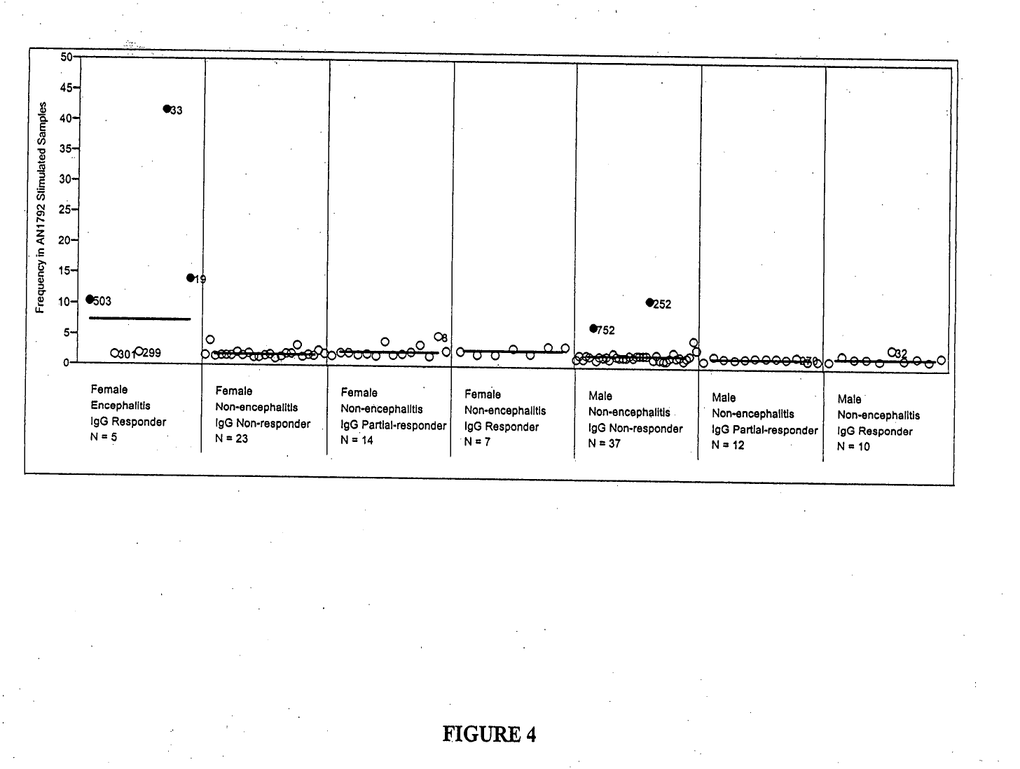

In another embodiment of the invention, the differential expression levels in encephalitis developers as compared to encephalitis nondevelopers for at least one or more of the following genes in a cultured patient sample is associated with the development of encephalitis, as further illustrated in FIGS. 4-13: TPR; NKTR; XTP2; SRPK2; THOC2; PSME3; DAB2; SCAP2; furin; and ICAM1 (CD54). In another embodiment of the invention, the difference in expression levels in encephalitis developers as compared to encephalitis nondevelopers for at least one or more of the following genes in a cultured patient sample is associated with the development of encephalitis: TPR; NKTR; SRPK2; DAB2; SCAP2; and furin (PACE).

In another embodiment, the differential expression levels of one or more genes in cultured patient samples are associated with neither a favorable or adverse clinical response, i.e., these genes are upregulated or downregulated in cultured patient samples procured from AD patients who did not develop an IgG antibody response, i.e., IgG nonresponders, compared to those in cultured patient samples procured from AD patients who did develop an IgG response. Preferably, the gene expression pattern of IgG nonresponders includes a moderate to high level of expression of at least one of the genes listed in Table 18 in cultured patient samples as having “higher” average expression in IgG nonresponders, and/or a low level of at least one of the genes listed in Table 18 as having “lower” average expression in IgG nonresponders. As used herein, moderate to high levels of expression means any statistically significant increase in expression in IgG nonresponders as compared to IgG responders, and low levels means any statistically significant decrease in expression in IgG nonresponders as compared to IgG responders. More preferably, the gene expression pattern of IgG nonresponders includes a moderate to high level of expression of at least one of the genes selected from the group consisting of granulin and FCGRT, and/or a low level of expression of at least one of the genes selected from the group consisting of IARS and MCM3.

The genes listed in Table 24 (and discussed in Example 2.3.2) are associated with the development of a favorable clinical response, i.e., a protective immune response, particularly an IgG antibody response, and have an odds ratio for IgG association (as calculated with meningoencephalitics) of at least three-fold between IgG responders and others, and are either upregulated (e.g., have an odds ratio ≧3) or downregulated (e.g., have an odds ratio ≦0.33) in unstimulated patient samples procured from AD patients who developed an IgG antibody response to administration of AN1792 (i.e., IgG responders), as compared to unstimulated patient samples procured from AD patients who did not develop an IgG antibody response (IgG nonresponders) or patient samples procured from AD patients who developed an IgG antibody response but also developed an adverse clinical response, particularly inflammation leading to encephalitis (i.e., IgG responder and meningoencephalitic). In other words, IgG responders may have increased or decreased expression levels of these genes compared to IgG nonresponders and/or IgG responders and meningoencephalitics.

In one embodiment, increased gene expression levels of one or more of the genes listed in Tables 25-27 having a three-fold increase in odds ratios (e.g., genes listed in Tables 25-27 as having an odds ratio ≧3) in an unstimulated patient sample are associated with the development of a protective IgG response (see Example 2.3.3). In another embodiment, decreased gene expression levels of one or more of the genes listed in Tables 25-27 having a three-fold decrease in odds ratio (e.g., genes listed in Tables 25-27 as having an odds ratio≦0.33) are associated with the development of a favorable protective IgG response (see Example 2.3.3).

In another embodiment of the invention, the differential expression levels in patients who developed an IgG antibody response to AN1792 as compared to patients who did not develop an IgG antibody response or who did develop an IgG antibody response but also developed an adverse clinical response, e.g., inflammation leading to encephalitis, for at least one of the genes listed in Tables 28 and 30 in an unstimulated patient sample is associated with the development of a favorable IgG immune response. In other words, the upregulation of expression of one or more genes listed in Tables 28-31 listed as having an odds ratio ≧3) and/or the downregulation of expression of one or more genes in Tables 28 and 30 listed as having an odds ratio ≦0.33) in an unstimulated patient sample may be associated with a favorable IgG immune response.

The genes listed in Table 32 (and discussed in Example 2.3.5) are associated with the development of encephalitis and are either upregulated (i.e., have an odds ratio for association with encephalitis ≧3) or downregulated (i.e., have an odds ratio for association with encephalitis ≦0.33) in unstimulated patient samples procured from encephalitis developers.

In one embodiment, increased gene expression levels of one or more of the genes listed in Table 34 (including the subset of genes listed in Table 35), e.g., genes listed in Table 34 or 35 as having an odds ratio for association with encephalitis ≧3, in an unstimulated patient sample are associated with the development of encephalitis (see Example 2.3.6). In another embodiment, decreased gene expression levels of one or more of the genes listed in Table 34 (including the subset of genes listed in Table 35), e.g., genes listed in Table 34 or 35 as having an odds ratio for association with encephalitis ≦0.33, are associated with the development of encephalitis (see Example 2.3.6).

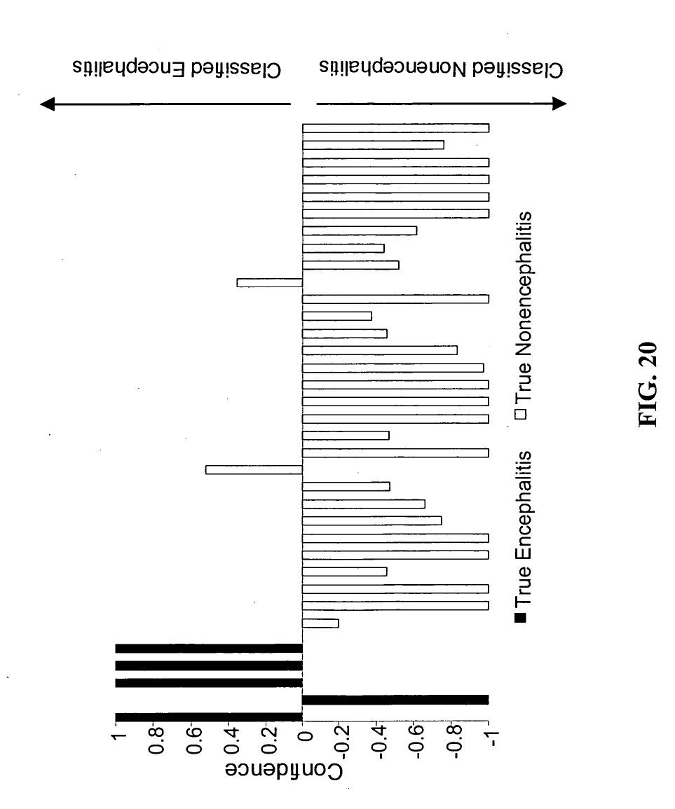

In another embodiment of the invention, the differential expression levels in encephalitis developers as compared to encephalitis nondevelopers for at least one or more of the genes listed in Table 36 in an unstimulated patient sample is associated with the development of encephalitis (see also FIG. 20). In other words, an upregulated expression of one or more genes listed in Table 36 as having an odds ratio for encephalitis ≧3, and/or a downregulated expression of one or more genes listed in Table 36 as having an odds ratio for encephalitis ≦0.33, in a patient sample may be associated with the development of encephalitis.

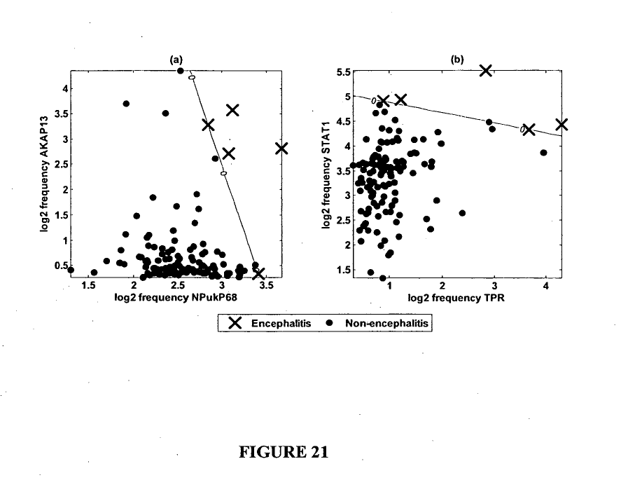

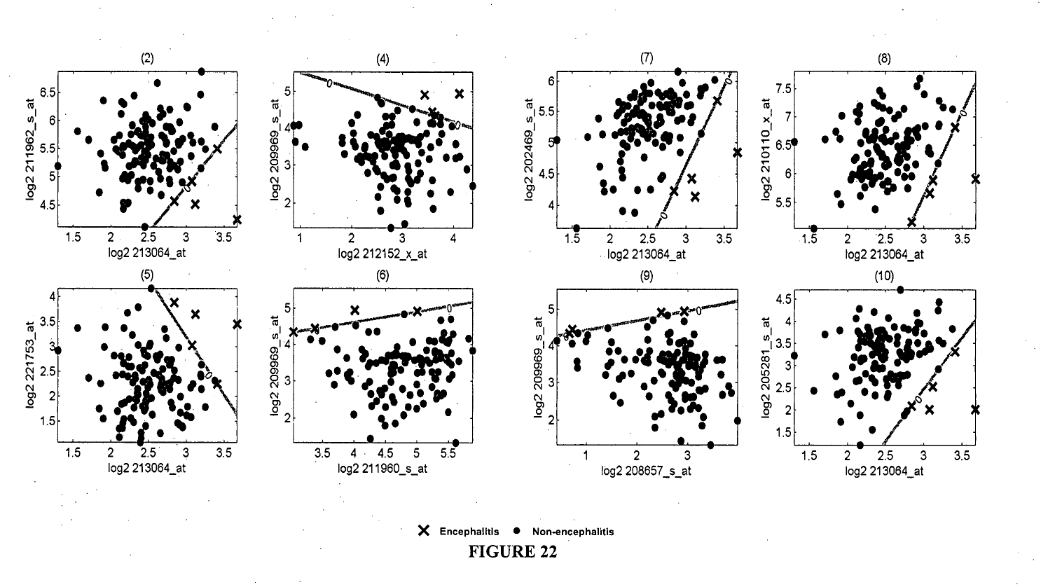

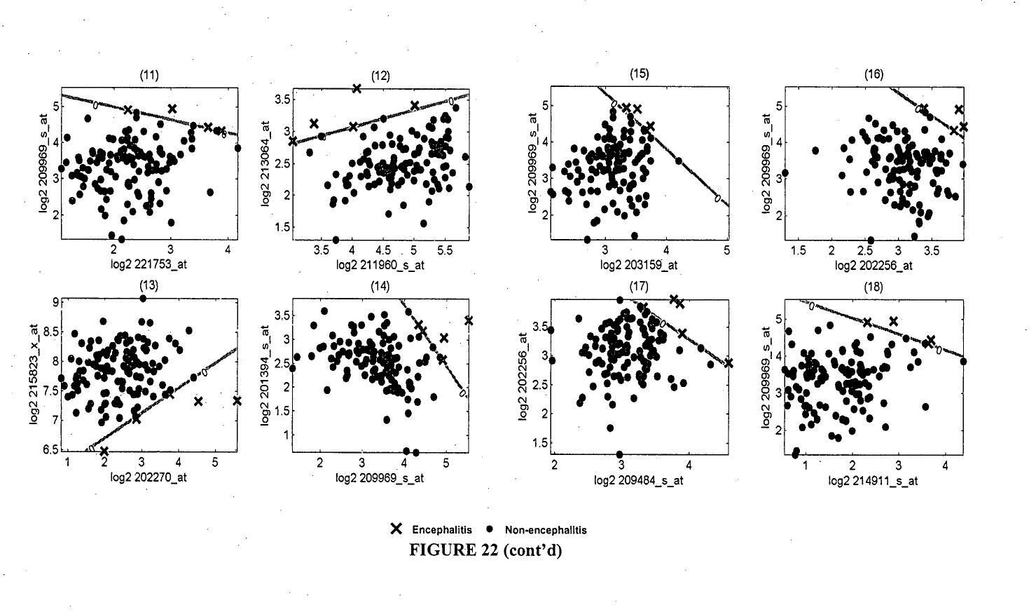

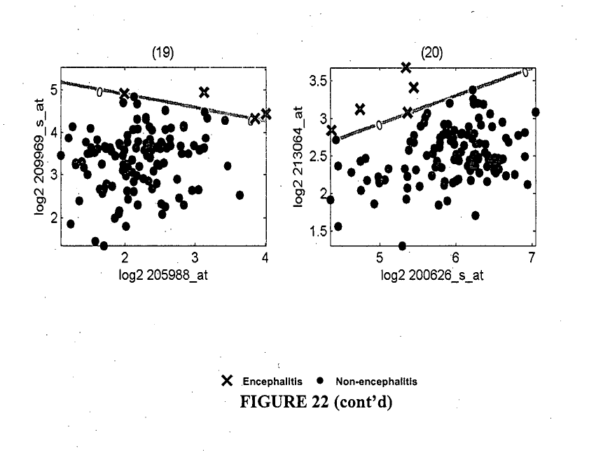

In another embodiment of the invention, the differential expression level of one or more pairs of genes, e.g., those pairs listed in Table 37, in a patient sample distinguishes encephalitis developers from encephalitis nondevelopers (see Example 2.3.7). As depicted in FIGS. 21 and 22, whether the differential expression levels of one or more pairs of genes is associated with encephalitis development or encephalitis nondevelopment in a patient is dependent on where the expression levels of the two genes within a pair of genes (e.g., as noted on the X and Y axes of the graphs in FIGS. 21 and 22) are in relation to the decision boundary (e.g., the solid line in a graph in FIG. 21 or FIG. 22) for that pair.

Polynucleotides of the Invention

Polynucleotides encoding the genes involved with unique gene expression patterns of the present invention may be used as hybridization probes and primers to identify and isolate nucleic acids having sequences identical to or similar to the disclosed genes. Hybridization methods for identifying and isolating nucleic acids include polymerase chain reaction (PCR), Southern hybridizations, in situ hybridization and Northern hybridization, and are well known to those skilled in the art.

Hybridization reactions can be performed under conditions of different stringency. The stringency of a hybridization reaction includes the difficulty with which any two nucleic acid molecules will hybridize to one another. Preferably, each hybridizing polynucleotide hybridizes to its corresponding polynucleotide under reduced stringency conditions, more preferably stringent conditions, and most preferably highly stringent conditions. Examples of stringency conditions are shown in Table 1 below: highly stringent conditions are those that are at least as stringent as, for example, conditions A-F; stringent conditions are at least as stringent as, for example, conditions G-L; and reduced stringency conditions are at least as stringent as, for example, conditions M-R.

Polynucleotides associated with genes of the present invention may be used as hybridization probes and primers to identify and isolate DNA having sequences encoding allelic variants of the disclosed genes. Allelic variants are naturally occurring alternative forms of polynucleotides that encode polypeptides that are identical to or have significant similarity to the polypeptides encoded by the polynucleotides associated with the disclosed genes. Preferably, allelic variants have at least 90% sequence identity (more preferably, at least 95% identity; most preferably, at least 99% identity) with the polynucleotides associated with the disclosed genes.

Polynucleotides associated with the disclosed genes of the present invention may also be used as hybridization probes and primers to identify and isolate DNAs having sequences encoding polypeptides homologous to the disclosed genes. These homologs are polynucleotides and polypeptides isolated from a different species than that of the polypeptides and polynucleotides associated with the disclosed genes, or within the same species, but with significant sequence similarity to the polynucleotides and polypeptides associated with the disclosed genes. Preferably, polynucleotide homologs have at least 50% sequence identity (more preferably, at least 75% identity; most preferably, at least 90% identity) with the polynucleotides associated with the disclosed genes, whereas polypeptide homologs have at least 30% sequence identity (more preferably, at least 45% identity; most preferably, at least 60% identity) with the polypeptides associated with the disclosed genes. Preferably, homologs of the polynucleotides and polypeptides associated with the disclosed genes are those isolated from mammalian species. Polynucleotides associated with the disclosed genes of the present invention may also be used as hybridization probes and primers to identify cells and tissues that express polypeptides associated with the disclosed genes of the present invention and the conditions under which they are expressed.

Panels and Kits

A unique gene expression pattern may comprise the expression level of one gene that may be considered individually, although it is within the scope of the invention that a unique gene expression pattern may comprise the expression levels of two or more genes to increase the confidence of the analysis. In one embodiment, the invention provides a unique gene expression pattern that comprises a panel of genes. A panel may comprise 2-5, 5-15, 15-35, 35-50, 50-100, or more than genes. In one embodiment, a panel may comprise 15-20 genes.

In another embodiment, panels of genes are selected such that the genes within any one panel share certain features. As a nonlimiting example, the genes of a first panel may each have high expression levels in a unique gene expression pattern associated with a particular clinical response. Alternatively, genes of a second panel may each exhibit differential expression as compared to a first panel. Similarly, different panels of genes may be composed of genes that are from different functional categories (i.e., proteolysis, signal transduction, transcription, etc.), or may be selected to represent different stages of, e.g., an immune response. Panels of genes may be made by selecting genes involved in a unique gene expression pattern associated with a particular clinical response. As a nonlimiting example, a panel may comprise genes selected from, e.g., Table 24. Panels may also be made by combining genes selected from those listed in Table 10-12, 18, and 24-37. In one embodiment, a panel comprises genes listed in Table 36. In another embodiment, a panel comprises a pair of genes, e.g., any of the pairs of genes listed in Table 37.

In addition to providing unique gene expression patterns that may comprise one gene or a panel of genes, it is within the scope of the invention to provide kits for detecting one or a panel of genes involved in a unique gene expression pattern of the invention. These kits may comprise one or more polynucleotides, each capable of hybridizing under stringent conditions to an RNA transcript, or the complement thereof, of a gene differentially expressed in a unique gene expression pattern of the invention; and/or one or more antibodies, each capable of binding to a polynucleotide encoded by a gene differentially expressed in a unique gene expression of the invention.

Additionally, the kits of the invention may comprise one or more polynucleotides and/or one or more antibodies for the detection of one or more genes involved in a gene expression pattern of the invention, wherein the one or more polynucleotides and/or antibodies are conveniently coupled to a solid support. For example, polynucleotides of genes involved in a unique gene expression pattern of the invention may be coupled to an array (e.g., a biochip for hybridization analysis), to a resin (e.g., a resin that can be packed into a column for column chromatography), or a matrix (e.g., a nitrocellulose matrix for Northern blot analysis). By providing such support, discrete analysis of the expression level(s) of each gene selected for the panel may be detected. For example, in an array, polynucleotides complementary to each gene of a unique gene expression pattern comprising a panel of gene may be individually attached to different known locations on the array. The array may be hybridized with, for example, polynucleotides extracted from a sample (e.g., a blood sample) from a subject. The hybridization of polynucleotides from the sample with the array at any location on the array can be detected, and thus the expression level of the gene in the sample can be ascertained. Thus, not only tissue specificity, but also the level of expression of a panel of genes in the tissue is ascertainable. In one embodiment, an array based on a biochip is employed. Similarly, ELISA analyses may be performed on immobilized antibodies specific for different polypeptide biomarkers hybridized to a protein sample from a subject. Methods of making and using such arrays, including the use of such arrays with computer readable media comprising genes of the invention and/or databases, e.g., a relational database, are well known in the art.

In another embodiment, a reporter nucleic acid is utilized to detect the expression of one or more genes involved in a unique gene expression pattern. Such a reporter nucleic acid can be useful for high-throughput screens for agents that alter the expression profiles of peripheral blood mononuclear cells. The construction and use of such reporter assays are well known.

For example, the construction of a reporter for transcriptional regulation of a gene involved in a unique gene expression pattern of the invention generally requires a regulatory sequence of the gene, typically the promoter. The promoter can be obtained by a variety of routine methods. For example, a genomic library can be hybridized with a labeled probe consisting of the coding region of the nucleic acid to identify genomic library clones containing promoter sequences. The isolated clones can be sequenced to identify sequences upstream from the coding region. Another method is an amplification reaction using a primer that anneals to the 5′ end of the coding region of a polynucleotide for the gene. The amplification template can be, for example, restricted genomic nucleic acid to which anchor bubble adaptors have been ligated.

To construct the reporter, the promoter of the selected gene may be operably linked to the reporter nucleic acid, e.g., without utilizing the reading frame of the polynucleotide sequence of the selected gene. The nucleic acid construct is transformed into tissue culture cells, e.g., peripheral blood mononuclear cells, by a transfection protocol to generate reporter cells.

Many of the well-known reporter nucleic acids may be used. In one embodiment, the reporter nucleic acid is green fluorescent protein. In a second embodiment, the reporter is β-galactosidase. In other embodiments, the reporter nucleic acid is alkaline phosphatase, β-lactamase, luciferase, or chloramphenicol acetyltransferase. The reporter nucleic acid construct may be maintained on an episome or inserted into a chromosome by, for example, using targeted homologous recombination. Methods of making and using such reporter nucleic acids and others are well known.

Methods of Using a Gene Expression Pattern Associated with a Particular Clinical Response

Once at least one unique gene expression pattern of a patient sample is associated with a particular clinical response to a treatment for AD, the at least one unique gene expression pattern may be used to predict whether a patient will develop the particular clinical response to the treatment for AD, even if the AD patient had not been previously exposed to the treatment for AD. Thus the invention also provides methods of predicting whether a candidate patient who has not been previously exposed to a treatment for AD will develop a particular clinical response to a treatment for AD, the methods generally comprising (1) associating at least one unique gene expression pattern of a patient sample with a particular clinical response to the treatment for AD by methods of compiling pharmacogenomic information (2) procuring a test sample from the candidate patient who has not been previously exposed to the treatment for AD, and (3) determining whether the test sample procured from the candidate patient who has not been previously exposed to the treatment for AD has a test gene expression pattern that is substantially similar to the at least one gene expression pattern associated with the particular clinical response to the treatment for AD, wherein if it is determined that the test sample has a test gene expression pattern that is substantially similar to the at least one gene expression pattern associated with the particular clinical response, it may be predicted that the candidate patient will develop the particular clinical response. In one embodiment, the particular clinical response is neither favorable nor adverse. In one embodiment, the particular clinical response is either a favorable or adverse clinical response. In another embodiment, the particular clinical response is both a favorable and adverse clinical response.

In some embodiments, a database of unique gene expression patterns that are each associated with a particular clinical response to a treatment for AD will have been previously established. In such a case, the methods of predicting a clinical response of a candidate patient comprises the steps procuring a test sample from the candidate patient not previously exposed to the treatment for AD, and determining whether the test sample from the candidate patient not previously exposed to the treatment for AD has a test gene expression pattern that is substantially similar to a reference gene expression pattern that has been previously associated with a particular clinical response, wherein if it is determined that the test sample has a test gene expression pattern that is substantially similar to the reference gene expression pattern that has been previously associated with the particular clinical response, it may be predicted that the candidate patient will develop the particular clinical response. A skilled artisan will recognize that a particular clinical response may be a favorable clinical response, e.g., a protective immune response, an adverse clinical response, e.g., an inflammatory response, a clinical response that is neither favorable nor adverse, e.g., nonresponsiveness, or any combination of the three.

A skilled artisan will recognize that in the above-described methods of predicting the clinical response of a candidate AD patient, the test sample should be procured from the candidate AD patient in the same manner, or as close as possible to the same manner, as the procurement of the reference sample (i.e., the sample of which the gene expression pattern is associated a particular clinical response) from the reference AD patient. Additionally, a skilled artisan will recognize that in determining whether the test sample has a test gene expression pattern that is substantially similar to a reference gene expression pattern, i.e., a gene expression pattern that has been previously associated with a particular clinical response to the treatment for AD, a test gene expression pattern must be acquired from the test sample. Also, the test gene expression pattern should be acquired in a similar manner as the gene expression pattern that has been previously associated with a particular clinical response. Such methods of procuring a sample (or test sample) and acquiring a gene expression pattern (or test gene expression pattern) are well known in the art, as described above.

As a nonlimiting example, if the gene expression pattern associated with a particular clinical response was acquired via microarray analysis of a PBMC sample procured from an patient treated with a treatment for AD prior to the patient being exposed to the treatment for AD, the test gene expression pattern would also be acquired via microarray analysis of a PBMC sample procured from a candidate patient prior to the candidate patient being exposed to the treatment for AD. As another nonlimiting example, if the gene expression pattern previously associated with a particular clinical response was acquired from a patient sample that was placed under certain culture conditions after its procurement, the test gene expression pattern would be acquired from a test sample placed under similar culture conditions after its procurement. In other words, the timing of procuring a sample and a test sample in relation to exposure to a treatment for AD, the conditions in which the sample and the test sample are processed (e.g., unstimulated, cultured, etc.), the methods used to acquire the gene expression pattern previously associated with a particular clinical response and the test gene expression pattern, and the treatment administered to the AD patient treated with the treatment and the treatment for which candidate AD patient is a candidate, ideally would be similar or as similar as possible.

Since part of the invention associates unique gene expression patterns with particular clinical responses to AN1792 by AD patients to treatment with AN1792, the clinical response of a candidate patient to treatment with AN1792 may be predicted using the gene expression patterns described in Tables 10-12, 18, and 24-37. Therefore the present invention relates to a method of predicting whether a candidate patient will develop a particular clinical response when administered AN1792 by (1) compiling pharmacogenomic information to associate at least one unique gene expression pattern of a preimmunization patient sample procured from a patient who has been treated with AN1792 with a particular clinical response, (2) procuring a test sample from the candidate patient, and (3) determining whether the test sample has a test gene expression pattern that is substantially similar to the at least one unique gene expression pattern, wherein if the test sample has a test gene expression pattern that is substantially similar to the at least one gene expression pattern associated with the particular clinical response, it may be predicted that the candidate patient will develop the particular clinical response. In one embodiment, the particular clinical response is neither favorable nor adverse, e.g., nonresponsiveness. In another embodiment, the particular clinical response to AN1792 is a favorable clinical response, e.g., a protective immune response, e.g., an IgG antibody response. In another embodiment, the particular clinical response to AN1792 is an adverse clinical response, e.g., an inflammatory response, e.g., encephalitis.

For example, the invention is therefore further directed to a method for predicting whether a candidate AD patient will have an IgG response. Preferably, an AD patient treated with a treatment for AD, such as an immunotherapeutic, e.g., AN1792, will have a moderate to high level of IgG expression and will not develop an inflammatory response, such as encephalitis. As noted above, AN1792 is an immunotherapeutic for patients with AD. It presumably works by stimulating the immune system to “recognize” and attack the β-amyloid plaques in patients with AD, and does so by causing the production of antibodies against the β-amyloid protein. Therefore, a good IgG response after administration of AN1792 is desired. Accordingly, the present invention provides a method for predicting whether a candidate AD patient is likely to mount a moderate to high IgG response, either by determining that a test sample procured from the candidate AD patient does not express a unique gene expression pattern associated with nonresponsiveness or determining that a test sample procured from the candidate AD patient has another unique gene expression pattern associated with IgG responsiveness. Generally, the method comprises (1) obtaining a patient population previously exposed to AN1792, wherein the patient population includes IgG responders and IgG nonresponders and wherein IgG expression is associated with administration of AN1792, (2) determining whether there is a unique gene expression pattern associated with patient samples procured from IgG nonresponders that is not found in patient samples procured from IgG responders, and (3) determining whether a test patient sample procured from the candidate patient does not have the unique gene expression pattern associated with IgG nonresponders, wherein if the test sample does not have a test gene expression pattern that is substantially similar to the at least one gene expression pattern associated with IgG nonresponders, it may be predicted that the candidate patient will not be an IgG nonresponder, i.e., will be an IgG responder. More specifically, the method comprises (1) collecting blood from a patient population previously exposed to AN1792, wherein the patient population includes patients who mount a moderate to high IgG response to AN1792 and patients who mount a low or undetectable IgG response, i.e., IgG responders and IgG nonresponders, respectively, (2) purifying, e.g., total RNA from the blood sample, (3) assaying RNA expression levels to obtain gene expression patterns for the IgG responders and IgG nonresponders, (4) comparing the gene expression patterns of the IgG responders and IgG nonresponders to obtain a unique gene expression pattern for IgG nonresponders, and (5) determining whether a candidate patient not previously exposed to AN1792 has the unique gene expression pattern for IgG nonresponders, wherein the presence of the unique gene expression pattern in the candidate patient predicts a likelihood that the candidate patient will not mount an IgG response. If the candidate patient does not have the unique gene expression pattern associated with a poor IgG response, it is possible that the patient is a good candidate for treatment with AN1792. Similarly to the disclosure involving predicting whether a candidate patient will be an encephalitis developer or nondeveloper, IgG responders and nonresponders can also be predicted by assaying protein expression levels to obtain gene expression patterns. One of ordinary skill in the art will appreciate that the general disclosure related to treatment with AN1792 may also be used for treatments for Alzheimer's disease other than AN1792.

Preferably, the gene expression pattern of IgG nonresponders includes a moderate to high level of expression of at least one of the genes listed in Table 18 in cultured cells as having “higher” average expression in IgG nonresponders, and/or a low level of at least one of the genes listed in Table 18 as having “lower” average expression in IgG nonresponders. As used herein, moderate to high levels of expression means any statistically significant increase in expression in IgG nonresponders as compared to IgG responders, and low levels means any statistically significant decrease in expression in IgG nonresponders as compared to IgG responders. More preferably, the gene expression pattern of IgG nonresponders includes a moderate to high level of expression of at least one of the genes selected from the group consisting of granulin and FCGRT, and/or a low level of expression of at least one of the genes selected from the group consisting of IARS and MCM3.

A unique gene expression pattern may also be associated with a favorable clinical response, e.g., the production of antibodies, particularly IgG antibodies. The invention is thus further directed to methods for predicting that a candidate AD patient will have a favorable clinical response to treatment with AN1792, the method comprising (1) associating at least one gene expression pattern of a sample with a favorable clinical response to AN1792 by methods of compiling pharmacogenomic information, as described above, (2) procuring a test sample from the candidate AD patient not previously exposed to AN1792, and (3) determining that the test sample procured from the candidate AD patient not previously exposed to AN1792 has a test gene expression pattern that is substantially similar to the at least one gene expression pattern associated with a favorable clinical response AN1792. In one embodiment of the invention, a favorable clinical response to AN1792 includes a protective immune response. In another embodiment, a favorable clinical response to AN1792 includes the development of antibodies, e.g., IgG. Preferably, the gene expression pattern of IgG responders is acquired from unstimulated patient samples and includes a moderate to high level of expression of at least one of the genes listed in Tables 24-31 as having “higher” average expression in IgG responders, and/or a low level of at least one of the genes listed in Tables 24-31 as having “lower” average expression in IgG responders. As used herein, moderate to high levels of expression means any statistically significant increase in expression in IgG nonresponders as compared to IgG responders, and low levels means any statistically significant decrease in expression in IgG nonresponders as compared to IgG responders.

Along the same lines, the present invention provides a method for predicting whether a candidate patient is likely to develop inflammation in response to the administration of a treatment for AD comprising determining whether the candidate patient has a unique gene expression pattern associated with the development of inflammation in response to the treatment.

In one embodiment of the invention, the method predicts the likelihood of whether a candidate AD patient not previously exposed to a particular treatment for AD, such as AN1792, will develop an inflammatory response, such as encephalitis, to AN1792. In this embodiment, the method comprises (1) obtaining a nucleic acid sample from a patient population previously exposed to the treatment, wherein the patient population includes inflammation developers and inflammation nondevelopers, (2) using the nucleic acid sample to determine whether the inflammation developers of the patient population have a unique gene expression pattern not found in the inflammation nondevelopers, and (3) determining whether a candidate patient not previously exposed to the treatment has the unique gene expression pattern, wherein the presence of the unique gene expression pattern in the candidate patient predicts a likelihood that the candidate patient will develop inflammation. While inflammation is the adverse effect in this embodiment, any adverse effect is contemplated by the present invention.

In another embodiment of the invention, the method predicts that a candidate AD patient not previously exposed to AN1792 will develop an adverse clinical response to AN1792. In this embodiment, the method comprises (1) associating at least one gene expression pattern of a sample with an adverse clinical response to AN1792 by methods of compiling pharmacogenomic information, as described above, (2) procuring a test sample from the candidate AD patient not previously exposed to AN1792, and (3) determining that the test sample procured from the candidate AD patient not previously exposed to AN1792 has a test gene expression pattern that is substantially similar to the at least one gene expression pattern associated with an adverse clinical response AN1792. In one embodiment of the invention, an adverse clinical response to AN1792 includes an inflammatory response. In another embodiment, an adverse clinical response to AN1792 includes the development of encephalitis, e.g., meningoencephalitis. In another embodiment, the gene expression pattern associated with an adverse clinical response is procured from an unstimulated sample and includes a moderate to high level of expression at least one of the genes listed in Tables 32-37 as having a higher average expression in encephalitis developers and/or a low level of expression of at least one of the genes listed in Tables 32-37 as having lower expression in encephalitis developers.

The determination of gene expression patterns associated with the encephalitis response in AD patients to AN1792 is useful for predicting the likelihood that a patient will develop encephalitis. Therefore, the present invention relates to a method of predicting whether a patient will develop encephalitis when administered AN1792 by (1) determining whether patients who developed encephalitis during clinical trials have a unique (preimmunization) gene expression pattern associated with encephalitis, and (2) determining whether a candidate patient has the unique gene expression pattern, wherein the presence of the unique gene expression pattern indicates that the candidate patient is not a good candidate for AN1792 treatment and the absence of the unique gene expression pattern indicates that that candidate patient is (or may be) a good candidate for AN1792 treatment.

In one embodiment, the method comprises comparing gene expression patterns of AD patients who develop encephalitis in response to AN1792 treatment (encephalitis developers) and AD patients who do not develop encephalitis in response to AN1792 treatment (encephalitis nondevelopers) to define a unique gene expression pattern for encephalitis developers, and determining whether a candidate AD patient not previously exposed to AN1792 has the unique gene expression pattern, wherein the presence of the unique gene expression pattern in the candidate AD patient predicts a likelihood that the patient will develop encephalitis. Gene expression patterns may be determined by any means known in the art, including, but not limited to determining protein and/or RNA expression patterns in a sample, as described above. In another embodiment of the invention, the method comprises (1) assaying RNA expression levels to obtain gene expression patterns for the encephalitis developers and encephalitis nondevelopers, (2) comparing the gene expression patterns of the encephalitis developers and encephalitis nondevelopers to define a unique gene expression pattern for encephalitis developers, and (3) determining whether a candidate AD patient not previously exposed to AN1792 has the unique gene expression pattern, wherein the presence of the unique gene expression pattern in the candidate AD patient predicts a likelihood that the patient will develop encephalitis. If the candidate AD patient does not have the unique gene expression pattern associated with encephalitis, the patient is (or may be) a good candidate for treatment with AN1792. The method may further comprise collecting blood from a patient population previously exposed to AN1792, wherein the patient population includes encephalitis developers and encephalitis nondevelopers, and purifying total RNA from the blood sample. In another embodiment of the invention, the method comprises (1) assaying protein expression levels to obtain gene expression patterns for the encephalitis developers and encephalitis nondevelopers, (2) comparing the gene expression patterns of the encephalitis developers and encephalitis nondevelopers to define a unique gene expression pattern for encephalitis developers, and (3) determining whether a candidate AD patient not previously exposed to AN1792 has the unique gene expression pattern, wherein the presence of the unique gene expression pattern in the candidate AD patient predicts a likelihood that the patient will develop encephalitis. If the candidate AD patient does not have the unique gene expression pattern associated with encephalitis, the patient is (or may be) a good candidate for treatment with AN1792. Protein expression levels may be assayed by any means known in the art. The method may further comprise collecting blood from a patient population previously exposed to AN1792, wherein the patient population includes encephalitis developers and encephalitis nondevelopers, and obtaining protein from the blood sample.

Methods to Improve the Safety and Efficacy of a Treatment for AD

A skilled artisan will recognize that the ability to predict the clinical response of an AD patient to treatment for AD will enable methods to improve the safety and efficacy of the treatment for AD. Such methods include, but are not limited to, providing a treatment for AD to only candidate AD patients predicted to have favorable clinical response(s) to the treatment, modifying the gene expression pattern of a sample taken from a candidate AD patient to resemble a gene expression pattern associated with a favorable clinical response (i.e., modifying the ‘gene expression pattern’ of the patient to have the gene expression pattern of a later-procured sample resemble a gene expression pattern associated with a favorable clinical response), developing a genomically guided therapeutic product, etc.

I. Improving Clinical Response Profiles of Treatments for AD

Accordingly, the present invention provides methods for improving a response profile of a treatment for AD by increasing the chances that an AD patient develops a favorable clinical response to the treatment for AD, comprising (1) determining that the AD patient has a unique gene expression pattern associated with a favorable clinical response to the treatment for AD, and (2) administering the treatment for AD to the AD patient.

The present invention provides methods for improving a response profile of a treatment for AD by reducing the risk that an AD patient will develop an adverse clinical response to the treatment for AD, comprising (1) determining that the patient has a unique gene expression pattern associated with an adverse clinical response to the treatment for AD, and (2) not administering the treatment for AD to the AD patient. In one embodiment of the invention, the methods improve the response profile of treating AD with AN1792.

Accordingly, the present invention is also directed to an improved treatment for AD comprising administering AN1792 to a patient population, wherein the patient population has a gene expression pattern associated with a favorable clinical response and/or lacks another gene expression pattern associated with an adverse clinical response.

By targeting a population of AD patients who develop a favorable clinical response to AN1792, e.g., patients who are IgG responders (thus avoiding a population of AD patients who are IgG nonresponders), i.e., patients from whom patient samples that have at least one unique gene expression profile associated with a favorable clinical response to AN1792 are procured, the efficacy of AN1792 as a treatment for AD may be improved. Therefore, the present invention provides an improved method of treatment of AD comprising treating a population of AD patients with AN1792, wherein samples procured from the population of AD patients have a unique gene expression pattern associated with a favorable clinical response. Alternatively, it may be that the samples, e.g., after culture, do not express an appropriate level(s) of one or more of the above-indicated genes that is associated with IgG nonresponsiveness in Table 18. This method of treatment results in a reduction or elimination of AD patients who are treated with AN1792 that do not mount an IgG response, and thus improves the efficacy of AN1792.

In accordance with the invention, there is also provided a method for treating a population of AD patients with AN1792, wherein the population of patients does not express a gene expression pattern associated with an adverse clinical response, e.g., expresses different expression levels of one or more of the above-indicated genes as compared to encephalitis nondevelopers. The treatment results in a reduction or elimination of the incidence of adverse clinical responses, e.g., encephalitis, in the population of AD patients and improves the safety of AN1792.

The present invention also contemplates a method of targeting candidate AD patients who are not likely to develop an adverse clinical response, e.g., encephalitis, to AN1792 and are likely to develop a favorable clinical response, e.g., a protective immune (e.g., IgG) response to AN1792. The method comprises determining a unique gene expression pattern associated with patients who develop adverse or nonfavorable clinical responses, e.g., encephalitis developers and/or IgG nonresponders, respectively, and then determining whether the candidate AD patient has this unique gene expression pattern(s). Similarly, the invention relates to a method for treating an AD patient with AN1792, wherein the AN1792 has improved safety and efficacy profiles, comprising administering AN1792 to the candidate patient not having a gene expression pattern(s) associated with an adverse or a nonfavorable clinical response, e.g., an encephalitis developer and/or an IgG nonresponder, respectively.

II. Altering a Gene Expression Pattern Associated with an Adverse Clinical Response.

One or more genes included as part of a unique gene expression pattern may also be useful as a therapeutic agent(s) or a target(s) for a treatment. Therefore, without limitation as to mechanism, some of the methods of the invention are based, in part, on the principle that regulation of the expression level(s) of one or more genes involved in a unique expression pattern associated with a particular clinical response may promote a favorable clinical response to a treatment for AD when expressed at levels similar or substantially similar in patient samples isolated from patients who develop a favorable response to a treatment for AD. The discovery of these unique expression patterns for individual or panels of genes that may be associated with a favorable or clinical response allows for screening of test compounds with the goal of regulating a unique gene expression pattern associated with a particular clinical response; for example, screening can be done for compounds that will convert a unique gene expression pattern associated with an adverse clinical response to a unique gene expression pattern associated with a favorable clinical response.

For example, in relation to these embodiments, a unique gene expression pattern may comprise genes that are determined to have modulated activity or expression in response to a therapy regime. Alternatively, the modulation of the activity or expression of a unique gene expression pattern, or one or more genes of the gene expression pattern, may be correlated with a particular clinical outcome to a treatment for AD. In addition, regulatory agents affecting the expression level of at least one gene that is part of a unique gene expression pattern (associated polynucleotides and/or polypeptides, related associated polynucleotides and/or polypeptides (e.g., inhibitory polynucleotides, inhibitory polypeptides (e.g., antibodies), small molecules, etc.) may be administered as therapeutic drugs. In another embodiment of the invention, regulatory agents of the invention may be used in combination with one or more other therapeutic compositions of the invention. Formulation of such compounds into pharmaceutical compositions is described below. Administration of such a therapeutic regulatory agent may regulate the aberrant expression of at least one gene that is part of a unique gene expression pattern, and therefore may be used to increase the chances for a favorable clinical response and/or decrease the risk of an adverse clinical response to a treatment for AD.

Altered expression of the genes of the present invention may be achieved in a cell or organism through the use of various inhibitory polynucleotides, such as antisense polynucleotides and ribozymes that bind and/or cleave the mRNA transcribed from the genes involved in a unique gene expression pattern of the invention (see, e.g., Galderisi et al. (1999) J. Cell Physiol. 181:251-57; Sioud (2001) Curr. Mol. Med. 1:575-88). Such inhibitory polynucleotides may be useful in preventing or treating inflammation and similar or related disorders.

The antisense polynucleotides or ribozymes of the invention can be complementary to an entire coding strand of a gene of the invention, or to only a portion thereof. Alternatively, antisense polynucleotides or ribozymes can be complementary to a noncoding region of the coding strand of a gene of the invention. The antisense polynucleotides or ribozymes can be constructed using chemical synthesis and enzymatic ligation reactions using procedures well known in the art. The nucleoside linkages of chemically synthesized polynucleotides can be modified to enhance their ability to resist nuclease-mediated degradation, as well as to increase their sequence specificity. Such linkage modifications include, but are not limited to, phosphorothioate, methylphosphonate, phosphoroamidate, boranophosphate, morpholino, and peptide nucleic acid (PNA) linkages (Galderisi et al., supra; Heasman (2002) Dev. Biol. 243:209-14; Micklefield (2001) Curr. Med. Chem. 8:1157-79). Alternatively, these molecules can be produced biologically using an expression vector into which a polynucleotide of the present invention has been subcloned in an antisense (i.e., reverse) orientation.

The inhibitory polynucleotides of the present invention also include triplex-forming oligonucleotides (TFOs) that bind in the major groove of duplex DNA with high specificity and affinity (Knauert and Glazer (2001) Hum. Mol. Genet. 10:2243-51). Expression of the genes of the present invention can be inhibited by targeting TFOs complementary to the regulatory regions of the genes (i.e., the promoter and/or enhancer sequences) to form triple helical structures that prevent transcription of the genes.

In one embodiment of the invention, the inhibitory polynucleotides of the present invention are short interfering RNA (siRNA) molecules. These siRNA molecules are short (preferably 19-25 nucleotides; most preferably 19 or 21 nucleotides), double-stranded RNA molecules that cause sequence-specific degradation of target mRNA. This degradation is known as RNA interference (RNAi) (e.g., Bass (2001) Nature 411:428-29). Originally identified in lower organisms, RNAi has been effectively applied to mammalian cells and has recently been shown to prevent fulminant hepatitis in mice treated with siRNA molecules targeted to Fas mRNA (Song et al. (2003) Nature Med. 9:347-51). In addition, intrathecally delivered siRNA has recently been reported to block pain responses in two models (agonist-induced pain model and neuropathic pain model) in the rat (Dorn et al. (2004) Nucleic Acids Res. 32 (5):e49).

These siRNA molecules can be generated by annealing two complementary single-stranded RNA molecules together (one of which matches a portion of the target mRNA) (Fire et al., U.S. Pat. No. 6,506,559) or through the use of a single hairpin RNA molecule that folds back on itself to produce the requisite double-stranded portion (Yu et al. (2002) Proc. Natl. Acad. Sci. USA 99:6047-52). The siRNA molecules can be chemically synthesized (Elbashir et al. (2001) Nature 411:494-98) or produced by in vitro transcription using single-stranded DNA templates (Yu et al., supra). Alternatively, the siRNA molecules can be produced biologically, either transiently (Yu et al., supra; Sui et al. (2002) Proc. Natl. Acad. Sci. USA 99:5515-20) or stably (Paddison et al. (2002) Proc. Natl. Acad. Sci. USA 99:1443-48), using an expression vector(s) containing the sense and antisense siRNA sequences. Recently, reduction of levels of target mRNA in primary human cells, in an efficient and sequence-specific manner, was demonstrated using adenoviral vectors that express hairpin RNAs, which are further processed into siRNAs (Arts et al. (2003) Genome Res. 13:2325-32).

The siRNA molecules targeted to polynucleotides associated with the disclosed genes of the present invention can be designed based on criteria well known in the art (e.g., Elbashir et al. (2001) EMBO J. 20:6877-88). For example, the target segment of the target mRNA preferably should begin with AA (most preferred), TA, GA, or CA; the GC ratio of the siRNA molecule preferably should be 45-55%; the siRNA molecule preferably should not contain three of the same nucleotides in a row; the siRNA molecule preferably should not contain seven mixed G/Cs in a row; and the target segment preferably should be in the ORF region of the target mRNA and preferably should be at least 75 bp after the initiation ATG and at least 75 bp before the stop codon. Based on these criteria, or on other known criteria (e.g., Reynolds et al. (2004) Nature Biotechnol. 22:326-30), siRNA molecules can be designed by one of ordinary skill in the art.

III. Genomically Guided Therapeutics

Another embodiment of the present invention is a method for developing a genomically guided AN1792 (a genomically guided therapeutic product) comprising determining gene expression patterns for AD subjects who are not likely to develop encephalitis after administration of AN1792 and/or who are likely to develop an. IgG response after administration of AN1792. The method of the present invention is useful in making genomically guided AN1792 which comprises AN1792 and a label comprising an indication of a target population genomically defined to be not likely to develop encephalitis after administration of AN1792 and/or likely to develop an IgG response after administration of AN1792. As used herein a label comprising an indication of a target population genomically defined to be not likely to develop encephalitis and/or likely to develop an IgG response, is any type of medium that may be provided together with AN1792, such as a leaflet, a package insert, a list of instructions, an instruction manual, a computer readable medium, a label on a bottle, or any other type of medium which conveys to the pharmacist, physician, or any other healthcare provider, and/or the patient the desired target population.

The genomically guided AN1792 includes AN1792 having an improved therapeutic response profile for an individual or a group of individuals belonging to a genomically defined population selected from a nongenomically defined population having AD, wherein the genomically defined population is preidentified as having (or not having) a particular gene expression pattern and wherein the particular gene expression pattern is associated with an improved response to AN1792. The compositions of the present invention are administered to at least one individual of the genomically defined population and are capable of treating AD in the genomically defined population more effectively or safely than treating a nongenomically defined population of individuals having AD. As noted, the genomically defined population would typically be identified as part of the indication by information printed on the label or packaging of, or otherwise provided with, genomically guided AN1792.

In addition, the present invention is directed to a defined population of cells originating from and residing in diverse mammalian individuals, preferably human, wherein said population is formed by determining the presence of a gene expression pattern associated with a characteristic response to AN1792 and wherein the population of cells is exposed to a therapeutically effective amount of AN1792. The present invention is also directed to a defined and isolated population of cells originating from diverse mammalian individuals, preferably human, wherein said population comprises a gene expression pattern associated with a characteristic response to AN1792 and wherein the population of cells is exposed to a therapeutically effective amount of AN1792. Such cells may be cultured in vitro and may be useful for the study of AN1792 in vitro.

Another aspect of the invention relates to a method comprising the steps of providing at least one peripheral blood sample of an AD patient; and comparing an expression profile of one or more genes in the at least one peripheral blood sample to at least one reference expression profile from an AD patient treated with AN1792 of said one or more genes. Each of the genes is differentially expressed in peripheral blood mononuclear cells (PBMCs) of AD patients who developed encephalitis, or did not develop an IgG response, or both, in response to AN1792 treatment as compared to AD patients who did not develop encephalitis, or did develop an IgG response, or both, respectively, in response to AN1792 treatment.

Diagnostic or screening methods based on differentially expressed gene products are well known in the art. In accordance with one aspect of the present invention, the differential expression patterns of an AD patient likely to develop encephalitis and/or not develop an IgG response in response to AN1792 treatment can be determined by measuring the level of RNA transcripts of these genes in peripheral blood samples. Suitable methods for this purpose include, but are not limited to, RT-PCR, Northern Blot, in situ hybridization, Southern Blot, slot-blotting, nuclease protection assays and polynucleotide arrays. The peripheral blood samples can be either whole blood, or samples containing enriched PBMCs. In other embodiments of the invention, the source of genes can be a bodily fluids or a tissue other than blood.

In general, RNA isolated from peripheral blood samples can be amplified to cDNA or cRNA before detection and/or quantification. The isolated RNA can be either total RNA or mRNA. Suitable amplification methods include, but are not limited to, RT-PCR, isothermal amplification, ligase chain reaction, and Qbeta replicase. The amplified nucleic acid products can be detected and/or quantified through hybridization to labeled probes. Amplification primers or hybridization probes can be prepared from the gene sequence of differentially expressed genes using methods well known in the art.

The differential expression patterns of genes associated with the likelihood of developing encephalitis and/or of not developing an IgG response can also be determined by measuring the levels of polypeptides encoded by these genes in peripheral blood. Methods suitable for this purpose include, but are not limited to, immunoassays such as ELISA, RIA, FACS, dot blot, Western Blot, immunohistochemistry, and antibody-based radioimaging.

Suitable antibodies include, but are not limited to, polyclonal antibodies, monoclonal antibodies, chimeric antibodies, humanized antibodies, single chain antibodies, Fab fragments and fragments produced by Fab expression libraries. Such antibodies can be prepared by methods well known in the art. Available antibodies may also be used.

In a further aspect of the invention, there is provided a system comprising a computer readable memory that stores at least one reference expression profile of one or more genes in peripheral blood samples of a reference AD patient, wherein each of said one or more genes is differentially expressed in PBMCs of AD patients who are likely to develop encephalitis, or not likely to develop an IgG response, or both, respectively, in response to AN1792 treatment as compared to AD patients who are not likely to develop encephalitis, or are likely to develop an IgG response, or both, respectively, in response to AN1792 treatment. A program capable of comparing an expression profile of interest to the reference expression profile, and a processor capable of executing the program, is also provided in the system.

For the method of treatment for AD of the present invention, AN1792 is administered in a therapeutically effective amount. AN1792 may be administered orally, topically, parenterally, by inhalation or spray (e.g., nasally), or rectally in dosage unit formulations containing conventional nontoxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. Preferably, the AN1792 is administered as a pharmaceutical formulation comprising AN1792 and a pharmaceutically acceptable carrier. AN1792 may be present in association with one or more nontoxic pharmaceutically acceptable carriers and/or diluents and/or adjuvants, and, if desired, other active ingredients. The pharmaceutical compositions containing AN1792 may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain AN1792 in admixture with nontoxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the AN1792 is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain AN1792 in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending AN1792 in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide AN1792 in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil or a mineral oil or mixtures of these. Suitable emulsifying agents may be naturally occurring gums, for example gum acacia or gum tragacanth, naturally occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a nontoxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid find use in the preparation of injectables.

AN1792 may also be administered in the form of suppositories, e.g., for rectal administration of the drug. These compositions can be prepared by mixing the drug with a suitable nonirritating excipient that is solid at ordinary temperatures but liquid at the rectal temperature and will therefore melt in the rectum to release the drug. Such materials include cocoa butter and polyethylene glycols.

AN1792 may be administered parenterally in a sterile medium. AN1792, depending on the vehicle and concentration used, can either be suspended or dissolved in the vehicle. Advantageously, adjuvants, local anesthetics, preservatives and buffering agents can be dissolved in the vehicle.

In one embodiment, the AN1792 peptide antigen is provided as a sterile liquid suspension, which appears as a hazy, colorless liquid suspension and which includes 0.5 mg/mL, in 10 mM glycine, 10 mM sodium citrate, 0.4% polysorbate 80, 5% sucrose, at a pH of 6.0. The AN1792 is administered together with QS-21 adjuvant, which is provided as a sterile, clear solution, and includes 1.0 mg/mL, in phosphate buffered saline with 0.4% polysorbate 80 at a pH of 6.5.

QS-21 (Stimulon™; Antigenics, Inc., Framingham, Mass.; U.S. Pat. No. 5,057,540) is a naturally occurring saponin molecule purified from the South American tree Quillaja saponaria Molina. Numerous studies in laboratory animals have demonstrated the adjuvant activity of QS-21 and have established its safety profile. Rabbit toxicity studies with single or multiple injections of various doses of QS-21 alone or combined with various antigens have documented a pattern of mild to moderate inflammation (hemorrhage, necrosis and edema) at the injection site and no significant organ toxicity. Slight alterations in white blood cell counts (leukocytosis and leukopenia) and creatinine kinase are common. Pharmacokinetic data collected after a single IM injection of tritium-labeled QS-21 in rabbits show QS-21 highly concentrated in the lymph nodes draining the injection area. Excretion occurs primarily through the kidneys, and both QS-21 and its metabolites are found in the urine. Studies in mice, rabbits and monkeys with QS-21 adjuvanted immunotherapeutics show improvement in B and T cell effector function, especially an increase in achieved antibody titers, induction of antigen-specific cytotoxic T lymphocytes, immunoglobulin class switching, affinity maturation and broadening of antigen-primed B cell repertoire.

In another embodiment, polysorbate 80 is a component of the formulated drug product AN1792 and the adjuvant, QS-21. It is a nonionic surfactant used widely as an emulsifying agent in the preparation of stable oil-in-water pharmaceutical emulsions. It is also used as a solubilization agent or as a wetting agent in the formulation of oral and parenteral suspensions. There have been occasional reports of rare contact hypersensitivity to polysorbates following their topical use and reports of tuberculin type hypersensitivity following intramuscular injection in combination with vitamin A. Polysorbates have also been associated with serious adverse events, including some deaths in low-birth weight infants following intravenous administration of a vitamin E preparation containing a mixture of polysorbate 20 and 80.

The AN1792 and QS-21 are preferably administered by intramuscular injection into deltoid muscle. If multiple administrations are desired, sides may be alternated for each injection session. Several administrations may be necessary to achieve the best results; in one embodiment, administrations are given as follows: a first injection is given at day 1; one month later, a second injection is given; 2 months after injection 2, a third injection is given; 3 months after injection 3, a fourth injection is given; 3 months after injection 4, a fifth injection is given; and 3 months after injection 5, a sixth injection is given, for a total of six injections in one year.

At present, the anti-AN1792 titer necessary to achieve a beneficial therapeutic effect in human AD is unknown. Whereas the PDAPP (platelet-derived growth factor-driven amyloid precursor protein) transgenic mouse develops several AD-like neuropathologies, the progression of pathology in this model may very well take a more aggressive course than in human AD, as the changes occur in months and the expression levels APP/Aβ are several fold higher than in nontransgenic species. The lowest titers in PDAPP efficacy studies that have resulted in lessening of neuropathological progression have been in the range of 1-2,000. In addition, a fragment of Aβ(1-5) attached to a carrier protein and combined with complete Freund's adjuvant/incomplete Freund's adjuvant was effective in preventing neuropathology despite raising a peak geometric titer of only 2,400.

It will be understood, however, that the specific dose level and administration dosing schedule for any particular patient will depend upon a variety of factors including the activity of the AN1792 employed, the age, body weight, general health, sex, diet, time of administration, route of administration, and rate of excretion, drug combination and the severity of the particular disease undergoing therapy, as well as the antibody titer that is desired.

The following examples are intended to illustrate the invention and should not be construed as limiting the invention in any way

### EXAMPLES

An exploratory search for predictors of clinical responses to AN1792 immunization in the preimmunization gene expression patterns in PBMCs of patients with mild to moderate AD was undertaken. Accordingly, pharmacogenomic analyses have been performed with the intention of determining associations between gene expression patterns and clinical response parameters.

Predictors of response were sought because the incidence of antibody responsiveness in the Phase I study was relatively low (48%), an incidence that would have more than doubled the number of patients required in a Phase II evaluation of efficacy (as measured by cognitive function) associated with anti-AN1792 antibody response. Therefore, a wide and unbiased pharmacogenomic-based search for genes whose expression levels prior to immunization were significantly associated with postimmunization positive antibody titer was designed. Consequently, blood samples were obtained from 123 treated U.S. patients (five of which developed meningoencephalitis) and 30 patients in the placebo group. Simultaneous analysis of the expression levels of approximately 22,000 sequences in each preimmune blood sample obtained from all consenting subjects was performed using the Affymetrix U133A GeneChip®. In the Phase Ia trials of AN1792, by the time encephalitis was recognized as a severe adverse event, preimmune blood samples from five of the six U.S. encephalitis patients had been collected for pharmacogenomic studies. (The sixth U.S. encephalitis patient had not consented to the pharmacogenomic portion of the study, and therefore no blood sample was available from this patient for the pharmacogenomics study).

In summary, as developed below, associations between preimmunization gene expression patterns in peripheral blood mononuclear cells of AD patients, that were either placed under in vitro culture conditions (Example 1) or unstimulated (Example 2), and postimmunization clinical responses have been found. Corroboration of these findings may be of interest and may be made by showing the same associations in a second (independent) sample set (e.g., samples from the European clinical trial patients).

### Example 1

### Association Between Gene Expression Patterns of in Vitro Stimulated (Cultured) Samples and Adverse Clinical Responses

### Example 1.1

### Materials and Methods—Sample Preparation

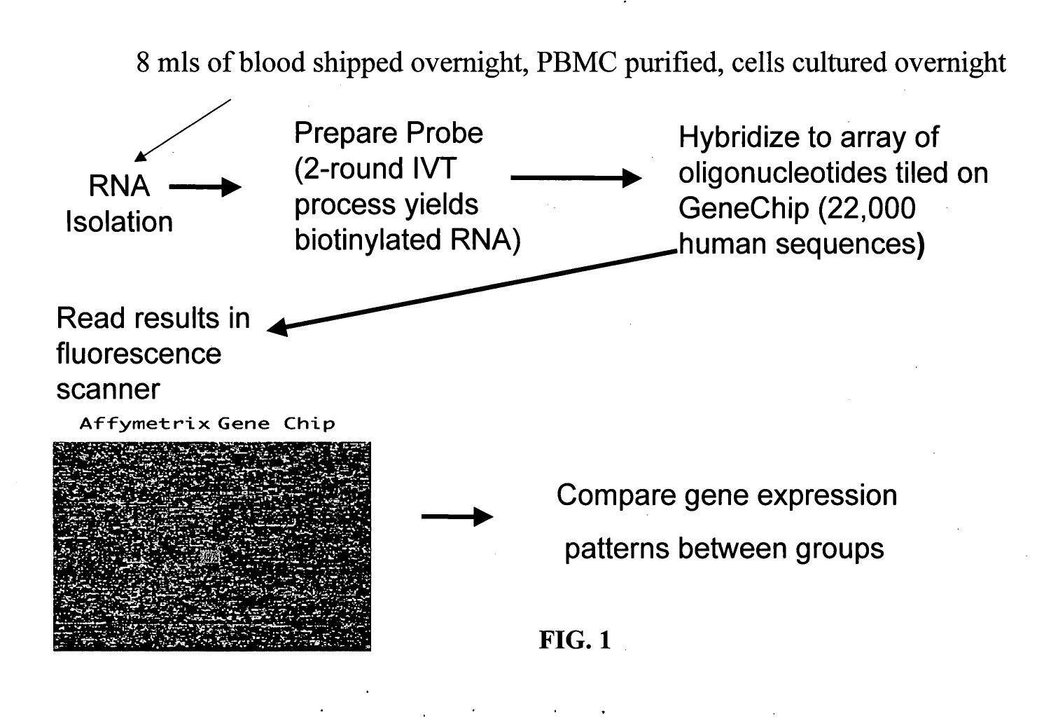

Consent to the pharmacogenomic study was optional and obtained after approval by local institutional review boards in the U.S. (E.U. patients were not included in the pharmacogenomic study). Blood was collected from patients in the U.S. at the screening visit and was shipped overnight at room temperature to the Pharmacogenomic Laboratory in Andover, Mass. For each sample, the peripheral blood mononuclear cell (PBMC) fraction was purified by CPT fractionation, as described below, and 2×10⁶ of these cells (the baseline sample, i.e., the first daughter sample for baseline measurements) were snap frozen; these represent cells that were not subject to in vitro culture (see. Example 1.1.3.1). The remaining cells were divided into four equal aliquots and cultured in vitro overnight in conditions described below. Cells were then harvested and snap frozen. The culturing step was performed because it was reasoned that preimmunization gene expression profiles in PBMCs associated with a postimmunization clinical response to AN1792 might most likely be revealed by exposing PBMCs to AN1792 as an antigen in culture. The hypothesis behind this reasoning was that immunotherapeutic responsiveness may reflect a state of “preexisting readiness” to respond to AN1792, and this state may be reflected in the gene expression profile of PBMCs prior to immunotherapy. Accordingly, both AN1792-stimulated and control cultures were set up for each sample. Total RNA was purified from each sample, and RNA expression levels of each of 22,000 sequences were assayed, as described below. Statistical analyses were performed to identify genes whose expression patterns showed a statistically significant association with antibody responsiveness, development of encephalitis or the presence of ApoE4 alleles. FIG. 1 shows a summary of the design of this Example 1.

### Example 1.1.1

### Purification of PBMCs by CPT Fractionation

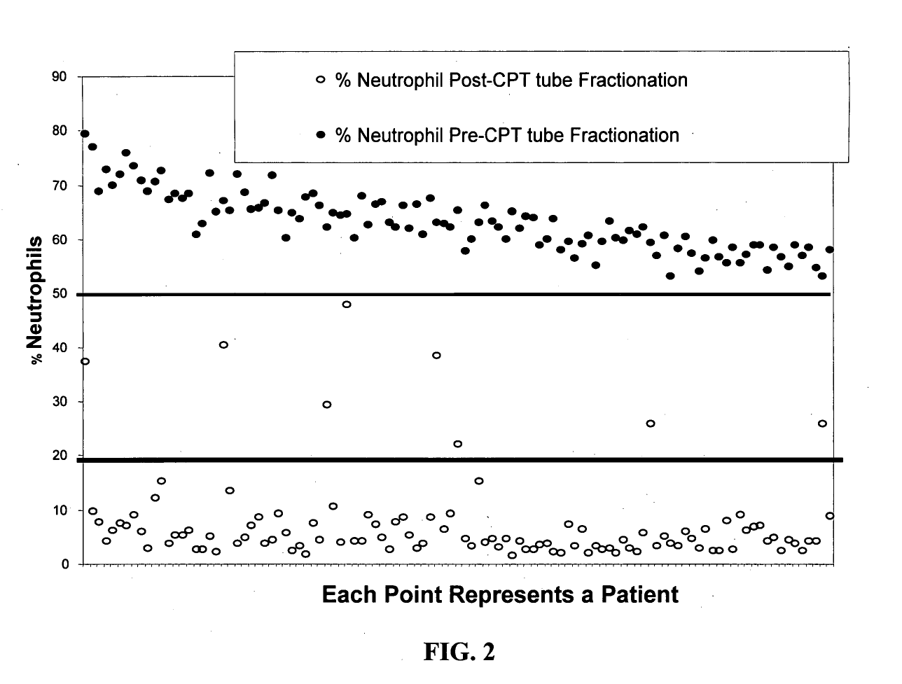

Fractionation of PBMCs by CPT (cell preparation tube) fractionation was performed using a single screening visit blood sample drawn into a CPT Cell Preparation Vacutainer Tube (BD Vacutainer Systems, Franklin Lakes, N.J.). The target volume was 8 ml, but in some cases this target was not reached. Samples that were not received at Pharmacogenomics Laboratory within a day of collection were excluded from the study. Upon receipt, differential cell counts were performed. The PBMC fraction was then purified according to the CPT protocol (BD Vacutainer Systems) and differential cell count performed on the purified PBMC fraction. CPT purification resulted in greater than 99% reduction in RBC representation in all 141 study samples. CPT purification did not alter by more than 15% the percentage of monocytes relative to PBMCs. The efficiency of removal of neutrophils by CPT fractionation is shown in FIG. 2. For the samples of FIG. 2, CPT tubes were inverted gently eight times, 300 μl was removed in a counting vial for the Pentra 60 C+ analyzer (ABX Diagnostics; Montpellier, France) and differential counts performed. PBMC purification on the remaining sample was performed by centrifugation in a horizontal swinging rotor bucket at 1500×g for 20 minutes. The PBMC fraction was removed and washed by adding 5 ml phosphate buffered saline (PBS), gently inverting eight times, and transferring into a 15 ml conical tube. This procedure was repeated using 3 ml PBS. The PBMC fraction was then pelleted at 450×g for 5 minutes. The supernatant (PBS) was discarded, cells were resuspended in 3 ml PBS, and 300 μl of this was removed for cell differential counts using a Pentra 60 C+ analyzer. Closed symbols represent the percentage neutrophils before CPT fractionation; open symbols represent the percentage neutrophils after CPT fractionation.

Post-CPT fractionation, the percentage of neutrophils averaged 11% of the neutrophil percentage before fractionation, with a standard deviation of 11. As seen in FIG. 2, in eight cases (patients 17, 23, 36, 44, 271, 288, 311, and 756) CPT fractionation failed to reduce the percentage of neutrophils to less than 20%. (As shown in Table 2 (see also Table 6), one of these eight patients, patient 311, was removed from analysis due to an operator error identified during QC review.) It has been reported (Schmielau and Finn (2001) Cancer Res. 61:4756-60) that changes in neutrophils upon activation cause them to sediment aberrantly and copurify with PBMCs, suggesting that density change is a marker of their activation. Therefore it is likely that the seven samples included in data analysis that have a relatively high number of neutrophils in the post-CPT PBMC fraction came from patients with a higher than normal percentage of activated neutrophils. Since this parameter (activated neutrophils) could potentially impact gene expression profiles, upon unblinding of the samples, the characteristics of these seven samples among the patient groups were analyzed to determine whether there was an over- or under-representation of samples with high neutrophil content in any of the patient groups. Table 2 lists characteristics of samples with post-CPT fractionation neutrophil content >20%, and shows that patients with high neutrophil content are represented in both the antibody responding and nonresponding groups. None of the five patients who developed encephalitis are among the patients with high post-CPT fractionation neutrophil content.

Of the seven patients with high postfractionation neutrophil content, one received placebo, four are IgM nonresponders and three are IgM responders. As mentioned above, data from patient 311 was removed from analysis due to an operator error identified during QC review.

### Example 1.1.2

### Overnight Culture Conditions

All in vitro culture was done in upright tissue culture flasks (Falcon, catalog number 353108; Fischer Scientific, Pittsburgh, Pa.) in complete culture media consisting of RPMI 1640, 10% heat inactivated fetal calf serum (0.9 EU/ml), 100 u/ml penicillin and 100 μg/ml streptomycin (GIBCO/BRL; Gaithersburg, Md.), 2 mM glutamine (GIBCO/BRL), 5×10⁻⁵ M 2-mercaptoethanol. Cultures were incubated at 37° C. with 5% CO₂ overnight. In cases where at least 1×10⁷ cells were available, 2.5×10⁶ cells were added to 5 ml of treatment group stimulation media for each of four culture groups. (Stimulation media for each of the four groups is described below.) In cases where cell number was <1×10⁷, 25% of the available cells were added to 5 ml of treatment group stimulation media for each of the four treatment groups.

### Example 1.1.3

### Generation of Five Daughter Samples from Each Patient Sample

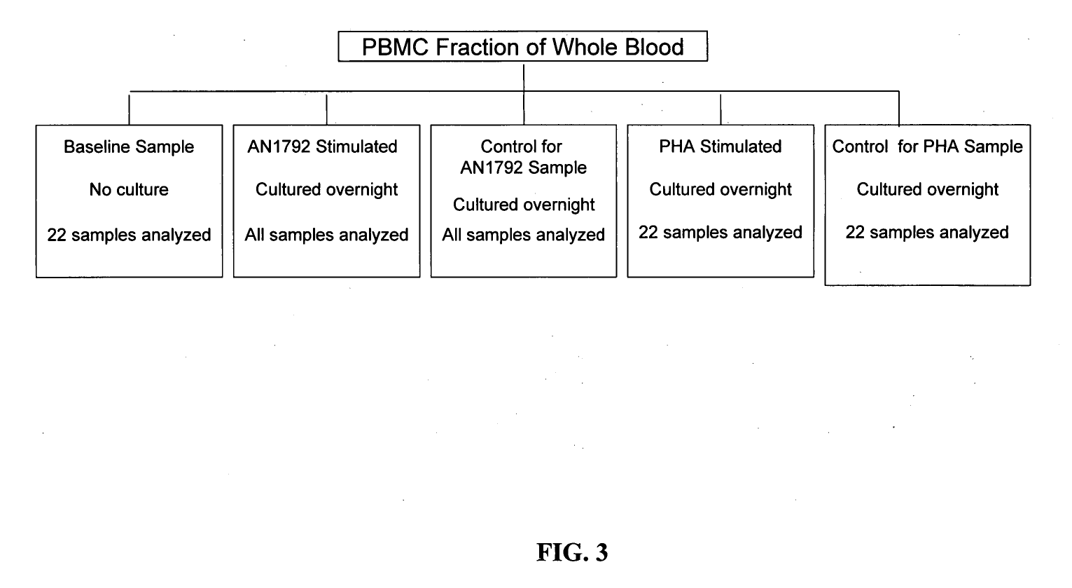

Five daughter samples were generated from each patient sample received. FIG. 3 provides a summary of the samples generated and the samples selected for analysis. As detailed below, five daughter samples were generated from each available purified PBMC sample. One of these daughter samples was not placed in culture (first daughter sample). The other four daughter samples were cultured overnight as described above (second through fifth daughter samples).

### Example 1.1.3.1

### Baseline (First Daughter) Samples—(Unstimulated)

An aliquot consisting of 2×10⁶ cells was removed from the purified PBMC fraction, pelleted by centrifugation, resuspended in 300 μl RLT Buffer (Qiagen, Valencia, Calif.) containing 2-mercaptoethanol (the starting buffer for RNA purification), snap frozen, and stored at −80° C. Initially, gene expression analysis was performed on a small subset (22) of the baseline samples. The remaining samples were retained pending the results derived from the in vitro-stimulated samples. Analysis of the entire set of baseline (unstimulated) samples (independent of the analysis provided in this Example 1) is addressed in Example 2.

### Example 1.1.3.2

### AN1792-Stimulated (Second Daughter) Samples

Cells cultured in media supplemented with AN1792 (10 μg/ml) and a cocktail of immune stimulatory adjuvants consisting of 10 U/ml rhIL-12 (Wyeth, Cambridge, Mass.), 1.5 ng/ml rhIL-2 (R&D Systems, Minneapolis, Minn.), 1.5 ng/ml rhIL-6 (R&D Systems), 10 ng/ml rhIL-7 (R&D), and 10 μg/ml hB7.2 IgG1 (Wyeth). Gene expression analysis was performed on all available samples from this culture condition.

### Example 1.1.3.3

### Control for AN1792-Stimulated (Third Daughter) Samples—(AN1792 Vehicle-Stimulated)

Cells were cultured under conditions identical to those for the AN1792-stimulated samples except that, as a placebo control, the buffer for AN1792 (10 mM glycine, 10 mM citrate, 5% sucrose, 0.4% PS-80, pH 6.0) was added at the same concentration as in the AN1792-stimulated samples. Gene expression analysis was performed on all available samples from this culture condition.

### Example 1.1.3.4

### PHA-Stimulated (Fourth Daughter) Samples

Cells were cultured in complete media with 1:150 dilution of Bacto PHA (Phytohemagglutinin P, DIFCO, Becton, Dickinson and Company, BD Biosciences, San Jose, Calif.: 1% solution in 0.85% saline). Gene expression analysis was performed on a small subset (22) of the samples from this culture condition.

### Example 1.1.3.5

### Control for PHA-Stimulated (Fifth Daughter) Samples—(PHA Vehicle-Stimulated)

Cells were cultured under conditions identical to those for the PHA-stimulated samples except that no PHA was added to the culture. Gene expression analysis was performed on a small subset (22) of the samples from this culture condition.

### Example 1.1.4

### Cell Harvest and RNA Purification

Nonadherent cells were harvested and pelleted. RLT buffer and 2-mercaptoethanol (350 μl) were added to the flask to allow for the harvest of adherent cells. This suspension was then added to the spun pellet of nonadherent cells. These suspensions were then snap frozen on dry ice and stored at −80° C. RNA purification was performed using QIAshredders and Qiagen RNeasy mini-kits.

### Example 1.1.5

### RNA Amplification and Generation of GeneChip Hybridization Probe

A probe for hybridization, i.e., biotinylated cRNA, was made from each sample by a two-cycle IVT amplification protocol (with biotinylated nucleotides incorporated during the second cycle). Due to the small amount of sample available, the two-cycle protocol was necessary for generation of sufficient biotinylated cRNA (10 μg of biotinylated cRNA from 50 ng of total RNA) for hybridization. The published Affymetrix two-cycle protocol was followed. Any sample for which the total RNA yield was <50 ng, or which yielded <10 μg of biotinylated cRNA after the IVT amplification reactions was excluded from further processing. Ten μg of biotinylated cRNA from each sample was fragmented to form a hybridization mixture. An eleven member standard curve, comprising gene fragments derived from cloned bacterial and bacteriophage sequences, was also included (spiked) in each hybridization mixture at concentrations ranging from 0.5 pM to 150 pM, representing RNA frequencies of approximately 3.3 to 1000 ppm (see Hill et al. (2001) Genome Biology 2 (12):research0055.1-0055.13). The biotinylated standard curve fragments were synthesized by T7-polymerase-driven IVT reactions from plasmid-based templates. The spiked biotinylated RNA fragments serve both as an internal standard to assess chip sensitivity and as a standard curve to convert measured fluorescent difference averages from individual genes into RNA frequencies in ppm. A reaction mixture (containing biotinylated cRNA and the 11 member standard curve) for each sample was hybridized for 16 hr at 45° C. to the Affymetrix HG-U133A oligonucleotide GeneChip, which interrogates the RNA levels of over 22,000 sequences.

### Example 1.2

### Materials and Methods—Determination of Expression Patterns

### Example 1.2.1

### Determination of Gene Expression Frequencies

The hybridization mixtures were removed and stored, and the arrays were washed and stained with streptavidin R-phycoerythrin (Molecular Probes, Inc., Eugene, Oreg.) using GeneChip Fluidics Station 400 (Affymetrix, Inc.) and scanned with a Hewlett Packard GeneArray Scanner (Hewlett Packard, Palo Alto, Calif.) following the manufacturer's instructions. Array images were processed using the Affymetrix MicroArray Suite 5.0 software (MAS 5.0; Affymetrix, Inc.) such that raw array image data (.dat files) produced by the array scanner were reduced to probe feature-level intensity summaries (.cel files) using the desktop version of MAS 5.0. Using the Gene Expression Data System (GEDS) as a graphical user interface, a sample description was provided to the Expression Profiling Information and Knowledge System (EPIKS) Oracle database, and the correct cel file was associated with the description. The database processes then invoked the MAS 5.0 software to create probeset summary values: probe intensities were summarized for each message using the Affymetrix Signal algorithm, and the Affymetrix Absolute Detection metric (Absent, Present, or Marginal, as defined by the MAS 5.0 software) for each probeset. MAS 5.0 was also used for the first pass normalization by scaling the trimmed mean to a value of 100. The database processes also calculated a series of chip QC (quality control) metrics and stored all the raw data and QC calculations back to the database.

### Example 1.2.2

### Inclusion Criteria for GeneChip Results

The EPIKS database contained all GeneChip results including those that must be excluded from the analysis. Excluded data consist of GeneChip results for: a) samples other than those stimulated in culture with AN1792 or its control, and b) replicate chips. Replicate GeneChip results were generated both when samples were rerun due to QC failure and when replicates were run to assess between-chip variability. To ensure equal weight per sample, only one chip (the last chip run for any given sample) per culture condition per patient sample was used in the analyses. All samples whose chips failed QC specifications were rerun and passed. Therefore no samples were lost to analysis due to GeneChip QC failure. Table 3 lists chip QC inclusion specifications used in this analysis (although other means of quality control for GeneChips or other DNA microarray chips may be used).

### Example 1.2.3

### Normalization and Filtering of Gene Expression Data

Frequency values for chips meeting inclusion criteria were normalized to control for chip-to-chip differences. The scaled frequency method of Hill et al. ((2001) Genome Biology 2 (12):research0055.1-0055.13) was used. Genes that do not have any relevant information were filtered from the dataset. This occurred in two stages: 1) any gene that was called Absent on all GeneChips (as determined by the Affymetrix Absolute Detection metric in MAS 5.0) was removed from the dataset; and 2) any gene that was expressed at a normalized frequency of <10 ppm on all GeneChips was removed from the dataset to ensure that any gene kept in the analysis set was detected at a frequency of at least 10 ppm at least once. (In previous studies, high variability had been observed in frequency measurements below 10.) The total number of genes in the analysis after these filtering steps were performed was 10,168.

### Example 1.2.4

### Identification and Reporting of Outlier Samples

To identify outlier samples, we computed the square of the pairwise Pearson correlation coefficients (r²) among all pairs of samples using Splus (Version 5.1) (ITC Computer Systems, University of Virginia). Specifically, we started from the G×S matrix of expression values, where G is the total number of genes and S is the total number of samples. We calculated r² between all pairs of columns (samples) in this matrix. The result was a symmetric S×S matrix of r² values (see Weinstein et al. (1997) “An information-intensive approach to the molecular pharmacology of cancer,” Science 275:343-49). This matrix measures the similarity between each sample and all other samples in the analysis. Since all of these samples come from (relatively) elderly human PBMCs treated according to common protocols, the expectation is that the correlation coefficients reveal a high degree of similarity in general (i.e., the expression levels of the majority of the 10,168 transcripts are similar in all samples analyzed). To summarize the similarity of samples, for each sample the average of the r² values between that sample and the other samples studied in this Example 1 was calculated (Table 4).

The closer the value of average r² is to 1, the more alike the sample is to the other samples within the analysis. Low average r² values indicate that the gene expression profile of the sample is an “outlier” in terms of overall gene expression patterns. Outlier status can indicate either that the sample has a gene expression profile that deviates significantly from the other samples within the analysis, or that the technical quality of the sample was inferior. Therefore, the pharmacogenomic supplemental statistical analysis plan of this study stipulated the step of identifying any outliers (average r² value <0.75) and conducting an analysis of the individual gene expression profile of each outlier. There are a total of seven samples (listed in Table 5) that meet this criterion.

The r² outlier samples identified in Table 5 include one particularly critical sample: the AN1792-stimulated sample from patient 33. Patient 33 is one of five encephalitis patients. The gene expression profiles of the seven r² outlier samples were examined, and it was determined that they all contain sequences that are expressed throughout the linear range of the standard curve. None of the samples shows gene expression frequencies either uniformly lower or higher than average. Therefore, it is highly unlikely that the r² status of these outliers is due to a technical failure of the in vitro transcription (IVT) reactions or other factors related to sample quality.

### Example 1.2.5

### Merging of Clinical and Gene Expression Data

Relevant clinical data received from StatProbe, Inc. (Ann Arbor, Mich.) (pertaining to treatment group, maximum IgG titer for all visits, maximum IgM titer for all visits, ApoE4 type, and encephalitis status), along with demographic data and treatment group, were merged with the gene expression data by donor identification number (the randomization number that was assigned to each patient in the study).

### Example 1.2.6

### Samples Analyzed for Gene Expression Levels

### Example 1.2.6.1

### Sample Inclusion Criteria

Inclusion in the study required 1) that samples arrive at the Pharmacogenomics Laboratory within one day of collection, 2) that culture conditions were within specifications, 3) an RNA yield >50 ng, and 4) an IVT yield >10 μg. Table 6 accounts for all samples received for this Example 1, and identifies the number of patients in this study. Of the 172 enrolled U.S. patients, 167 consented to inclusion in the pharmacogenomic portion of the study. Of the 167 samples, six did not meet shipping specifications, and an additional 12 did not meet culture and storage specifications. Eight samples yielded insufficient product for chip hybridization, and an additional eight samples were removed due to an operator error identified during QC review. Therefore, the total number of AN1792-stimulated samples analyzed in this Example 1 is 133.

### Example 1.2.6.2

### Demographics of Patients

Sixty-four (64) of the patients in this Example 1 were female and 69 were male. Ages ranged from 53 to 87 years. Patient demographics are shown in Table 7.

The vast majority of patients (86%) were Caucasian. Hispanic (9%), Black (3%), Asian (1%), and unknown (2%) comprised the remainder. Gender representation was balanced within these groups and is shown in Table 8. All five encephalitis patients are Caucasian females born between August 1918 and December 1929.

The pharmacogenomic supplemental statistical analysis plan of this Example 1 defines IgG responders as having a maximum titer≧2200 at any time point. The maximum titer of partial IgG responders was >200<2200, and of nonresponders was ≦200. Patients with an IgM titer>100 at any time point are defined as IgM responders. Table 9 gives a breakdown of study samples by gender, response category, and ApoE type.

### Example 1.2.6.3

### Overview of Approach to Statistical Analyses (Pharmacogenomic Supplemental Statistical Analysis Plan)

Two approaches, analysis of variance (ANOVA) and signal-to-noise metrics (described below), were used in this Example 1 to identify significant associations between preimmunization gene expression patterns of in vitro stimulated samples and patient antibody response, development of encephalitis, and ApoE4 type. These two approaches were designed to find different types of associations in complex sets of data, and therefore different relationships can be identified by the two methods.

Two types of gene expression metrics were used: the logarithm of the gene frequency of the AN1792-stimulated culture, and the logarithm of the ratio of the gene frequency of the AN1792-stimulated culture to the gene frequency of the control culture for each patient sample. This latter metric is equivalent to the difference between the logarithms of the gene frequencies for the two culture conditions.

### Example 1.2.6.3.1

### ANOVA

For each gene in the final data analysis set, ANOVA was used to determine whether there is a significant association between the gene frequency metric and 1) antibody response (IgM), 2) antibody response (IgG), 3) ApoE4 type, and 4) development of encephalitis. In the ANOVA analysis, raw p values were adjusted for multiplicity according to the false discovery rate (FDR) procedure of Benjamini and Hochberg ((1995) J. Royal Stat. Soc. B57:289-300), as well as the stepdown bootstrap procedure of Westfall and Young ((1993) Resampling-Based Multiple Testing: Examples and Methods for p-Value Adjustment. John Wiley and Sons, Inc., New York; p. 67). Genes with an FDR of <0.05 are reported. At this threshold, 5% of findings are likely to be false positives. The tables presenting the statistical data also provide the raw (unadjusted) p value for each of these genes. Because it has been reported (Xiao et al. (2002) BMC Genomics 3:28) that the genes identified through the FDR procedure are more likely to be of biological relevance than those identified by the stepdown bootstrap procedure of Westfall and Young, and because the analyses of these data support the same conclusion, the FDR procedure is the focus of the analysis.

### Example 1.2.6.3.2

### GeneCluster

The GeneCluster application chooses marker genes by a signal-to-noise metric and evaluates them for their association with a given response parameter using a weighted voting algorithm (Golub et al. (1999) Science 286:531-37). Genes are assigned a score, and the 95^{th} percentile scores in randomly permuted data are provided for comparison. Genes with a score greater than that reported in the 95^{th} percentile column for randomly permuted data are reported as showing a significant association with a patient group. The probability of seeing a gene that scores this high by chance is less than 0.05. In cases where the number of genes showing a significant association is greater than 100, only the first 100 genes are reported.

In analyses where no gene shows significance at the 0.05 level by GeneCluster, but ANOVA did identify genes at the 0.05 significance level, the top 50 genes in GeneCluster showing significance at the 0.1 level are reported for the purposes of discussion and comparison with the genesets identified through ANOVA analysis.

### Example 1.3

### Materials and Methods—Data Analysis

### Example 1.3.1

### Metrics of Data Submitted for Analysis

For each of the four clinical parameters (IgG response, IgM response, ApoE4 type, and encephalitis outcome), two distinct sets of analyses were done for the cultured samples: analysis using the gene frequency in AN1792-stimulated samples, and analysis using the ratio (fold change of frequency) of the AN1792-stimulated sample and its control-stimulated sample. Two distinct sets of genes were submitted for these two types of analyses: 1) only those genes where the ratio of the maximum gene frequency to the minimum gene frequency is >2 (the metric used for this analysis is the frequency of samples stimulated in culture with AN1792); and 2) all genes that passed the filtering criteria (called Present, and with at least one frequency >10 ppm). The metric for this set of genes is the ratio of the frequency of the AN1792 cultures sample to the frequency for the diluent control sample.

### Example 1.3.2

### Definitions of Groups Compared

### Example 1.3.2.1

### Analysis of Association Between Gene Expression Metric and Development of Encephalitis

The five female encephalitis patients were compared to the 44 treated female nonencephalitis patients.

### Example 1.3.2.2

### Analysis of Association Between Gene Expression Metric and IgG Titer

The 22 treated patients with a maximum IgG titer≧2200 (responders) were compared to the 60 treated patients with a maximum IgG titer≦200 (nonresponders). (Data from patients with maximum titers between 200 and 2200 were not used in the identification of statistically significant associations, but were analyzed once the statistical programs had identified genes of interest.)

### Example 1.3.2.3

### Analysis of Association Between Gene Expression Metric and IgM Titer

The 81 treated patients with a maximum IgM titer>100 (responders) were compared to the 27 treated patients with a maximum IgM titer<100 (nonresponders).

### Example 1.3.2.4

### Analysis of Association Between Gene Expression Metric and ApoE4

All patients (treated and placebo) for which ApoE4 typing is known (104 patients) were included in this analysis. The 70 ApoE4 positive patients (homozygous and heterozygous) were compared to the 34 ApoE4 negative patients.

### Example 1.4

### Results—Gene Expression Association with Encephalitis

### Example 1.4.1

### Gene Expression Levels Showing Association with Encephalitis Using the Metric of Gene Frequency in AN1792-Stimulated Cultures

The logarithm of the gene frequency of the AN1792-stimulated culture was calculated for each gene for each of the five female encephalitis patients and each of the 44 female nonencephalitis patients receiving immunotherapy. ANOVA and GeneCluster analyses were conducted comparing these two groups.

### Example 1.4.1.1

### ANOVA

In the ANOVA analysis of the frequencies of genes in the AN1792-stimulated samples, 118 probesets had an association with encephalitis with a false discovery rate (FDR)<0.05. The unadjusted p values for these genes with FDR<0.05 ranged from 0.000001 to ≦0.0006. These 118 probesets represent 96 genes of known function and 17 sequences whose functions are not yet known. The balance (five probesets) represents genes tiled more than once on the U133A chip, and thus identified more than once by ANOVA. The 113 genes associated with encephalitis by ANOVA with FDR<0.05 are listed in alphabetical order in Table 10.

### Example 1.4.1.2

### GeneCluster Analysis

Using GeneCluster, genes with elevated expressions most closely associated with encephalitis were identified, and 162 of these genes had a permutation-based p value <0.05. None had a permutation-based p value <0.01. The narrow range of permutation-based p values for the 162 genes identified (>0.01, <0.05) reflects the small sample size of the encephalitis group and the similarity in expression patterns of a large number of the genes identified (discussed in more detail below). The 100 genes with the top scores in GeneCluster for association between increased expression and encephalitis (out of the aforementioned 162 genes) are shown in Table 11.

Using GeneCluster, no gene whose decreased expression was closely associated with encephalitis had a permutation-based p value <0.05, although there were a large number of genes that just missed this cutoff. However, the results indicate that there are genes associated with decreased expression levels in encephalitis both by ANOVA (FDR<0.05) and by GeneCluster (if the GeneCluster permutation-based p value criterion is relaxed to <0.1). For the purposes of discussion and for comparison with ANOVA, therefore, the list of genes selected by GeneCluster as associated with a decreased level of expression in encephalitis patients (permutation-based p value <0.1) were compiled and analyzed. The 50 genes most closely associated with decreased levels of expression in encephalitis patients (all of which met the permutation-based p value <0.1 criterion) are shown in Table 12.

### Example 1.4.1.3

### Comparison of Genes Identified through ANOVA and GeneCluster Analyses

To assess the overlap in the list of genes identified by ANOVA and GeneCluster, the list of 113 genes identified by ANOVA with FDR<0.05 (Table 10) was compared to the lists of genes associated with encephalitis by GeneCluster analyses. Of the 200 genes identified in GeneCluster as most closely associated with elevated levels of expression in encephalitis patients, 59 overlapped with the 68 genes identified by ANOVA as having elevated levels of expression in encephalitis patients and FDR<0.05. Of the 200 genes identified in GeneCluster as most closely associated with decreased levels of expression in encephalitis patients, 44 overlapped with the 45 genes identified by ANOVA as having decreased levels of expression in encephalitis patients and FDR<0.05. By this method of assessing overlap, therefore, 91% (103 out of 113) of the most significant genes identified by ANOVA analysis were also selected by the GeneCluster application.

### Example 1.4.1.4

### Expression Patterns of Genes Associated with Encephalitis by ANOVA and GeneCluster

A detailed examination of the expression patterns of the genes listed in Tables 10, 11, and 12 reveals relevant information that is not apparent through mere survey of the p values. First, the gene expression profiles of the five encephalitis patients appear to fall into two fairly distinct patterns. The expression profiles of encephalitis patients 19, 33, and 503 are more similar to each other than they are to the profiles of encephalitis patients 299 and 301. In addition, the profiles of patients 19, 33 and 503 deviate from normal more often than those of patients 299 and 301. For approximately 73% of the genes shown in Table 10, at least three encephalitis patients (usually patients 19, 33, and 503) express at levels associated with encephalitis. Examples of this expression pattern are shown in FIGS. 4-9. (For many of the remaining 27% of genes listed in Table 10, abnormal gene expression levels were observed in only one or two of the encephalitis patients. These genes are not addressed further.)

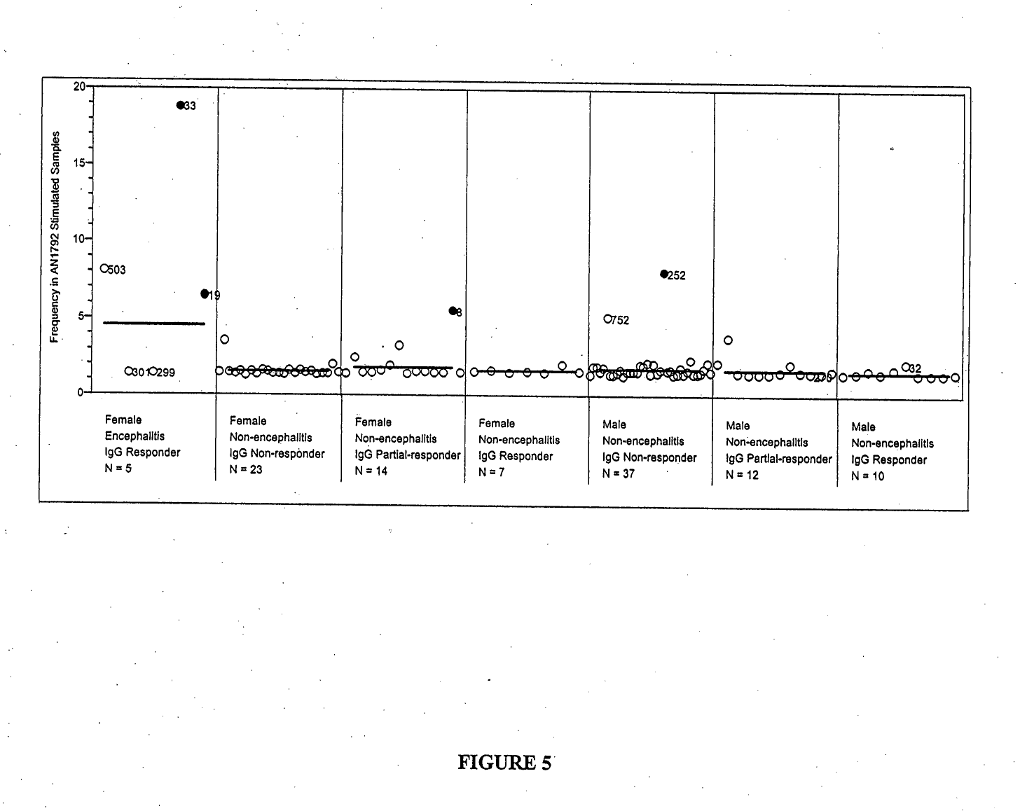

FIG. 4 shows the expression pattern of TPR in samples stimulated overnight in culture with AN1792 and the cocktail of immune adjuvants described in materials and methods (Example 1.1.3). TPR, translocated promoter region, also called tumor-potentiating region, has been implicated in oncogenesis involving the met oncogene. For this figure, as for subsequent figures (FIGS. 5-13), the following description applies. Frequency values are reported as ppm. The horizontal line represents the geometric mean frequency for that group. The vertical lines separate patient groups. The seven patients groups are: 1) female encephalitis patients, 2) immunized IgG titer negative (i.e., maximum titer≦200) females, 3) immunized female patients with maximum IgG titer>200<2200, 4) immunized female patients with maximum IgG titer≧2200, 5) immunized IgG titer negative (i.e., maximum titer≦200) males, 6) immunized male patients with maximum IgG titer>200<2200, and 7) immunized male patients with maximum IgG titer≧2200. The open circles represent absent calls; the closed circles represent present calls. Note the high probability of false absent calls; an increased number of false negative calls (transcripts called absent when actually present) results from the extreme 3′ bias introduced by the two-round IVT protocol. Due to the small amounts of sample available, the two-round IVT protocol was necessary.

FIG. 5 shows the expression pattern of NKTR in samples stimulated overnight in culture with AN1792 and the cocktail of immune adjuvants described above in Example 1.1.3. NKTR, natural killer tumor recognition sequence, also known as natural killer triggering receptor, is involved in the activation of the innate immune system.

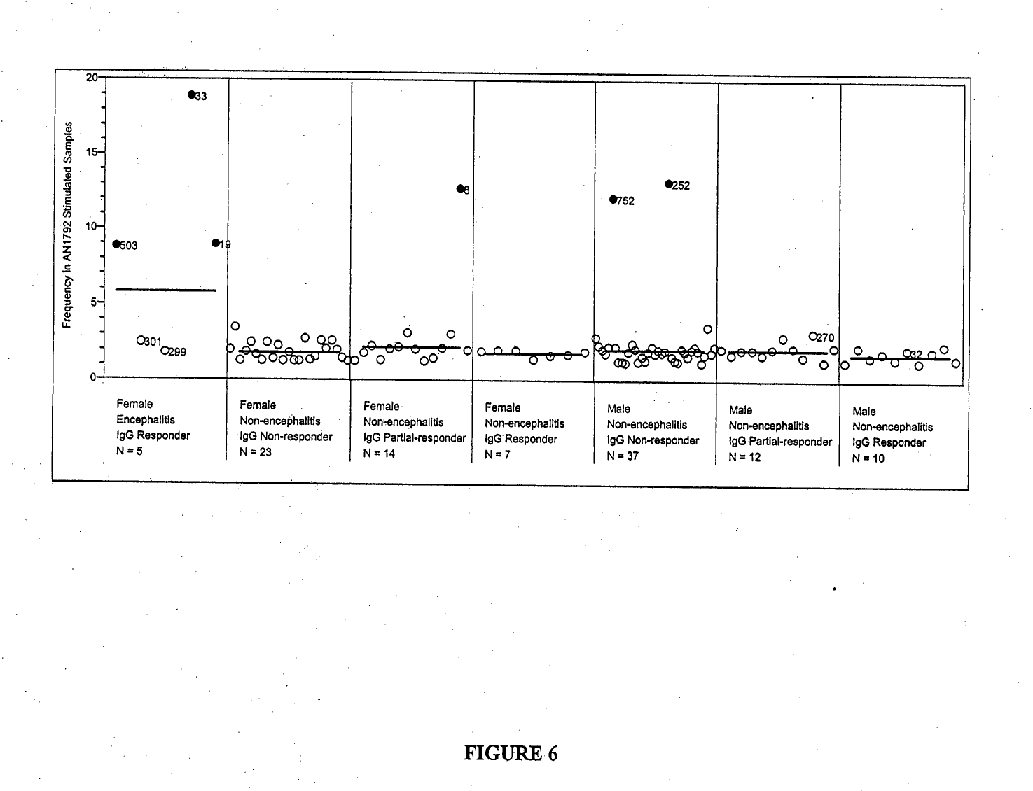

FIG. 6 shows the expression pattern of XTP2 in samples stimulated overnight in culture with AN1792 and the cocktail of immune adjuvants described above in Example 1.1.3. XTP2, HbxAg transactivating protein 2, is thought to be implicated in cell activation events associated with hepatitis B virus infection.

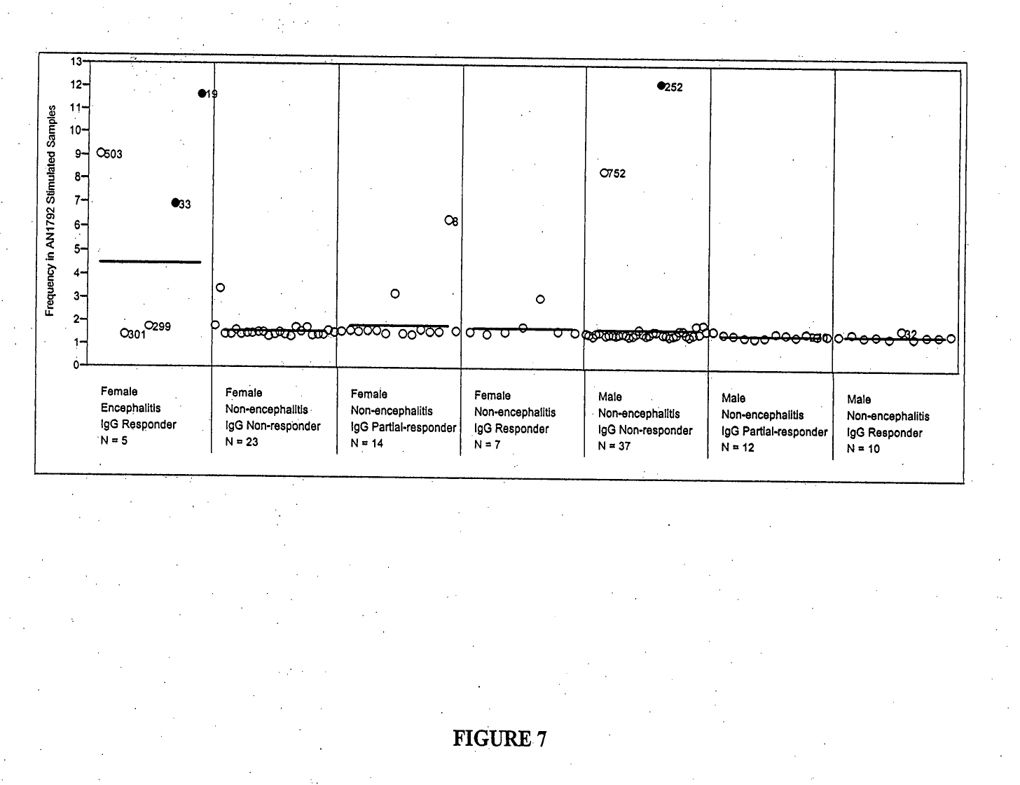

FIG. 7 shows the expression pattern of SRPK2 in samples stimulated overnight in culture with AN1792 and the cocktail of immune adjuvants described above in Example 1.1.3. SRPK2, SFRS protein kinase 2 (protein kinase, arginine/serine splicing factor 2), has been implicated in posttranscriptional regulation of gene expression.

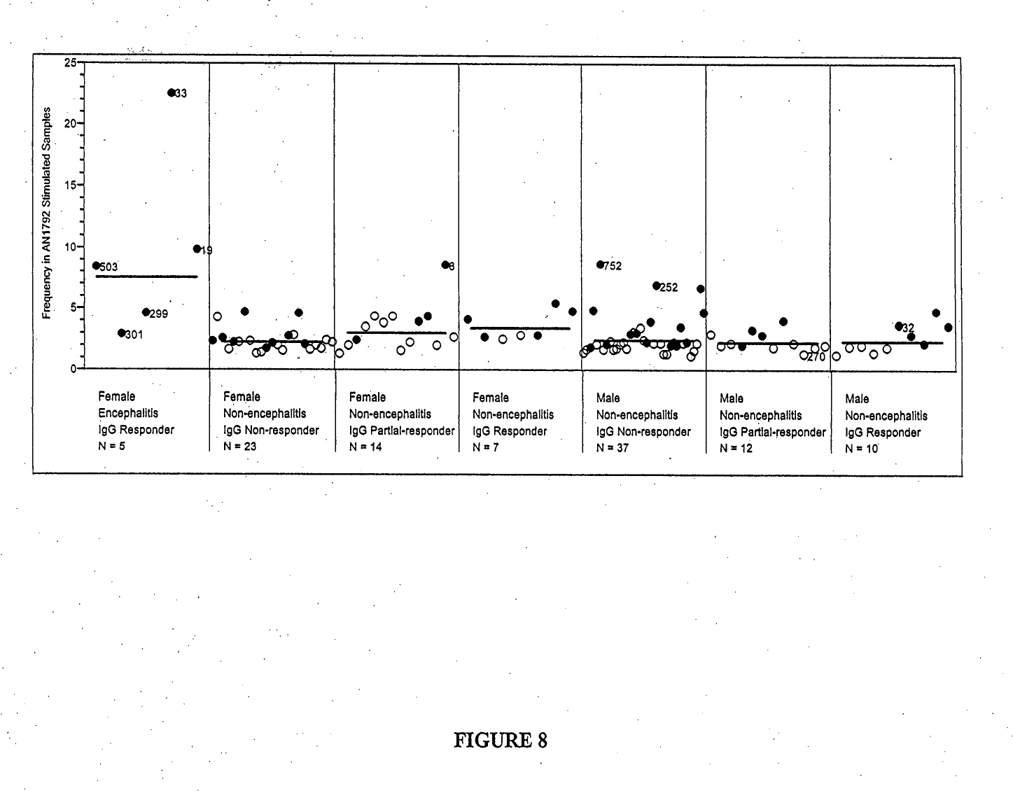

FIG. 8 shows the expression pattern of THOC2 in samples stimulated overnight in culture with AN1792 and the cocktail of immune adjuvants described in Example 1.1.3. THOC2, THO complex 2, has been implicated in the control of gene transcription.

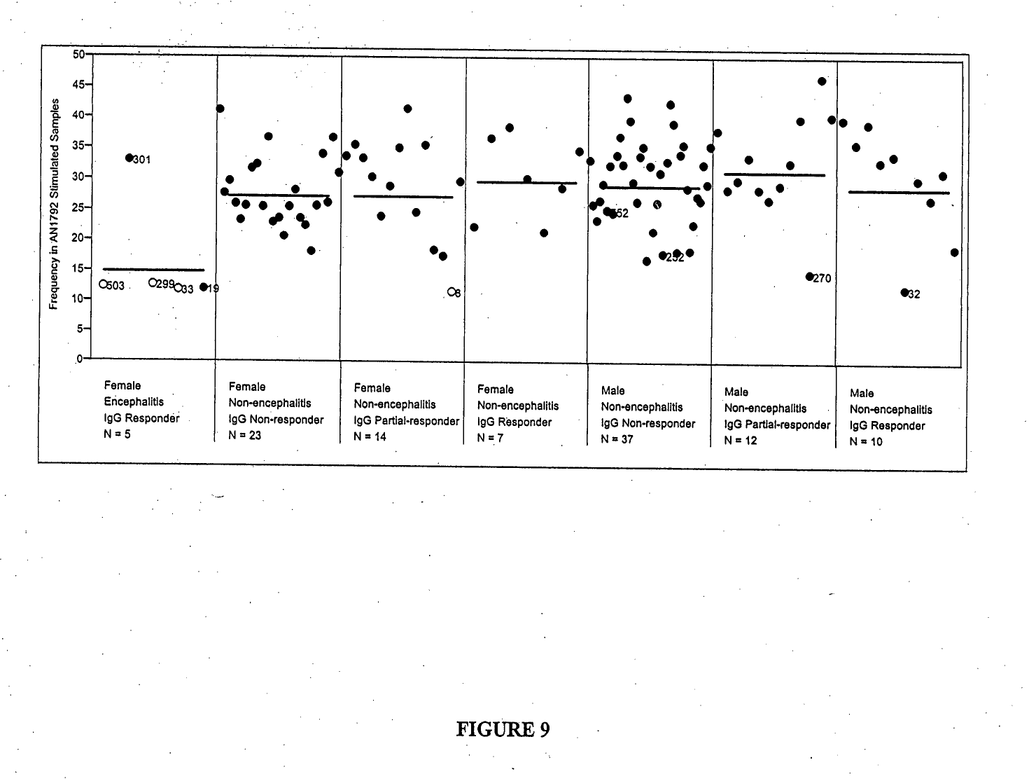

FIG. 9 shows the expression pattern of PSME3 in samples stimulated overnight in culture with AN1792 and the cocktail of immune adjuvants described in Example 1.1.3. PSME3, proteasome activator subunit 3, is a subunit of the protease responsible for the generation of peptides loaded onto MHC class I molecules.

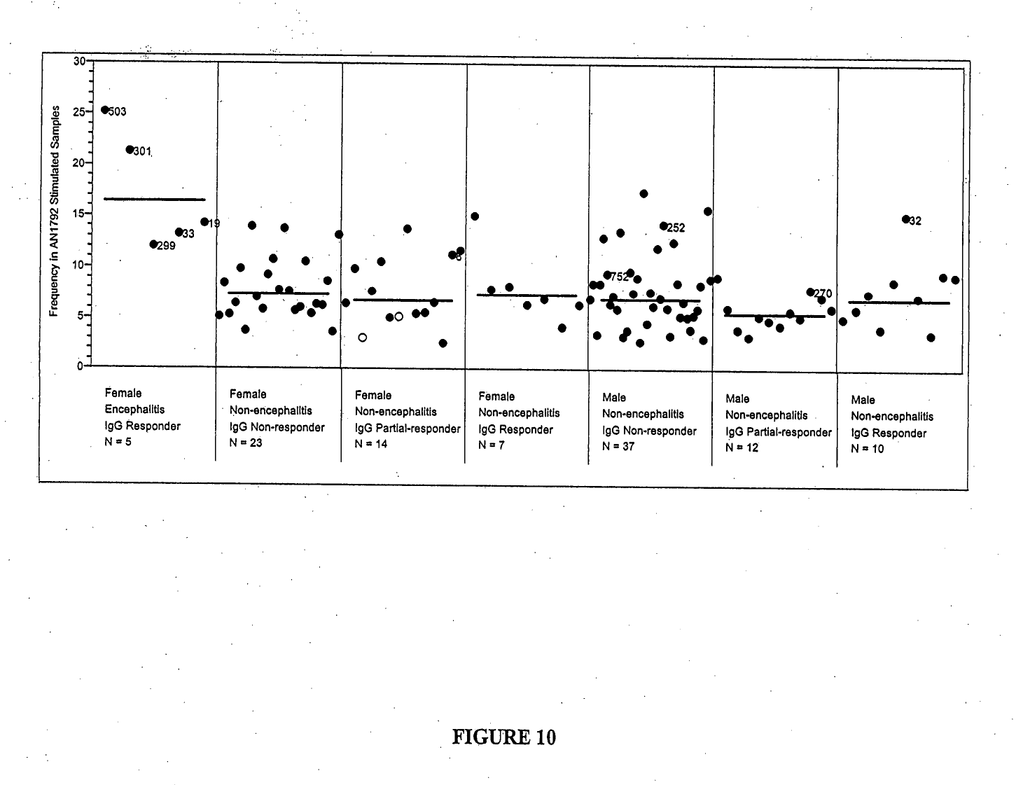

Four of the encephalitis patients (usually patients 19, 33, 299 and 503) express 23% of the genes listed in Table 10 at levels associated with encephalitis. Patient 301 is much less clearly distinguishable from nonencephalitis patients by gene-expression profile. A total of 14 (12%) of the genes listed in Table 10 are expressed by all five patients at levels associated with encephalitis. However, the expression levels associated with encephalitis for these 14 genes are less distinct between the encephalitis and nonencephalitis groups than for genes that capture only three or four of the encephalitis patients. These 14 genes are listed in Table 13. Examples of the expression patterns for four of these genes are shown in FIGS. 10 through 13.

FIG. 10 shows the expression pattern of DAB2 in samples stimulated overnight in culture with AN1792 and the cocktail of immune adjuvants described in Example 1.1.3. DAB2, disabled homologue 2, mitogen-responsive phosphoprotein, competes with SOS for binding to GRB2 and thus is implicated in control of growth rate.

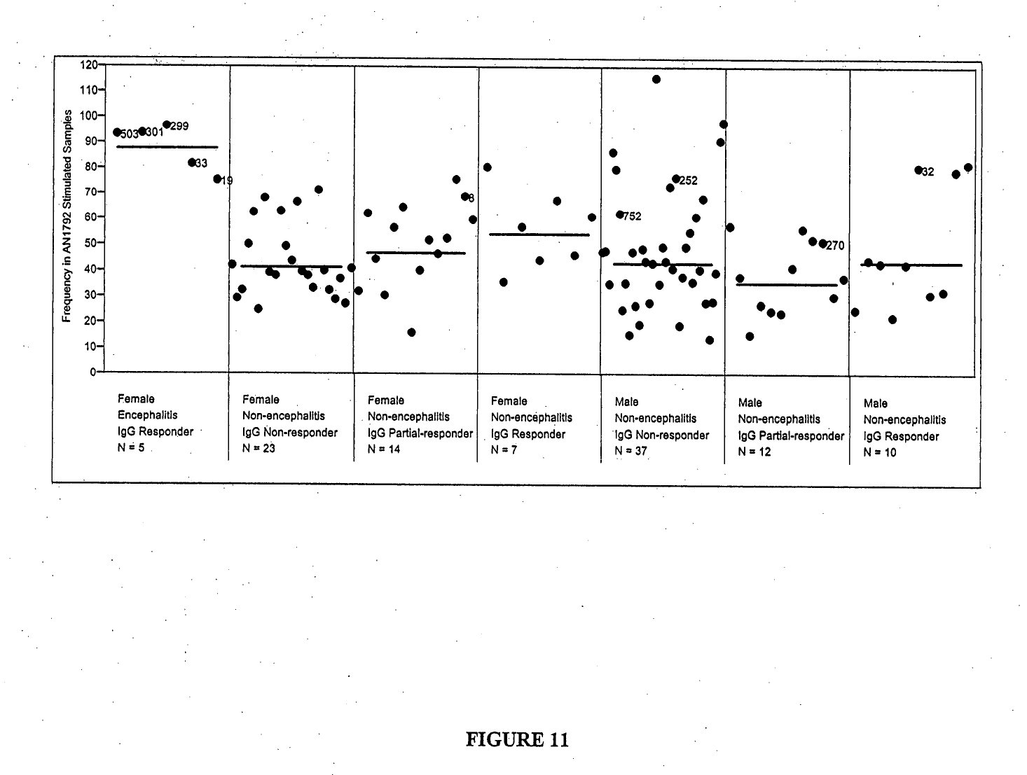

FIG. 11 shows the expression pattern of SCAP2 in samples stimulated overnight in culture with AN1792 and the cocktail of immune adjuvants described in Example 1.1.3. SCAP2, src family-associated phosphoprotein 2, is an adaptor protein thought to play an essential role in the src-signaling pathway.

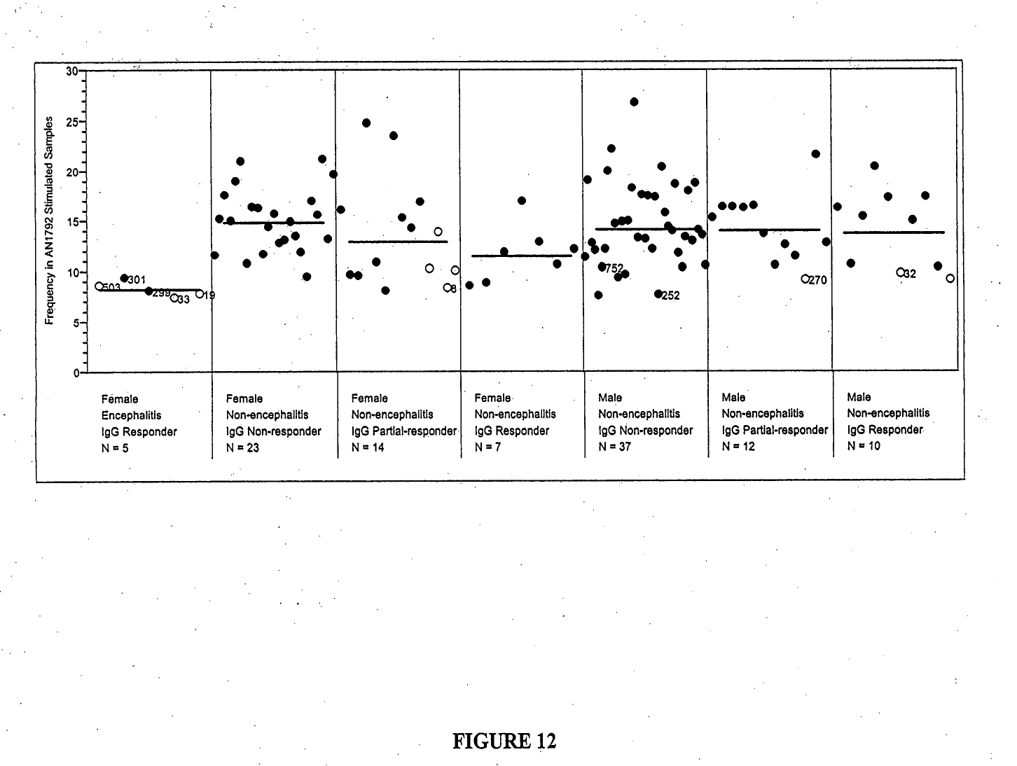

FIG. 12 shows the expression pattern of furin in samples stimulated overnight in culture with AN1792 and the cocktail of immune adjuvants described in Example 1.1.3. Furin is a processing enzyme involved in activation of TGF1, an anti-inflammatory cytokine.

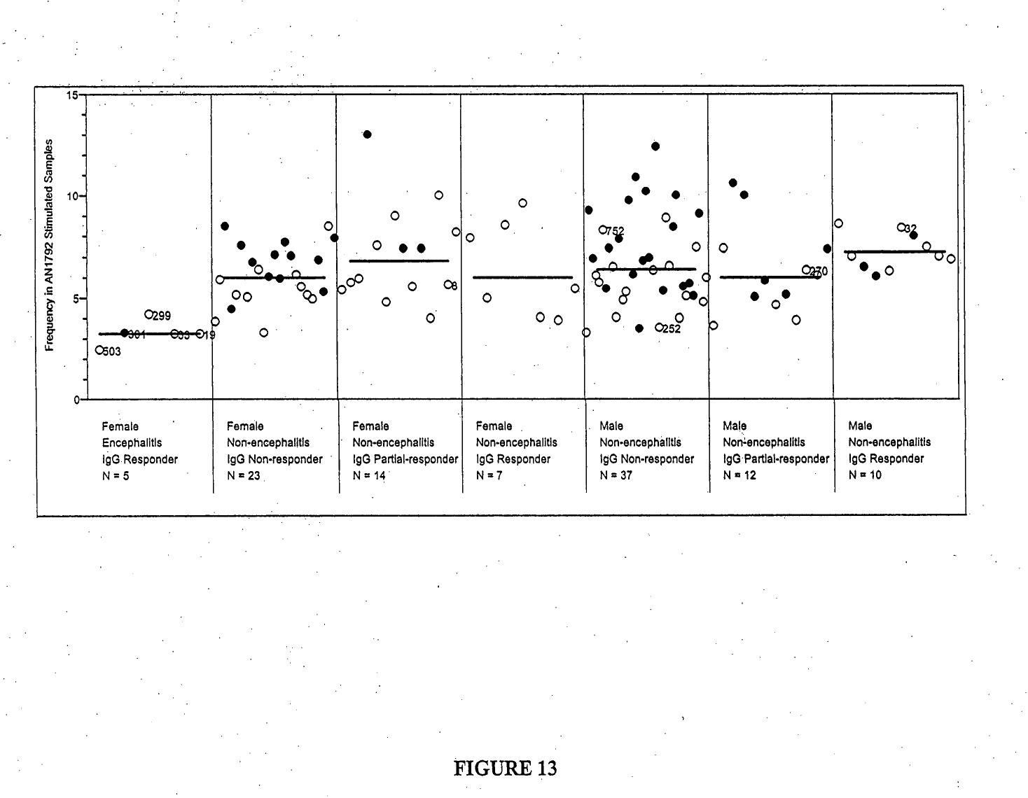

FIG. 13 shows the expression pattern of CD54 (ICAM1) in samples stimulated overnight in culture with AN1792 and the cocktail of immune adjuvants described in Example 1.1.3. CD54, intracellular adhesion molecule 1 (ICAM1), is a ligand for lymphocyte function-associated antigens and is involved in response to antigen.

As encephalitis patient 301 expresses only 12% of the genes listed in Table 10 at levels associated with encephalitis, the expression profile of this patient can be considered more “normal” than the profiles of the other encephalitis patients. Of the five encephalitis patients, patient 33 expressed the most genes (105 of 113) listed in Table 10 at levels associated with encephalitis. The ranking of encephalitis patients in terms of most genes expressed at levels associated with encephalitis is: 33, 19, 503, 299 and 301.

A second trend in gene expression profiles that is not apparent through survey of the statistical associations emerges from the examination of the expression levels of genes associated with encephalitis in individual AN1792 nonencephalitis patients. Data from males were not used to identify genes associated with encephalitis, because all the encephalitis patients in the study were female and the comparator group used was the 44 female AN1792 nonencephalitis patients. Although all the encephalitis patients were female in this study, it is not believed at this time that gender plays a role in predicting whether a patient will develop encephalitis, because in the European Phase IIA clinical trials several males developed encephalitis. Examination of the profiles in males, therefore, offers an opportunity to assess whether samples that were not used to identify associations with encephalitis have profiles consistent with those identified through analysis of the female samples. Table 14 depicts the level of agreement in terms of gene expression profile and clinical diagnosis of encephalitis when the data are analyzed with the inclusion of male nonencephalitis patients (Table 14 is discussed further below in Example 1.8.1).

Using the genes that capture the three most severe encephalitis patients (19, 33, and 503), the false positives are restricted to a few (three or four) patients, and it is often the same three or four patients captured. IgG nonresponding male patients 252 and 752, and partial responding female patient 8 (maximum IgG titer 208) express many of the genes most closely associated with encephalitis at or close to the levels associated with encephalitis. As seen in Table 14 and discussed above, genes that capture all five encephalitis patients also capture an increased number of nonmeningoencephalitic patients, and IgG responders are among the nonencephalitis patients captured. (For example, patients 5, 12, 32, 508, and 755 are IgG responding nonencephalitis patients who express some genes at levels associated with encephalitis.) Another set of genes is the set consisting of the three genes that correctly classify 60% of the encephalitis developer patients and incorrectly classify 4% of the encephalitis nondeveloper patients (i.e., SRPK2, TPR, and NKTR). Another set of genes is the set consisting of the three genes that correctly classify 100% of the encephalitis developer patients, and incorrectly classify 25% of the encephalitis nondeveloper patients (i.e., SCAP2, PACE (furin), and DAB2). Another set of genes is the set consisting of SRPK2, TPR, NKTR, SCAP2, PACE (furin), and DAB2.

### Example 1.4.1.5

### Comparison of Gene Expression Patterns in AN1792-Stimulated and Control Cultures

The identification of gene expression patterns associated with encephalitis in cultures stimulated with AN1792 raised the question of whether in vitro stimulation with AN1792 was required for detection of encephalitis-associated gene expression patterns. To answer this question, the expression patterns in control cultures of the genes associated with encephalitis by the metric of gene frequency in AN1792-stimulated cultures were analyzed. Table 15 shows the association between encephalitis and the metric of frequency in control cultures for 23 of the genes most closely associated with encephalitis by the metric of frequency in the control cultures (for genes that are also shown in Table 10).

This result indicates that detection of statistically significant associations between preimmunization gene expression and postimmunization development of encephalitis may not require in vitro stimulation with AN1792. Of the 113 genes associated with encephalitis using the metric of gene frequency in AN1792-stimulated cultures, 64 genes also show an association using the metric of gene frequency in control cultures (setting the cutoff at raw p<0.005 (FDR<0.18)). The detection of the association with encephalitis in both the AN1792-stimulated and control cultures is evidence both that the associations can be detected without in vitro exposure to AN1792 and that, since the associations have been detected in two sets of samples, the associations have sound technical and statistical support.

The analysis of the control cultures also reveal genes that, whereas associated with encephalitis using the AN1792-stimulated culture frequency metric, show absolutely no association using the metric of frequency in control cultures. The 12 most extreme examples of this gene expression pattern are shown in Table 16. Note that two of the genes in Table 16, PSMF1 and TAP2, are functionally related to antigen processing.

### Example 1.4.2

### Using the Metric of Ratio of the Frequency in AN1792-Stimulated Samples to the Frequency in Control Culture Samples to Identify Gene Expression Levels with Association to Encephalitis

The logarithm of the ratio of the gene frequency of the AN1792-stimulated culture to the gene frequency of the control was calculated for each gene for the five female encephalitis patients and the 44 treated nonencephalitis female patients. This is equivalent to the difference between the logarithms of the gene frequencies for the two culture conditions.

### Example 1.4.2.1

### ANOVA

By this ratio metric, ANOVA found no association with encephalitis with FDR<0.05. The lowest (best association) was FDR=0.104, and there were five genes at this FDR value. This result indicates that the association found by ANOVA did not reach the level of statistical significance (0.05) stipulated in the pharmacogenomic supplemental statistical analysis plan of this study. This finding is consistent with the result (noted above) indicating the detection of strong associations between encephalitis and gene expression levels in control (i.e., without AN1792) stimulated cultures.

### Example 1.4.2.2

### GeneCluster Analysis

By the ratio metric, GeneCluster identified 13 genes that were associated with encephalitis with a permutation-based p value <0.05. The permutation-based p value was >0.01 for all 13 genes listed. These 13 genes, along with their associated raw (unadjusted) p and FDR values by ANOVA, are shown in Table 17. For all genes listed, AN1792 stimulation resulted in a decrease in gene expression frequency. Note that the associations with encephalitis detected using the ratio metric are much weaker (both by ANOVA and GeneCluster) than the associations detected using the frequency metric, again indicating that exposure to antigen (AN1792) in vitro may play a minor role in revealing the associations between gene expression and postimmunization development of encephalitis.

### Example 1.5

### Results—Gene Expression Association with IgG Responsiveness

### Example 1.5.1

### Gene Expression Levels Showing Association with IgG Responsiveness Using the Metric of Gene Frequency in AN1792-Stimulated Cultures

The goal of the search for correlates with antibody response was to identify markers that would allow the preimmunization identification of likely nonresponders in what was, at the onset of this study, a planned Phase III study. If the incidence of nonresponders could be lowered through a prescreening test, the power of the clinical trial could be increased.

### Example 1.5.1.1

### ANOVA

ANOVA was performed by comparing data from the 60 nonresponders (maximum titer≦200) to the 22 IgG responders (maximum titer≧2200) and the 60 nonresponders to the 26 IgG partial (or low) responders (maximum IgG titer>200 and <2200). ANOVA identified 375 genes associated with IgG responsiveness with FDR<0.05 (raw p<0.000919). These data indicate numerous statistically significant differences between IgG responders and nonresponders in the preimmunization PBMC gene expression profiles. However, this number of genes far exceeds the number required to reach the goal of identifying a small geneset associated with likely nonresponsiveness; thus, Table 18 lists only the 15 genes associated with IgG responsiveness by ANOVA with FDR<0.011. The adjusted p values (by Westfall and Young stepdown bootstrap procedure for multiplicity adjustment) for these 15 genes are also shown in Table 18. Note that 11 of the genes listed show an association with IgG response with adjusted p≦0.05.

### Example 1.5.1.2

### GeneCluster Analysis

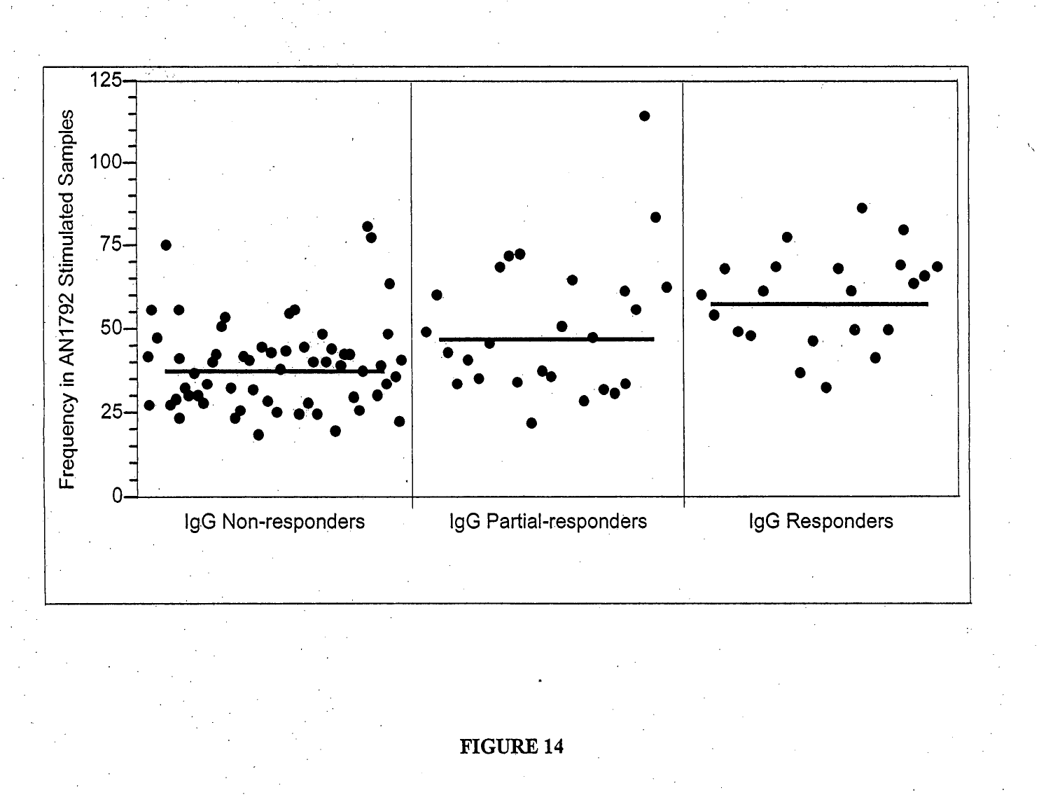

By GeneCluster analysis, more than 500 genes showed an association between gene expression level and IgG response at the 0.01 level of significance. For a more focused analysis, genes associated with a permutation-based p value <0.00005 were selected. (This significance level indicates that the GeneCluster score for the gene is higher than observed in the top 0.005 percentile of randomly permuted data.) At this extremely stringent level of significance, four genes showed association with IgG response. These were granulin, FC fragment of IgG receptor transporter alpha (FCGRT), isoleucine-tRNA synthetase (IARS), and minichromosome maintenance, S. cerevisiae homolog 3 (MCM3). These four genes were also among the 11 most significant associations identified through ANOVA (see Table 18). The gene expression frequencies of the four genes significant at the 0.00005 level by GeneCluster analysis are shown in FIGS. 14-17 for each of the patients in the analysis.

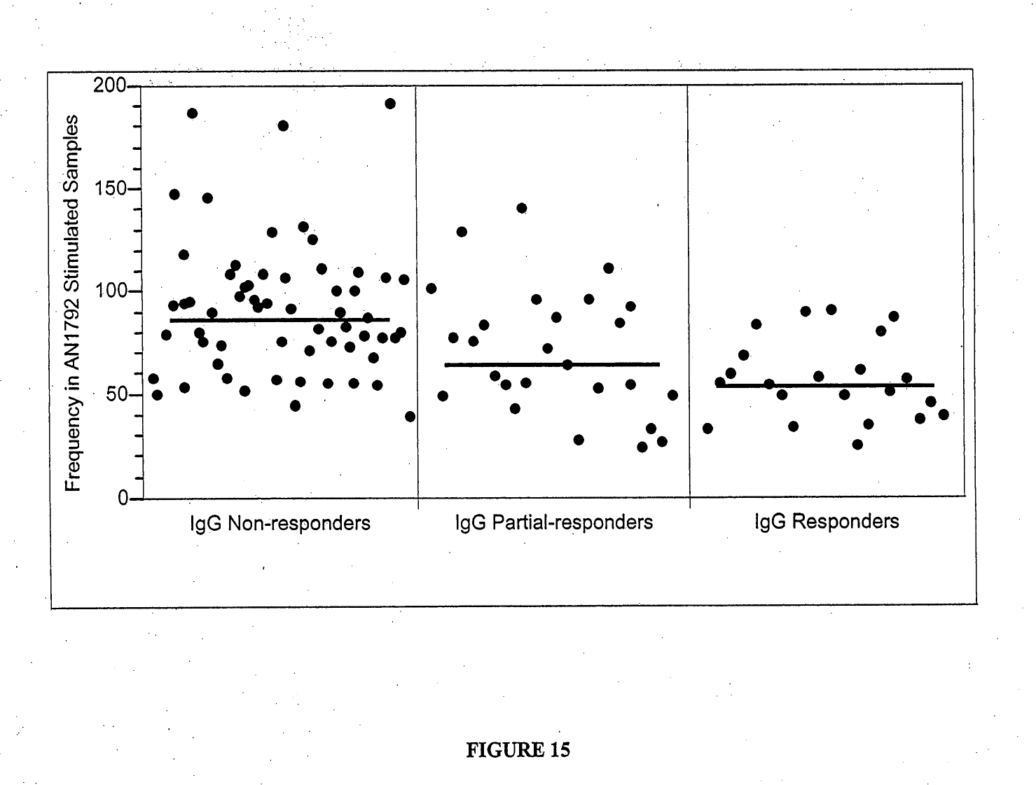

FIG. 14 shows the gene expression levels of IARS, isoleucine-tRNA synthetase, (in individual patients by response group) in samples stimulated overnight in culture with AN1792 and the cocktail of immune adjuvants described in Example 1.1.3. For this figure, as for subsequent figures (FIGS. 15-17), the following description applies. Frequency values are reported as ppm. The horizontal line represents the geometric mean frequency for that group. IgG nonresponders: maximum titer≦200; partial IgG responders: maximum titer>200<2200; IgG responders: maximum titer≧2200.

FIG. 15 shows the gene expression levels of FCGRT, Fc fragment of IgG receptor transporter alpha, in samples stimulated overnight in culture with AN1792 and the cocktail of immune adjuvants described in Example 1.1.3.

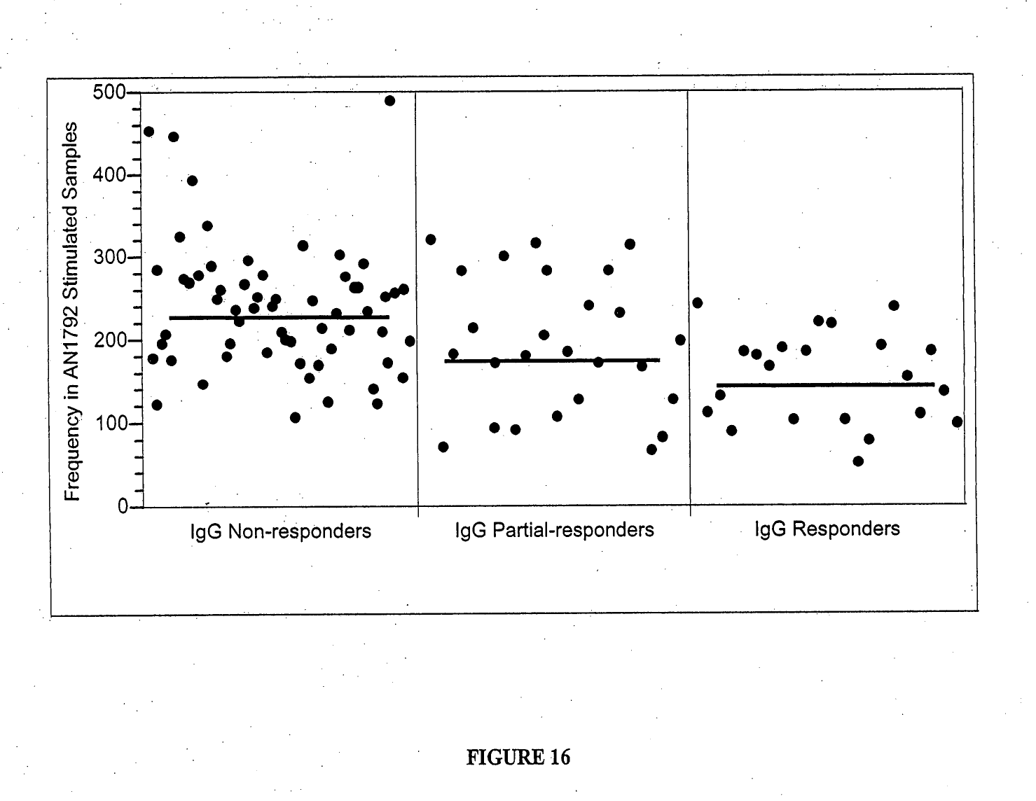

FIG. 16 shows the gene expression levels of granulin in samples stimulated overnight in culture with AN1792 and the cocktail of immune adjuvants described in Example 1.1.3.

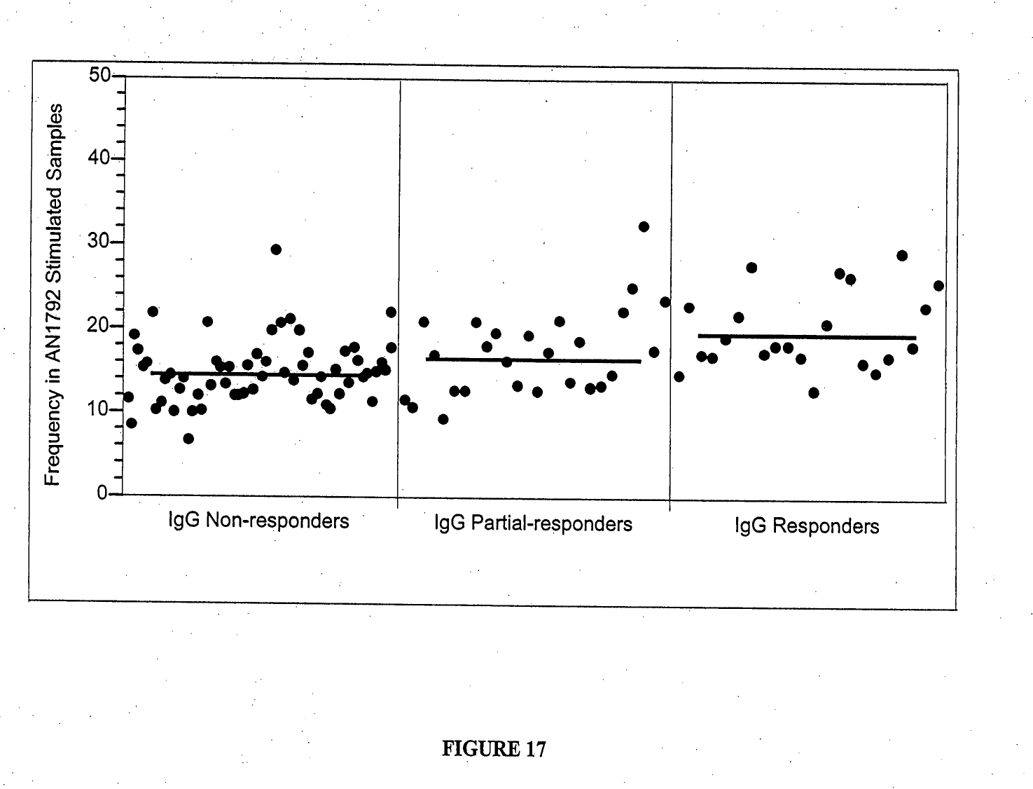

FIG. 17 shows the gene expression levels of MCM3 (thought to be involved in the DNA replication process) in samples stimulated overnight in culture with AN1792 and the cocktail of immune adjuvants described in Example 1.1.3.

FIGS. 14 through 17 show that, for the set of samples in this study at least, nonresponsiveness is associated with high expression levels of granulin and FCGRT and low expression levels of IARS and MCM3. Expression levels of partial responders (maximum IgG titer>200<2200) are intermediate between nonresponders and responders.

Increasing the permutation-based p value from 0.00005 (four genes identified) to 0.00007 in GeneCluster results in an increase of 226 in the number of genes identified. The large number of genes identified at the 0.00007 level of significance (also an extremely stringent criterion) reflects numerous differences in gene expression between the IgG responder and nonresponder groups. Of the 230 genes identified in GeneCluster at the 0.0007 significance level, 217 were also identified as associated by ANOVA, indicating a high concordance between the genes identified by the two applications. This level of concordance is similar to that observed for the associations identified between gene expression profiles and encephalitis.

### Example 1.5.1.3

### Correlation Between Expression Levels and IgG Response Group

The data in Table 18 and FIGS. 14-17 suggest that preimmunization gene expression profiling has the potential to identify a fraction of the population least likely to respond. Therefore, using the four genes identified by GeneCluster analysis, the correlation between expression levels and IgG response groups was assessed. Table 19 shows the correlation between expression pattern and IgG responsiveness of the individual genes for the four genes identified by GeneCluster analysis.

### Example 1.5.2

### Using the Metric of Ratio of the Frequency in AN1792-Stimulated Samples to the Frequency in Control Culture Samples to Identify Gene Expression Levels With Association to IgG Responsiveness

ANOVA and GeneCluster analyses were run using the metric of the ratio of gene frequency values in AN1792-stimulated cultures to gene frequency values in control cultures. Neither analysis revealed association that met the 0.05 significance level cutoff. These data indicate that the associations found using the gene frequency metric were not dependent on in vitro stimulation with AN1792.

### Example 1.6

### Results—Lack of Association Between Gene Expression Pattern and IgM Responsiveness

ANOVA and GeneCluster analyses were performed comparing treated IgM responders and nonresponders. Both the metric of gene frequency in AN1792-stimulated samples and the metric of the ratio of the gene frequency of the AN1792-stimulated culture to the gene frequency of the control were used in these analyses. No association was found in which FDR<0.05 (ANOVA) or permutation-based p value <0.05 (GeneCluster).

### Example 1.7

### Results—Lack of Association Between Gene Expression Pattern and the Presence of the ApoE4 Allele

ANOVA was performed comparing gene expression patterns of ApoE4 homozygous, ApoE4 heterozygous, and ApoE4 negative patients. Data from both treated and placebo patients were included in this analysis. GeneCluster analysis was performed comparing ApoE4 negative patients to ApoE4 positive patients. Both the metric of gene frequency in AN1792-stimulated samples and the metric of the ratio of the gene frequency of the AN1792-stimulated culture to the gene frequency of the control were used in these analyses. No association was found that met the 0.05 level of significance. In fact, the two top scoring genes detected by GeneCluster were gender-specific genes encoded by the Y chromosome. The identification of Y chromosome-encoded genes reflects the fact that there are 12 more males than females in the ApoE4 negative group (see Table 9). Therefore, no significant correlation between gene expression pattern in PBMCs and ApoE4 type was detected by this study.

### Example 1.8

### Discussion

### Example 1.8.1

### Gene Expression Patterns Associated with Encephalitis

Based on the evidence showing strong associations between preimmunization gene expression patterns and postimmunization development of encephalitis, there is evidence to suggest that certain genes may be associated with development of encephalitis. These results can be viewed as providing a basis for the formulation of hypotheses that may help explain why some patients were susceptible to the development of encephalitis. The data are consistent with the hypothesis that the patients who developed encephalitis were predisposed to do so because some pathways related to immune function were in a state of increased activation. In assessing this information on gene expression profiles associated with encephalitis, it should be noted that the sample set of five encephalitis patients is extremely small and contains considerable diversity. Also, as treatment was halted after two or three immunizations, it is not known whether other patients would have developed encephalitis had immunizations continued. For example, speculation on the data in Table 14 could either favor the interpretation that gene SCAP2 incorrectly groups 11 of 103 treated nonencephalitis patients with the encephalitis group, or that these 11 additional patients may be at increased risk of developing encephalitis. This study does not provide sufficient data to distinguish between these possibilities. It is also possible that increased risk of encephalitis is correlated with a gene expression profile that requires some combination of genes to be expressed at levels associated with encephalitis. However, as noted above, regardless of the interpretation, the analysis would result in the prediction that certain nonencephalitis-prone patients would likely develop encephalitis, rather than the prediction that encephalitis-prone patients would not get encephalitis. Because the goal of the present invention is to ensure that patients at risk of encephalitis be identified in order to avoid an adverse reaction to immunotherapy and to provide a targeted therapeutic for AN1792, excluding a small percentage of patients that would otherwise be good candidates is within the goal of the present invention.

The results disclosed here do suggest that certain gene expression patterns may be useful in preimmunization assessment of the relative risk of encephalitis. The number of genes associated with FDR<0.05 is large (113 genes), and there is variation among these genes with respect to both the number of encephalitis patients that express at levels associated with encephalitis, and the number of nonencephalitis patients that express at levels associated with encephalitis. Therefore, as an illustrative example, or exercise, regarding the potential for using these data to classify patients, three criteria for inclusion on a selected list of six encephalitis-association genes useful in classification were set; inclusion on the list required meeting either the first and third criteria or the second and third criteria. The first criterion was belonging to the group of genes that capture three of the five encephalitis patients (see, e.g., FIGS. 4-8). The second criterion was belonging to the group of genes that capture all five encephalitis patients (see, e.g., FIGS. 10, 11 and 13). The third criterion was belonging to the group of genes for which statistically significant associations with encephalitis have been observed both in AN1792-stimulated and control cultures (see Table 15). This last criterion increases the likelihood that genes with true associations are selected by requiring that both sets of data pass a rigorous statistical filter.

Using these criteria for inclusion on the list of genes with potential as “risk assessment genes,” the genes TPR, NKTR, XTP-2, and SRPK2 are examples of genes that were included because they met the first and third criteria. DAB2 and SCAP2 are examples that were included because they met the second and third criteria. A list of genes containing these six genes only results in the accurate classification of five out of five encephalitis patients, and incorrectly classifies about 25-30% (depending on the cutoff) of nonencephalitis patients (see also Table 14).

ASRGL1 is the gene most closely associated with encephalitis by ANOVA (see Table 10), and also shows an extremely strong association by GeneCluster analysis (see Table 11). Inclusion of this single gene on the list of potential “risk assessment genes” would raise the misclassification rate among nonencephalitis patients to about 40%. However, as noted in the footnotes to Table 14, the preponderance of the misclassified patients are male. (With a cutoff of F>20, 100% of the patients misclassified by this gene are male. With a cut-off of F>12, 64% of the misclassified patients are male.) To a great extent, two facts explain the high false positive rate when ASRGL1 is included in the set of genes used for risk assessment: (1) that data from female patients only was used to calculate the strength of the association with encephalitis, and (2) that high levels of expression in nonencephalitis patients are strongly associated with being male. These issues call into question the true strength of the association between ASRGL1 and encephalitis. Three possibilities regarding why high levels of ASRGL1 are extremely strongly associated with encephalitis in females but not in males are: (1) the data reflect a true gender difference, (2) identification of ASRGL1 is a false positive (noting that the FDR<0.05 cutoff allows for the false identification of about six genes), and (3) the association exists but is much less strong than when calculated excluding males.

The findings by GeneCluster are consistent with the findings by ANOVA in that both show numerous differences in gene expression between the meningoencephalitic and nonmeningoencephalitic groups. Genes selected by ANOVA are not expected to be identical to genes selected by GeneCluster due to the differences in algorithms used to select the genes and the nonequivalent methods of calculating p values. However, it is of interest to compare the lists of genes identified by ANOVA and GeneCluster because the level of overlap between the gene lists gives both an indication of the robustness of the methods and an understanding of differing weights given to pattern recognition by each of the approaches. GeneCluster places greater weight than ANOVA on the requirement that all five encephalitis patients group together with respect to the expression frequency of the identified gene. ANOVA places greater weight than GeneCluster on outliers (compared to nonencephalitis patients) even if only one or two of the encephalitis patients express at levels deviant from normal. Therefore, as a result of the different algorithms used by the two applications, both applications identify as associated with encephalitis genes where three of the five encephalitis patients express at levels outside the normal range, but ANOVA will tend to identify the encephalitis association more strongly than will GeneCluster. GeneCluster, on the other hand, will rank more highly genes that are expressed at similar levels by all five encephalitis patients, even if the average expression level in encephalitis patients falls at the outer limits of the range within normal patients.

Many of the statistically significant associations between gene expression patterns that were observed in the gene frequencies in cultures stimulated in vitro with AN1792 were also observed in control cultures that were not exposed to AN1792. This result indicates that detection of many aspects of the gene expression profile associated with a predisposition to the development of encephalitis does not require in vitro exposure to AN1792. This conclusion is also consistent with the results using the ratio metric (fold change in frequency in AN1792-stimulated cultures as compared with control cultures). The ratio metric revealed no association meeting the FDR<0.05 level by ANOVA, and the associations revealed by GeneCluster were much less robust than those identified using the frequency metric.

### Example 1.8.2

### Biological Pathways Associated with Encephalitis

Caution must be exercised in drawing conclusions on biological mechanisms based solely on gene expression profiles. The gene expression profiles of the encephalitis patients indicate that these patients may be prone to process and react differently to antigen. Examination of the expression levels of ICAM1 (FIG. 13), PSME3 (FIG. 9) and XTP2 (FIG. 6) illustrate this point. There may also be differences in the innate immunity pathway (see FIG. 5 for the NKTR expression profile).

Many of the genes showing the most significant association with encephalitis are functionally related to the control of transcription. The identified differences in gene expression patterns could therefore be the result of activation (or deactivation) of genes under common transcriptional control. This interpretation fits with the observation that certain genesets show a consistent pattern in certain patients (for example patients 8, 19, 33, 252, 503, and 752), hinting that these genesets are behaving as a correlated set in a small number of patients. This type of correlation is well recognized in gene expression analysis, and is factored in the algorithms used by GeneCluster.

There is also some suggestion within the data that patients that express a significant number of genes at levels associated with encephalitis may be at reduced risk if they do not develop a significant IgG titer (≧2200). Patients 8, 252 and 752 fall into this category. This hypothesis fits with the clinical information that, whereas IgG responders most often do not develop encephalitis, those patients who do develop encephalitis are likely to have significant IgG titers.

The genes identified as associated with encephalitis by the ratio metric of frequency in AN1792-stimulated cultures to frequency in control cultures are functionally related to immune function including response to cytokines, control of apoptosis and chemotaxis, signal transduction and control of proliferation. These data are consistent with a difference between nonencephalitis and encephalitis patients in terms of immune system response to exposure to AN1792, but the associations found are relatively weak.

### Example 1.8.3

### Gene Expression Profile Associations with IgG Nonresponsiveness

Both GeneCluster and ANOVA indicate that there are numerous statistically significant differences between the preimmunization gene expression profiles of IgG responders and nonresponders. These numerous differences may be a reflection of a few different biological pathways being activated in the two groups. This kind of difference can result in activation and deactivation of genes that are under common transcriptional control and consequently behave as correlated sets. This type of correlation is well recognized in gene expression analysis, and is factored in the algorithms used by GeneCluster. Many of the genes showing the most significant association with IgG nonresponsiveness are functionally related to the control of transcription.

The association between high levels of FCGRT with IgG nonresponsiveness is an intriguing finding. This gene is believed to function in the transport of IgG in some forms of immunity. The association of low levels of IARS with nonresponsiveness is another fascinating and unexpected finding. The autoimmune diseases polymyositis and dermatomyositis are a consequence of autoantibodies directed against one or more of the aminoacyl-tRNA synthetases with subsequent lymphocytic destruction of myocytes. Six of 20 human aminoacyl-tRNA synthetases have been identified as targets in these autoimmune diseases. In light of this information, the association identified in this study between low levels of IARS and IgG nonresponsiveness suggests that high levels of IARS may be associated with hyperresponsiveness, and the destruction observed in autoimmune disease might be an adaptive response aimed at controlling high activity of this gene. The MCM3 gene is thought to be involved in DNA replication. Thus it is possible that the gene may function in the replication of lymphocytes known to be necessary for T and B cell responses. Low levels of this gene are associated with nonresponsiveness, a finding consistent with the hypothesis that this gene functions in the proliferative phase of the in vivo immune response.

No gene associated with IgG responsiveness was identified by the ratio metric of frequency in AN1792-stimulated cultures to frequency in control cultures. This finding indicates that the gene expression patterns associated with IgG responsiveness are intrinsic characteristics of the patients that do not depend for detection on in vitro exposure to AN1792. It is possible, therefore, that the gene expression profiles associated with IgG responsiveness in this study are general surrogate markers for the ability to respond to immunotherapy. Such markers have not been identified, and these findings, if validated, could help in understanding the incidence of immunotherapeutic nonresponsiveness in general, and especially in the elderly.

No statistically significant association was found between gene expression profiles and IgM response, although the same trend that is statistically significant in the IgG analysis is detectable in the IgM analysis (but does not reach statistical significance). For example, the four highest expressors of FCGRT are IgM nonresponders and IgG nonresponders. The same is true for the four highest expressors of granulin and the five highest expressors of CST3.

### Example 1.9

### Conclusions

By ANOVA and GeneCluster analyses, statistically significant associations have been detected between the gene expression profiles of PBMCs of patients prior to immunization with AN1792 and the postimmunization development of encephalitis. In addition, statistically significant associations were found between the preimmunization gene expression profile in PBMCs and postimmunization development of IgG response.

No statistically significant associations were found between gene expression profiles and either IgM response or ApoE4 type. For many of the genes associated with IgG responsiveness, however, a similar trend is present in the comparison of IgM responders and nonresponders, but the trend does not reach statistical significance for a single gene.

### Example 2

### Association of Gene Expression Profiles of Unstimulated Samples with Either Favorable or Adverse Clinical Responses

### Example 2.1

### Materials and Methods—Sample Collection and Preparation

### Example 2.1.1

### Sample Collection

Consent to the pharmacogenomic portion of the study was optional and obtained after approval by local institutional review boards in the U.S. (E.U. patients were not included in the pharmacogenomic study). All gene expression analyses were conducted on RNA purified from peripheral blood mononuclear cells (PBMCs) collected prior to immunization. Blood samples were collected from consenting subjects at the screening visit (between 9 and 54 days prior to the first immunization) and were shipped overnight at room temperature to the Clinical Pharmacogenomic Laboratory at Wyeth Research in Andover, Mass., and PBMCs were purified as described in Examples 1.1 and 1.1.1 above (see also Burczynski et al. (2005) Clin. Cancer Res. 11:1181-89). CPT purification resulted in greater than 99% reduction in RBC representation in all 153 study samples, and CPT purification did not alter by more than 15% the percentage of monocytes relative to PBMCs. The efficiency of removal of neutrophils by CPT fractionation is shown in FIG. 2 and discussed in Example 1.1.1 (see also Table 2; see generally Example 1.1.3.1). A fraction of the PBMCs (2×10⁶ cells) was pelleted and frozen on dry ice for the isolation of RNA samples. The remaining PBMCs were consigned to in vitro studies (described in Example 1).

### Example 2.1.2

### Sample Preparation: RNA Purification

The purified PBMC fraction was pelleted by centrifugation, resuspended in 300 μl RLT Buffer (Qiagen, Valencia, Calif.) containing 2-mercaptoethanol (the starting buffer for RNA purification), snap frozen and stored at −80° C. prior to gene expression analysis. RNA was purified using QIA shredders and Qiagen RNeasy® mini-kits. In particular, labeled targets for oligonucleotide arrays were prepared using 50 ng of total RNA. Biotinylation of cRNA (generated using two-cycle IVT amplification), hybridization to the HG-U133A Affymetrix GeneChip Array®, and conversion of signal values to normalized parts per million (Hill et al. (2001) Genome Biol. 2:research0055.1-0055.13) are described below. Data for 9,678 probesets that were called ‘present’ and with frequency ≧10 parts per million in at least one of the samples were subjected to the statistical analyses described below, while probesets that did not meet these criteria were excluded. SAS was used for all analyses unless otherwise noted.

### Example 2.1.3

### Sample Preparation: Microarray Targets Labeling

Labeled cRNA for hybridization to microarrays was prepared using a two-round in vitro transcription (IVT) amplification procedure. The two-round procedure was necessary because the RNA yield (from 2×10⁶ starting PBMCs) was less than 1 μg in some cases. Total RNA was converted to 1^{st strand cDNA by priming with} 40 pmol of T7-(dT)₂₄ primer (Genset Corp). Primer and total RNA were incubated at 70° C. for 10 minutes and then held at 50° C. until the addition of first-strand buffer [250 mM Tris-HCl (pH 8.3), 375 mM KCl, 15 mM MgCl₂], 10 mM DTT, 500 μM each of dNTP mix, and 40 U RNAseOUT (all from Invitrogen). Samples were then incubated at 50° C. for 2 minutes followed by the addition of the 200 U of SuperScript™ II Reverse Transcriptase (Invitrogen) and incubation at 50° C. for 1 hour.

Double-stranded cDNA was synthesized by incubating the 1^{st} strand cDNA at 16° C. for 2 hours with second-strand buffer plus, 200 μM of each dNTP, 10 U of E. coli DNA ligase, 40 U of E. coli DNA Polymerase I, 2 U of E. coli Rnase H, (all from Invitrogen), and DEPC-treated water (Ambion) to a final volume of 150 μl. Six units of T4 DNA Polymerase (BioLabs) were then added and samples were incubated for 5 minutes at 16° C. The reaction was stopped by the addition of 20 mM EDTA (Ambion), and samples were placed on ice.

Using paramagnetic beads (Polysciences, Inc.) and a 3-in-1 magnetic particle separator (CPG, Inc), cDNA was purified by solid-phase reversible immobilization (DeAngelis et al. (1995) Nucleic Acids Res. 23:4742-43). Purified cDNA (10 μl) was transcribed into nonlabeled cRNA in an IVT reaction in 0.8×IVT buffer (Ambion), 2.9 mM each of rNTP mix (Amersham), 40 U of RNase Inhibitor (Ambion), 4.3 mM DTT (Invitrogen), 450 U T7 Polymerase (Epicentre) and DEPC-treated water (Ambion) to a final volume of 35 μl and incubation at 37° C. for at least 16 hours.

The nonlabeled cRNA was purified using the Qiagen RNeasy® Mini Kit and RNA cleanup protocol (according to manufacturer's protocol). For the second round of amplification, samples were lyophilized to 10 μl. cRNA was then reverse-transcribed into cDNA using 150 ng of random hexamer (Wyeth) at 70° C. for 10 minutes, and then held at 50° C.

First strand cDNA synthesis for the second IVT procedure was performed in first strand buffer [250 mM Tris-HCl (pH 8.3), 375 mM KCl, 15 mM MgCl₂], 10 mM DTT, 500 μM of each dNTP mix, and 40 U RNAseOUT (all from Invitrogen) with incubation at 37° C. for 2 minutes followed by addition of 200 U SuperScript™ II Reverse Transcriptase (Invitrogen) to a final volume of 20 μl. Synthesis was completed at 37° C. for 1 hour. Two units of E. coli RNase H (Invitrogen) were added and the mixture was incubated at 37° C. for 20 minutes and 95° C. for 2 minutes, and then chilled on ice. Samples were then primed with 20 pmol of T7-(dT)₂₄ Primer (Genset Corp.) at 70° C. for 10 minutes and chilled on ice.

Second strand cDNA synthesis for the second IVT procedure was initiated using second-strand buffer plus, 200 μM each of dNTP, 40 U of E. coli Polymerase I, 2 U of E. coli RNase H, (all from Invitrogen) and DEPC-treated water (Ambion) to a final volume of 150 μl, and incubated at 16° C. for 2 hours. Six units of T4 DNA polymerase (BioLabs) were added and sample was incubated for 5 minutes at 16° C. The reaction was stopped by addition of 20 mM EDTA (Ambion) and samples were placed on ice. cDNA was purified by binding paramagnetic beads as described above. Second-round purified cDNA (10 μl) was transcribed into biotin-labeled cRNA by IVT using 1×IVT buffer (Ambion), rNTP mix containing 3 mM of GTP, 1.5 mM of ATP and 1.2 mM each of CTP and UTP (Amersham), 0.4 mM each of Bio-11 CTP and Bio-11 UTP (Perkin Elmer), 40 U of RNase Inhibitor (Ambion), 10 mM DTT (Invitrogen), 2,500 U T7 Polymerase (Epicentre) and water (Ambion) in a final volume of 60 μl followed by incubation at 37° C. for at least 16 hours. The biotin-labeled cRNA was purified using the Qiagen Rneasy® Mini-kit and RNA cleanup protocol according to manufacturer's instructions. Quantification of cRNA yield was performed using UV absorbance 280/260. Ten μg of labeled cRNA was fragmented in 40 mM Tris-acetate pH 8.0, 100 mM KOAc, 30 mM MgOAc for 33 minutes at 94° C. in a final volume of 40 μl. This labeled target was hybridized with MES buffer, 30 μg herring sperm DNA, 150 μg acetylated BSA, 50 pM Bio 948, and RNase free water to a final volume of 300 μl, then incubated at 99° C. for 10 minutes, and then held at 45° C. for 5 minutes.

### Example 2.1.4

### Sample Preparation: Hybridization of Labeled cRNA to Microarray

Biotinylated cRNA was hybridized to the Affymetrix HG-U133A GeneChip array as described in the Affymetrix Technical Manual.

### Example 2.2

### Materials and Methods—Determination of Gene Expression Patterns

### Example 2.2.1

### Determination of Gene Expression Frequencies

Gene expression frequencies of unstimulated patient samples procured from patients who were IgG and/or IgM (antibody) responders (titer≧2200), partial antibody responders (200≦titer<2,200), antibody nonresponders (titer<200), encephalitis developers and/or encephalitis nondevelopers in response to AN1792 were determined as described above (Example 1.2.1) according to certain inclusion criteria for GeneChip Results, also described above (Example 1.2.2 and Table 3). Briefly, MAS 5.0 software was used to compute signal values (i.e., probe intensities) and absent/present calls for each probeset on each array (marginal calls were counted as absent calls due the filter criteria). MAS 5.0 was also used for the first pass normalization by scaling the trimmed mean to a value of 100. The database processes also calculated a series of chip QC (quality control) metrics and stored all the raw data and QC calculations back to the database. QC metrics were stored with the raw data in the database, e.g., as in Example 1.2.2. The signal values for each probeset were converted to frequency values representative of the number of transcripts present in 10⁶ transcripts (ppm) by reference to a standard curve (see, e.g., Example 1.2.3). Data for 9,678 probesets that were called ‘present’ and with frequency ≧10 ppm in at least one of the samples were included in the study. GeneChip data that passed all quality control criteria, as described in Example 1.2.2, were generated from 123 treated and 30 placebo groups (see Table 3 for GeneChip quality control criteria for study inclusion). SAS was used for all analyses unless otherwise noted.

### Example 2.2.2

### Merging of Clinical and Gene Expression Data

Relevant clinical data pertaining to treatment group, maximum IgG titer for all visits, maximum IgM titer for all visits, encephalitis status, and demographic data were received from StatProbe, Inc. (Ann Arbor, Mich.). The clinical data were merged with the gene expression data by donor identification number. (See also, generally, Example 1.2.5.)

### Example 2.2.3

### Sample Inclusion Criteria and Patient Demographics

### Example 2.2.3.1

### Sample Inclusion Criteria

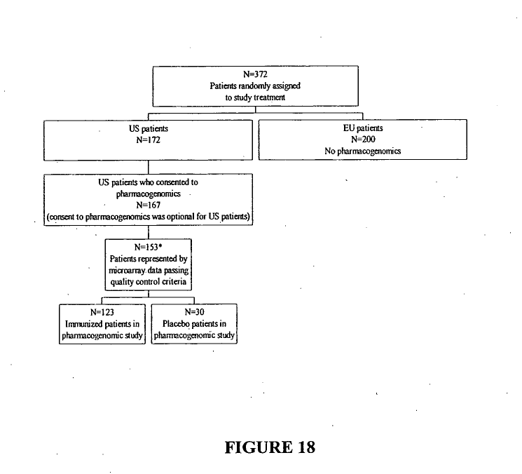

Inclusion for study in this Example 2 required 1) that samples arrive at the Pharmacogenomics Laboratory within one day of collection, 2) an RNA yield >50 ng, and 3) an IVT yield >10 μg. Table 20 accounts for all samples received, and identifies the number of patients in this study (see also FIG. 18). Of the 172 enrolled U.S. patients, 167 consented to inclusion in the pharmacogenomic portion of the study. Of the 167 samples, six did not meet shipping specifications and eight samples yielded insufficient product for chip hybridization. Of the 153 samples remaining, 123 samples were procured from patients treated with AN1792 and 30 samples were procured from placebo patients; note that the 30 samples from the placebo patients were irrelevant to the analysis presented herein for this Example 2.

### Example 2.2.3.2

### Demographics of Patients

Seventy-five (75) of the patients in the study of this Example 2 were female and 78 were male. The average age was 73 years. Patient demographics for the 123 treated patients are shown in Table 21.

Subjects were assigned to response groups based on postimmunization maximum titer during follow-up. For both IgM and IgG the response groups were: 1) nonresponders, (titer<200); 2) partial responders (200≦titer<2,200); and 3) responders (titer≧2200). Table 22 gives a breakdown of study samples by gender and response category.

### Example 2.2.4

### Materials and Methods—Pharmacogenomic Statistical Analysis Plan

### Example 2.2.4.1

### Identification and Removal of Genes Significantly Associated with Covariates

Analyses were conducted to identify factors that might have confounding effects on associations between gene expression levels and response groups. Preimmunization differential blood cell counts and gender were two such factors investigated, and both were identified as significant covariates. For each gene, analysis of covariance (ANCOVA) was used to test for associations of expression level with these two covariates (i.e., with gender; monocyte:lymphocyte ratio). Log-transformed expression was modeled as a function of sex and the monocyte:lymphocyte ratio. To avoid potential confounding with IgG response or the development of encephalitis, these ANCOVAs were run using data only from IgG nonresponders (n=70). Genes were considered significantly associated with either sex or the monocyte:lymphocyte ratio if the unadjusted F-test p value for the respective effect was <0.01. Because all five encephalitis patients for these analyses were female, genes significantly associated with gender were not included in further analyses. Genes identified as having a significant linear association between expression levels and the CPT monocyte:lymphocyte ratio were also removed from further analyses. It is recognized that genes removed from analysis for these reasons may have been associated both with the identified covariable and the response class. Therefore genes associated with response class could be under-reported. Removal of these genes resulted in 8,239 remaining probesets to be further analyzed.

### Example 2.2.4.2

### Criteria for Selection of Genes Associated with Antibody Responsiveness

Subjects were assigned prior to unblinding to response groups based on postimmunization maximum titer during follow-up. As described above, for both IgM and IgG the response groups were: 1) nonresponders, (titer<200); 2) partial responders (200≦titer<2,200); and 3) responders (titer≧2200). The numbers of patients in each of these groups are shown in Table 22. The proportional odds logistic regression model was used to determine if significant associations existed between preimmunization gene expression levels and postimmunization response groups. The analyses were run using both all immunized subjects in the study (n=123), and with the exclusion of the five encephalitis patients (n=118). It should be noted that all patients from the U.S. that developed meningoencephalitis were IgG responders and all patients but one from the E.U. that developed meningoencephalitis were IgG responders, and distinction was sought between genes related to risk of encephalitis and those associated with IgG responsiveness. Raw p values were adjusted for multiplicity according to the false discovery rate (FDR) procedure of Benjamini and Hochberg ((1995) J. Roy. Stat. Soc. B. 57:289-300; see also, Xiao et al. (2002) BMC Genomics 3:28). Genes were selected as significantly associated with response if: a) the FDR for association with response was <0.1, a criterion that allows for an estimated 10% false positive identifications; b) the odds ratio between responders and others (nonresponders plus partial responders) was >3 fold; c) the FDR from the analysis excluding meningoencephalitis patients was at least twice as significant as the FDR for association with meningoencephalitis; and d) the FDR for association with encephalitis was >0.1. These selection steps identified genes with an odds ratio of at least 3 between responders and others, where the chance of a false positive association was at most 10%, with genes most significantly associated with encephalitis excluded. No genes were found to be significantly associated with the IgM response groups.

### Example 2.2.4.3

### Identification of Genes Associated with Risk of Encephalitis

The binary logistic regression model was used to determine if significant associations existed between preimmunization gene expression levels and postimmunization development of meningoencephalitis. Treated patients who developed meningoencephalitis (n=5) were compared to those who did not (n=118). The small number of meningoencephalitic subjects resulted in large odds ratios (>10) with some exceedingly wide confidence intervals (2 to 3 orders of magnitude). Because all encephalitis subjects were also IgG antibody responders, genes associated with antibody response (with the encephalitis patients excluded) were filtered from the list of encephalitis-associated genes. Genes were selected as significantly associated with encephalitis if: a) the odds ratio between meningoencephalitics and nonmeningoencephalitics was >3 fold; b) the FDR was <0.1; c) the odds ratio for association with meningoencephalitis was at least two times greater than that for association with IgG response; d) the FDR for association with IgG response was >0.1; and e) the odds ratio for IgG response was less than 2 fold. Due to the observation that some genes with IgG odds ratios between 2 and 4 fold had meningoencephalitis odds ratios up to hundreds fold higher, exceptions were made to filtering rule (e) when the odds ratio for association with meningoencephalitis was at least five-fold greater than the odds ratio for association with IgG response. These selection steps identified genes associated with an odds ratio of at least 3 between the meningoencephalitics and nonmeningoencephalitics, where the chance of a false positive association was at most 10%, with genes most significantly associated with an IgG response excluded.

### Example 2.2.4.4

### Use of GeneCluster to Select Best Gene Subset

GeneCluster (see www.broad.mit.edu/cancer/software/genecluster2/gc2.html) (Golub et al. (1999) Science 286:531-37) was used both as a method of demonstrating associations between the expression levels of the 8,239 probesets remaining (see Example 2.2.4.1) and response group using ANOVA-based methods, and to select gene expression patterns that most accurately assigned samples to the correct response class (i.e., correct response group). Gene selection was based on weighted voting. Statistical significance was assessed by a permutation-based p value. For the analysis of antibody response groups, partial responders were excluded from this analysis. Classifiers for encephalitis were chosen using data from all immunized subjects.

### Example 2.2.4.5

### Selection of Two-Gene Combinations that Most Accurately Segregate Meningoencephalitics from Nonmeningoencephalitics

The ability of two-gene models to discriminate between meningoencephalitics and nonmeningoencephalitics was evaluated by logistic regression models using as covariates all 287,661 pairwise combinations of genes meeting the criteria for association with meningoencephalitis. For each model, the sum of the absolute values of the log-odds for all subjects was used as a ranking measure to indicate the strength of the discrimination. To estimate the FDRs for this large set of logistic regression models, the full analysis was rerun 200 times with random permutation of the class labels to compute resampling-based FDRs (Reiner et al. (2003) Bioinformatics 19:368-75). These analyses were carried out using R statistics package 1.9.1, which can be found at www.R-project.org (R Development Core Team (2004) R Foundation for Statistical Computing).

### Example 2.2.4.6

### Pathway Analysis

These data were generated through the use of Ingenuity Pathways Analysis (Summer 04 Release V1), a web-delivered application that explores networks such as gene expression array data sets (see www.ingenuity.com). Biological functions were assigned to the overall analysis by using findings that have been extracted from the scientific literature and stored in the Ingenuity Pathways Knowledge Base. The biological functions assigned to the analysis are ranked according to the significance of that biological function to the analysis. A Fischer's exact test is used to calculate a p value determining the probability that the biological function assigned to the analysis is explained by chance alone.

### Example 2.3

### Results

A total of 372 patients, 172 from the U.S. and 200 from the E.U., were enrolled in the clinical trial. Participation in the pharmacogenomic portion of the study was optional and offered to U.S. patients only, and 97% agreed to participate. Consent was obtained after approval by local institutional review boards. FIG. 18 shows the disposition of patients with respect to the pharmacogenomic portion of the study. GeneChips that passed quality control inclusion criteria (detailed in Table 3) were generated from 123 treated and 30 placebo patients. The search for gene expression levels associated with response to immunization was conducted by comparing preimmunization expression levels between subjects grouped according to postimmunization response (as measured by maximum anti-AN1792 titer or the development of meningoencephalitis). Of the 6 U.S. patients who ultimately developed meningoencephalitis, 5 had consented to pharmacogenomics; there were 12 E.U. patients who developed meningoencephalitis.

### Example 2.3.1

### Identification and Removal from Analysis of Genes Associated with Monocyte Proportion and Gender Covariables

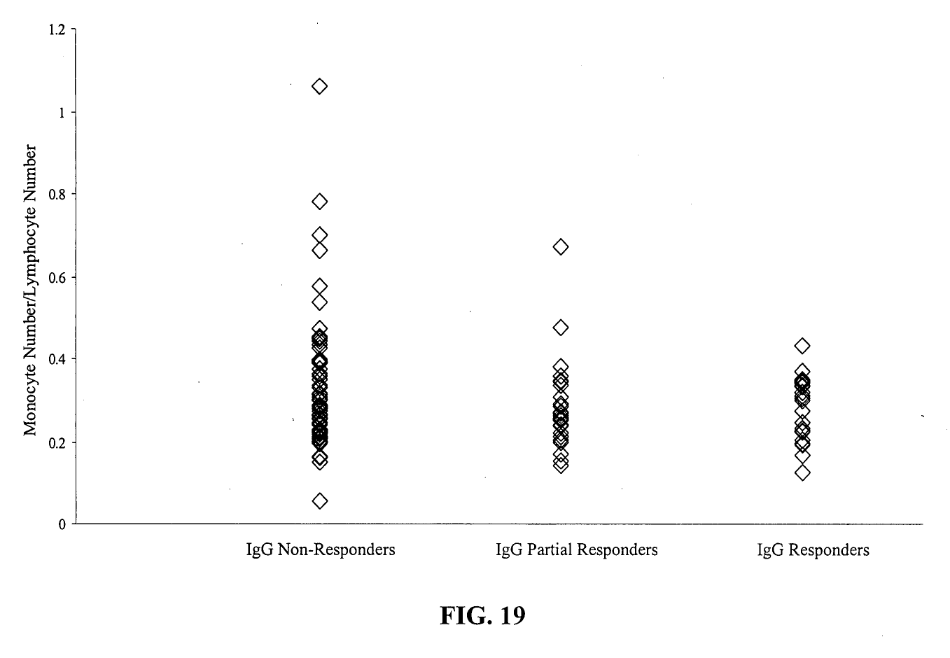

A statistically significant correlation (p=0.012) was detected between monocyte-to-lymphocyte ratio and IgG responsiveness, with a high proportion of monocytes associated with nonresponsiveness. The top 16 samples for this metric fell within the nonresponder group (see FIG. 19). The association between monocyte proportion and IgM response groups was not statistically significant, and trended in the opposite direction from the association with IgG responsiveness. Despite the statistically significant association between the IgG response and proportion of monocytes, however, the monocyte-to-lymphocyte ratio was not itself a useful biomarker of likely nonresponsiveness because the majority (77%) of nonresponders fell within the range of responders (see FIG. 18). The significance of the association did, nevertheless, point to the need to account for monocyte proportion covariate in analyses of associations between IgG responsiveness and gene expression. Another concern was that, although there were males among the E.U. patients who developed meningoencephalitis, all five U.S. encephalitis patients in the pharmacogenomic study were female, precipitating the need to account for sex-related differences in analyses of associations between encephalitis and gene expression. Sequences significantly associated with monocyte proportion and/or sex were identified by ANCOVA and removed from further analysis; they are listed in alphabetical order in Table 23. It is recognized that genes removed from analyses for these reasons may have been associated both with the identified covariate and the response class, and therefore, genes associated with response class could be under-reported. It should be noted that although the genes listed in Table 23 are excluded from Tables 24-37 (i.e., in Example 2), some genes listed in Table 23 may be included in Tables 10-12 and 18 (i.e., some of the genes listed in Tables 10-12 and 18 (see Example 1) are included in Table 23 as associated with covariates). After removal of these genes significantly associated with monocyte-to-lymphocyte ratio and/or sex, 8,239 probesets remained for further analysis.

### Example 2.3.2

### Identification of Predictive Biomarkers of IgG Response

The search for gene expression levels associated with antibody response was conducted by comparing preimmunization expression levels between subjects grouped according to postimmunization maximum IgM and IgG titer. No genes met the criteria for significant association of preimmunization gene expression levels and postimmunization IgM titer. In contrast, there were 366 sequences (from 318 genes and 17 unmapped sequences) that met the selection criteria for association with IgG response. MRPS31 (mitochondrial ribosomal protein 31) had the smallest (most significant) false discovery rate (FDR=0.0003, with a p value unadjusted for multiplicity of 1.07E⁻⁷ and odds ratio encephalitis=5.5). The highest observed odds ratio was 10.3 (for PTMA, prothymosin, alpha), indicating that elevated expression of this gene was strongly associated with IgG response. The lowest odds ratio (calculated with encephalitics) was 0.098 (GLUD1, glutamate dehydrogenase 1), indicating that decreased expression of this gene was strongly associated with IgG response. The FDRs and odds ratios for genes identified as associated with IgG response are shown in Table 24.

### Example 2.3.3

### Biological Pathways Associated with IgG Response

Pathway analyses indicate that, prior to immunization, the ability to mount an IgG response is highly correlated with expression patterns of genes directly involved in the protein synthesis machinery. Ingenuity Global Analysis reports highly significant (p value=9.53E⁻¹² to 1.29E⁻³) associations with the protein synthesis categories (a measure of the likelihood that genes that participate in protein synthesis are biomarkers associated with IgG responsiveness). In addition to the genes identified by Ingenuity, 22 additional genes were identified that directly participate in translational events. All of the IgG response-associated genes directly involved in the protein synthetic machinery were expressed at higher levels in IgG responders. The most significant of these genes are shown in Table 25. In contrast, 42% of the IgG response-associated genes involved in other functions were expressed at lower levels in IgG responders. Functions significantly represented among these genes were transcription, cell cycle, cell growth and proliferation, protein trafficking, DNA repair and recombination, and protein synthesis regulation. A selection of these genes is shown in Table 26. The annotation of IgG response-associated genes is shown in Table 27.

### Example 2.3.4

### Selection of Genes that Accurately Classify IgG Responders

Using the weighted voting algorithm as implemented in GeneCluster, a set of 24 sequences (from the 7,479 sequences remaining after removal from 9,678 probesets of genes significantly associated with monocyte-to-lymphocyte ratio and/or sex (see Example 2.3.1) and of genes significantly associated with encephalitis (see Example 2.3.5)) were identified as the most accurate classifier. All 24 sequences had a permutation-based p value <0.01, and all but one (RAB3-GAP150) had a permutation-based p value <0.001. Table 28 lists the descriptions of the 24 genes, and respective odds ratios and FDRs for IgG and encephalitis, that are best at accurate classification of the IgG responders (the 24 genes identify 76 patients correctly and 19 patients incorrectly; of the incorrectly identified patients, 6 are IgG responders). Table 29 lists the classification of each patient (i.e., patients that were IgG responders or IgG nonresponders) and the confidence score using these 24 classifier genes. Table 30 is a list of the 6 best classifiers of an IgG response (a subset of the 24 genes in Table 28); this set correctly identifies 75 patients but incorrectly identifies 20 patients. Table 31 lists the classification of each patient and the confidence score using these 6 classifier genes.

### Example 2.3.5

### Identification of Predictive Biomarkers for Development of Encephalitis

There were 760 sequences (from 689 genes and 8 unmapped sequences) that met the selection criteria for association with encephalitis. These associations were identified by comparing the gene expression levels of the 5 patients who developed meningoencephalitis to the gene expression levels of the 118 treated patients who did not. The gene most significantly associated (unadjusted p=5.07E⁻⁷, FDR=0.004, odds ratio=230) with encephalitis was STAT1, a critical gene in a proinflammatory signal transduction pathway. The highest odds ratio observed was 3,136 (for NHP2L1, with increased expression associated with encephalitis). The lowest odds ratio was 1.0E⁻⁴ (for HEAB, with decreased expression associated with encephalitis). For 364 sequences (48%) of the 760 meningoencephalitis-associated sequences, the odds ratios were greater than 10 fold (greater than 10 or less than 0.1), but the confidence limits were often very broad due to the small size of the encephalitis group and the heterogeneity within it. The development of encephalitis was associated with the decreased expression of 41% of the sequences. The FDRs and odds ratios for the meningoencephalitis-associated sequences are shown in Table 32.

### Example 2.3.6

### Genes and Biological Pathways Associated with Development of Encephalitis

Of the 760 sequences associated with encephalitis, 63 were replicate identifications (i.e., multiple probesets mapping to the same gene). The majority of these sequences were mapped by Ingenuity; among the unmapped sequences, five subsequently were mapped to known genes by homology search. Ingenuity Global Analysis assigns 56% of encephalitis-associated genes to “High Level Functions” and “Global Canonical Pathways.” Significantly represented were genes related to the control of apoptosis and proinflammatory immune response, or to the downstream functions of control of cell cycle, cell proliferation, protein synthesis and protein trafficking (see Table 33 for annotation of genes associated with meningoencephalitis). Ingenuity Pathway Analysis reports p values for the significance of the link between encephalitis-associated genes and cell death categories as ranging from 7.46E⁻⁷ to 4.65E⁻², and for the link between associated genes and cell cycle functions as ranging from 4.35E⁻⁹ to 4.65E⁻². Genes related to TNF/Fas, TGFβ and p53 pathways were highly represented among genes related to the control of cell death (see Table 34). A selection of these genes and their association with meningoencephalitis is shown in Table 35. While the encephalitis-associated genes in Table 35 were selected on the basis of known involvement in TNF and/or Fas pathways and other immune response-related cell death and cell activation pathways, the list does not encompass all such genes.

### Example 2.3.7

### Selection of Genes that Accurately Classify Patients Who Develop Encephalitis

Using the frequency data from all immunized subjects, eight genes (selected from the 760 encephalitis-associated sequences, and shown in Table 36) that accurately assigned 4 of 5 encephalitis patients and 111 (94%) of nonencephalitis patients were identified using weighted voting and leave-one-out cross-validation in GeneCluster. The confidence scores for the classification of the five encephalitis patients and a representative selection of nonencephalitis patients are shown in FIG. 20. The one encephalitis patient who was assigned to the incorrect group was assigned with the highest possible confidence score. Therefore, additional analyses were conducted to determine whether a model weighted toward the capture of all five encephalitis patients would correctly classify this patient as among those who developed encephalitis.

Selection of optimal classifiers by the pairwise combination logistic regression approach was designed to find the two-gene combinations that best distinguished the meningoencephalitics from nonmeningoencephalitics. No functional annotation is available on nuclear protein ukP68 (NpukP68), which was one of the two genes in the top ranked logistic regression-based classifier pair. STAT1 appeared in the third-highest ranked two-gene classifier, with an odds ratio for association with encephalitis of 230.4. Remarkably, for 18 of the top 20 two-gene combinations (listed in Table 37), one of the genes in the two-gene combination was either STAT1 or NpukP68, indicating a very strong association between high expression of either of these two genes and the development of encephalitis. FIG. 21 shows expression level plots of the top ranked and third ranked gene combinations (pairs). FIG. 22 shows the expression level plots for the remaining 18 top-ranked gene pairs. Both FIG. 21 and FIG. 22 display the association of expression profiles for the pairs of genes listed in Table 37 with either the clinical response of encephalitis development or encephalitis nondevelopment.

### Example 2.4

### Discussion

This invention identified 318 genes whose expression levels prior to immunization with AN1792 are significantly associated with IgG responsiveness to AN1792 immunization (i.e., can be also be used to assess IgG nonresponsiveness). No such risk factors were identified for IgM nonresponsiveness. Expression levels of genes associated with IgG response in partial responders (200≦titer<2,200) were consistently intermediate between nonresponders (titer<200) and responders (titer≧2200), a trend that provides additional evidence of the relationship between preimmunization gene expression pattern and IgG response.

The vast majority of genes associated with IgG response are related to biological functions (protein synthesis and trafficking, RNA processing, cellular assembly and organization, and cell cycle control) that are not specific to the immune system. The incidence of responsiveness in this study was relatively low (53 of 123 with titer>200), and the patients were elderly (mean age 74 years). Since responsiveness to immunization is known to decline with age (Westmoreland et al. (1990) Epidemiol. Infect. 104:499-509; Looney et al. (2001) J. Clin. Immunol. 21:30-36; Rey (1997) Bull. Soc. Pathol. Exot. 90 (4):245-52; Arreaza et al (1993) Clin. Exp. Immunol. 92:169-73; Salvador et al. (2003) Immunol. Allergy Clin. North Am. 23 (1):133-48), age may influence the expression levels of genes directly involved in protein synthesis and the other functions identified by this invention as associated with IgG response.

The invention identified 689 genes whose expression levels prior to immunization with AN1792 are significantly associated with development of encephalitis following immunization. These risk factors were identified by comparing the gene expression levels of the five patients who developed encephalitis to the levels of the 118 treated patients who did not develop encephalitis. In contrast to the IgG associated genes, functional annotation of genes associated with encephalitis indicated a preponderance of genes of particular importance in pathways related to the control of the immune system and inflammation. Those who developed encephalitis had, prior to immunization, detectable perturbations in pathways controlling the TNF and other proinflammatory and apoptotic cascades. Perturbations favoring both anti-apoptotic and pro-apoptotic activities were detected, possibly suggesting compensatory activation to counteract deleterious effects of perturbation in apoptosis. This is also supported by perturbations in a large number of cell cycle, growth, and proliferation genes. The STAT gene family plays a central role in proinflammatory cytokine activation and in apoptotic cascades. Perturbation in the expression levels of STAT1, STAT3 (3′ untranslated region), and STAT5 were found to be highly significant risk factors for encephalitis. High expression of a variety of other genes involved in proinflammatory cascades, such as IL-9, IL-19, IL-25, IL-27R, and CD80, were also associated with encephalitis. Elevated expression of the coding region and decreased expression of the 3′ untranslated region of STAT5B were associated with development of meningoencephalitis, suggesting that variants of STAT5B mRNA make different contributions to the “meningoencephalitis-prone” gene expression pattern.

All five encephalitis patients for whom gene expression data were available were IgG responders. It is therefore notable that IgG responders who developed encephalitis expressed some protein synthesis and trafficking genes at levels significantly lower than nonmeningoencephalitic IgG responders. Remarkably, for a number of genes (RPS7, RPLP1, RPS24, and RPL9), lower expression levels were associated with development of encephalitis, while higher expression levels were associated with IgG response. Another distinction between the IgG response associated genes and the meningoencephalitis-associated genes is that, although protein synthesis is identified as a significant category among both sets, the preponderance (˜80%) of IgG response-associated genes in this category are directly involved in the protein synthetic machinery, and that all of these were expressed at higher levels in IgG responders. In contrast, the majority of meningoencephalitis-associated genes categorized as involved in protein synthesis regulate protein expression, with only approximately half expressed at higher levels in the meningoencephalitis group. These data provide an additional line of evidence that preimmunization gene expression patterns associated with risk of encephalitis are distinguishable from those associated with IgG response.

Logistic regression using pairwise combinations of genes was applied to identify the most accurate two-gene combination classifier of patients at risk of developing meningoencephalitis. This analytical approach identified the combination of expression levels of NPukP68 and AKAP13 (PRKA anchor protein 13 anchor) as the top biomarkers for separating all 5 meningoencephalitics from nonmeningoencephalitics. No functional annotation is available on NPukP68, but elevated expression was associated with an odds ratio of 651. Either NPukP68 or STAT1 (odds ratio of 230.4) appears as one of the genes listed in eighteen of the 20 top ranked pairwise combinations.

Of the five meningoencephalitis patients, encephalitis, one expressed the vast majority of 760 meningoencephalitis associated sequences at levels associated with the nonmeningoencephalitis group. However, this patient expressed numerous genes at levels associated with encephalitis following 24-hour in vitro stimulation with a stimulatory cytokine cocktail and the AN1792 antigen (i.e., the protocol in Example 1; see patient 33, e.g., in FIGS. 4-13). These observations together suggest that a small number of critical genes may profoundly influence the consequences of both in vivo and in vitro immune stimulation.

The inventors have identified highly significant associations between PBMC preimmunization gene expression patterns and postimmunization anti-AN1792 IgG responses and postimmunization development of meningoencephalitis. These results may be of use in identifying patients at risk of developing a severe adverse event in active immunotherapy for Alzheimer's disease, and in identifying those patients that are likely to respond to immunotherapy.

All references cited in this application are incorporated by reference in their entireties as if fully set forth herein.

TABLE 1
Stringency examples

| Stringency | Polynucleotide | Hybrid | Hybridization Temperature | Wash Temperature |
|---|---|---|---|---|
| Condition | Hybrid | Length (bp)¹ | and Buffer² | and Buffer² |
| A | DNA:DNA | >50 | 65° C.; 1X SSC -or- | 65° C.; 0.3X SSC |
| | | | 42° C.; 1X SSC, 50% | |
| | | | formamide | |
| B | DNA:DNA | <50 | T_{B}*; 1X SSC | T_{B}*; 1X SSC |
| C | DNA:RNA | >50 | 67° C.; 1X SSC -or- | 67° C.; 0.3X SSC |
| | | | 45° C.; 1X SSC, 50% | |
| | | | formamide | |
| D | DNA:RNA | <50 | T_{D}*; 1X SSC | T_{D}*; 1X SSC |
| E | RNA:RNA | >50 | 70° C.; 1X SSC -or- | 70° C.; 0.3X SSC |
| | | | 50° C.; 1X SSC, 50% | |
| | | | formamide | |
| F | RNA:RNA | <50 | T_{F}*; 1X SSC | T_{F}*; 1X SSC |
| G | DNA:DNA | >50 | 65° C.; 4X SSC -or- | 65° C.; 1X SSC |
| | | | 42° C.; 4X SSC, 50% | |
| | | | formamide | |
| H | DNA:DNA | <50 | T_{H}*; 4X SSC | T_{H}*; 4X SSC |
| I | DNA:RNA | >50 | 67° C.; 4X SSC -or- | 67° C.; 1X SSC |
| | | | 45° C.; 4X SSC, 50% | |
| | | | formamide | |
| J | DNA:RNA | <50 | T_{J}*; 4X SSC | T_{J}*; 4X SSC |
| K | RNA:RNA | >50 | 70° C.; 4X SSC -or- | 67° C.; 1X SSC |
| | | | 50° C.; 4X SSC, 50% | |
| | | | formamide | |
| L | RNA:RNA | <50 | T_{L}*; 2X SSC | T_{L}*; 2X SSC |
| M | DNA:DNA | >50 | 50° C.; 4X SSC -or- | 50° C.; 2X SSC |
| | | | 40° C.; 6X SSC, 50% | |
| | | | formamide | |
| N | DNA:DNA | <50 | T_{N}*; 6X SSC | T_{N}*; 6X SSC |
| O | DNA:RNA | >50 | 55° C.; 4X SSC -or- | 55° C.; 2X SSC |
| | | | 42° C.; 6X SSC, 50% | |
| | | | formamide | |
| P | DNA:RNA | <50 | Tₚ*; 6X SSC | Tₚ*; 6X SSC |
| Q | RNA:RNA | >50 | 60° C.; 4X SSC -or- | 60° C.; 2X SSC |
| | | | 45° C.; 6X SSC, 50% | |
| | | | formamide | |
| R | RNA:RNA | <50 | T_{R}*; 4X SSC | T_{R}*; 4X SSC |
| ¹The hybrid length is that anticipated for the hybridized region(s) of the hybridizing polynucleotides. When hybridizing a polynucleotide to a target polynucleotide of unknown sequence, the hybrid length is | | | | |
| # assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ²SSPE (1xSSPE is 0.15 M NaCl, 10 mM NaH₂PO₄, and 1.25 mM EDTA, pH 7.4) can be substituted for SSC (1xSSC is 0.15 M NaCl and 15 mM sodium citrate) in the hybridization and wash buffers; washes are performed for 15 minutes after hybridization is complete. | | | | |
| T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids | | | | |
| # less than 18 base pairs in length, Tₘ(° C.) = 2(# of A + T bases) + 4(# of G + C bases). For hybrids between 18 and 49 base pairs in length, Tₘ(° C.) = 81.5 + 16.6(log₁₀Na⁺) + 0.41(% G + C) − (600/N), where N is the number of bases in the hybrid, and | | | | |
| Na⁺ is the concentration of sodium ions in the hybridization buffer (Na⁺ for 1X SSC = 0.165 M). Additional examples of stringency conditions for polynucleotide hybridization are provided in Sambrook et al., Molecular | | | | |
| # Cloning: A Laboratory Manual, Chs. 9 & 11, Cold Spring Harbor Laboratory Press, Cold Spring Harbor, NY (1989), and Ausubel et al., eds., Current Protocols in Molecular Biology, Sects. 2.10 & 6.3-6.4, John Wiley & Sons, Inc. (1995), herein incorporated by reference. | | | | |

TABLE 2
Characteristics of samples with % neutrophils >20%
post CPT fractionation

| | | | | Maxi- | Maxi- |
|---|---|---|---|---|---|
| | | Post-CPT | | mum | mum |
| | Pre-CPT | % Neutro- | Treatment | IgG | IgM |
| Patient | % Neutrophils | phils | Group | Titer | Titer |
| 17 | 63 | 38 | Immunotherapy | 50 | 25 |
| 23 | 64 | 47 | Immunotherapy | 50 | 25 |
| 36 | 62 | 29 | Immunotherapy | 4111 | 13275 |
| 44 | 53 | 25 | Immunotherapy | 126 | 957 |
| 271 | 79 | 37 | Immunotherapy | 172 | 28554 |
| 288 | 59 | 25 | Placebo | 50 | 25 |
| 756 | 67 | 40 | Immunotherapy | 50 | 25 |

TABLE 3
Criteria for chip inclusion in the final dataset

| | |
|---|---|
| Chip sensitivity | <6.1 |
| Raw Q | <7 |
| Scale factor | <4 and >1/4 |
| Cell saturation ratio | <0.00005 |
| QC P probability frequency | <20 |
| QC P probability average difference | <250 |
| Number of outliers across the array | <1600 |
| Defect on visual inspection | Absent |

TABLE 4
r² values for all study samples

| | AN1792- | | Control | |
|---|---|---|---|---|
| Patient | stimulated | S.D. | culture | S.D. |
| number | sample | AN1792 | sample | control |
| 2 | 0.90 | 0.05 | 0.84 | 0.07 |
| 3 | 0.92 | 0.05 | 0.88 | 0.06 |
| 5 | 0.89 | 0.05 | 0.87 | 0.06 |
| 6 | 0.90 | 0.06 | 0.88 | 0.06 |
| 7 | 0.89 | 0.04 | 0.81 | 0.06 |
| 8 | 0.88 | 0.05 | 0.79 | 0.06 |
| 9 | 0.90 | 0.05 | | N.A. |
| 12 | 0.92 | 0.04 | 0.89 | 0.05 |
| 14 | 0.86 | 0.05 | 0.86 | 0.06 |
| 15 | 0.90 | 0.05 | 0.85 | 0.06 |
| 17 | 0.90 | 0.05 | 0.82 | 0.06 |
| 18 | 0.87 | 0.06 | 0.87 | 0.05 |
| 19 | 0.84 | 0.06 | 0.84 | 0.07 |
| 20 | 0.91 | 0.05 | 0.88 | 0.06 |
| 22 | 0.90 | 0.05 | 0.72 | 0.08 |
| 23 | 0.88 | 0.05 | 0.88 | 0.04 |
| 24 | 0.88 | 0.05 | 0.87 | 0.07 |
| 25 | 0.86 | 0.06 | 0.83 | 0.06 |
| 26 | 0.88 | 0.06 | 0.86 | 0.06 |
| 27 | 0.81 | 0.08 | 0.82 | 0.07 |
| 29 | 0.91 | 0.05 | 0.89 | 0.06 |
| 30 | 0.87 | 0.05 | 0.85 | 0.05 |
| 31 | 0.88 | 0.04 | 0.88 | 0.05 |
| 32 | 0.88 | 0.05 | 0.87 | 0.06 |
| 33 | 0.72 | 0.06 | 0.89 | 0.06 |
| 34 | 0.91 | 0.04 | 0.89 | 0.05 |
| 36 | 0.88 | 0.05 | 0.87 | 0.06 |
| 37 | 0.91 | 0.04 | | N.A. |
| 40 | 0.88 | 0.06 | 0.86 | 0.07 |
| 41 | 0.89 | 0.06 | 0.88 | 0.06 |
| 43 | 0.92 | 0.04 | 0.88 | 0.05 |
| 44 | 0.90 | 0.05 | 0.89 | 0.05 |
| 45 | 0.90 | 0.05 | 0.88 | 0.05 |
| 46 | 0.91 | 0.04 | 0.88 | 0.06 |
| 49 | 0.80 | 0.05 | 0.71 | 0.07 |
| 50 | 0.88 | 0.06 | 0.84 | 0.07 |
| 52 | 0.90 | 0.05 | 0.87 | 0.06 |
| 53 | 0.90 | 0.05 | 0.89 | 0.05 |
| 54 | 0.89 | 0.05 | 0.87 | 0.07 |
| 55 | 0.91 | 0.04 | | N.A. |
| 56 | 0.86 | 0.05 | 0.88 | 0.06 |
| 60 | 0.90 | 0.05 | 0.78 | 0.07 |
| 61 | 0.89 | 0.04 | 0.88 | 0.05 |
| 62 | 0.88 | 0.05 | 0.86 | 0.07 |
| 64 | 0.88 | 0.04 | 0.88 | 0.06 |
| 65 | 0.88 | 0.05 | | N.A. |
| 66 | 0.89 | 0.04 | 0.85 | 0.07 |
| 67 | 0.89 | 0.04 | 0.86 | 0.07 |
| 68 | 0.85 | 0.06 | 0.87 | 0.06 |
| 69 | 0.92 | 0.04 | | N.A. |
| 71 | 0.89 | 0.05 | 0.87 | 0.07 |
| 73 | 0.85 | 0.04 | 0.88 | 0.06 |
| 251 | 0.92 | 0.04 | 0.89 | 0.05 |
| 252 | 0.89 | 0.04 | 0.86 | 0.06 |
| 254 | 0.90 | 0.05 | 0.88 | 0.05 |
| 255 | 0.91 | 0.04 | 0.83 | 0.06 |
| 256 | 0.88 | 0.06 | 0.87 | 0.06 |
| 257 | 0.90 | 0.04 | 0.83 | 0.06 |
| 258 | 0.90 | 0.05 | 0.88 | 0.06 |
| 259 | 0.90 | 0.05 | 0.90 | 0.05 |
| 260 | 0.89 | 0.06 | 0.89 | 0.05 |
| 262 | 0.89 | 0.04 | 0.88 | 0.05 |
| 264 | 0.90 | 0.06 | 0.89 | 0.05 |
| 266 | 0.91 | 0.05 | 0.88 | 0.06 |
| 267 | 0.90 | 0.05 | 0.89 | 0.05 |
| 268 | 0.89 | 0.05 | 0.69 | 0.06 |
| 269 | 0.88 | 0.06 | 0.87 | 0.06 |
| 270 | 0.87 | 0.05 | 0.88 | 0.06 |
| 271 | 0.88 | 0.05 | 0.85 | 0.06 |
| 272 | 0.90 | 0.06 | 0.88 | 0.06 |
| 273 | 0.91 | 0.04 | 0.87 | 0.05 |
| 274 | 0.92 | 0.04 | 0.88 | 0.05 |
| 275 | 0.89 | 0.05 | 0.88 | 0.06 |
| 277 | 0.89 | 0.05 | 0.85 | 0.06 |
| 279 | 0.88 | 0.05 | 0.89 | 0.05 |
| 280 | 0.90 | 0.05 | 0.89 | 0.06 |
| 281 | 0.91 | 0.04 | 0.86 | 0.06 |
| 282 | 0.90 | 0.05 | 0.88 | 0.05 |
| 283 | 0.84 | 0.06 | 0.73 | 0.06 |
| 284 | 0.91 | 0.05 | 0.86 | 0.06 |
| 285 | 0.87 | 0.06 | 0.87 | 0.06 |
| 286 | 0.89 | 0.05 | 0.88 | 0.05 |
| 287 | 0.91 | 0.04 | 0.89 | 0.06 |
| 288 | 0.91 | 0.04 | 0.87 | 0.06 |
| 289 | 0.90 | 0.05 | 0.89 | 0.06 |
| 290 | 0.90 | 0.04 | 0.82 | 0.06 |
| 291 | 0.91 | 0.04 | 0.84 | 0.07 |
| 292 | 0.90 | 0.05 | 0.87 | 0.06 |
| 293 | 0.89 | 0.05 | 0.86 | 0.07 |
| 294 | 0.90 | 0.05 | 0.86 | 0.06 |
| 295 | 0.91 | 0.04 | 0.90 | 0.05 |
| 296 | 0.91 | 0.04 | 0.89 | 0.05 |
| 297 | 0.89 | 0.06 | 0.86 | 0.07 |
| 299 | 0.87 | 0.06 | 0.89 | 0.06 |
| 300 | 0.91 | 0.04 | 0.89 | 0.06 |
| 301 | 0.88 | 0.05 | 0.83 | 0.06 |
| 303 | 0.84 | 0.06 | 0.86 | 0.07 |
| 304 | 0.90 | 0.05 | 0.87 | 0.06 |
| 306 | 0.89 | 0.04 | 0.85 | 0.07 |
| 307 | 0.87 | 0.05 | | N.A. |
| 308 | 0.79 | 0.07 | | N.A. |
| 309 | 0.90 | 0.05 | 0.88 | 0.06 |
| 310 | 0.90 | 0.04 | 0.84 | 0.06 |
| 312 | 0.91 | 0.04 | 0.86 | 0.06 |
| 313 | 0.89 | 0.05 | | N.A. |
| 314 | 0.88 | 0.05 | 0.89 | 0.06 |
| 315 | 0.90 | 0.04 | 0.88 | 0.05 |
| 316 | 0.88 | 0.04 | 0.70 | 0.06 |
| 317 | 0.89 | 0.05 | 0.88 | 0.06 |
| 318 | 0.89 | 0.04 | 0.88 | 0.06 |
| 319 | 0.88 | 0.06 | | N.A. |
| 502 | 0.91 | 0.05 | 0.88 | 0.05 |
| 503 | 0.84 | 0.06 | 0.82 | 0.07 |
| 504 | 0.89 | 0.05 | 0.88 | 0.05 |
| 505 | 0.89 | 0.06 | 0.84 | 0.05 |
| 508 | 0.89 | 0.05 | 0.86 | 0.06 |
| 510 | 0.91 | 0.04 | 0.89 | 0.05 |
| 511 | 0.89 | 0.05 | 0.86 | 0.06 |
| 513 | 0.90 | 0.05 | 0.87 | 0.06 |
| 514 | 0.90 | 0.05 | 0.87 | 0.06 |
| 515 | 0.85 | 0.06 | 0.84 | 0.06 |
| 752 | 0.90 | 0.05 | 0.88 | 0.06 |
| 753 | 0.91 | 0.05 | 0.88 | 0.06 |
| 755 | 0.90 | 0.05 | 0.90 | 0.06 |
| 756 | 0.89 | 0.04 | 0.86 | 0.06 |
| 758 | 0.90 | 0.05 | 0.87 | 0.06 |
| 759 | 0.91 | 0.05 | 0.87 | 0.05 |
| 760 | 0.90 | 0.05 | 0.89 | 0.05 |
| 761 | 0.90 | 0.05 | 0.86 | 0.06 |
| 762 | 0.90 | 0.04 | 0.89 | 0.05 |
| 763 | 0.87 | 0.06 | 0.78 | 0.09 |
| 764 | 0.90 | 0.05 | 0.86 | 0.07 |
| 765 | 0.88 | 0.04 | 0.74 | 0.06 |

TABLE 5
Responder status of r² value outlier samples

| | Patient | | IgG | |
|---|---|---|---|---|
| Patient | treatment | | response | IgM response |
| number | group | Culture condition | group | group |
| 33 | AN1792 | AN1792- | IgG responder | IgM responder |
| | | stimulated | and | |
| | | | meningoen- | |
| | | | cephalitic | |
| 22 | AN1792 | Diluent control- | IgG Responder | IgM responder |
| | | stimulated | | |
| 49 | Placebo | Diluent control- | Not applicable | Not applicable |
| | | stimulated | | |
| 268 | Placebo | Diluent control- | Not applicable | Not applicable |
| | | stimulated | | |
| 283 | Placebo | Diluent control- | Not applicable | Not applicable |
| | | stimulated | | |
| 316 | AN1792 | Diluent control- | Nonresponder | Nonresponder |
| | | stimulated | | |
| 765 | AN1792 | Diluent control- | Nonresponder | Nonresponder |
| | | stimulated | | |

TABLE 6
Samples received by pharmacogenomic laboratory

| | Number |
|---|---|
| Enrolled U.S. patients | 172 |
| Enrolled patients who consented to | 167 |
| pharmacogenomic portion of study | |
| Samples within shipping specifications | 161 |
| AN1792-stimulated samples within | 149 |
| culture and storage specifications | |
| AN1792-stimulated samples with >50 ng RNA | 141 |
| AN1792-stimulated samples with >10 μg IVT | 141 |
| AN1792-stimulated samples removed | 8 |
| due to operator error identified | |
| during QC review | |
| Total number of AN1792-stimulated | 133 |
| samples in study | |
| Patients represented by paired | 124 |
| (antigen-stimulated and | |
| diluent control) samples | |
| Diluent control samples unavailable | 9 |
| due to insufficient | |
| yield of mRNA or IVT | |

TABLE 7
Patients in study, by year of birth

| | | Number of | Number of |
|---|---|---|---|
| Year of Birth | Number of Patients | Female Patients | Male Patients |
| 1915-1920 | 27 | 17 | 10 |
| 1921-1925 | 30 | 19 | 11 |
| 1926-1930 | 30 | 9 | 21 |
| 1931-1935 | 20 | 6 | 14 |
| 1936-1940 | 19 | 7 | 12 |
| 1941-1945 | 2 | 1 | 1 |
| 1946-1950 | 4 | 4 | 0 |
| Unknown | 1 | 1 | 0 |
| Cumulative total | 133 | 64 | 69 |

TABLE 8
Gender of patients in study, by race

| | Caucasian | Hispanic | Black | Asian | Unknown |
|---|---|---|---|---|---|
| Females | 56 | 6 | 2 | 0 | 0 |
| Males | 58 | 6 | 2 | 1 | 2 |

TABLE 9
Samples in pharmacogenomic study

| | Male | Female | Total |
|---|---|---|---|
| Placebo | 11 | 14 | 25 |
| Treated | 59 | 49 | 108 |
| Typed ApoE4 negative | 23 | 11 | 34 |
| Typed ApoE4 positive | 34 | 36 | 70 |
| Treated IgG responders | 10 | 12 | 22 |
| Treated IgG partial responders | 12 | 14 | 26 |
| Treated IgG nonresponders | 37 | 23 | 60 |
| Treated IgM responders | 40 | 41 | 81 |
| Treated IgM nonresponders | 19 | 8 | 27 |
| Meningoencephalitis patients | 0 | 5 | 5 |

TABLE 10
GENES ASSOCIATED WITH MENINGOENCEPHALITIS BY ANOVA
(sorted alphabetically by gene name)

| | | | | | Average |
|---|---|---|---|---|---|
| | | | | | expression in |
| | | | | | meningoencephalitis |
| | | | | | patients relative to |
| | | | | | average in |
| Accession | | | Unadjusted | | nonencephalitis |
| number | Gene description | Gene name | p value | FDR | patients |
| NM_005736 | ARP1 actin-related protein | ACTR1A | 0.000088 | 0.0203 | lower |
| | 1 homolog A, centractin | | | | |
| | alpha (yeast) | | | | |
| NM_015999 | Adiponectin receptor 1 | ADIPOR1 | 0.000158 | 0.0271 | lower |
| AK021586 | Agrin | AGRN | 0.000042 | 0.0169 | lower |
| M90360 | A kinase (PRKA) anchor | AKAP13 | 0.000037 | 0.0163 | higher |
| | protein 13 | | | | |
| NM_014481 | APEX nuclease | APEX2 | 0.000221 | 0.0330 | lower |
| | (apurinic/apyrimidinic | | | | |
| | endonuclease) 2 | | | | |
| NM_001655 | archain 1 | ARCN1 | 0.000016 | 0.0092 | higher |
| BC005851 | Rho GDP dissociation | ARHGDIA | 0.000344 | 0.0390 | lower |
| | inhibitor (GDI) alpha | | | | |
| NM_012099 | CD3-epsilon-associated | ASE-1 | 0.000314 | 0.0389 | lower |
| | protein; antisense to | | | | |
| | ERCC-1 | | | | |
| NM_025080 | asparaginase like 1 | ASRGL1 | 0.000001 | 0.0087 | higher |
| U26455 | ataxia telangiectasia | ATM | 0.000232 | 0.0338 | higher |
| | mutated (includes | | | | |
| | complementation groups | | | | |
| | A, C and D) | | | | |
| NM_001687 | ATP synthase, H+ | ATP5D | 0.000476 | 0.0448 | lower |
| | transporting, mitochondrial | | | | |
| | F1 complex, delta subunit | | | | |
| M62762 | ATPase, H+ transporting, | ATP6V0C | 0.000526 | 0.0468 | lower |
| | lysosomal 16 kDa, V0 | | | | |
| | subunit c | | | | |
| NM_001693 | ATPase, H+ transporting, | ATP6V1B2 | 0.000085 | 0.0203 | lower |
| | lysosomal 56/58 kDa, V1 | | | | |
| | subunit B, isoform 2 | | | | |
| NM_016311 | ATPase inhibitory factor 1 | ATPIF1 | 0.000414 | 0.0413 | higher |
| NM_017450 | BAI1-associated protein 2 | BAIAP2 | 0.000160 | 0.0271 | lower |
| NM_004640 | HLA-B associated | BAT1 | 0.000323 | 0.0389 | lower |
| | transcript 1 | | | | |
| AA102574 | bromodomain adjacent to | BAZ1A | 0.000010 | 0.0087 | higher |
| | zinc finger domain, 1A | | | | |
| NM_001707 | B-cell CLL/lymphoma 7B | BCL7B | 0.000069 | 0.0184 | lower |
| NM_004634 | bromodomain and PHD | BRPF1 | 0.000149 | 0.0266 | higher |
| | finger containing, 1 | | | | |
| NM_018944 | chromosome 21 open | C21ORF45 | 0.000253 | 0.0352 | higher |
| | reading frame 45 | | | | |
| AL545982 | chaperonin containing | CCT2 | 0.000231 | 0.0338 | lower |
| | TCP1, subunit 2 (beta) | | | | |
| AF098641 | CD44 antigen (homing | CD44 | 0.000152 | 0.0266 | lower |
| | function and Indian blood | | | | |
| | group system) | | | | |
| NM_001783 | CD79A antigen | CD79A | 0.000484 | 0.0449 | lower |
| | (immunoglobulin- | | | | |
| | associated alpha) | | | | |
| AB017493 | core promoter element | COPEB | 0.000063 | 0.0184 | higher |
| | binding protein | | | | |
| U69546 | CUG triplet repeat, RNA | CUGBP2 | 0.000393 | 0.0412 | higher |
| | binding protein 2 | | | | |
| BE046443 | cylindromatosis (turban | CYLD | 0.000384 | 0.0408 | higher |
| | tumor syndrome) | | | | |
| NM_001343 | disabled homolog 2, | DAB2 | 0.000065 | 0.0184 | higher |
| | mitogen-responsive | | | | |
| | phosphoprotein | | | | |
| | (Drosophila) | | | | |
| BG530850 | DEAD (Asp-Glu-Ala-Asp) | DDX18 | 0.000189 | 0.0305 | higher |
| | box polypeptide 18 | | | | |
| BE963238 | DEAD (Asp-Glu-Ala-Asp) | DDX52 | 0.000404 | 0.0413 | higher |
| | box polypeptide 52 | | | | |
| AW081113 | SR rich protein | DKFZP564B0769 | 0.000008 | 0.0087 | higher |
| NM_001961 | eukaryotic translation | EEF2 | 0.000106 | 0.0225 | lower |
| | elongation factor 2 | | | | |
| BG481972 | eukaryotic translation | EIF5 | 0.000006 | 0.0087 | higher |
| | initiation factor 5 | | | | |
| BF445047 | epithelial membrane | EMP1 | 0.000461 | 0.0442 | lower |
| | protein 1 | | | | |
| NM_004459 | fetal Alzheimer antigen | FALZ | 0.000059 | 0.0184 | higher |
| NM_012179 | F-box only protein 7 | FBXO7 | 0.000025 | 0.0123 | lower |
| NM_018115 | hypothetical protein | FLJ10498 | 0.000183 | 0.0300 | lower |
| | FLJ10498 | | | | |
| NM_024845 | hypothetical protein | FLJ14154 | 0.000049 | 0.0184 | lower |
| | FLJ14154 | | | | |
| NM_017736 | hypothetical protein | FLJ20274 | 0.000080 | 0.0203 | higher |
| | FLJ20274 | | | | |
| NM_017775 | hypothetical protein | FLJ20343 | 0.000315 | 0.0389 | higher |
| | FLJ20343 | | | | |
| AU145053 | formin binding protein 1 | FNBP1 | 0.000409 | 0.0413 | higher |
| NM_002030 | formyl peptide receptor- | FPRL2 | 0.000501 | 0.0459 | lower |
| | like 2 | | | | |
| NM_002569 | furin (paired basic amino | FURIN | 0.000264 | 0.0363 | lower |
| | acid cleaving, enzyme) | | | | |
| BE439987 | growth arrest-specific 7 | GAS7 | 0.000240 | 0.0344 | higher |
| BE646414 | golgi associated, gamma | GGA2 | 0.000271 | 0.0364 | higher |
| | adaptin ear containing, | | | | |
| | ARF binding protein 2 | | | | |
| BG420237 | heat shock 90 kDa protein | HSPCA | 0.000207 | 0.0318 | higher |
| | 1, alpha | | | | |
| AA284705 | intercellular adhesion | ICAM1 | 0.000010 | 0.0087 | lower |
| | molecule 1 (CD54), human | | | | |
| | rhinovirus receptor | | | | |
| BG261322 | translation initiation factor | IF2 | 0.000041 | 0.0169 | higher |
| | IF2 | | | | |
| NM_016281 | STE20-like kinase | JIK | 0.000250 | 0.0352 | higher |
| BF382924 | joined to JAZF1 | JJAZ1 | 0.000327 | 0.0389 | higher |
| NM_003772 | jerky homolog-like | JRKL | 0.000425 | 0.0420 | higher |
| | (mouse) | | | | |
| D26488 | KIAA0007 protein | KIAA0007 | 0.000070 | 0.0184 | higher |
| AI673812 | KIAA0553 protein | KIAA0553 | 0.000014 | 0.0087 | higher |
| AU153525 | KIAA0652 gene product | KIAA0652 | 0.000558 | 0.0485 | lower |
| AI629033 | KIAA0872 protein | KIAA0872 | 0.000063 | 0.0184 | lower |
| BF223224 | kinesin family member 5B | KIF5B | 0.000063 | 0.0184 | higher |
| BF673699 | v-Ki-ras2 Kirsten rat | KRAS2 | 0.000127 | 0.0252 | higher |
| | sarcoma 2 viral oncogene | | | | |
| | homolog | | | | |
| AK001105 | LAG 1 longevity assurance | LASS2 | 0.000062 | 0.0184 | lower |
| | homolog 2 (S. cerevisiae) | | | | |
| NM_017526 | leptin receptor | LEPR | 0.000346 | 0.0390 | higher |
| U82276 | leukocyte | LILRA2 | 0.000131 | 0.0252 | lower |
| | immunoglobulin-like | | | | |
| | receptor, subfamily A | | | | |
| | (with TM domain), | | | | |
| | member 2 | | | | |
| BF965566 | leucine rich repeat (in | LRRFIP1 | 0.000011 | 0.0087 | higher |
| | FLII) interacting protein 1 | | | | |
| AI972475 | LYRIC/3D3 | LYRIC | 0.000129 | 0.0252 | higher |
| AI566096 | likely ortholog of mouse | M96 | 0.000411 | 0.0413 | higher |
| | metal response element | | | | |
| | binding transcription factor 2 | | | | |
| AF067173 | mago-nashi homolog, | MAGOH | 0.000472 | 0.0448 | higher |
| | proliferation-associated | | | | |
| | (Drosophila) | | | | |
| AI471665 | MYC-associated zinc | MAZ | 0.000451 | 0.0436 | lower |
| | finger protein (purine- | | | | |
| | binding transcription factor | | | | |
| AL556619 | methyl-CpG binding | MBD4 | 0.000333 | 0.0389 | higher |
| | domain protein 4 | | | | |
| NM_014763 | mitochondrial ribosomal | MRPL19 | 0.000320 | 0.0389 | higher |
| | protein L19 | | | | |
| BC001165 | N-ethylmaleimide- | NAPA | 0.000564 | 0.0486 | lower |
| | sensitive factor attachment | | | | |
| | protein, alpha | | | | |
| AI361805 | natural killer-tumor | NKTR | 0.000006 | 0.0087 | higher |
| | recognition sequence | | | | |
| BC004952 | likely ortholog of mouse | NSPC1 | 0.000008 | 0.0087 | lower |
| | nervous system polycomb 1 | | | | |
| NM_022731 | nuclear ubiquitous casein | NUCKS | 0.000198 | 0.0310 | higher |
| | kinase and cyclin- | | | | |
| | dependent kinase substrate | | | | |
| NM_005022 | profilin 1 | PFN1 | 0.000148 | 0.0266 | lower |
| NM_024165 | PHD finger protein 1 | PHF1 | 0.000486 | 0.0449 | lower |
| NM_004279 | peptidase (mitochondrial | PMPCB | 0.000180 | 0.0300 | higher |
| | processing) beta | | | | |
| NM_004774 | PPAR binding protein | PPARBP | 0.000296 | 0.0386 | higher |
| J03223 | proteoglycan 1, secretory | PRG1 | 0.000216 | 0.0328 | lower |
| | granule | | | | |
| BC001423 | proteasome (prosome, | PSME3 | 0.000021 | 0.0108 | lower |
| | macropain) activator | | | | |
| | subunit 3 (PA28 gamma; | | | | |
| | Ki) | | | | |
| BG029917 | proteasome (prosome, | PSMF1 | 0.000543 | 0.0476 | lower |
| | macropain) inhibitor | | | | |
| | subunit 1 (PI31) | | | | |
| AF348514 | prothymosin, alpha (gene | PTMA | 0.000083 | 0.0203 | higher |
| | sequence 28) | | | | |
| NM_002872 | ras-related C3 botulinum | RAC2 | 0.000088 | 0.0203 | lower |
| | toxin substrate 2 (rho | | | | |
| | family, small GTP binding | | | | |
| | protein Rac2) | | | | |
| NM_021039 | S100 calcium binding | S100A11 | 0.000329 | 0.0389 | lower |
| | protein A11 (calgizzarin) | | | | |
| NM_014845 | Sac domain-containing | SAC3 | 0.000342 | 0.0390 | higher |
| | inositol phosphatase 3 | | | | |
| NM_003930 | src family associated | SCAP2 | 0.000095 | 0.0213 | higher |
| | phosphoprotein 2 | | | | |
| NM_012430 | SEC22 vesicle trafficking | SEC22L2 | 0.000146 | 0.0266 | lower |
| | protein-like 2 (S. cerevisiae) | | | | |
| AV702810 | SET translocation | SET | 0.000070 | 0.0184 | higher |
| | (myeloid leukemia- | | | | |
| | associated) | | | | |
| NM_031286 | SH3 domain binding | SH3BGRL3 | 0.000150 | 0.0266 | lower |
| | glutamic acid-rich protein | | | | |
| | like 3 | | | | |
| NM_020239 | small protein effector 1 of | SPEC1 | 0.000071 | 0.0184 | higher |
| | Cdc42 | | | | |
| AW149364 | SFRS protein kinase 2 | SRPK2 | 0.000004 | 0.0087 | higher |
| M25077 | Sjogren syndrome antigen | SSA2 | 0.000328 | 0.0389 | higher |
| | A2 (60 kDa, | | | | |
| | ribonucleoprotein | | | | |
| | autoantigen SS-A/Ro) | | | | |
| NM_004760 | serine/threonine kinase 17a | STK17A | 0.000096 | 0.0213 | lower |
| | (apoptosis-inducing) | | | | |
| NM_016930 | syntaxin 18 | STX18 | 0.000105 | 0.0225 | higher |
| NM_000544 | transporter 2, ATP-binding | TAP2 | 0.000378 | 0.0408 | lower |
| | cassette, sub-family B | | | | |
| | (MDR/TAP) | | | | |
| NM_006521 | transcription factor binding | TFE3 | 0.000385 | 0.0408 | lower |
| | to IGHM enhancer 3 | | | | |
| AL031651 | tranglutaminase 2 | TGM2 | 0.000018 | 0.0099 | lower |
| BG403671 | THO complex 2 | THOC2 | 0.000015 | 0.0088 | higher |
| NM_003807 | tumor necrosis factor | TNFSF14 | 0.000195 | 0.0310 | higher |
| | (ligand) superfamily, | | | | |
| | member 14 | | | | |
| BF110993 | translocated promoter | TPR | 0.000003 | 0.0087 | higher |
| | region (to activated MET | | | | |
| | oncogene) | | | | |
| U84404 | ubiquitin protein ligase | UBE3A | 0.000066 | 0.0184 | higher |
| | E3A (human papilloma | | | | |
| | virus E6-associated | | | | |
| | protein, Angelman | | | | |
| | syndrome) | | | | |
| AI557312 | Unknown | Unknown | 0.000011 | 0.0087 | higher |
| AW301861 | Unknown | Unknown | 0.000014 | 0.0087 | higher |
| AV726646 | Unknown | Unknown | 0.000027 | 0.0123 | higher |
| BE737027 | Unknown | Unknown | 0.000047 | 0.0182 | higher |
| AA910371 | Unknown | Unknown | 0.000057 | 0.0184 | higher |
| BF680255 | Unknown | Unknown | 0.000116 | 0.0237 | higher |
| BE857772 | Unknown | Unknown | 0.000272 | 0.0364 | higher |
| AI345238 | Unknown | Unknown | 0.000292 | 0.0385 | higher |
| BF984434 | Unknown | Unknown | 0.000349 | 0.0390 | higher |
| AA292281 | Unknown | Unknown | 0.000377 | 0.0408 | higher |
| BF796940 | Unknown | Unknown | 0.000408 | 0.0413 | higher |
| U82278 | Unknown | Unknown | 0.000450 | 0.0436 | lower |
| AV753392 | Unknown | Unknown | 0.000529 | 0.0468 | higher |
| U79458 | WW domain binding | WBP2 | 0.000013 | 0.0087 | higher |
| | protein 2 | | | | |
| BE729523 | HbxAg transactivated | XTP2 | 0.000012 | 0.0087 | higher |
| | protein 2 | | | | |
| BC002323 | Zyxin | ZYX | 0.000309 | 0.0389 | lower |

TABLE 11
TOP 100 GENES IDENTIFIED BY GENECLUSTER AS ASSOCIATED WITH INCREASED EXPRESSION LEVELS
IN MENINGOENCEPHALITIS PATIENTS

| | | | | 5% | |
|---|---|---|---|---|---|
| Accession | Gene name (sorted | | | Permuted | |
| number | by ANOVA FDR) | Gene description | Score | Score | ANOVA FDR |
| NM_025080 | ASRGL1 | asparaginase like 1 | 1.47 | 1.40 | 0.009 |
| NM_013448 | BAZ1A | bromodomain adjacent to zinc finger domain, 1A | 0.77 | 0.76 | 0.009 |
| NM_001969 | EIF5 | eukaryotic translation initiation factor 5 | 0.91 | 0.89 | 0.009 |
| AK025600 | KIAA0553 | KIAA0553 protein | 0.72 | 0.70 | 0.009 |
| NM_004735 | LRRFIP1 | leucine rich repeat (in FLII) interacting protein 1 | 0.74 | 0.72 | 0.009 |
| NM_003138 | SRPK2 | SFRS protein kinase 2 | 0.82 | 0.81 | 0.009 |
| XM_211847 | Unknown | Unknown | 0.92 | 0.90 | 0.009 |
| NM_001862 | Unknown | Unknown | 0.77 | 0.76 | 0.009 |
| XM_047325 | THOC2 | THO complex 2 | 0.74 | 0.72 | 0.009 |
| BQ772224 | Unknown | Unknown | 0.86 | 0.85 | 0.012 |
| NM_006738 | AKAP13 | A kinase (PRKA) anchor protein 13 | 0.75 | 0.74 | 0.016 |
| NM_020239 | SPEC1 | Small protein effector 1 of Cdc42 | 0.88 | 0.86 | 0.018 |
| NM_130839 | UBE3A | ubiquitin protein ligase E3A (human papilloma virus E6-associated protein, | 1.35 | 1.31 | 0.018 |
| | | Angelman syndrome) | | | |
| NM_002823 | PTMA | prothymosin, alpha (gene sequence 28) | 0.89 | 0.88 | 0.020 |
| NM_003930 | SCAP2 | src family associated phosphoprotein 2 | 1.65 | 1.46 | 0.021 |
| NM_016930 | STX18 | syntaxin 18 | 1.31 | 1.20 | 0.022 |
| NM_001015 | Unknown | Unknown | 0.77 | 0.76 | 0.024 |
| NM_033360 | KRAS2 | v-Ki-ras2 Kirsten rat sarcoma 2 viral oncogene homolog | 0.73 | 0.71 | 0.025 |
| NM_004634 | BRPF1 | bromodomain and PHD finger containing, 1 | 0.92 | 0.90 | 0.027 |
| NM_004279 | PMPCB | Peptidase (mitochondrial processing) beta | 0.83 | 0.82 | 0.030 |
| NM_005348 | HSPCA | heat shock 90 kDa protein 1, alpha | 0.73 | 0.71 | 0.032 |
| NM_005890 | GAS7 | growth arrest-specific 7 | 0.72 | 0.70 | 0.034 |
| NM_004774 | PPARBP | PPAR binding protein | 1.37 | 1.34 | 0.039 |
| NM_015355 | JJAZ1 | joined to JAZF1 | 0.92 | 0.90 | 0.039 |
| NM_014763 | MRPL19 | mitochondrial ribosomal protein L19 | 0.89 | 0.88 | 0.039 |
| NM_004600 | SSA2 | Sjogren syndrome antigen A2 (60 kDa, ribonucleoprotein autoantigen SS- | 0.9 | 0.89 | 0.039 |
| | | A/Ro) | | | |
| NM_014845 | SAC3 | Sac domain-containing inositol phosphatase 3 | 0.76 | 0.74 | 0.039 |
| NM_001328 | Unknown | Unknown | 0.75 | 0.74 | 0.039 |
| NM_016311 | ATPIF1 | ATPase inhibitory factor 1 | 0.73 | 0.71 | 0.041 |
| AK022200 | DDX52 | DEAD (Asp-Glu-Ala-Asp) box polypeptide 52 | 0.84 | 0.83 | 0.041 |
| NM_007358 | M96 | likely ortholog of mouse metal response element binding transcription factor 2 | 0.92 | 0.92 | 0.041 |
| NM_002370 | MAGOH | mago-nashi homolog, proliferation-associated (Drosophila) | 0.75 | 0.73 | 0.045 |
| NM_012201 | GLG1 | Golgi apparatus protein 1 | 0.76 | 0.75 | 0.057 |
| NM_004539 | NARS | asparaginyl-tRNA synthetase | 0.83 | 0.83 | 0.062 |
| NM_012385 | P8 | p8 protein (candidate of metastasis 1) | 0.9 | 0.88 | 0.062 |
| NM_000988 | Unknown | Unknown | 0.82 | 0.82 | 0.062 |
| NM_006649 | SDCCAG16 | serologically defined colon cancer antigen 16 | 0.74 | 0.72 | 0.063 |
| NM_006024 | TAX1BP1 | Tax1 (human T-cell leukemia virus type I) binding protein 1 | 0.73 | 0.71 | 0.067 |
| NM_003328 | TXK | TXK tyrosine kinase | 0.78 | 0.78 | 0.068 |
| CA313371 | MBP | myelin basic protein | 0.77 | 0.76 | 0.069 |
| NM_000376 | VDR | vitamin D (1,25-dihydroxyvitamin D3) receptor | 0.91 | 0.89 | 0.070 |
| NM_004623 | TTC4 | tetratricopeptide repeat domain 4 | 0.93 | 0.92 | 0.074 |
| NM_019071 | ING3 | inhibitor of growth family, member 3 | 0.88 | 0.87 | 0.076 |
| NM_002823 | PTMA | prothymosin, alpha (gene sequence 28) | 0.85 | 0.85 | 0.076 |
| NM_014170 | HSPC135 | HSPC135 protein | 0.74 | 0.73 | 0.079 |
| NM_000181 | GUSB | glucuronidase, beta | 0.77 | 0.77 | 0.082 |
| NM_004871 | GOSR1 | golgi SNAP receptor complex member 1 | 0.76 | 0.75 | 0.084 |
| NM_001004 | Unknown | Unknown | 0.73 | 0.70 | 0.084 |
| BC010161 | ALU | ALU Sequence | 0.78 | 0.77 | 0.084 |
| AI478300 | ALU | ALU Sequence | 0.78 | 0.77 | 0.088 |
| NM_014810 | CAP350 | centrosome-associated protein 350 | 0.81 | 0.81 | 0.088 |
| CB530067 | CTSB | cathepsin B | 0.76 | 0.76 | 0.088 |
| NM_004442 | EPHB2 | EphB2 | 0.75 | 0.73 | 0.088 |
| NM_005102 | FEZ2 | fasciculation and elongation protein zeta 2 (zygin II) | 0.77 | 0.76 | 0.088 |
| NM_014171 | CRIPT | postsynaptic protein CRIPT | 0.74 | 0.72 | 0.090 |
| NM_001412 | EIF1A | eukaryotic translation initiation factor 1A | 0.92 | 0.90 | 0.094 |
| AB095946 | IPO9 | importin 9 | 0.78 | 0.78 | 0.094 |
| NM_152227 | SNX5 | sorting nexin 5 | 0.73 | 0.71 | 0.094 |
| NM_144498 | OSBPL2 | oxysterol binding protein-like 2 | 0.76 | 0.76 | 0.096 |
| NM_015523 | DKFZP566E144 | small fragment nuclease | 0.76 | 0.76 | 0.096 |
| BC036583 | PRKAR2A | protein kinase, cAMP-dependent, regulatory, type II, alpha | 0.84 | 0.83 | 0.097 |
| AK021482 | ALAD | aminolevulinate, delta-, dehydratase | 0.78 | 0.78 | 0.097 |
| NM_001637 | AOAH | acyloxyacyl hydrolase (neutrophil) | 0.82 | 0.81 | >0.1 |
| NM_005104 | BRD2 | bromodomain containing 2 | 0.75 | 0.74 | >0.1 |
| NM_003796 | C19ORF2 | chromosome 19 open reading frame 2 | 0.88 | 0.87 | >0.1 |
| NM_006807 | CBX1 | Chromobox homolog 1 (HP1 beta homolog Drosophila) | 0.82 | 0.81 | >0.1 |
| NM_016052 | CGI-115 | CGI-115 protein | 0.77 | 0.76 | >0.1 |
| NM_022802 | CTBP2 | C-terminal binding protein 2 | 0.82 | 0.82 | >0.1 |
| NM_006565 | CTCF | CCCTC-binding factor (zinc finger protein) | 0.75 | 0.74 | >0.1 |
| AW984453 | CUGBP1 | CUG triplet repeat, RNA binding protein 1 | 0.72 | 0.70 | >0.1 |
| NM_001363 | DKC1 | dyskeratosis congenita 1, dyskerin | 0.73 | 0.71 | >0.1 |
| NM_015497 | DKFZP564G2022 | DKFZP564G2022 protein | 0.83 | 0.82 | >0.1 |
| NM_173801 | FLJ12178 | hypothetical protein FLJ12178 | 0.88 | 0.87 | >0.1 |
| AF271783 | FLJ21174 | hypothetical protein FLJ21174 | 0.92 | 0.91 | >0.1 |
| NM_002027 | FNTA | farnesyltransferase, CAAX box, alpha | 0.76 | 0.75 | >0.1 |
| NM_177442 | FTSJ2 | FtsJ homolog 2 (E. coli) | 0.73 | 0.72 | >0.1 |
| NM_024629 | KLIP1 | KSHV latent nuclear antigen interacting protein 1 | 0.8 | 0.80 | >0.1 |
| XM_300615 | KNS2 | kinesin 2 60/70 kDa | 0.74 | 0.72 | >0.1 |
| NM_018479 | LOC55862 | uncharacterized hypothalamus protein HCDASE | 0.73 | 0.71 | >0.1 |
| NM_014462 | LSM1 | LSM1 homolog, U6 small nuclear RNA associated (S. cerevisiae) | 0.74 | 0.73 | >0.1 |
| NM_016019 | LUC7L2 | LUC7-like 2 (S. cerevisiae) | 0.74 | 0.73 | >0.1 |
| NM_018848 | MKKS | McKusick-Kaufman syndrome | 0.82 | 0.81 | >0.1 |
| NM_018657 | MYNN | Myoneurin | 0.74 | 0.73 | >0.1 |
| NM_017852 | NALP2 | NACHT, LRR and PYD containing protein 2 | 0.75 | 0.73 | >0.1 |
| NM_002484 | NUBP1 | nucleotide binding protein 1 (MinD homolog, E. coli) | 0.76 | 0.75 | >0.1 |
| NM_002552 | ORC4L | Origin recognition complex, subunit 4-like (yeast) | 0.81 | 0.81 | >0.1 |
| NM_002815 | PSMD11 | proteasome (prosome, macropain) 26S subunit, non-ATPase, 11 | 0.77 | 0.76 | >0.1 |
| NM_014676 | PUM1 | pumilio homolog 1 (Drosophila) | 0.73 | 0.71 | >0.1 |
| AK024423 | RNASEH1 | ribonuclease H1 | 0.83 | 0.82 | >0.1 |
| NM_006414 | RPP38 | ribonuclease P (38 kD) | 0.76 | 0.75 | >0.1 |
| NM_013306 | SNX15 | sorting nexin 15 | 0.76 | 0.74 | >0.1 |
| NM_003563 | SPOP | speckle-type POZ protein | 0.76 | 0.75 | >0.1 |
| NM_003765 | STX10 | syntaxin 10 | 0.84 | 0.84 | >0.1 |
| NM_006351 | TIMM44 | Translocase of inner mitochondrial membrane 44 homolog (yeast) | 0.83 | 0.82 | >0.1 |
| NM_003313 | TSTA3 | Tissue specific transplantation antigen P35B | 0.81 | 0.81 | >0.1 |
| NM_003314 | TTC1 | tetratricopeptide repeat domain 1 | 0.75 | 0.73 | >0.1 |
| NM_180699 | U1SNRNPBP | U1-snRNP binding protein homolog | 0.72 | 0.70 | >0.1 |
| AK092175 | Unknown | Unknown | 0.73 | 0.70 | >0.1 |
| NM_017528 | WBSCR22 | Williams Beuren syndrome chromosome region 22 | 0.73 | 0.71 | >0.1 |
| NM_018253 | YAP | YY1 associated protein | 0.77 | 0.77 | >0.1 |

TABLE 12
TOP 50 GENES IDENTIFIED BY GENECLUSTER AS
ASSOCIATED WITH LOWER EXPRESSION LEVELS
IN MENINGOENCEPHALITIS PATIENTS

| | Gene name | | | |
|---|---|---|---|---|
| Accession | (sorted by | | | ANOVA |
| number | ANOVA FDR) | Gene description | Score | FDR |
| NM_032673 | NSPC1 | likely ortholog of mouse nervous | 1.01 | 0.0087 |
| | | system polycomb 1 | | |
| NM_012478 | WBP2 | WW domain binding protein 2 | 0.89 | 0.0087 |
| NM_000201 | ICAM1 | intercellular adhesion molecule 1 | 0.87 | 0.0087 |
| | | (CD54), human rhinovirus receptor | | |
| NM_001655 | ARCN1 | archain 1 | 0.88 | 0.0092 |
| BQ581290 | TGM2 | transglutaminase 2 | 0.91 | 0.0099 |
| XM_300774 | AGRN | agrin | 0.91 | 0.0169 |
| NM_024845 | FLJ14154 | hypothetical protein FLJ14154 | 0.88 | 0.0184 |
| NM_001707 | BCL7B | B-cell CLL/lymphoma 7B | 0.86 | 0.0184 |
| NM_014940 | KIAA0872 | KIAA0872 protein | 0.86 | 0.0184 |
| NM_001693 | ATP6V1B2 | ATPase, H+ transporting, lysosomal | 0.9 | 0.0203 |
| | | 56/58 kDa, V1 subunit B, isoform 2 | | |
| NM_017450 | BAIAP2 | BAI1-associated protein 2 | 1.09 | 0.0271 |
| NM_018115 | FLJ10498 | hypothetical protein FLJ10498 | 0.89 | 0.0300 |
| NM_002727 | PRG1 | proteoglycan 1, secretory granule | 0.92 | 0.0328 |
| NM_014481 | APEX2 | APEX nuclease | 1.05 | 0.0330 |
| | | (apurinic/apyrimidinic endonuclease) 2 | | |
| NM_002569 | FURIN | furin (paired basic amino acid | 1.16 | 0.0363 |
| | | cleaving enzyme) | | |
| NM_000544 | TAP2 | transporter 2, ATP-binding cassette, | 1.09 | 0.0408 |
| | | sub-family B (MDR/TAP) | | |
| NM_006521 | TFE3 | transcription factor binding to IGHM | 0.88 | 0.0408 |
| | | enhancer 3 | | |
| NM_005620 | S100A11 | S100 calcium binding protein A11 | 0.87 | 0.0408 |
| | | (calgizzarin) | | |
| BM930256 | EMP1 | epithelial membrane protein 1 | 0.89 | 0.0442 |
| NM_024165 | PHF1 | PHD finger protein 1 | 0.94 | 0.0449 |
| NM_014741 | KIAA0652 | KIAA0652 gene product | 0.84 | 0.0485 |
| CD644158 | RAB5B | RAB5B, member RAS oncogene | 0.88 | 0.0596 |
| | | family | | |
| NM_004309 | ARHGDIA | Rho GDP dissociation inhibitor | 0.83 | 0.0619 |
| | | (GDI) alpha | | |
| AI911044 | PDI2 | peptidyl arginine deiminase, type II | 0.84 | 0.0651 |
| XM_114002 | NY-REN-24 | NY-REN-24 antigen | 0.89 | 0.0659 |
| NM_002355 | M6PR | mannose-6-phosphate receptor | 0.86 | 0.0705 |
| | | (cation dependent) | | |
| CD643922 | STK17B | serine/threonine kinase 17b | 0.87 | 0.0711 |
| | | (apoptosis-inducing) | | |
| NM_012075 | C16ORF35 | chromosome 16 open reading frame | 0.91 | 0.0742 |
| | | 35 | | |
| AB002368 | XPO6 | exportin 6 | 0.92 | 0.0793 |
| NM_148175 | PPIL2 | peptidylprolyl isomerase | 0.86 | 0.0836 |
| | | (cyclophilin)-like 2 | | |
| NM_022060 | ABHD4 | abhydrolase domain containing 4 | 0.96 | 0.0884 |
| NM_014734 | KIAA0247 | KIAA0247 gene product | 0.93 | 0.0884 |
| NM_078467 | CDKN1A | cyclin-dependent kinase inhibitor 1A | 0.84 | 0.0884 |
| | | (p21, Cip1) | | |
| NM_002530 | NTRK3 | neurotrophic tyrosine kinase, | 1.07 | 0.0945 |
| | | receptor, type 3 | | |
| AK023816 | UNK_AK023816 | Homo sapiens cDNA FLJ13754 fis, | 0.86 | 0.0979 |
| | | clone PLACE3000362. | | |
| NM_000201 | ICAM1 | intercellular adhesion molecule 1 | 1.02 | >0.1 |
| | | (CD54), human rhinovirus receptor | | |
| BU627400 | CTSB | cathepsin B | 0.97 | >0.1 |
| BX117520 | FLJ13910 | hypothetical protein FLJ13910 | 0.94 | >0.1 |
| AW969709 | F2RL1 | coagulation factor II (thrombin) | 0.94 | >0.1 |
| | | receptor-like 1 | | |
| XM_114002 | NY-REN-24 | NY-REN-24 antigen | 0.91 | >0.1 |
| AB040972 | FLJ11560 | hypothetical protein FLJ11560 | 0.91 | >0.1 |
| NM_006865 | LILRA3 | leukocyte immunoglobulin-like | 0.9 | >0.1 |
| | | receptor, subfamily A (without TM | | |
| | | domain), member 3 | | |
| BI020084 | MKRN1 | makorin, ring finger protein, 1 | 0.9 | >0.1 |
| NM_170665 | ATP2A2 | ATPase, Ca++ transporting, cardiac | 0.89 | >0.1 |
| | | muscle, slow twitch 2 | | |
| NM_016525 | UBAP1 | ubiquitin associated protein 1 | 0.87 | >0.1 |
| NM_000167 | GK | glycerol kinase | 0.85 | >0.1 |
| NM_015444 | RIS1 | Ras-induced senescence 1 | 0.84 | >0.1 |
| NM_000270 | NP | nucleoside phosphorylase | 0.84 | >0.1 |
| NM_023079 | FLJ13855 | hypothetical protein FLJ13855 | 0.84 | >0.1 |

TABLE 13
GENES THAT CAPTURE FIVE-OUT-OF-FIVE
MENINGOENCEPHALITIS PATIENTS

| | | | Cutoff level for | | |
|---|---|---|---|---|---|
| Accession | | | association with | Unadjusted | |
| number | Gene name | Gene description | meningoencephalitis | p value | FDR |
| AA284705 | ICAM1 | intercellular adhesion | F < 5 | 0.000010 | 0.0087 |
| | | molecule 1 (CD54), | | | |
| | | human rhinovirus receptor | | | |
| NM_001343 | DAB2 | disabled homolog 2, | F > 11 | 0.000065 | 0.0184 |
| | | mitogen-responsive | | | |
| | | phosphoprotein | | | |
| | | (Drosophila) | | | |
| U84404 | UBE3A | ubiquitin protein ligase | F > 16 | 0.000066 | 0.0184 |
| | | E3A (human papilloma | | | |
| | | virus E6-associated | | | |
| | | protein, Angelman | | | |
| | | syndrome) | | | |
| AF348514 | PTMA | prothymosin, alpha (gene | F > 80 | 0.000083 | 0.0203 |
| | | sequence 28) | | | |
| NM_003930 | SCAP2 | src family associated | F > 75 | 0.000095 | 0.0213 |
| | | phosphoprotein 2 | | | |
| NM_016930 | STX18 | syntaxin 18 | F > 25 | 0.000105 | 0.0225 |
| AI972475 | LYRIC | LYRIC/3D3 | F > 20 | 0.000129 | 0.0252 |
| U26455 | ATM | ataxia telangiectasia | F > 24 | 0.000232 | 0.0338 |
| | | mutated (includes | | | |
| | | complementation groups | | | |
| | | A, C and D) | | | |
| NM_016281 | JIK | STE20-like kinase | F > 48 | 0.000250 | 0.0352 |
| NM_002569 | FURIN | furin (paired basic amino | F < 10 | 0.000264 | 0.0363 |
| | | acid cleaving enzyme; | | | |
| | | PACE) | | | |
| BE646414 | GGA2 | golgi associated, gamma | F > 256 | 0.000271 | 0.0364 |
| | | adaptin ear containing, | | | |
| | | ARF binding protein 2 | | | |
| NM_004774 | PPARBP | PPAR binding protein | F > 8 | 0.000296 | 0.0386 |
| NM_017775 | FLJ20343 | hypothetical protein | F > 39 | 0.000315 | 0.0389 |
| | | FLJ20343 | | | |
| NM_000544 | TAP2 | transporter 2, ATP- | F < 5 | 0.000378 | 0.0408 |
| | | binding cassette, | | | |
| | | subfamily B (MDR/TAP) | | | |

TABLE 14
Selected examples of accuracy of classification
using expression patterns associated
with meningoencephalitis

| | | Meningoen- | | |
|---|---|---|---|---|
| | | cephalitis | Nonencephalitis | |
| | | patients | patients | Identification |
| | | correctly | incorrectly | numbers |
| | | identified^{a}/ | identified/ | of patients |
| Gene | Metric | total (%) | total (%) | incorrectly |
| name | applied | (5 in group) | (103 in group) | classified |
| SRPK2 | F > 5 | 3/(60%) | 3/(3%) | 8, 252, 752 |
| NKTR | F > 5 | 3/(60%) | 3/(3%) | 8, 252, 752 |
| TPR | present | 3/(60%) | 4/(4%) | 8, 14, 252, 752 |
| ASRGL1 | F > 20 | 4/(80%) | 5/(5%) | 43, 53, 273, |
| | | | | 297, 316* |
| ASRGL1 | F > 12 | 5/(100%) | 22/(21%) | 2, 6, 8, 15, 22, |
| | | | | 23, 24, 25, 36, |
| | | | | 40, 43, 53, 60, |
| | | | | 254, 270, 271, |
| | | | | 273, 295, 297, |
| | | | | 312, 316, |
| | | | | 753** |
| SCAP2 | F > 75 | 5/(100%) | 11/(11%) | 5, 7, 12, 14, 24, |
| | | | | 32, 258, 271, |
| | | | | 753, 755, 758 |
| DAB2 | F > 11 | 5/(100%) | 13/(13%) | 6, 8, 14, 15, 32, |
| | | | | 50, 254, 271, |
| | | | | 281, 300, |
| | | | | 316, 753, 755 |
| ^{a}Frequency cutoffs were selected as capturing the number of meningoencephalitis patients indicated. The total number of nonencephalitis patients incorrectly classified due to expressing at least one gene | | | | |
| # above the cutoff is 36 (out of 103). If the requirement to capture encephalitis patient 301 is dropped, the total number of nonencephalitis patients misclassified due to expressing at least one gene above cutoff is 9. | | | | |

*All of these patients are male. Therefore, data for these patients were not considered in calculating the statistical significance of the association of this gene with meningoencephalitis.
**Of these patients, 14 are male. Data from males were not used in calculating the association between expression level and meningoencephalitis.

TABLE 15
Examples of genes that show an association with meningoencephalitis
in both AN1792-stimulated and control cultures

| | | p value metric: | FDR metric: | | |
|---|---|---|---|---|---|
| | | gene | gene | | |
| | | frequency in | frequency in | p value metric: | FDR metric: |
| | Gene name | antigen- | antigen- | gene frequency in | gene frequency in |
| Accession | (sorted | positive | positive | antigen-negative | antigen-negative |
| number | alphabetically) | cultures | cultures | cultures | cultures |
| M90360 | AKAP13 | 0.000037 | 0.016 | 0.000035 | 0.017 |
| NM_025080 | ASRGL1 | 0.000001 | 0.009 | 0.000076 | 0.024 |
| AA102574 | BAZ1A | 0.000010 | 0.009 | 0.000021 | 0.013 |
| NM_001343 | DAB2 | 0.000065 | 0.018 | 0.000286 | 0.055 |
| BG530850 | DDX18 | 0.000189 | 0.030 | 0.000372 | 0.066 |
| AW081113 | DKFZP564B0769 | 0.000008 | 0.009 | 0.000001 | 0.002 |
| BG481972 | EIF5 | 0.000006 | 0.009 | 0.000019 | 0.013 |
| NM_004459 | FALZ | 0.000059 | 0.018 | 0.000054 | 0.020 |
| NM_017736 | FLJ20274 | 0.000080 | 0.020 | 0.000027 | 0.014 |
| AU145053 | FNBP1 | 0.000409 | 0.041 | 0.000067 | 0.024 |
| BE646414 | GGA2 | 0.000271 | 0.036 | 0.000809 | 0.092 |
| BG420237 | HSPCA | 0.000207 | 0.032 | 0.000081 | 0.024 |
| NM_000201 | ICAM1 | 0.000010 | 0.009 | 0.001032 | 0.100 |
| AI673812 | KIAA0553 | 0.000014 | 0.009 | 0.000002 | 0.003 |
| BF673699 | KRAS2 | 0.000127 | 0.025 | 0.000083 | 0.024 |
| BF965566 | LRRFIP1 | 0.000011 | 0.009 | 0.000056 | 0.020 |
| AI972475 | LYRIC | 0.000129 | 0.025 | 0.000007 | 0.006 |
| AI566096 | M96 | 0.000411 | 0.041 | 0.000205 | 0.045 |
| AI361805 | NKTR | 0.000006 | 0.009 | 0.000017 | 0.013 |
| AW149364 | SRPK2 | 0.000004 | 0.009 | 0.000025 | 0.013 |
| BG403671 | THOC2 | 0.000015 | 0.009 | 0.001291 | 0.108 |
| BF110993 | TPR | 0.000003 | 0.009 | 0.000000 | 0.002 |
| BE729523 | XTP2 | 0.000012 | 0.009 | 0.000001 | 0.002 |

TABLE 16
Examples of genes that show an association with meningo-
encephalitis in AN1792-stimulated cultures and no
association in control cultures

| | | p value | FDR: | p value | FDR |
|---|---|---|---|---|---|
| | | metric: | metric: | metric: | metric: |
| | | gene | gene | gene | gene |
| | Gene | frequency | frequency | frequency | frequency |
| | name | in | in | in | in |
| | (sorted | antigen- | antigen- | antigen- | antigen- |
| Accession | alpha- | positive | positive | negative | negative |
| number | betically) | cultures | cultures | cultures | cultures |
| NM_014481 | APEX2 | 0.000221 | 0.033002 | 0.863468 | 0.959865 |
| NM_017450 | BAIAP2 | 0.00016 | 0.027081 | 0.853139 | 0.957957 |
| NM_001707 | BCL7B | 0.000069 | 0.018403 | 0.540282 | 0.827374 |
| AF098641 | CD44 | 0.000152 | 0.026582 | 0.705258 | 0.903721 |
| NM_018115 | FLJ10498 | 0.000183 | 0.029962 | 0.919492 | 0.979341 |

TABLE 17
Genes associated with meningoencephalitis by GeneCluster analysis using the ratio metric

| | Gene name | | | |
|---|---|---|---|---|
| | (sorted | | | |
| Accession | alpha- | | Unadjusted | ANOVA |
| number | betically) | Gene description | p value | FDR |
| NM_014576 | ACF | apobec-1 complementation factor | 0.000329 | 0.223274 |
| U48705 | DDR1 | discoidin domain receptor family, | 0.00004 | 0.10434 |
| | | member 1 | | |
| NM_000173 | GP1BA | glycoprotein Ib (platelet), alpha | 0.000694 | 0.301068 |
| | | polypeptide | | |
| AK024651 | GPR107 | G protein-coupled receptor 107 | 0.000131 | 0.111287 |
| BC002842 | HIST1H2BD | histone 1, H2bd | 0.000051 | 0.10434 |
| BE888744 | IFIT2 | interferon-induced protein with | | |
| | | tetratricopeptide repeats 2 | 0.000504 | 0.269983 |
| AU150943 | LOC221061 | hypothetical protein LOC221061 | 0.000062 | 0.105681 |
| NM_005446 | P2RXL1 | purinergic receptor P2X-like 1, | 0.001729 | 0.342444 |
| | | orphan receptor | | |
| AL512687 | PM5 | pM5 protein | 0.001386 | 0.319676 |
| M57399 | PTN | pleiotrophin (heparin binding growth | 0.00206 | 0.342444 |
| | | factor 8, neurite growth-promoting | | |
| | | factor 1) | | |
| NM_021908 | ST7 | suppression of tumorigenicity 7 | 0.000025 | 0.10434 |
| AK023816 | UNK_AK023816 | Homo sapiens cDNA FLJ13754 fis, clone | 0.000098 | 0.110884 |
| | | PLACE3000362. | | |
| X93006 | Unknown | Unknown | 0.001095 | 0.301068 |

TABLE 18
Genes associated with IgG responsiveness by ANOVA using gene frequency metric
(FDR < 0.011)

| | | | | | | Average |
|---|---|---|---|---|---|---|
| | | | | | | expression in |
| | | | | | | IgG |
| | | | | | | nonresponders |
| | Gene name | | | | | relative to |
| Accession | (sorted by | | Unadjusted | ANOVA | Adjusted | average in IgG |
| number | p value) | Gene description | p value | FDR | p value | responders |
| NM_013417 | IARS | isoleucine-tRNA | 6.389E−07 | 0.004997 | 0.01 | lower |
| | | synthetase | | | | |
| NM_004279 | PMPCB | peptidase (mitochondrial | 9.394E−07 | 0.004997 | 0.01 | lower |
| | | processing) beta | | | | |
| NM_173638 | Unknown | Unknown | 0.000001 | 0.004997 | 0.01 | lower |
| NM_005736 | ACTR1A | ARP1 actin-related protein | 0.000002 | 0.004997 | 0.02 | higher |
| | | 1 homolog A, centractin | | | | |
| | | alpha (yeast) | | | | |
| NM_000099 | CST3 | cystatin C (amyloid | 0.000002 | 0.004997 | 0.02 | higher |
| | | angiopathy and cerebral | | | | |
| | | hemorrhage) | | | | |
| NM_004107 | FCGRT | Fc fragment of IgG, | 0.000002 | 0.004997 | 0.02 | higher |
| | | receptor, transporter, alpha | | | | |
| XM_086398 | Unknown | Unknown | 0.000002 | 0.004997 | 0.02 | lower |
| NM_003328 | TXK | TXK tyrosine kinase | 0.000003 | 0.007541 | 0.04 | lower |
| NM_000754 | COMT | catechol-O- | 0.000004 | 0.007702 | 0.05 | higher |
| | | methyltransferase | | | | |
| NM_002087 | GRN | Granulin | 0.000004 | 0.007541 | 0.04 | higher |
| NM_002388 | MCM3 | MCM3 minichromosome | 0.000005 | 0.007702 | 0.05 | lower |
| | | maintenance deficient 3 | | | | |
| | | (S. cerevisiae) | | | | |
| NM_024835 | LZK1 | C3HC4-type zinc finger | 0.000006 | 0.009032 | 0.07 | lower |
| | | protein | | | | |
| NM_005216 | DDOST | dolichyl- | 0.000009 | 0.010942 | 0.09 | higher |
| | | diphosphooligosaccharide- | | | | |
| | | protein glycosyltransferase | | | | |
| NM_005022 | PFN1 | profilin 1 | 0.000009 | 0.010942 | 0.09 | higher |
| XM_295598 | SF3A1 | splicing factor 3a, subunit | 0.000009 | 0.010942 | 0.09 | lower |
| | | 1, 120 kDa | | | | |

TABLE 19
Response groups as segregated by the four genes most strongly
associated with IgG responsiveness by GeneCluster

| | Patients in 70^{th} percentile of gene frequency level | | | |
|---|---|---|---|---|
| | associated with IgG responsiveness | | | |
| | Average | | | |
| | expression | | | |
| | in IgG | | | |
| | nonresponders | IgG non- | IgG | IgG partial |
| | relative | responders/ | responders/ | responders/ |
| | to average | (%) (60 | (%) (22 | (%) (26 |
| Gene | in IgG | patients | patients | patients |
| name | responders | in group) | in group) | in group) |
| Granulin | Higher | 25/(42%) | 0/(0%) | 7/(27%) |
| FCGRT | Higher | 26/(43%) | 0/(0%) | 6/(23%) |
| IARS | Lower | 25/(42%) | 1/(5%) | 6/(23%) |
| MCM3 | Lower | 23/(38%) | 1/(5%) | 9/(34%) |

TABLE 20
Samples Received By Pharmacogenomic Laboratory

| | Number |
|---|---|
| Enrolled U.S. patients | 172 |
| Enrolled patients who consented to pharmacogenomic | 167 |
| portion of study | |
| Samples within shipping specifications | 161 |
| Samples that generated data from chips that | 153 |
| met all QC criteria | |

TABLE 21
Patients in Study by Year of Birth

| Age (years) | Number of Patients |
|---|---|
| ≧80 | 29 |
| 70-80 | 61 |
| 60-70 | 29 |
| 50-60 | 4 |
| Cumulative total | 123 |

TABLE 22
Samples in Pharmacogenomic Study

| | Male | Female | Total |
|---|---|---|---|
| Placebo | 12 | 18 | 30 |
| Treated | 66 | 57 | 123 |
| Treated IgG responders | 12 | 13 | 25 |
| Treated IgG partial responders | 12 | 16 | 28 |
| Treated IgG nonresponders | 42 | 28 | 70 |
| Treated IgM responders | 20 | 20 | 40 |
| Treated IgM partial responders | 18 | 21 | 39 |
| Treated IgM nonresponders | 28 | 16 | 44 |
| Meningoencephalitis patients | 0 | 5 | 5 |

TABLE 23

| Affymetrix | |
|---|---|
| Qualifier | Gene Name |
| 213266_at | 76P |
| 209993_at | ABCB1 |
| 202804_at | ABCC1 |
| 213485_s_at | ABCC10 |
| 214033_at | ABCC6 |
| 201873_s_at | ABCE1 |
| 200965_s_at | ABLIM1 |
| 49452_at | ACACB |
| 222011_s_at | ACAT2 |
| 221641_s_at | ACATE2 |
| 201630_s_at | ACP1 |
| 204393_s_at | ACPP |
| 207275_s_at | ACSL1 |
| 202422_s_at | ACSL4 |
| 200720_s_at | ACTR1A |
| 219623_at | ACTR5 |
| 204639_at | ADA |
| 202604_x_at | ADAM10 |
| 202381_at | ADAM9 |
| 202912_at | ADM |
| 204183_s_at | ADRBK2 |
| 211071_s_at | AF1Q |
| 203566_s_at | AGL |
| 201491_at | AHSA1 |
| 212980_at | AHSA2 |
| 215051_x_at | AIF1 |
| 209901_x_at | AIF1 |
| 213095_x_at | AIF1 |
| 212543_at | AIM1 |
| 202587_s_at | AK1 |
| 201675_at | AKAP1 |
| 203156_at | AKAP11 |
| 210517_s_at | AKAP12 |
| 201425_at | ALDH2 |
| 214221_at | ALMS1 |
| 214366_s_at | ALOX5 |
| 204446_s_at | ALOX5 |
| 202125_s_at | ALS2CR3 |
| 204294_at | AMT |
| 218575_at | ANAPC1 |
| 206385_s_at | ANK3 |
| 212289_at | ANKRD12 |
| 213005_s_at | ANKRD15 |
| 213035_at | ANKRD28 |
| 202888_s_at | ANPEP |
| 201012_at | ANXA1 |
| 201590_x_at | ANXA2 |
| 210427_x_at | ANXA2 |
| 208816_x_at | ANXA2P2 |
| 201302_at | ANXA4 |
| 200782_at | ANXA5 |
| 205639_at | AOAH |
| 221937_at | AP1GBP1 |
| 64418_at | AP1GBP1 |
| 203300_x_at | AP1S2 |
| 211047_x_at | AP2S1 |
| 203410_at | AP3M2 |
| 202442_at | AP3S1 |
| 209870_s_at | APBA2 |
| 209871_s_at | APBA2 |
| 221492_s_at | APG3L |
| 201687_s_at | API5 |
| 211404_s_at | APLP2 |
| 208702_x_at | APLP2 |
| 214875_x_at | APLP2 |
| 208703_s_at | APLP2 |
| 221087_s_at | APOL3 |
| 202268_s_at | APPBP1 |
| 202630_at | APPBP2 |
| 39248_at | AQP3 |
| 205568_at | AQP9 |
| 201526_at | ARF5 |
| 202211_at | ARFGAP3 |
| 57082_at | ARH |
| 221790_s_at | ARH |
| 38149_at | ARHGAP25 |
| 37117_at | ARHGAP8 |
| 213039_at | ARHGEF18 |
| 208736_at | ARPC3 |
| 211963_s_at | ARPC5 |
| 210980_s_at | ASAH1 |
| 213902_at | ASAH1 |
| 212818_s_at | ASB1 |
| 206743_s_at | ASGR1 |
| 206130_s_at | ASGR2 |
| 204244_s_at | ASK |
| 205047_s_at | ASNS |
| 218987_at | ATF7IP |
| 208758_at | ATIC |
| 212672_at | ATM |
| 203454_s_at | ATOX1 |
| 207522_s_at | ATP2A3 |
| 211755_s_at | ATP5F1 |
| 207809_s_at | ATP6AP1 |
| 200078_s_at | ATP6V0B |
| 212041_at | ATP6V0D1 |
| 200096_s_at | ATP6V0E |
| 201972_at | ATP6V1A |
| 201089_at | ATP6V1B2 |
| 201527_at | ATP6V1F |
| 209903_s_at | ATR |
| 208002_s_at | BACH |
| 221234_s_at | BACH2 |
| 217911_s_at | BAG3 |
| 219667_s_at | BANK1 |
| 202121_s_at | BC-2 |
| 203053_at | BCAS2 |
| 214390_s_at | BCAT1 |
| 202030_at | BCKDK |
| 219528_s_at | BCL11B |
| 203685_at | BCL2 |
| 205681_at | BCL2A1 |
| 203140_at | BCL6 |
| 206465_at | BG1 |
| 204493_at | BID |
| 210201_x_at | BIN1 |
| 210538_s_at | BIRC3 |
| 202592_at | BLOC1S1 |
| 206126_at | BLR1 |
| 211729_x_at | BLVRA |
| 203773_x_at | BLVRA |
| 215460_x_at | BRD1 |
| 205715_at | BST1 |
| 204901_at | BTRC |
| 202096_s_at | BZRP |
| 218889_at | C10ORF117 |
| 220147_s_at | C12ORF14 |
| 219099_at | C12ORF5 |
| 218422_s_at | C13ORF10 |
| 218852_at | C14ORF10 |
| 218139_s_at | C14ORF108 |
| 212460_at | C14ORF147 |
| 219526_at | C14ORF169 |
| 219316_s_at | C14ORF58 |
| 221940_at | C18B11 |
| 222099_s_at | C19ORF13 |
| 214173_x_at | C19ORF2 |
| 213390_at | C19ORF7 |
| 218456_at | C1QDC1 |
| 202878_s_at | C1QR1 |
| 217835_x_at | C20ORF24 |
| 212996_s_at | C21ORF108 |
| 203996_s_at | C21ORF2 |
| 221984_s_at | C2ORF17 |
| 213615_at | C3F |
| 210054_at | C4ORF15 |
| 214661_s_at | C4ORF9 |
| 220751_s_at | C5ORF4 |
| 220088_at | C5R1 |
| 218195_at | C6ORF211 |
| 219006_at | C6ORF66 |
| 218877_s_at | C6ORF75 |
| 218116_at | C9ORF78 |
| 219147_s_at | C9ORF95 |
| 221631_at | CACNA1I |
| 211984_at | CALM1 |
| 210349_at | CAMK4 |
| 218309_at | CAMKIINALPHA |
| 212252_at | CAMKK2 |
| 201850_at | CAPG |
| 201238_s_at | CAPZA2 |
| 201949_x_at | CAPZB |
| 37012_at | CAPZB |
| 218929_at | CARF |
| 211208_s_at | CASK |
| 206011_at | CASP1 |
| 211367_s_at | CASP1 |
| 209970_x_at | CASP1 |
| 207467_x_at | CAST |
| 207625_s_at | CBFA2T2 |
| 209682_at | CBLB |
| 212914_at | CBX7 |
| 204655_at | CCL5 |
| 1405_i_at | CCL5 |
| 200953_s_at | CCND2 |
| 208796_s_at | CCNG1 |
| 213743_at | CCNT2 |
| 221511_x_at | CCPG1 |
| 205098_at | CCR1 |
| 206337_at | CCR7 |
| 201947_s_at | CCT2 |
| 206587_at | CCT6B |
| 201743_at | CD14 |
| 203645_s_at | CD163 |
| 215049_x_at | CD163 |
| 208653_s_at | CD164 |
| 205789_at | CD1D |
| 205831_at | CD2 |
| 206545_at | CD28 |
| 209555_s_at | CD36 |
| 206488_s_at | CD36 |
| 213539_at | CD3D |
| 206804_at | CD3G |
| 210031_at | CD3Z |
| 216942_s_at | CD58 |
| 211744_s_at | CD58 |
| 205173_x_at | CD58 |
| 213958_at | CD6 |
| 200663_at | CD63 |
| 203507_at | CD68 |
| 209795_at | CD69 |
| 214049_x_at | CD7 |
| 210895_s_at | CD86 |
| 205758_at | CD8A |
| 206761_at | CD96 |
| 205627_at | CDA |
| 213151_s_at | CDC10 |
| 221556_at | CDC14B |
| 209658_at | CDC16 |
| 201853_s_at | CDC25B |
| 207318_s_at | CDC2L5 |
| 209288_s_at | CDC42EP3 |
| 209286_at | CDC42EP3 |
| 218157_x_at | CDC42SE1 |
| 204995_at | CDK5R1 |
| 218315_s_at | CDK5RAP1 |
| 209501_at | CDR2 |
| 204029_at | CELSR2 |
| 204066_s_at | CENTG2 |
| 205642_at | CEP1 |
| 202195_s_at | CGI-100 |
| 218102_at | CGI-26 |
| 219590_x_at | CGI-30 |
| 214426_x_at | CHAF1A |
| 219049_at | CHGN |
| 214665_s_at | CHP |
| 204065_at | CHST10 |
| 221059_s_at | CHST6 |
| 221058_s_at | CKLF |
| 219161_s_at | CKLF |
| 212752_at | CLASP1 |
| 219947_at | CLECSF6 |
| 208659_at | CLIC1 |
| 221042_s_at | CLMN |
| 200743_s_at | CLN2 |
| 204050_s_at | CLTA |
| 207270_x_at | CMRF35 |
| 203291_at | CNOT4 |
| 203642_s_at | COBLL1 |
| 203073_at | COG2 |
| 208818_s_at | COMT |
| 221676_s_at | CORO1C |
| 203663_s_at | COX5A |
| 211025_x_at | COX5B |
| 201943_s_at | CPD |
| 201940_at | CPD |
| 210069_at | CPT1B |
| 208146_s_at | CPVL |
| 201200_at | CREG |
| 210766_s_at | CSE1L |
| 203104_at | CSF1R |
| 203591_s_at | CSF3R |
| 202332_at | CSNK1E |
| 204619_s_at | CSPG2 |
| 215646_s_at | CSPG2 |
| 211571_s_at | CSPG2 |
| 204620_s_at | CSPG2 |
| 221731_x_at | CSPG2 |
| 201201_at | CSTB |
| 212905_at | CSTF2T |
| 203947_at | CSTF3 |
| 218924_s_at | CTBS |
| 200765_x_at | CTNNA1 |
| 210844_x_at | CTNNA1 |
| 200839_s_at | CTSB |
| 200838_at | CTSB |
| 201487_at | CTSC |
| 200766_at | CTSD |
| 203657_s_at | CTSF |
| 202295_s_at | CTSH |
| 202087_s_at | CTSL |
| 202902_s_at | CTSS |
| 209665_at | CYB561D2 |
| 203922_s_at | CYBB |
| 203923_s_at | CYBB |
| 201066_at | CYC1 |
| 208923_at | CYFIP1 |
| 215785_s_at | CYFIP2 |
| 221903_s_at | CYLD |
| 213295_at | CYLD |
| 201926_s_at | DAF |
| 201678_s_at | DC12 |
| 203799_at | DCL-1 |
| 204246_s_at | DCTN3 |
| 218013_x_at | DCTN4 |
| 214909_s_at | DDAH2 |
| 202262_x_at | DDAH2 |
| 203409_at | DDB2 |
| 212690_at | DDHD2 |
| 201241_at | DDX1 |
| 204977_at | DDX10 |
| 208149_x_at | DDX11 |
| 208159_x_at | DDX11 |
| 208896_at | DDX18 |
| 200694_s_at | DDX24 |
| 218819_at | DDX26 |
| 215693_x_at | DDX27 |
| 219108_x_at | DDX27 |
| 221780_s_at | DDX27 |
| 205000_at | DDX3Y |
| 220890_s_at | DDX47 |
| 202447_at | DECR1 |
| 215158_s_at | DEDD |
| 205382_s_at | DF |
| 203385_at | DGKA |
| 217989_at | DHRS8 |
| 212674_s_at | DHX30 |
| 205726_at | DIAPH2 |
| 219374_s_at | DIBD1 |
| 201479_at | DKC1 |
| 221541_at | DKFZP434B044 |
| 202560_s_at | DKFZP547E1010 |
| 213657_s_at | DKFZP547K1113 |
| 37590_g_at | DKFZP547K1113 |
| 212333_at | DKFZP564F0522 |
| 221265_s_at | DKFZP564O1664 |
| 210006_at | DKFZP564O243 |
| 208092_s_at | DKFZP566A1524 |
| 221970_s_at | DKFZP586L0724 |
| 213199_at | DKFZP586P0123 |
| 36552_at | DKFZP586P0123 |
| 214247_s_at | DKK3 |
| 212727_at | DLG3 |
| 201681_s_at | DLG5 |
| 218794_s_at | DLP |
| 212730_at | DMN |
| 203301_s_at | DMTF1 |
| 205963_s_at | DNAJA3 |
| 200666_s_at | DNAJB1 |
| 202867_s_at | DNAJB12 |
| 202500_at | DNAJB2 |
| 213088_s_at | DNAJC9 |
| 212538_at | DOCK9 |
| 208872_s_at | DP1 |
| 203717_at | DPP4 |
| 204646_at | DPYD |
| 200762_at | DPYSL2 |
| 217868_s_at | DREV1 |
| 204751_x_at | DSC2 |
| 203635_at | DSCR3 |
| 208892_s_at | DUSP6 |
| 208891_at | DUSP6 |
| 208893_s_at | DUSP6 |
| 57532_at | DVL2 |
| 202968_s_at | DYRK2 |
| 218660_at | DYSF |
| 218482_at | E(Y)2 |
| 219551_at | EAF2 |
| 204858_s_at | ECGF1 |
| 220048_at | EDAR |
| 204642_at | EDG1 |
| 212830_at | EGFL5 |
| 222221_x_at | EHD1 |
| 218935_at | EHD3 |
| 201018_at | EIF1AX |
| 201017_at | EIF1AX |
| 201016_at | EIF1AX |
| 201019_s_at | EIF1AX |
| 204409_s_at | EIF1AY |
| 209429_x_at | EIF2B4 |
| 212351_at | EIF2B5 |
| 201142_at | EIF2S1 |
| 201530_x_at | EIF4A1 |
| 31845_at | ELF4 |
| 220386_s_at | EML4 |
| 201324_at | EMP1 |
| 201325_s_at | EMP1 |
| 207610_s_at | EMR2 |
| 201313_at | ENO2 |
| 209473_at | ENTPD1 |
| 207691_x_at | ENTPD1 |
| 204076_at | ENTPD4 |
| 212375_at | EP400 |
| 204505_s_at | EPB49 |
| 200843_s_at | EPRS |
| 202176_at | ERCC3 |
| 202414_at | ERCC5 |
| 201328_at | ETS2 |
| 201329_s_at | ETS2 |
| 204328_at | EVER1 |
| 217838_s_at | EVL |
| 204714_s_at | F5 |
| 209271_at | FALZ |
| 203974_at | FAM16AX |
| 203184_at | FBN2 |
| 209696_at | FBP1 |
| 213145_at | FBXL14 |
| 209004_s_at | FBXL5 |
| 212231_at | FBXO21 |
| 218432_at | FBXO3 |
| 204232_at | FCER1G |
| 214511_x_at | FCGR1A |
| 216950_s_at | FCGR1A |
| 203561_at | FCGR2A |
| 218831_s_at | FCGRT |
| 205237_at | FCN1 |
| 201798_s_at | FER1L3 |
| 205418_at | FES |
| 219069_at | FGIF |
| 204834_at | FGL2 |
| 206492_at | FHIT |
| 201540_at | FHL1 |
| 219117_s_at | FKBP11 |
| 200709_at | FKBP1A |
| 58780_s_at | FLJ10357 |
| 218274_s_at | FLJ10415 |
| 218993_at | FLJ10581 |
| 221806_s_at | FLJ10707 |
| 217884_at | FLJ10774 |
| 222132_s_at | FLJ10842 |
| 218125_s_at | FLJ10853 |
| 218347_at | FLJ10900 |
| 218552_at | FLJ10948 |
| 209688_s_at | FLJ10996 |
| 218307_at | FLJ11164 |
| 213694_at | FLJ11220 |
| 218633_x_at | FLJ11342 |
| 39650_s_at | FLJ11383 |
| 219361_s_at | FLJ12484 |
| 219765_at | FLJ12586 |
| 218312_s_at | FLJ12895 |
| 218370_s_at | FLJ12903 |
| 218532_s_at | FLJ20152 |
| 219734_at | FLJ20174 |
| 219646_at | FLJ20186 |
| 219809_at | FLJ20195 |
| 220306_at | FLJ20202 |
| 218652_s_at | FLJ20265 |
| 218710_at | FLJ20272 |
| 219460_s_at | FLJ20507 |
| 219258_at | FLJ20516 |
| 217961_at | FLJ20551 |
| 221229_s_at | FLJ20628 |
| 218932_at | FLJ20729 |
| 217895_at | FLJ20758 |
| 218366_x_at | FLJ20859 |
| 219315_s_at | FLJ20898 |
| 218483_s_at | FLJ21827 |
| 65635_at | FLJ21865 |
| 212918_at | FLJ22028 |
| 219435_at | FLJ22170 |
| 222143_s_at | FLJ22405 |
| 221081_s_at | FLJ22457 |
| 219359_at | FLJ22635 |
| 218454_at | FLJ22662 |
| 218754_at | FLJ23323 |
| 218776_s_at | FLJ23375 |
| 208903_at | FLJ46061 |
| 210607_at | FLT3LG |
| 212232_at | FNBP4 |
| 200090_at | FNTA |
| 204829_s_at | FOLR2 |
| 206015_s_at | FOXJ3 |
| 203064_s_at | FOXK2 |
| 214148_at | FOXM1 |
| 202945_at | FPGS |
| 205119_s_at | FPR1 |
| 210773_s_at | FPRL1 |
| 210772_at | FPRL1 |
| 209702_at | FTO |
| 205324_s_at | FTSJ1 |
| 213594_x_at | FUSIP1 |
| 209893_s_at | FUT4 |
| 209892_at | FUT4 |
| 217897_at | FXYD6 |
| 210105_s_at | FYN |
| 202812_at | GAA |
| 200645_at | GABARAP |
| 219013_at | GALNT11 |
| 218885_s_at | GALNT12 |
| 213049_at | GARNL1 |
| 211067_s_at | GAS7 |
| 204793_at | GASP |
| 209603_at | GATA3 |
| 209602_s_at | GATA3 |
| 209604_s_at | GATA3 |
| 203765_at | GCA |
| 218912_at | GCC1 |
| 212139_at | GCN1L1 |
| 202182_at | GCN5L2 |
| 206589_at | GFI1 |
| 202722_s_at | GFPT1 |
| 208914_at | GGA2 |
| 209249_s_at | GHITM |
| 204222_s_at | GLIPR1 |
| 204221_x_at | GLIPR1 |
| 209276_s_at | GLRX |
| 217807_s_at | GLTSCR2 |
| 35820_at | GM2A |
| 205349_at | GNA15 |
| 214157_at | GNAS |
| 204000_at | GNB5 |
| 201921_at | GNG10 |
| 207157_s_at | GNG5 |
| 212335_at | GNS |
| 212334_at | GNS |
| 208798_x_at | GOLGIN-67 |
| 210425_x_at | GOLGIN-67 |
| 210279_at | GPR18 |
| 200736_s_at | GPX1 |
| 220864_s_at | GRIM19 |
| 204396_s_at | GRK5 |
| 211284_s_at | GRN |
| 216041_x_at | GRN |
| 200678_x_at | GRN |
| 200696_s_at | GSN |
| 201912_s_at | GSPT1 |
| 205541_s_at | GSPT2 |
| 205770_at | GSR |
| 201470_at | GSTO1 |
| 205930_at | GTF2E1 |
| 202605_at | GUSB |
| 214501_s_at | H2AFY |
| 209818_s_at | HABP4 |
| 202282_at | HADH2 |
| 211699_x_at | HBA1 |
| 202300_at | HBXIP |
| 218345_at | HCA112 |
| 219484_at | HCFC2 |
| 218450_at | HEBP1 |
| 218603_at | HECA |
| 212815_at | HELIC1 |
| 218306_s_at | HERC1 |
| 201944_at | HEXB |
| 203020_at | HHL |
| 38340_at | HIP1R |
| 209558_s_at | HIP1R |
| 204512_at | HIVEP1 |
| 205936_s_at | HK3 |
| 205671_s_at | HLA-DOB |
| 214438_at | HLX1 |
| 206074_s_at | HMGA1 |
| 203665_at | HMOX1 |
| 204112_s_at | HNMT |
| 211732_x_at | HNMT |
| 209068_at | HNRPDL |
| 204647_at | HOMER3 |
| 208470_s_at | HPR |
| 202854_at | HPRT1 |
| 219403_s_at | HPSE |
| 218092_s_at | HRB |
| 203202_at | HRB2 |
| 209971_x_at | HRI |
| 218508_at | HSA275986 |
| 213598_at | HSA9761 |
| 204405_x_at | HSA9761 |
| 200941_at | HSBP1 |
| 209657_s_at | HSF2 |
| 221771_s_at | HSMPP8 |
| 221597_s_at | HSPC171 |
| 212493_s_at | HYPB |
| 218805_at | IAN4L1 |
| 204744_s_at | IARS |
| 210439_at | ICOS |
| 203596_s_at | IFIT5 |
| 204785_x_at | IFNAR2 |
| 202727_s_at | IFNGR1 |
| 201642_at | IFNGR2 |
| 201393_s_at | IGF2R |
| 210095_s_at | IGFBP3 |
| 212827_at | IGHM |
| 206420_at | IGSF6 |
| 202491_s_at | IKBKAP |
| 209575_at | IL10RB |
| 204773_at | IL11RA |
| 201888_s_at | IL13RA1 |
| 201887_at | IL13RA1 |
| 203679_at | IL1RL1LG |
| 212657_s_at | IL1RN |
| 221658_s_at | IL21R |
| 220054_at | IL23A |
| 205291_at | IL2RB |
| 217804_s_at | ILF3 |
| 208594_x_at | ILT8 |
| 203126_at | IMPA2 |
| 205376_at | INPP4B |
| 203006_at | INPP5A |
| 204706_at | INPP5E |
| 213792_s_at | INSR |
| 200995_at | IPO7 |
| 200993_at | IPO7 |
| 205995_x_at | IQCB1 |
| 220034_at | IRAK3 |
| 33304_at | ISG20 |
| 201656_at | ITGA6 |
| 205055_at | ITGAE |
| 210213_s_at | ITGB4BP |
| 211339_s_at | ITK |
| 202747_s_at | ITM2A |
| 202746_at | ITM2A |
| 203723_at | ITPKB |
| 201189_s_at | ITPR3 |
| 206700_s_at | JARID1D |
| 212496_s_at | JMJD2B |
| 202138_x_at | JTV1 |
| 201464_x_at | JUN |
| 212192_at | KCTD12 |
| 200700_s_at | KDELR2 |
| 203712_at | KIAA0020 |
| 212789_at | KIAA0056 |
| 213483_at | KIAA0073 |
| 212510_at | KIAA0089 |
| 203492_x_at | KIAA0092 |
| 203493_s_at | KIAA0092 |
| 213006_at | KIAA0146 |
| 212844_at | KIAA0179 |
| 212733_at | KIAA0226 |
| 212735_at | KIAA0226 |
| 212053_at | KIAA0251 |
| 212621_at | KIAA0286 |
| 40016_g_at | KIAA0303 |
| 212356_at | KIAA0323 |
| 203288_at | KIAA0355 |
| 203049_s_at | KIAA0372 |
| 202713_s_at | KIAA0391 |
| 203959_s_at | KIAA0478 |
| 36545_s_at | KIAA0542 |
| 212946_at | KIAA0564 |
| 212675_s_at | KIAA0582 |
| 212579_at | KIAA0650 |
| 212663_at | KIAA0674 |
| 212311_at | KIAA0746 |
| 212314_at | KIAA0746 |
| 212546_s_at | KIAA0826 |
| 212548_s_at | KIAA0826 |
| 212570_at | KIAA0830 |
| 36888_at | KIAA0841 |
| 212402_at | KIAA0853 |
| 209760_at | KIAA0922 |
| 213407_at | KIAA0931 |
| 209654_at | KIAA0947 |
| 216996_s_at | KIAA0971 |
| 213092_x_at | KIAA0974 |
| 201270_x_at | KIAA1068 |
| 213271_s_at | KIAA1117 |
| 209379_s_at | KIAA1128 |
| 209378_s_at | KIAA1128 |
| 212453_at | KIAA1279 |
| 203086_at | KIF2 |
| 203087_s_at | KIF2 |
| 221219_s_at | KLHDC4 |
| 221221_s_at | KLHL3 |
| 206785_s_at | KLRC2 |
| 211954_s_at | KPNB3 |
| 211955_at | KPNB3 |
| 201003_x_at | KUA-UEV |
| 204385_at | KYNU |
| 210663_s_at | KYNU |
| 217388_s_at | KYNU |
| 203041_s_at | LAMP2 |
| 203042_at | LAMP2 |
| 202020_s_at | LANCL1 |
| 217933_s_at | LAP3 |
| 200673_at | LAPTM4A |
| 200618_at | LASP1 |
| 211005_at | LAT |
| 209881_s_at | LAT |
| 207734_at | LAX |
| 221011_s_at | LBH |
| 204891_s_at | LCK |
| 204012_s_at | LCMT2 |
| 201030_x_at | LDHB |
| 221558_s_at | LEF1 |
| 202594_at | LEPROTL1 |
| 202595_s_at | LEPROTL1 |
| 201105_at | LGALS1 |
| 208949_s_at | LGALS3 |
| 208934_s_at | LGALS8 |
| 202726_at | LIG1 |
| 210660_at | LILRA1 |
| 211100_x_at | LILRA2 |
| 207857_at | LILRA2 |
| 211101_x_at | LILRA2 |
| 210146_x_at | LILRB2 |
| 207697_x_at | LILRB2 |
| 210225_x_at | LILRB3 |
| 211133_x_at | LILRB3 |
| 211135_x_at | LILRB3 |
| 220036_s_at | LIMR |
| 206440_at | LIN7A |
| 201847_at | LIPA |
| 212697_at | LOC162427 |
| 214838_at | LOC375035 |
| 221249_s_at | LOC81558 |
| 214791_at | LOC93349 |
| 47560_at | LPHN1 |
| 212276_at | LPIN1 |
| 202460_s_at | LPIN2 |
| 220532_s_at | LR8 |
| 211596_s_at | LRIG1 |
| 200785_s_at | LRP1 |
| 209841_s_at | LRRN3 |
| 202245_at | LSS |
| 214574_x_at | LST1 |
| 211582_x_at | LST1 |
| 210629_x_at | LST1 |
| 207339_s_at | LTB |
| 203005_at | LTBR |
| 217842_at | LUC7L2 |
| 205859_at | LY86 |
| 215967_s_at | LY9 |
| 206584_at | LY96 |
| 202625_at | LYN |
| 218437_s_at | LZTFL1 |
| 203362_s_at | MAD2L1 |
| 206363_at | MAF |
| 209014_at | MAGED1 |
| 218176_at | MAGEF1 |
| 218573_at | MAGEH1 |
| 210092_at | MAGOH |
| 204777_s_at | MAL |
| 210017_at | MALT1 |
| 214180_at | MAN1C1 |
| 209166_s_at | MAN2B1 |
| 204089_x_at | MAP3K4 |
| 214339_s_at | MAP4K1 |
| 206296_x_at | MAP4K1 |
| 210449_x_at | MAPK14 |
| 202788_at | MAPKAPK3 |
| 201669_s_at | MARCKS |
| 205819_at | MARCO |
| 214363_s_at | MATR3 |
| 209332_s_at | MAX |
| 218440_at | MCCC1 |
| 35147_at | MCF2L |
| 212246_at | MCFD2 |
| 201930_at | MCM6 |
| 219952_s_at | MCOLN1 |
| 219066_at | MDS018 |
| 219698_s_at | METTL4 |
| 201126_s_at | MGAT1 |
| 219797_at | MGAT4A |
| 222120_at | MGC13138 |
| 214696_at | MGC14376 |
| 221756_at | MGC17330 |
| 221904_at | MGC21688 |
| 222064_s_at | MGC2744 |
| 221255_s_at | MGC2963 |
| 212313_at | MGC29816 |
| 204699_s_at | MGC29875 |
| 204700_x_at | MGC29875 |
| 202365_at | MGC5139 |
| 221580_s_at | MGC5306 |
| 218750_at | MGC5306 |
| 200899_s_at | MGEA5 |
| 204168_at | MGST2 |
| 204917_s_at | MLLT3 |
| 200644_at | MLP |
| 204959_at | MNDA |
| 209583_s_at | MOX2 |
| 212885_at | MPHOSPH10 |
| 215731_s_at | MPHOSPH9 |
| 212197_x_at | M-RIP |
| 214771_x_at | M-RIP |
| 218027_at | MRPL15 |
| 208787_at | MRPL3 |
| 201717_at | MRPL49 |
| 209609_s_at | MRPL9 |
| 211594_s_at | MRPL9 |
| 218259_at | MRTF-B |
| 210356_x_at | MS4A1 |
| 217418_x_at | MS4A1 |
| 219607_s_at | MS4A4A |
| 219666_at | MS4A6A |
| 41220_at | MSF |
| 202911_at | MSH6 |
| 218773_s_at | MSRB |
| 213511_s_at | MTMR1 |
| 216095_x_at | MTMR1 |
| 218716_x_at | MTO1 |
| 203774_at | MTR |
| 210386_s_at | MTX1 |
| 207727_s_at | MUTYH |
| 202431_s_at | MYC |
| 201960_s_at | MYCBP2 |
| 209124_at | MYD88 |
| 212082_s_at | MYL6 |
| 213733_at | MYO1F |
| 202423_at | MYST3 |
| 212462_at | MYST4 |
| 48612_at | N4BP1 |
| 221867_at | N4BP1 |
| 212653_s_at | NACSIN |
| 202944_at | NAGA |
| 218231_at | NAGK |
| 218189_s_at | NANS |
| 204749_at | NAP1L3 |
| 201414_s_at | NAP1L4 |
| 37005_at | NBL1 |
| 219079_at | NCB5OR |
| 209949_at | NCF2 |
| 207677_s_at | NCF4 |
| 205147_x_at | NCF4 |
| 203315_at | NCK2 |
| 219231_at | NCOA6IP |
| 214181_x_at | NCR3 |
| 211583_x_at | NCR3 |
| 208759_at | NCSTN |
| 210817_s_at | NDP52 |
| 214867_at | NDST2 |
| 203621_at | NDUFB5 |
| 211752_s_at | NDUES7 |
| 203413_at | NELL2 |
| 217722_s_at | NEUGRIN |
| 211105_s_at | NFATC1 |
| 202584_at | NFX1 |
| 202215_s_at | NFYC |
| 217963_s_at | NGFRAP1 |
| 218240_at | NKIRAS2 |
| 200902_at | NM_004261.1 |
| 205006_s_at | NMT2 |
| 200875_s_at | NOL5A |
| 217962_at | NOLA3 |
| 211951_at | NOLC1 |
| 217950_at | NOSIP |
| 213775_x_at | NP220 |
| 209798_at | NPAT |
| 200701_at | NPC2 |
| 200063_s_at | NPM1 |
| 203814_s_at | NQO2 |
| 204791_at | NR2C1 |
| 204651_at | NRF1 |
| 217850_at | NS |
| 210023_s_at | NSPC1 |
| 213061_s_at | NTAN1 |
| 217802_s_at | NUCKS |
| 207545_s_at | NUMB |
| 209073_s_at | NUMB |
| 218768_at | NUP107 |
| 212247_at | NUP205 |
| 213945_s_at | NUP210 |
| 202900_s_at | NUP88 |
| 214945_at | NY-REN-7 |
| 201599_at | OAT |
| 201364_s_at | OAZ2 |
| 201365_at | OAZ2 |
| 200790_at | ODC1 |
| 203569_s_at | OFD1 |
| 206323_x_at | OPHN1 |
| 202074_s_at | OPTN |
| 210028_s_at | ORC3L |
| 204957_at | ORC5L |
| 218556_at | ORMDL2 |
| 209627_s_at | OSBPL3 |
| 209626_s_at | OSBPL3 |
| 202780_at | OXCT1 |
| 210401_at | P2RX1 |
| 210448_s_at | P2RX5 |
| 218589_at | P2RY5 |
| 208051_s_at | PAIP1 |
| 202759_s_at | PALM2 |
| 218771_at | PANK4 |
| 213534_s_at | PASK |
| 216945_x_at | PASK |
| 212825_at | PAXIP1L |
| 205353_s_at | PBP |
| 214177_s_at | PBXIP1 |
| 214512_s_at | PC4 |
| 209361_s_at | PCBP4 |
| 214937_x_at | PCM1 |
| 210156_s_at | PCMT1 |
| 218014_at | PCNT1 |
| 212422_at | PDCD11 |
| 212593_s_at | PDCD4 |
| 202731_at | PDCD4 |
| 222317_at | PDE3B |
| 204735_at | PDE4A |
| 204491_at | PDE4D |
| 212390_at | PDE4DIP |
| 214099_s_at | PDE4DIP |
| 214129_at | PDE4DIP |
| 208690_s_at | PDLIM1 |
| 202671_s_at | PDXK |
| 219132_at | PELI2 |
| 218472_s_at | PELO |
| 218590_at | PEO1 |
| 55616_at | PERLD1 |
| 204992_s_at | PFN2 |
| 200886_s_at | PGAM1 |
| 208454_s_at | PGCP |
| 200737_at | PGK1 |
| 200738_s_at | PGK1 |
| 219394_at | PGS1 |
| 222125_s_at | PH-4 |
| 212660_at | PHF15 |
| 218517_at | PHF17 |
| 203691_at | PI3 |
| 212506_at | PICALM |
| 205452_at | PIGB |
| 212120_at | PIGF |
| 212240_s_at | PIK3R1 |
| 219788_at | PILRA |
| 222218_s_at | PILRA |
| 220954_s_at | PILRB |
| 204269_at | PIM2 |
| 201192_s_at | PITPN |
| 218667_at | PJA1 |
| 204612_at | PKIA |
| 202732_at | PKIG |
| 201251_at | PKM2 |
| 60528_at | PLA2G4B |
| 206214_at | PLA2G7 |
| 205372_at | PLAG1 |
| 207002_s_at | PLAGL1 |
| 203471_s_at | PLEK |
| 201136_at | PLP2 |
| 202430_s_at | PLSCR1 |
| 202446_s_at | PLSCR1 |
| 214081_at | PLXDC1 |
| 219700_at | PLXDC1 |
| 208890_s_at | PLXNB2 |
| 213241_at | PLXNC1 |
| 213677_s_at | PMS1 |
| 218224_at | PNMA1 |
| 203366_at | POLG |
| 218016_s_at | POLR3E |
| 203782_s_at | POLRMT |
| 32502_at | PP1665 |
| 212199_at | PP784 |
| 200661_at | PPGB |
| 203063_at | PPM1F |
| 216347_s_at | PPP1R13B |
| 41577_at | PPP1R16B |
| 212750_at | PPP1R16B |
| 201877_s_at | PPP2R5C |
| 32541_at | PPP3CC |
| 207000_s_at | PPP3CC |
| 206174_s_at | PPP6C |
| 200975_at | PPT1 |
| 201494_at | PRCP |
| 203057_s_at | PRDM2 |
| 218329_at | PRDM4 |
| 201619_at | PRDX3 |
| 201858_s_at | PRG1 |
| 202741_at | PRKACB |
| 213093_at | PRKCA |
| 209048_s_at | PRKCBP1 |
| 209049_s_at | PRKCBP1 |
| 210038_at | PRKCQ |
| 210039_s_at | PRKCQ |
| 38269_at | PRKD2 |
| 204061_at | PRKX |
| 206279_at | PRKY |
| 204447_at | PROSAPIP1 |
| 209440_at | PRPS1 |
| 221036_s_at | PSFL |
| 209337_at | PSIP1 |
| 208805_at | PSMA6 |
| 200039_s_at | PSMB2 |
| 201400_at | PSMB3 |
| 202353_s_at | PSMD12 |
| 218371_s_at | PSPC1 |
| 211178_s_at | PSTPIP1 |
| 219938_s_at | PSTPIP2 |
| 206278_at | PTAFR |
| 206631_at | PTGER2 |
| 205171_at | PTPN4 |
| 206687_s_at | PTPN6 |
| 202897_at | PTPNS1 |
| 204960_at | PTPRCAP |
| 203554_x_at | PTTG1 |
| 204020_at | PURA |
| 201608_s_at | PWP1 |
| 201607_at | PWP1 |
| 221666_s_at | PYCARD |
| 202990_at | PYGL |
| 205174_s_at | QPCT |
| 201482_at | QSCN6 |
| 219622_at | RAB20 |
| 209514_s_at | RAB27A |
| 210951_x_at | RAB27A |
| 217763_s_at | RAB31 |
| 217764_s_at | RAB31 |
| 204214_s_at | RAB32 |
| 207405_s_at | RAD17 |
| 212646_at | RAFTLIN |
| 218337_at | RAI16 |
| 202100_at | RALB |
| 201711_x_at | RANBP2 |
| 210676_x_at | RANBP2L1 |
| 212842_x_at | RANBP2L1 |
| 212127_at | RANGAP1 |
| 209284_s_at | RAP140 |
| 209285_s_at | RAP140 |
| 204070_at | RARRES3 |
| 205590_at | RASGRP1 |
| 203185_at | RASSF2 |
| 201092_at | RBBP7 |
| 212331_at | RBL2 |
| 203250_at | RBM16 |
| 218593_at | RBM28 |
| 213852_at | RBM8A |
| 204098_at | RBMX2 |
| 212820_at | RC3 |
| 213878_at | RECQL |
| 202296_s_at | RER1 |
| 220570_at | RETN |
| 204023_at | RFC4 |
| 216834_at | RGS1 |
| 202988_s_at | RGS1 |
| 209324_s_at | RGS16 |
| 201453_x_at | RHEB |
| 204951_at | RHOH |
| 214700_x_at | RIF1 |
| 209684_at | RIN2 |
| 218598_at | RINT-1 |
| 209941_at | RIPK1 |
| 213338_at | RIS1 |
| 209882_at | RIT1 |
| 218269_at | RNASE3L |
| 213397_x_at | RNASE4 |
| 213566_at | RNASE6 |
| 207735_at | RNF125 |
| 215031_x_at | RNF126 |
| 217865_at | RNF130 |
| 219104_at | RNF141 |
| 204040_at | RNF144 |
| 219035_s_at | RNF34 |
| 212696_s_at | RNF4 |
| 218286_s_at | RNF7 |
| 203160_s_at | RNF8 |
| 202683_s_at | RNMT |
| 208270_s_at | RNPEP |
| 210479_s_at | RORA |
| 210426_x_at | RORA |
| 217559_at | RPL10L |
| 200809_x_at | RPL12 |
| 221726_at | RPL22 |
| 213084_x_at | RPL23A |
| 212039_x_at | RPL3 |
| 211073_x_at | RPL3 |
| 213689_x_at | RPL5 |
| 200908_s_at | RPLP2 |
| 201011_at | RPN1 |
| 205562_at | RPP38 |
| 214001_x_at | RPS10 |
| 200949_x_at | RPS20 |
| 201909_at | RPS4Y1 |
| 212928_at | RPS5P1 |
| 204171_at | RPS6KB1 |
| 218909_at | RPS6KC1 |
| 221524_s_at | RRAGD |
| 212589_at | RRAS2 |
| 212590_at | RRAS2 |
| 201477_s_at | RRM1 |
| 219549_s_at | RTN3 |
| 211509_s_at | RTN4 |
| 36129_at | RUTBC1 |
| 200660_at | S100A11 |
| 205863_at | S100A12 |
| 203186_s_at | S100A4 |
| 202917_s_at | S100A8 |
| 203535_at | S100A9 |
| 213262_at | SACS |
| 32099_at | SAFB2 |
| 204900_x_at | SAP30 |
| 218854_at | SART2 |
| 200069_at | SART3 |
| 213988_s_at | SAT |
| 203408_s_at | SATB1 |
| 39835_at | SBF1 |
| 209146_at | SC4MOL |
| 211423_s_at | SC5DL |
| 205790_at | SCAP1 |
| 218217_at | SCPEP1 |
| 202541_at | SCYE1 |
| 202071_at | SDC4 |
| 212607_at | SDCCAG8 |
| 202228_s_at | SDFR1 |
| 219349_s_at | SEC5L1 |
| 218265_at | SECISBP2 |
| 219351_at | SEDL |
| 204563_at | SELL |
| 210124_x_at | SEMA4F |
| 208939_at | SEPHS1 |
| 212414_s_at | SEPT6 |
| 213666_at | SEPT6 |
| 212413_at | SEPT6 |
| 214298_x_at | SEPT6 |
| 212415_at | SEPT6 |
| 217977_at | SEPX1 |
| 202833_s_at | SERPINA1 |
| 212268_at | SERPINB1 |
| 213572_s_at | SERPINB1 |
| 206034_at | SERPINB8 |
| 218346_s_at | SESN1 |
| 200687_s_at | SF3B3 |
| 213370_s_at | SFMBT1 |
| 212001_at | SFRS14 |
| 201129_at | SFRS7 |
| 200044_at | SFRS9 |
| 201698_s_at | SFRS9 |
| 220642_x_at | SH120 |
| 201312_s_at | SH3BGRL |
| 201311_s_at | SH3BGRL |
| 204019_s_at | SH3YL1 |
| 221519_at | SHFM3 |
| 221833_at | SIAH1 |
| 201998_at | SIAT1 |
| 52940_at | SIGIRR |
| 211761_s_at | SIP |
| 201381_x_at | SIP |
| 220485_s_at | SIRPB2 |
| 218878_s_at | SIRT1 |
| 205484_at | SIT |
| 206181_at | SLAMF1 |
| 210422_x_at | SLC11A1 |
| 206600_s_at | SLC16A5 |
| 209003_at | SLC25A11 |
| 202433_at | SLC35B1 |
| 218826_at | SLC35F2 |
| 218237_s_at | SLC38A1 |
| 212110_at | SLC39A14 |
| 211030_s_at | SLC6A6 |
| 201195_s_at | SLC7A5 |
| 203579_s_at | SLC7A6 |
| 204588_s_at | SLC7A7 |
| 202983_at | SMARCA3 |
| 210357_s_at | SMOX |
| 205596_s_at | SMURF2 |
| 218788_s_at | SMYD3 |
| 213447_at | SNRPN |
| 201522_x_at | SNRPN |
| 206042_x_at | SNURF |
| 218404_at | SNX10 |
| 210648_x_at | SNX3 |
| 216841_s_at | SOD2 |
| 212807_s_at | SORT1 |
| 212780_at | SOS1 |
| 207777_s_at | SP140 |
| 216274_s_at | SPC18 |
| 217827_s_at | SPG21 |
| 202524_s_at | SPOCK2 |
| 202523_s_at | SPOCK2 |
| 204011_at | SPRY2 |
| 214925_s_at | SPTAN1 |
| 215235_at | SPTAN1 |
| 203127_s_at | SPTLC2 |
| 217995_at | SQRDL |
| 210959_s_at | SRD5A1 |
| 211056_s_at | SRD5A1 |
| 214789_x_at | SRP46 |
| 207040_s_at | ST13 |
| 204150_at | STAB1 |
| 208992_s_at | STAT3 |
| 206118_at | STAT4 |
| 202693_s_at | STK17A |
| 202695_s_at | STK17A |
| 202786_at | STK39 |
| 211106_at | SUPT3H |
| 201483_s_at | SUPT4H1 |
| 212894_at | SUPV3L1 |
| 209447_at | SYNE1 |
| 202761_s_at | SYNE2 |
| 205691_at | SYNGR3 |
| 212828_at | SYNJ2 |
| 205804_s_at | T3JAM |
| 216925_s_at | TAL1 |
| 201463_s_at | TALDO1 |
| 212978_at | TA-LRRP |
| 204770_at | TAP2 |
| 202813_at | TARBP1 |
| 37278_at | TAZ |
| 203386_at | TBC1D4 |
| 213400_s_at | TBL1X |
| 208130_s_at | TBXAS1 |
| 202396_at | TCERG1 |
| 209153_s_at | TCF3 |
| 205255_x_at | TCF7 |
| 212764_at | TCF8 |
| 217909_s_at | TCFL4 |
| 203303_at | TCTE1L |
| 200803_s_at | TEGT |
| 219131_at | TERE1 |
| 203611_at | TERF2 |
| 218104_at | TEX10 |
| 206715_at | TFEC |
| 210215_at | TFR2 |
| 208249_s_at | TGDS |
| 201506_at | TGFBI |
| 204731_at | TGFBR3 |
| 212910_at | THAP11 |
| 218492_s_at | THAP7 |
| 204064_at | THOC1 |
| 202393_s_at | TIEG |
| 201666_at | TIMP1 |
| 203167_at | TIMP2 |
| 208838_at | TIP120A |
| 208700_s_at | TKT |
| 208699_x_at | TKT |
| 206472_s_at | TLE3 |
| 212769_at | TLE3 |
| 204924_at | TLR2 |
| 209150_s_at | TM9SF1 |
| 212194_s_at | TM9SF4 |
| 218113_at | TMEM2 |
| 202644_s_at | TNFAIP3 |
| 203508_at | TNFRSF1B |
| 219423_x_at | TNFRSF25 |
| 210847_x_at | TNFRSF25 |
| 211841_s_at | TNFRSF25 |
| 206150_at | TNFRSF7 |
| 210314_x_at | TNFSF13 |
| 209499_x_at | TNFSF13 |
| 211495_x_at | TNFSF13 |
| 209500_x_at | TNFSF13 |
| 207892_at | TNFSF5 |
| 212261_at | TNRC15 |
| 201870_at | TOMM34 |
| 201519_at | TOMM70A |
| 221601_s_at | TOSO |
| 221602_s_at | TOSO |
| 204529_s_at | TOX |
| 201690_s_at | TPD52 |
| 201379_s_at | TPD52L2 |
| 200822_x_at | TPI1 |
| 201731_s_at | TPR |
| 210972_x_at | TRA@ |
| 209671_x_at | TRA@ |
| 205599_at | TRAF1 |
| 204352_at | TRAF5 |
| 201391_at | TRAP1 |
| 219434_at | TREM1 |
| 218425_at | TRIAD3 |
| 202478_at | TRIB2 |
| 218145_at | TRIB3 |
| 217147_s_at | TRIM |
| 213009_s_at | TRIM37 |
| 209390_at | TSC1 |
| 221493_at | TSPYL1 |
| 210645_s_at | TTC3 |
| 208073_x_at | TTC3 |
| 208195_at | TTN |
| 214983_at | TTTY15 |
| 218184_at | TULP4 |
| 203246_s_at | TUSC4 |
| 208864_s_at | TXN |
| 208959_s_at | TXNDC4 |
| 207668_x_at | TXNDC7 |
| 204122_at | TYROBP |
| 213876_x_at | U2AF1L2 |
| 200058_s_at | U5-200KD |
| 219192_at | UBAP2 |
| 221839_s_at | UBAP2 |
| 211764_s_at | UBE2D1 |
| 203109_at | UBE2M |
| 218011_at | UBL5 |
| 202706_s_at | UMPS |
| 220998_s_at | UNC93B1 |
| 213274_s_at | UNK_AA020826 |
| 212993_at | UNK_AA114166 |
| 221728_x_at | UNK_AA628440 |
| 214686_at | UNK_AA868898 |
| 211563_s_at | UNK_AB006572 |
| 222108_at | UNK_AC004010 |
| 211796_s_at | UNK_AF043179 |
| 211429_s_at | UNK_AF119873 |
| 217473_x_at | UNK_AF229163 |
| 222001_x_at | UNK_AI160126 |
| 202969_at | UNK_AI216690 |
| 50376_at | UNK_AI278629 |
| 213152_s_at | UNK_AI343248 |
| 64064_at | UNK_AI435089 |
| 217526_at | UNK_AI478300 |
| 213161_at | UNK_AI583393 |
| 212239_at | UNK_AI680192 |
| 215399_s_at | UNK_AI683900 |
| 221918_at | UNK_AI742210 |
| 204860_s_at | UNK_AI817801 |
| 221850_x_at | UNK_AI826075 |
| 221973_at | UNK_AI983904 |
| 217028_at | UNK_AJ224869 |
| 216044_x_at | UNK_AK027146 |
| 40446_at | UNK_AL021366 |
| 202789_at | UNK_AL022394 |
| 212642_s_at | UNK_AL023584 |
| 213540_at | UNK_AL031228 |
| 203608_at | UNK_AL031230 |
| 212636_at | UNK_AL031781 |
| 209733_at | UNK_AL034399 |
| 212234_at | UNK_AL034550 |
| 213213_at | UNK_AL035669 |
| 212400_at | UNK_AL043266 |
| 213817_at | UNK_AL049435 |
| 214948_s_at | UNK_AL050136 |
| 216199_s_at | UNK_AL109942 |
| 212430_at | UNK_AL109955 |
| 212098_at | UNK_AL134724 |
| 212737_at | UNK_AL513583 |
| 212606_at | UNK_AL536319 |
| 213193_x_at | UNK_AL559122 |
| 212501_at | UNK_AL564683 |
| 212222_at | UNK_AU143855 |
| 221876_at | UNK_AU151157 |
| 214218_s_at | UNK_AV699347 |
| 202124_s_at | UNK_AV705253 |
| 212274_at | UNK_AV705559 |
| 215633_x_at | UNK_AV713720 |
| 202073_at | UNK_AV757675 |
| 213839_at | UNK_AW028110 |
| 214735_at | UNK_AW166711 |
| 212429_s_at | UNK_AW194657 |
| 210926_at | UNK_AY014272 |
| 211474_s_at | UNK_BC004948 |
| 211725_s_at | UNK_BC005884 |
| 213564_x_at | UNK_BE042354 |
| 213281_at | UNK_BE327172 |
| 203640_at | UNK_BE328496 |
| 212693_at | UNK_BE670928 |
| 221971_x_at | UNK_BE672818 |
| 208988_at | UNK_BE675843 |
| 208785_s_at | UNK_BE893893 |
| 204276_at | UNK_BE895437 |
| 215438_x_at | UNK_BE906054 |
| 213503_x_at | UNK_BE908217 |
| 213189_at | UNK_BE966695 |
| 212114_at | UNK_BE967207 |
| 212071_s_at | UNK_BE968833 |
| 221842_s_at | UNK_BE972394 |
| 213011_s_at | UNK_BF116254 |
| 212638_s_at | UNK_BF131791 |
| 212624_s_at | UNK_BF339445 |
| 213567_at | UNK_BF431965 |
| 202405_at | UNK_BF432532 |
| 212037_at | UNK_BF508848 |
| 212509_s_at | UNK_BF968134 |
| 209815_at | UNK_BG054916 |
| 202515_at | UNK_BG251175 |
| 214658_at | UNK_BG286537 |
| 222280_at | UNK_BG491393 |
| 205038_at | UNK_BG540504 |
| 211902_x_at | UNK_L34703 |
| 209670_at | UNK_M12959 |
| 210915_x_at | UNK_M15564 |
| 212237_at | UNK_N64780 |
| 203580_s_at | UNK_NM_003983 |
| 203130_s_at | UNK_NM_004522 |
| 205961_s_at | UNK_NM_004682 |
| 204474_at | UNK_NM_005081 |
| 203501_at | UNK_NM_006102 |
| 202475_at | UNK_NM_006326 |
| 203062_s_at | UNK_NM_014641 |
| 206003_at | UNK_NM_014645 |
| 205340_at | UNK_NM_014797 |
| 205953_at | UNK_NM_014813 |
| 203674_at | UNK_NM_014877 |
| 204568_at | UNK_NM_014924 |
| 206053_at | UNK_NM_014930 |
| 203956_at | UNK_NM_014941 |
| 204411_at | UNK_NM_017596 |
| 220486_x_at | UNK_NM_017698 |
| 218873_at | UNK_NM_017710 |
| 218829_s_at | UNK_NM_017780 |
| 218331_s_at | UNK_NM_017782 |
| 205510_s_at | UNK_NM_017976 |
| 218594_at | UNK_NM_018072 |
| 220452_x_at | UNK_NM_021031 |
| 208540_x_at | UNK_NM_021039 |
| 201963_at | UNK_NM_021122 |
| 218764_at | UNK_NM_024064 |
| 219253_at | UNK_NM_024121 |
| 219431_at | UNK_NM_024605 |
| 218505_at | UNK_NM_024673 |
| 220251_at | UNK_NM_024998 |
| 220999_s_at | UNK_NM_030778 |
| 214328_s_at | UNK_R01140 |
| 58308_at | UNK_R71157 |
| 211612_s_at | UNK_U62858 |
| 49485_at | UNK_W22625 |
| 203519_s_at | UPF2 |
| 203234_at | UPP1 |
| 201903_at | UQCRC1 |
| 210053_at | USMG5 |
| 208723_at | USP11 |
| 203965_at | USP20 |
| 220419_s_at | USP25 |
| 201498_at | USP7 |
| 221513_s_at | UTP14A |
| 203992_s_at | UTX |
| 208067_x_at | UTY |
| 219675_s_at | UXS1 |
| 201337_s_at | VAMP3 |
| 211749_s_at | VAMP3 |
| 202550_s_at | VAPB |
| 204254_s_at | VDR |
| 208623_s_at | VIL2 |
| 208622_s_at | VIL2 |
| 217949_s_at | VKORC1 |
| 220990_s_at | VMP1 |
| 205922_at | VNN2 |
| 212323_s_at | VPS13D |
| 203856_at | VRK1 |
| 213773_x_at | WBSCR20A |
| 213670_x_at | WBSCR20C |
| 214100_x_at | WBSCR20C |
| 213460_x_at | WBSCR20C |
| 221581_s_at | WBSCR5 |
| 218882_s_at | WDR3 |
| 212533_at | WEE1 |
| 34225_at | WHSC2 |
| 213836_s_at | WIPI49 |
| 203827_at | WIPI49 |
| 205667_at | WRN |
| 201760_s_at | WSB2 |
| 209375_at | XPC |
| 218767_at | XPMC2H |
| 211946_s_at | XTP2 |
| 213077_at | YTHDC2 |
| 204787_at | Z39IG |
| 214032_at | ZAP70 |
| 203026_at | ZBTB5 |
| 213051_at | ZC3HAV1 |
| 220104_at | ZC3HAV1 |
| 212704_at | ZCCHC11 |
| 213853_at | ZCSL3 |
| 218078_s_at | ZDHHC3 |
| 202978_s_at | ZF |
| 201368_at | ZFP36L2 |
| 203556_at | ZHX2 |
| 202136_at | ZMYND11 |
| 219854_at | ZNF14 |
| 200050_at | ZNF146 |
| 204327_s_at | ZNF202 |
| 218005_at | ZNF22 |
| 213934_s_at | ZNF23 |
| 204937_s_at | ZNF274 |
| 209494_s_at | ZNF278 |
| 209431_s_at | ZNF278 |
| 219228_at | ZNF331 |
| 214760_at | ZNF337 |
| 40569_at | ZNF42 |
| 219848_s_at | ZNF432 |
| 215359_x_at | ZNF44 |
| 214482_at | ZNF46 |
| 218735_s_at | ZNF544 |
| 221645_s_at | ZNF83 |
| 206572_x_at | ZNF85 |
| 200808_s_at | ZYX |
| 212893_at | ZZZ3 |

TABLE 24

| | | | | | | | | In Global | |
|---|---|---|---|---|---|---|---|---|---|
| | | Unadjusted p | Odds Ratio | Odds Ratio | | | | Analysis | |
| | | value for IgG | for IgG | for IgG | | | | (functional | In Key |
| | | association, | association, | association, | | | | categories | High |
| | | calculated | calculated | calculated | Affymetrix | | | and | Level |
| | FDR IgG | with | with | without | probeset | | Multiple/ | canonical | Functional |
| Gene | association | encephalitics | encephalitics | encephalitics | qualifier | Description | Single | pathways) | categories |
| PTBP1 | 0.00203 | 3.57E-05 | 0.112 | 0.096 | 211270_x_at | polypyrimidine | multiple | Yes | Yes |
| | | | | | | tract binding | | | |
| | | | | | | protein 1 | | | |
| GLUD1 | 0.0151 | 1.26E-03 | 0.098 | 0.110 | 200946_x_at | glutamate | single | Yes | No |
| | | | | | | dehydrogenase | | | |
| | | | | | | 1 | | | |
| MKNK1 | 0.0192 | 1.85E-03 | 0.147 | 0.121 | 209467_s_at | MAP kinase | single | Yes | Yes |
| | | | | | | interacting | | | |
| | | | | | | serine/threonine | | | |
| | | | | | | kinase 1 | | | |
| SLC12A9 | 0.000732 | 1.67E-06 | 0.117 | 0.125 | 220371_s_at | solute carrier | single | No | No |
| | | | | | | family 12 | | | |
| | | | | | | (potassium/ | | | |
| | | | | | | chloride | | | |
| | | | | | | transporters), | | | |
| | | | | | | member 9 | | | |
| HDGF | 0.00241 | 5.05E-05 | 0.137 | 0.155 | 216484_x_at | hepatoma- | single | Yes | No |
| | | | | | | derived | | | |
| | | | | | | growth factor | | | |
| | | | | | | (high-mobility | | | |
| | | | | | | group protein | | | |
| | | | | | | 1-like) | | | |
| ACTR1A | 0.00671 | 3.03E-04 | 0.171 | 0.156 | 200721_s_at | ARP1 actin- | single | Yes | No |
| | | | | | | related protein | | | |
| | | | | | | 1 homolog A, | | | |
| | | | | | | centractin | | | |
| | | | | | | alpha (yeast) | | | |
| GORASP2 | 0.00703 | 3.40E-04 | 0.219 | 0.176 | 207812_s_at | golgi | single | No | No |
| | | | | | | reassembly | | | |
| | | | | | | stacking | | | |
| | | | | | | protein 2, | | | |
| | | | | | | 55kDa | | | |
| BLCAP | 0.00456 | 1.56E-04 | 0.175 | 0.179 | 201032_at | bladder cancer | single | No | No |
| | | | | | | associated | | | |
| | | | | | | protein | | | |
| DKFZP564J157 | 0.0018 | 2.85E-05 | 0.158 | 0.187 | 217794_at | DKFZp564J157 | single | No | No |
| | | | | | | protein | | | |
| FLJ10315 | 0.00139 | 1.16E-05 | 0.174 | 0.191 | 218770_s_at | hypothetical | single | No | No |
| | | | | | | protein | | | |
| | | | | | | FLJ10315 | | | |
| CRKL | 0.0192 | 1.86E-03 | 0.180 | 0.192 | 212180_at | v-crk sarcoma | single | Yes | No |
| | | | | | | virus CT10 | | | |
| | | | | | | oncogene | | | |
| | | | | | | homolog | | | |
| | | | | | | (avian)-like | | | |
| EXT2 | 0.0261 | 3.14E-03 | 0.198 | 0.192 | 202012_s_at | exostoses | single | Yes | No |
| | | | | | | (multiple) 2 | | | |
| CDC40 | 0.00559 | 2.21E-04 | 0.177 | 0.203 | 203376_at | cell division | single | Yes | No |
| | | | | | | cycle 40 | | | |
| | | | | | | homolog | | | |
| | | | | | | (yeast) | | | |
| FLJ11560 | 0.00151 | 1.90E-05 | 0.189 | 0.203 | 211433_x_at | KIAA1539 | multiple | No | No |
| OAZIN | 0.0208 | 2.12E-03 | 0.210 | 0.204 | 212461_at | ornithine | single | Yes | No |
| | | | | | | decarboxylase | | | |
| | | | | | | antizyme | | | |
| | | | | | | inhibitor | | | |
| COPS7A | 0.0333 | 4.47E-03 | 0.258 | 0.207 | 209029_at | COP9 | single | No | No |
| | | | | | | constitutive | | | |
| | | | | | | photomorpho- | | | |
| | | | | | | genic homolog | | | |
| | | | | | | subunit 7A | | | |
| | | | | | | (Arabidopsis) | | | |
| STOM | 0.0322 | 4.29E-03 | 0.235 | 0.208 | 201060_x_at | stomatin | single | No | No |
| NPEPPS | 0.0187 | 1.77E-03 | 0.266 | 0.209 | 201454_s_at | aminopeptidase | single | Yes | No |
| | | | | | | puromycin | | | |
| | | | | | | sensitive | | | |
| SGPL1 | 0.027 | 3.31E-03 | 0.191 | 0.209 | 212321_at | sphingosine-1- | multiple | Yes | No |
| | | | | | | phosphate | | | |
| | | | | | | lyase 1 | | | |
| MAP2K3 | 0.00282 | 6.47E-05 | 0.195 | 0.214 | 215499_at | mitogen- | single | Yes | Yes |
| | | | | | | activated | | | |
| | | | | | | protein kinase | | | |
| | | | | | | kinase 3 | | | |
| SEC31L1 | 0.0134 | 1.02E-03 | 0.190 | 0.215 | 210616_s_at | SEC31-like 1 | single | Yes | No |
| | | | | | | (S.cerevisiae) | | | |
| ATP6V0A1 | 0.00387 | 1.15E-04 | 0.175 | 0.217 | 212383_at | ATPase, H+ | single | No | No |
| | | | | | | transporting, | | | |
| | | | | | | lysosomal V0 | | | |
| | | | | | | subunit a | | | |
| | | | | | | isoform 1 | | | |
| CBARA1 | 0.0114 | 7.57E-04 | 0.192 | 0.217 | 216903_s_at | calcium | single | No | No |
| | | | | | | binding atopy- | | | |
| | | | | | | related | | | |
| | | | | | | autoantigen 1 | | | |
| TXNRD1 | 0.0573 | 1.06E-02 | 0.285 | 0.218 | 201266_at | thioredoxin | single | Yes | Yes |
| | | | | | | reductase 1 | | | |
| TM9SF2 | 0.0206 | 2.08E-03 | 0.194 | 0.222 | 201078_at | transmembrane 9 | single | Yes | No |
| | | | | | | superfamily | | | |
| | | | | | | member 2 | | | |
| SH3BP2 | 0.00404 | 1.24E-04 | 0.207 | 0.223 | 209370_s_at | SH3-domain | single | Yes | No |
| | | | | | | binding | | | |
| | | | | | | protein 2 | | | |
| VCP | 0.00283 | 6.67E-05 | 0.172 | 0.226 | 208648_at | valosin- | single | Yes | No |
| | | | | | | containing | | | |
| | | | | | | protein | | | |
| KIAA0676 | 0.0326 | 4.34E-03 | 0.224 | 0.227 | 215994_x_at | KIAA0676 | single | No | No |
| | | | | | | protein | | | |
| FLJ10307 | 0.00537 | 2.01E-04 | 0.209 | 0.228 | 218753_at | hypothetical | single | No | No |
| | | | | | | protein | | | |
| | | | | | | FLJ10307 | | | |
| PAFAH1B1 | 0.00451 | 1.53E-04 | 0.212 | 0.228 | 200815_s_at | platelet- | single | Yes | Yes |
| | | | | | | activating | | | |
| | | | | | | factor | | | |
| | | | | | | acetylhydrolase, | | | |
| | | | | | | isoform Ib, | | | |
| | | | | | | alpha subunit | | | |
| | | | | | | 45kDa | | | |
| EIF4A1 | 0.0299 | 3.79E-03 | 0.221 | 0.231 | 211787_s_at | eukaryotic | single | Yes | Yes |
| | | | | | | translation | | | |
| | | | | | | initiation | | | |
| | | | | | | factor 4A, | | | |
| | | | | | | isoform 1 | | | |
| MFN2 | 0.00115 | 6.12E-06 | 0.205 | 0.236 | 201155_s_at | mitofusin 2 | single | Yes | No |
| ACTR1B | 0.0317 | 4.14E-03 | 0.220 | 0.239 | 202135_s_at | ARP1 actin- | single | Yes | No |
| | | | | | | related protein | | | |
| | | | | | | 1 homolog B, | | | |
| | | | | | | centractin beta | | | |
| | | | | | | (yeast) | | | |
| MGC10433 | 0.00363 | 1.01E-04 | 0.192 | 0.239 | 205740_s_at | hypothetical | single | No | No |
| | | | | | | protein | | | |
| | | | | | | MGC10433 | | | |
| CANX | 0.0265 | 3.23E-03 | 0.226 | 0.242 | 200068_s_at | calnexin | single | Yes | No |
| LOC285148 | 0.0072 | 3.50E-04 | 0.204 | 0.244 | 213532_at | hypothetical | single | No | No |
| | | | | | | protein | | | |
| | | | | | | LOC285148 | | | |
| ATP6V0C | 0.00358 | 9.87E-05 | 0.215 | 0.245 | 36994_at | ATPase, H+ | single | Yes | No |
| | | | | | | transporting, | | | |
| | | | | | | lysosomal | | | |
| | | | | | | 16kDa, V0 | | | |
| | | | | | | subunit c | | | |
| DAG1 | 0.014 | 1.10E-03 | 0.256 | 0.246 | 205417_s_at | dystroglycan 1 | single | Yes | Yes |
| | | | | | | (dystrophin- | | | |
| | | | | | | associated | | | |
| | | | | | | glycoprotein | | | |
| | | | | | | 1) | | | |
| K-ALPHA-1 | 0.0116 | 7.87E-04 | 0.225 | 0.246 | 211058_x_at | tubulin, alpha, | multiple | No | No |
| | | | | | | ubiquitous | | | |
| PLOD | 0.00146 | 1.68E-05 | 0.245 | 0.246 | 200827_at | procollagen- | single | Yes | No |
| | | | | | | lysine, 2- | | | |
| | | | | | | oxoglutarate | | | |
| | | | | | | 5-dioxygenase | | | |
| | | | | | | (lysine | | | |
| | | | | | | hydroxylase, | | | |
| | | | | | | Ehlers-Danlos | | | |
| | | | | | | syndrome type | | | |
| | | | | | | VI) | | | |
| PLOD3 | 0.0236 | 2.65E-03 | 0.277 | 0.246 | 202185_at | procollagen- | single | Yes | No |
| | | | | | | lysine, 2- | | | |
| | | | | | | oxoglutarate | | | |
| | | | | | | 5-dioxygenase | | | |
| | | | | | | 3 | | | |
| COBRA1 | 0.00336 | 8.60E-05 | 0.257 | 0.249 | 202757_at | cofactor of | single | Yes | No |
| | | | | | | BRCA1 | | | |
| NPL4 | 0.0243 | 2.81E-03 | 0.284 | 0.249 | 217796_s_at | nuclear protein | single | Yes | No |
| | | | | | | localization 4 | | | |
| SDF2 | 0.0965 | 2.31E-02 | 0.227 | 0.250 | 203090_at | stromal cell- | single | Yes | No |
| | | | | | | derived factor | | | |
| | | | | | | 2 | | | |
| PPP2R4 | 0.00261 | 5.83E-05 | 0.237 | 0.251 | 208874_x_at | protein | single | Yes | No |
| | | | | | | phosphatase | | | |
| | | | | | | 2A, regulatory | | | |
| | | | | | | subunit B′(PR | | | |
| | | | | | | 53) | | | |
| DNASE1L1 | 0.00182 | 2.98E-05 | 0.231 | 0.255 | 203912_s_at | deoxyribonuc1 | single | Yes | No |
| | | | | | | ease I-like 1 | | | |
| LASS2 | 0.00355 | 9.71E-05 | 0.259 | 0.255 | 222212_s_at | LAG1 | single | No | No |
| | | | | | | longevity | | | |
| | | | | | | assurance | | | |
| | | | | | | homolog 2 (S. | | | |
| | | | | | | cerevisiae) | | | |
| XPO7 | 0.00287 | 6.85E-05 | 0.298 | 0.256 | 212166_at | exportin 7 | single | Yes | Yes |
| GBA | 0.0116 | 7.92E-04 | 0.264 | 0.260 | 209093_s_at | glucosidase, | single | No | No |
| | | | | | | beta; acid | | | |
| | | | | | | (includes | | | |
| | | | | | | glucosyl- | | | |
| | | | | | | ceramidase) | | | |
| PLAGL2 | 0.0343 | 4.72E-03 | 0.269 | 0.260 | 202924_s_at | pleiomorphic | single | Yes | Yes |
| | | | | | | adenoma | | | |
| | | | | | | gene-like 2 | | | |
| MGC16824 | 0.0163 | 1.40E-03 | 0.226 | 0.262 | 203173_s_at | esophageal | single | No | No |
| | | | | | | cancer | | | |
| | | | | | | associated | | | |
| | | | | | | protein | | | |
| MGC13024 | 0.0238 | 2.71E-03 | 0.273 | 0.266 | 221864_at | hypothetical | single | No | No |
| | | | | | | protein | | | |
| | | | | | | MGC13024 | | | |
| KIAA0494 | 0.0111 | 7.20E-04 | 0.228 | 0.269 | 201776_s_at | K1AA0494 | single | No | No |
| | | | | | | gene product | | | |
| SMP1 | 0.0833 | 1.87E-02 | 0.278 | 0.272 | 217766_s_at | small | single | No | No |
| | | | | | | membrane | | | |
| | | | | | | protein 1 | | | |
| HK1 | 0.0468 | 7.87E-03 | 0.320 | 0.273 | 200697_at | hexokinase 1 | single | Yes | No |
| KIAA1193 | 0.0115 | 7.74E-04 | 0.251 | 0.275 | 44822_s_at | KIAA1193 | single | No | No |
| FLJ13910 | 0.0297 | 3.77E-03 | 0.257 | 0.276 | 212482_at | hypothetical | single | No | No |
| | | | | | | protein | | | |
| | | | | | | FLJ13910 | | | |
| NUP214 | 0.0176 | 1.61E-03 | 0.264 | 0.278 | 202155_s_at | nucleoporin | single | Yes | Yes |
| | | | | | | 214kDa | | | |
| SH3GLB1 | 0.0764 | 1.65E-02 | 0.279 | 0.279 | 209090_s_at | SH3-domain | single | Yes | No |
| | | | | | | GRB2-like | | | |
| | | | | | | endophilin B1 | | | |
| TM6SF1 | 0.0138 | 1.07E-03 | 0.271 | 0.279 | 219892_at | transmembrane 6 | single | No | No |
| | | | | | | superfamily | | | |
| | | | | | | member 1 | | | |
| XPO6 | 0.0161 | 1.39E-03 | 0.237 | 0.279 | 211982_x_at | exportin 6 | single | No | No |
| C21ORF97 | 0.0686 | 1.40E-02 | 0.286 | 0.280 | 218019_s_at | | single | No | No |
| SMARCD2 | 0.0211 | 2.20E-03 | 0.268 | 0.283 | 201827_at | SWI/SNF | single | Yes | No |
| | | | | | | related, matrix | | | |
| | | | | | | associated, | | | |
| | | | | | | actin | | | |
| | | | | | | dependent | | | |
| | | | | | | regulator of | | | |
| | | | | | | chromatin, | | | |
| | | | | | | subfamily d, | | | |
| | | | | | | member 2 | | | |
| ETHE1 | 0.00865 | 4.76E-04 | 0.289 | 0.285 | 204034_at | ethylmalonic | single | No | No |
| | | | | | | encephalopath | | | |
| | | | | | | y1 | | | |
| DCTN1 | 0.0065 | 2.82E-04 | 0.300 | 0.287 | 211780_x_at | dynactin 1 | single | Yes | No |
| | | | | | | (p150, glued | | | |
| | | | | | | homolog, | | | |
| | | | | | | Drosophila) | | | |
| | 0.0439 | 7.18E-03 | 0.280 | 0.289 | 213184_at | | | N/A | N/A |
| TUBA6 | 0.0104 | 6.38E-04 | 0.301 | 0.290 | 211750_x_at | tubulin alpha 6 | multiple | No | No |
| FURIN | 0.0106 | 6.68E-04 | 0.290 | 0.291 | 201945_at | furin (paired | single | Yes | No |
| | | | | | | basic amino | | | |
| | | | | | | acid cleaving | | | |
| | | | | | | enzyme) | | | |
| RAB2L | 0.0477 | 8.06E-03 | 0.249 | 0.292 | 209110_s_at | ral guanine | single | Yes | No |
| | | | | | | nucleotide | | | |
| | | | | | | dissociation | | | |
| | | | | | | stimulator- | | | |
| | | | | | | like 2 | | | |
| UBE2G1 | 0.0373 | 5.41E-03 | 0.274 | 0.294 | 209141_at | ubiquitin- | single | No | No |
| | | | | | | conjugating | | | |
| | | | | | | enzyme E2G 1 | | | |
| | | | | | | (UBC7 | | | |
| | | | | | | homolog, C. | | | |
| | | | | | | elegans) | | | |
| CENTA1 | 0.00887 | 4.95E-04 | 0.255 | 0.295 | 90265_at | centaurin, | single | Yes | No |
| | | | | | | alpha 1 | | | |
| DR1 | 0.0412 | 6.43E-03 | 0.252 | 0.297 | 207654_x_at | down- | single | Yes | No |
| | | | | | | regulator of | | | |
| | | | | | | transcription | | | |
| | | | | | | 1, TBP- | | | |
| | | | | | | binding | | | |
| | | | | | | (negative | | | |
| | | | | | | cofactor 2) | | | |
| MAPK7 | 0.00208 | 3.76E-05 | 0.255 | 0.297 | 35617_at | mitogen- | single | Yes | No |
| | | | | | | activated | | | |
| | | | | | | protein | | | |
| | | | | | | kinase 7 | | | |
| MPST | 0.00964 | 5.58E-04 | 0.259 | 0.297 | 203524_s_at | mercapto- | single | No | No |
| | | | | | | pyruvate | | | |
| | | | | | | sulfur- | | | |
| | | | | | | transferase | | | |
| MXD4 | 0.00144 | 1.27E-05 | 0.254 | 0.297 | 212346_s_at | MAX | single | Yes | No |
| | | | | | | dimerization | | | |
| | | | | | | protein 4 | | | |
| PEMT | 0.00144 | 1.39E-05 | 0.259 | 0.298 | 207621_s_at | phosphatidylet | single | Yes | No |
| | | | | | | hanolamine N- | | | |
| | | | | | | methyl- | | | |
| | | | | | | transferase | | | |
| DULLARd | 0.00939 | 5.35E-04 | 0.263 | 0.299 | 200035_at | dullard | single | No | No |
| | | | | | | homolog | | | |
| | | | | | | (Xenopus | | | |
| | | | | | | laevis) | | | |
| GDI1 | 0.00287 | 6.90E-05 | 0.283 | 0.302 | 201864_at | GDP | single | Yes | No |
| | | | | | | dissociation | | | |
| | | | | | | inhibitor 1 | | | |
| ARPC1B | 0.00202 | 3.49E-05 | 0.276 | 0.304 | 201954_at | actin related | single | Yes | No |
| | | | | | | protein ⅔ | | | |
| | | | | | | complex, | | | |
| | | | | | | subunit 1B, | | | |
| | | | | | | 41kDa | | | |
| IMPDH1 | 0.00256 | 5.63E-05 | 0.274 | 0.304 | 204169_at | IMP (inosine | single | Yes | No |
| | | | | | | monophosphate) | | | |
| | | | | | | dehydrogenase | | | |
| | | | | | | 1 | | | |
| PABPC4 | 0.00456 | 1.56E-04 | 0.256 | 0.304 | 201064_s_at | poly(A) | single | Yes | Yes |
| | | | | | | binding | | | |
| | | | | | | protein, | | | |
| | | | | | | cytoplasmic 4 | | | |
| | | | | | | (inducible | | | |
| | | | | | | form) | | | |
| KDELR2 | 0.0192 | 1.85E-03 | 0.297 | 0.305 | 200698_at | KDEL (Lys- | single | Yes | Yes |
| | | | | | | Asp-Glu-Leu) | | | |
| | | | | | | endoplasmic | | | |
| | | | | | | reticulum | | | |
| | | | | | | protein | | | |
| | | | | | | retention | | | |
| | | | | | | receptor 2 | | | |
| BAT3 | 0.0147 | 1.20E-03 | 0.326 | 0.306 | 201255_x_at | HLA-B | single | No | No |
| | | | | | | associated | | | |
| | | | | | | transcript 3 | | | |
| JWA | 0.067 | 1.34E-02 | 0.320 | 0.306 | 200760_s_at | cytoskeleton | single | Yes | No |
| | | | | | | related | | | |
| | | | | | | vitamin | | | |
| | | | | | | A responsive | | | |
| | | | | | | protein | | | |
| MGC5508 | 0.0558 | 1.02E-02 | 0.323 | 0.306 | 201361_at | hypothetical | single | No | No |
| | | | | | | protein | | | |
| | | | | | | MGC5508 | | | |
| PEPD | 0.00541 | 2.06E-04 | 0.273 | 0.308 | 202108_at | peptidase D | single | No | No |
| CLIC4 | 0.00564 | 2.25E-04 | 0.318 | 0.312 | 201560_at | chloride | single | Yes | No |
| | | | | | | intracellular | | | |
| | | | | | | channel 4 | | | |
| GRINA | 0.00167 | 2.47E-05 | 0.281 | 0.313 | 212090_at | glutamate | single | No | No |
| | | | | | | receptor, | | | |
| | | | | | | ionotropic, N- | | | |
| | | | | | | methyl D- | | | |
| | | | | | | asparate- | | | |
| | | | | | | associated | | | |
| | | | | | | protein 1 | | | |
| | | | | | | (glutamate | | | |
| | | | | | | binding) | | | |
| GTF2A2 | 0.0277 | 3.44E-03 | 0.277 | 0.314 | 202678_at | general | single | No | No |
| | | | | | | transcription | | | |
| | | | | | | factor IIA, 2, | | | |
| | | | | | | 12kDa | | | |
| ELK1 | 0.0999 | 2.44E-02 | 0.324 | 0.315 | 203617_x_at | ELK1, | single | Yes | No |
| | | | | | | member of | | | |
| | | | | | | ETS oncogene | | | |
| | | | | | | family | | | |
| RELA | 0.00702 | 3.33E-04 | 0.260 | 0.315 | 201783_s_at | v-rel | single | Yes | Yes |
| | | | | | | reticuloendoth | | | |
| | | | | | | eliosis viral | | | |
| | | | | | | oncogene | | | |
| | | | | | | homolog A, | | | |
| | | | | | | nuclear factor | | | |
| | | | | | | of kappa light | | | |
| | | | | | | polypeptide | | | |
| | | | | | | gene enhancer | | | |
| | | | | | | in B-cells 3, | | | |
| | | | | | | p65 (avian) | | | |
| AMFR | 0.0543 | 9.67E-03 | 0.313 | 0.319 | 202204_s_at | autocrine | single | Yes | No |
| | | | | | | motility factor | | | |
| | | | | | | receptor | | | |
| SLC9A8 | 0.0217 | 2.32E-03 | 0.315 | 0.321 | 212947_at | solute carrier | single | No | No |
| | | | | | | family 9 | | | |
| | | | | | | (sodium/hydro | | | |
| | | | | | | gen exchanger), | | | |
| | | | | | | isoform 8 | | | |
| APG4B | 0.0383 | 5.72E-03 | 0.326 | 0.322 | 212280_x_at | APG4 | single | Yes | No |
| | | | | | | autophagy 4 | | | |
| | | | | | | homolog B (S. | | | |
| | | | | | | cerevisiae) | | | |
| POLR2L | 0.00358 | 9.92E-05 | 0.296 | 0.322 | 211730_s_at | polymerase | single | Yes | No |
| | | | | | | (RNA) II | | | |
| | | | | | | (DNA | | | |
| | | | | | | directed) | | | |
| | | | | | | polypeptide L, | | | |
| | | | | | | 7.6kDa | | | |
| SNX27 | 0.0131 | 9.63E-04 | 0.314 | 0.325 | 221498_at | sorting nexin | single | No | No |
| | | | | | | family | | | |
| | | | | | | member 27 | | | |
| ADRM1 | 0.00547 | 2.14E-04 | 0.284 | 0.326 | 201281_at | adhesion | single | No | No |
| | | | | | | regulating | | | |
| | | | | | | molecule 1 | | | |
| FLJ10521 | 0.079 | 1.73E-02 | 0.319 | 0.326 | 221656_s_at | hypothetical | single | No | No |
| | | | | | | protein | | | |
| | | | | | | FLJ10521 | | | |
| KIAA0121 | 0.0265 | 3.22E-03 | 0.327 | 0.328 | 212399_s_at | vestigial like 4 | single | No | No |
| | | | | | | (Drosophila) | | | |
| MYO9B | 0.0442 | 7.28E-03 | 0.284 | 0.328 | 214780_s_at | myosin IXB | single | Yes | No |
| LENG4 | 0.0101 | 6.00E-04 | 0.302 | 0.329 | 205634_x_at | leukocyte | single | Yes | No |
| | | | | | | receptor | | | |
| | | | | | | cluster (LRC) | | | |
| | | | | | | member 4 | | | |
| SGSH | 0.0139 | 1.08E-03 | 0.311 | 0.331 | 35626_at | N-sulfoglu- | single | Yes | No |
| | | | | | | cosamine | | | |
| | | | | | | sulfohydrolase | | | |
| | | | | | | (sulfamidase) | | | |
| CORO1B | 0.00144 | 1.48E-05 | 0.303 | 0.333 | 64486_at | coronin, actin | single | No | No |
| | | | | | | binding | | | |
| | | | | | | protein,1B | | | |
| RRAGD | 0.00512 | 1.87E-04 | 0.276 | 0.334 | 221523_s_at | Ras-related | single | No | No |
| | | | | | | GTP binding | | | |
| | | | | | | D | | | |
| XBP1 | 0.0599 | 1.13E-02 | 0.292 | 0.334 | 200670_at | X-box binding | single | Yes | No |
| | | | | | | protein 1 | | | |
| CKAP4 | 0.0365 | 5.21E-03 | 0.302 | 0.335 | 200998_s_at | cytoskeleton- | single | No | No |
| | | | | | | associated | | | |
| PP9099 | 0.0122 | 8.61E-04 | 0.319 | 0.335 | 204436_at | PH domain- | single | No | No |
| | | | | | | containing | | | |
| | | | | | | protein | | | |
| ARF3 | 0.00478 | 1.68E-04 | 0.320 | 0.338 | 200011_s_at | ADP- | single | Yes | Yes |
| | | | | | | ribosylation | | | |
| | | | | | | factor 3 | | | |
| LOC51257 | 0.024 | 2.74E-03 | 0.332 | 0.338 | 210075_at | hypothetical | single | No | No |
| | | | | | | protein | | | |
| | | | | | | LOC51257 | | | |
| PXN | 0.0174 | 1.57E-03 | 0.296 | 0.342 | 201087_at | paxillin | single | Yes | No |
| SNN | 0.00635 | 2.74E-04 | 0.300 | 0.344 | 218033_s_at | stannin | single | Yes | No |
| CAMTA2 | 0.0276 | 3.41E-03 | 0.301 | 0.350 | 212948_at | calmodulin | single | No | No |
| | | | | | | binding | | | |
| | | | | | | transcription | | | |
| | | | | | | activator 2 | | | |
| C20ORF35 | 0.0209 | 2.16E-03 | 0.306 | 0.351 | 218094_s_at | chromosome | single | No | No |
| | | | | | | 20 open | | | |
| | | | | | | reading frame | | | |
| | | | | | | 35 | | | |
| FLJ13725 | 0.0586 | 1.10E-02 | 0.322 | 0.354 | 45749_at | hypothetical | single | No | No |
| | | | | | | protein | | | |
| | | | | | | FLJ13725 | | | |
| GRK6 | 0.0194 | 1.89E-03 | 0.311 | 0.355 | 210981_s_at | G protein- | single | Yes | No |
| | | | | | | coupled | | | |
| | | | | | | receptor | | | |
| | | | | | | kinase 6 | | | |
| FLJ12287 | 0.0101 | 6.18E-04 | 0.332 | 0.357 | 219259_at | sema domain, | single | No | No |
| | | | | | | immunoglobulin | | | |
| | | | | | | domain (Ig), | | | |
| | | | | | | transmembrane | | | |
| | | | | | | domain (TM) | | | |
| | | | | | | and short | | | |
| | | | | | | cytoplasmic | | | |
| | | | | | | domain, | | | |
| | | | | | | (semaphorin) | | | |
| | | | | | | 4A | | | |
| CDKN1A | 0.00396 | 1.20E-04 | 0.325 | 0.365 | 202284_s_at | cyclin- | single | Yes | Yes |
| | | | | | | dependent | | | |
| | | | | | | kinase | | | |
| | | | | | | inhibitor 1A | | | |
| | | | | | | (p21, Cip1) | | | |
| KPNA6 | 0.0117 | 8.02E-04 | 0.320 | 0.365 | 212101_at | karyopherin | single | Yes | Yes |
| | | | | | | alpha 6 | | | |
| | | | | | | (importin | | | |
| | | | | | | alpha 7) | | | |
| CAB45 | 0.00387 | 1.16E-04 | 0.328 | 0.367 | 217855_x_at | calcium | single | No | No |
| | | | | | | binding | | | |
| | | | | | | protein Cab45 | | | |
| | | | | | | precursor | | | |
| GALNACT-2 | 0.0196 | 1.92E-03 | 0.315 | 0.367 | 222235_s_at | chondroitin | single | Yes | No |
| | | | | | | sulfate | | | |
| | | | | | | GalNAcT-2 | | | |
| WDR13 | 0.0114 | 7.58E-04 | 0.326 | 0.371 | 222138_s_at | WD repeat | single | No | No |
| | | | | | | domain 13 | | | |
| OS-9 | 0.0184 | 1.73E-03 | 0.329 | 0.374 | 200714_x_at | amplified in | single | No | No |
| | | | | | | osteosarcoma | | | |
| SRF | 0.0116 | 7.97E-04 | 0.3 19 | 0.374 | 202401_s_at | serum | single | Yes | Yes |
| | | | | | | response | | | |
| | | | | | | factor (c-fos | | | |
| | | | | | | serum | | | |
| | | | | | | response | | | |
| | | | | | | element- | | | |
| | | | | | | binding | | | |
| | | | | | | transcnption | | | |
| | | | | | | factor) | | | |
| CHK | 0.0114 | 7.68E-04 | 0.327 | 0.376 | 204266_s_at | choline kinase | single | Yes | No |
| | | | | | | alpha | | | |
| TM9SF4 | 0.0399 | 6.06E-03 | 0.332 | 0.376 | 212198_s_at | transmembrane | single | No | No |
| | | | | | | 9 superfamily | | | |
| | | | | | | protein | | | |
| | | | | | | member 4 | | | |
| CLN2 | 0.00611 | 2.55E-04 | 0.331 | 0.379 | 200742_s_at | ceroid- | single | Yes | No |
| | | | | | | lipofuscinosis, | | | |
| | | | | | | neuronal 2, | | | |
| | | | | | | late infantile | | | |
| | | | | | | (Jansky- | | | |
| | | | | | | Bielschowsky | | | |
| | | | | | | disease) | | | |
| LOC92482 | 0.037 | 5.31E-03 | 3.028 | 2.691 | 213220_at | hypothetical | single | No | No |
| | | | | | | protein | | | |
| | | | | | | LOC92482 | | | |
| SS18L2 | 0.0277 | 3.43E-03 | 3.151 | 2.704 | 218283_at | synovial | single | No | No |
| | | | | | | sarcoma | | | |
| | | | | | | translocation | | | |
| | | | | | | gene on | | | |
| | | | | | | chromosome | | | |
| | | | | | | 18-like 2 | | | |
| AKR1C1 | 0.0586 | 1.10E-02 | 3.098 | 2.712 | 216594_x_at | aldo-keto | single | Yes | No |
| | | | | | | reductase | | | |
| | | | | | | family 1, | | | |
| | | | | | | member C1 | | | |
| | | | | | | (dihydrodiol | | | |
| | | | | | | dehydrogenase | | | |
| | | | | | | 1; 20-alpha | | | |
| | | | | | | 3-alpha) | | | |
| | | | | | | hydroxysteroid | | | |
| | | | | | | dehydrogenase) | | | |
| CALM1 | 0.0214 | 2.26E-03 | 3.136 | 2.722 | 209563_x_at | calmodulin 1 | single | Yes | No |
| | | | | | | (phosphorylase | | | |
| | | | | | | kinase, delta) | | | |
| DHX15 | 0.0435 | 7.09E-03 | 3.049 | 2.726 | 201385_at | DEAH (Asp- | single | No | No |
| | | | | | | Glu-Ala-His) | | | |
| | | | | | | box | | | |
| | | | | | | polypeptide 15 | | | |
| KIAA0252 | 0.0407 | 6.31E-03 | 3.099 | 2.786 | 212302_at | K1AA0252 | single | No | No |
| NDUFA4 | 0.0501 | 8.60E-03 | 3.034 | 2.790 | 217773_s_at | NADH | single | Yes | No |
| | | | | | | dehydrogenase | | | |
| | | | | | | (ubiquinone) 1 | | | |
| | | | | | | alpha | | | |
| | | | | | | subcomplex, | | | |
| | | | | | | 4, 9kDa | | | |
| FLJ10460 | 0.00426 | 1.38E-04 | 3.085 | 2.791 | 220071_x_at | hypothetical | single | No | No |
| | | | | | | protein | | | |
| | | | | | | FLJ10460 | | | |
| LSM5 | 0.00482 | 1.71E-04 | 3.126 | 2.815 | 211747_s_at | LSM5 | single | No | No |
| | | | | | | homolog, U6 | | | |
| | | | | | | small nuclear | | | |
| | | | | | | RNA associated | | | |
| | | | | | | (S. cerevisiae) | | | |
| ALMS1 | 0.00371 | 1.08E-04 | 3.045 | 2.829 | 214707_x_at | Alstrom | single | Yes | No |
| | | | | | | syndrome 1 | | | |
| | 0.00211 | 3.97E-05 | 3.099 | 2.874 | 216006_at | | | N/A | N/A |
| | 0.00283 | 6.67E-05 | 3.291 | 2.879 | 217713_x_at | | | N/A | N/A |
| LAMR1 | 0.00169 | 2.59E-05 | 3.016 | 2.903 | 216806_at | laminin | single | Yes | No |
| | | | | | | receptor 1 | | | |
| | | | | | | (ribosomal | | | |
| | | | | | | protein SA, | | | |
| | | | | | | 67kDa) | | | |
| GTF2H2 | 0.00283 | 6.56E-05 | 3.033 | 2.926 | 221540_x_at | general | single | No | No |
| | | | | | | transcription | | | |
| | | | | | | factor IIH, | | | |
| | | | | | | polypeptide 2, | | | |
| | | | | | | 44kDa | | | |
| TLE1 | 0.0399 | 6.07E-03 | 3.140 | 2.932 | 203221_at | transducin-like | single | Yes | No |
| | | | | | | enhancer of | | | |
| | | | | | | split 1 (E(sp1) | | | |
| | | | | | | homolog, | | | |
| | | | | | | Drosophila) | | | |
| XRCC2 | 0.00165 | 2.33E-05 | 3.033 | 2.934 | 207598_x_at | X-ray repair | single | Yes | Yes |
| | | | | | | complementing | | | |
| | | | | | | defective | | | |
| | | | | | | repair in | | | |
| | | | | | | Chinese | | | |
| | | | | | | hamster cells 2 | | | |
| DSPP | 0.0227 | 2.51E-03 | 3.374 | 2.943 | 221681_s_at | dentin | single | Yes | No |
| | | | | | | sialophospho | | | |
| | | | | | | protein | | | |
| EIF3S1 | 0.0395 | 5.97E-03 | 3.184 | 2.948 | 208264_s_at | eukaryotic | single | Yes | Yes |
| | | | | | | translation | | | |
| | | | | | | initiation | | | |
| | | | | | | factor 3, | | | |
| | | | | | | subunit 1 | | | |
| | | | | | | alpha, 35kDa | | | |
| SLC30A5 | 0.0274 | 3.38E-03 | 3.230 | 2.967 | 218989_x_at | solute carrier | single | Yes | No |
| | | | | | | family 30 | | | |
| | | | | | | (zinc | | | |
| | | | | | | transporter), | | | |
| | | | | | | member 5 | | | |
| ZNF261 | 0.0458 | 7.66E-03 | 3.022 | 2.995 | 207559_s_at | zinc finger | single | Yes | No |
| | | | | | | protein 261 | | | |
| NAB1 | 0.0838 | 1.89E-02 | 3.184 | 2.996 | 211139_s_at | NGFI-A | single | Yes | No |
| | | | | | | binding | | | |
| | | | | | | protein 1 | | | |
| | | | | | | (EGR1 | | | |
| | | | | | | binding | | | |
| | | | | | | protein 1) | | | |
| RIOK3 | 0.00115 | 5.92E-06 | 3.312 | 3.004 | 215588_x_at | RIO kinase | single | Yes | No |
| | | | | | | 3 (yeast) | | | |
| ZNF505 | 0.0193 | 1.87E-03 | 3.026 | 3.006 | 215758_x_at | zinc finger | single | No | No |
| | | | | | | protein 505 | | | |
| SNRPB2 | 0.00676 | 3.08E-04 | 3.596 | 3.015 | 202505_at | small nuclear | single | Yes | No |
| | | | | | | ribonucleo- | | | |
| | | | | | | protein | | | |
| | | | | | | polypeptide B″ | | | |
| UBL1 | 0.04 | 6.13E-03 | 3.159 | 3.033 | 211069_s_at | SMT3 | single | Yes | Yes |
| | | | | | | suppressor of | | | |
| | | | | | | mif two 3 | | | |
| | | | | | | homolog 1 | | | |
| | | | | | | (yeast) | | | |
| TBCA | 0.0071 | 3.44E-04 | 3.344 | 3.041 | 203667_at | tubulin- | single | No | No |
| | | | | | | specific | | | |
| | | | | | | chaperone a | | | |
| POLR1B | 0.0011 | 5.46E-06 | 3.590 | 3.049 | 220113_x_at | polymerase | single | Yes | No |
| | | | | | | (RNA) I | | | |
| | | | | | | polypeptide B, | | | |
| | | | | | | 128kDa | | | |
| TCEAL1 | 0.0241 | 2.77E-03 | 3.163 | 3.062 | 204045_at | transcription | single | Yes | No |
| | | | | | | elongation | | | |
| | | | | | | factor A (SII)- | | | |
| | | | | | | like 1 | | | |
| MGC48332 | 0.0165 | 1.43E-03 | 3.326 | 3.063 | 213256_at | hypothetical | single | No | No |
| | | | | | | protein | | | |
| | | | | | | MGC48332 | | | |
| SCD4 | 0.00773 | 3.96E-04 | 3.441 | 3.071 | 214036_at | | | N/A | N/A |
| FLJ22256 | 0.0113 | 7.39E-04 | 3.503 | 3.081 | 220856_x_at | | | N/A | N/A |
| TAX1BP1 | 0.0433 | 7.03E-03 | 3.335 | 3.085 | 200977_s_at | Tax1 (human | single | Yes | No |
| | | | | | | T-cell | | | |
| | | | | | | leukemia virus | | | |
| | | | | | | type I) binding | | | |
| | | | | | | protein 1 | | | |
| FLJ10287 | 0.0177 | 1.64E-03 | 3.262 | 3.091 | 219130_at | hypothetical | single | No | No |
| | | | | | | protein | | | |
| | | | | | | FLJ10287 | | | |
| CYCS | 0.0281 | 3.51E-03 | 3.257 | 3.092 | 208905_at | cytochrome c, | single | Yes | No |
| | | | | | | somatic | | | |
| CGI-12 | 0.00755 | 3.79E-04 | 3.378 | 3.117 | 219363_s_at | CGI-12 | single | No | No |
| | | | | | | protein | | | |
| RBBP6 | 0.00544 | 2.10E-04 | 3.065 | 3.128 | 212781_at | retinoblastoma | single | Yes | No |
| | | | | | | binding | | | |
| | | | | | | protein 6 | | | |
| GTSE1 | 0.0219 | 2.35E-03 | 3.070 | 3.133 | 211040_x_at | G-2 and S- | single | No | No |
| | | | | | | phase | | | |
| | | | | | | expressed 1 | | | |
| RPL26 | 0.00121 | 7.25E-06 | 3.342 | 3.133 | 222229_x_at | ribosomal | single | Yes | Yes |
| | | | | | | protein L26 | | | |
| PIGL | 0.042 | 6.64E-03 | 3.247 | 3.142 | 205873_at | phosphatidylin | single | Yes | No |
| | | | | | | ositol glycan, | | | |
| | | | | | | class L | | | |
| NOL5A | 0.0284 | 3.56E-03 | 3.760 | 3.146 | 200874_s_at | nucleolar | single | No | No |
| | | | | | | protein 5A | | | |
| | | | | | | (56kDa with | | | |
| | | | | | | KKE/D | | | |
| | | | | | | repeat) | | | |
| C1GALT1 | 0.0132 | 9.79E-04 | 3.612 | 3.150 | 219439_at | core 1 UDP- | single | No | No |
| | | | | | | galactose:N- | | | |
| | | | | | | acetylgalactos | | | |
| | | | | | | amine-alpha-R | | | |
| | | | | | | beta 1,3- | | | |
| | | | | | | galactosyl- | | | |
| | | | | | | transferase | | | |
| NIF3L1BP1 | 0.00355 | 9.67E-05 | 3.506 | 3.150 | 218334_at | Ngg1 | single | No | No |
| | | | | | | interacting | | | |
| | | | | | | factor 3 like | | | |
| | | | | | | 1 binding | | | |
| | | | | | | protein 1 | | | |
| P38IP | 0.0361 | 5.14E-03 | 3.621 | 3.160 | 220408_x_at | transcription | single | No | No |
| | | | | | | factor (p38 | | | |
| | | | | | | interacting | | | |
| | | | | | | protein) | | | |
| FTLL1 | 0.0128 | 9.29E-04 | 3.273 | 3.170 | 217703_x_at | | | N/A | N/A |
| NIP30 | 0.0382 | 5.68E-03 | 3.641 | 3.175 | 217896_s_at | NEFA- | single | No | No |
| | | | | | | interacting | | | |
| | | | | | | nuclear protein | | | |
| | | | | | | NIP30 | | | |
| LONP | 0.00209 | 3.81E-05 | 3.226 | 3.197 | 221834_at | peroxisomal | single | No | No |
| | | | | | | Ion protease | | | |
| DNAJC8 | 0.0997 | 2.43E-02 | 3.323 | 3.199 | 212491_s_at | DnaJ (Hsp40) | single | No | No |
| | | | | | | homolog, | | | |
| | | | | | | subfamily C, | | | |
| | | | | | | member 8 | | | |
| TCEB1 | 0.0777 | 1.70E-02 | 3.198 | 3.200 | 202824_s_at | transcription | single | Yes | No |
| | | | | | | elongation | | | |
| | | | | | | factor B (SIII), | | | |
| | | | | | | polypeptide I | | | |
| | | | | | | (15kDa, | | | |
| | | | | | | elongin C) | | | |
| OIP2 | 0.00874 | 4.81E-04 | 3.544 | 3.206 | 215136_s_at | exosome | single | No | No |
| | | | | | | component 8 | | | |
| C14ORF123 | 0.00368 | 1.05E-04 | 3.718 | 3.209 | 218571_s_at | chromosome | single | No | No |
| | | | | | | 14 open | | | |
| | | | | | | reading frame | | | |
| | | | | | | 123 | | | |
| MCAM | 0.0733 | 1.54E-02 | 3.745 | 3.222 | 211042_x_at | melanoma cell | single | Yes | No |
| | | | | | | adhesion | | | |
| | | | | | | molecule | | | |
| MPP2 | 0.0223 | 2.42E-03 | 3.682 | 3.243 | 207984_s_at | membrane | single | Yes | No |
| | | | | | | protein, | | | |
| | | | | | | palmitoylated | | | |
| | | | | | | 2 (MAGUK | | | |
| | | | | | | p55 subfamily | | | |
| | | | | | | member 2) | | | |
| LOC57149 | 0.0526 | 9.29E-03 | 3.251 | 3.244 | 203897_at | hypothetical | single | No | No |
| | | | | | | protein A- | | | |
| | | | | | | 211C6.1 | | | |
| FLJ23233 | 0.0505 | 8.72E-03 | 3.601 | 3.245 | 58367_s_at | hypothetical | single | No | No |
| | | | | | | protein | | | |
| | | | | | | FLJ23233 | | | |
| P29 | 0.00698 | 3.28E-04 | 3.659 | 3.247 | 202553_s_at | GCIP- | single | No | No |
| | | | | | | interacting | | | |
| | | | | | | protein p29 | | | |
| DNAH3 | 0.018 | 1.67E-03 | 3.282 | 3.250 | 209751_s_at | Mitogen | | N/A | N/A |
| | | | | | | Activated | | | |
| | | | | | | Protein | | | |
| | | | | | | Kinase | | | |
| | | | | | | Kinase | | | |
| SON | 0.0373 | 5.41E-03 | 3.843 | 3.258 | 214988_s_at | SON DNA | single | Yes | No |
| | | | | | | binding | | | |
| | | | | | | protein | | | |
| NONO | 0.0513 | 8.98E-03 | 3.745 | 3.275 | 210470_x_at | non-POU | single | No | No |
| | | | | | | domain | | | |
| | | | | | | containing, | | | |
| | | | | | | octamer- | | | |
| | | | | | | binding | | | |
| PGF | 0.000656 | 7.16E-07 | 3.555 | 3.285 | 215179_x_at | placental | single | Yes | No |
| | | | | | | growth factor, | | | |
| | | | | | | vascular | | | |
| | | | | | | endothelial | | | |
| | | | | | | growth factor- | | | |
| | | | | | | related protein | | | |
| MCM3AP | 0.00923 | 5.22E-04 | 3.627 | 3.293 | 215582_x_at | MCM3 | single | Yes | Yes |
| | | | | | | mini- | | | |
| | | | | | | chromosome | | | |
| | | | | | | maintenance | | | |
| | | | | | | deficient 3 | | | |
| | | | | | | (S. cerevisiae) | | | |
| | | | | | | associated | | | |
| | | | | | | protein | | | |
| WBSCR5 | 0.00211 | 4.04E-05 | 3.921 | 3.317 | 211768_at | Williams- | single | Yes | No |
| | | | | | | Beuren | | | |
| | | | | | | syndrome | | | |
| | | | | | | chromosome | | | |
| | | | | | | region 5 | | | |
| TMPO | 0.00976 | 5.74E-04 | 3.992 | 3.321 | 209753_s_at | thymopoietin | single | Yes | Yes |
| NTRK3 | 0.0148 | 1.21E-03 | 3.298 | 3.323 | 217033_x_at | neurotrophic | single | Yes | Yes |
| | | | | | | tyrosine | | | |
| | | | | | | kinase, | | | |
| | | | | | | receptor, | | | |
| | | | | | | type 3 | | | |
| SOD1 | 0.0301 | 3.83E-03 | 3.742 | 3.337 | 200642_at | superoxide | single | Yes | Yes |
| | | | | | | dismutase 1, | | | |
| | | | | | | soluble | | | |
| | | | | | | (amyotrophic | | | |
| | | | | | | lateral | | | |
| | | | | | | sclerosis 1 | | | |
| | | | | | | (adult)) | | | |
| TCTEL1 | 0.0433 | 7.05E-03 | 3.245 | 3.359 | 201999_s_at | t-complex- | single | Yes | No |
| | | | | | | associated- | | | |
| | | | | | | testis- | | | |
| | | | | | | expressed | | | |
| | | | | | | 1-like 1 | | | |
| GSTM3 | 0.00354 | 9.54E-05 | 3.641 | 3.363 | 202554_s_at | glutathione S- | single | Yes | No |
| | | | | | | transferase | | | |
| | | | | | | M3 (brain) | | | |
| C60RF62 | 0.0672 | 1.35E-02 | 3.245 | 3.390 | 208809_s_at | chromosome 6 | single | No | No |
| | | | | | | open reading | | | |
| | | | | | | frame 62 | | | |
| ZNF263 | 0.0237 | 2.68E-03 | 3.509 | 3.390 | 203707_at | zinc finger | single | Yes | No |
| | | | | | | protein 263 | | | |
| NEDD5 | 0.075 | 1.60E-02 | 3.861 | 3.393 | 200015_s_at | neural | single | No | No |
| | | | | | | precursor cell | | | |
| | | | | | | expressed, | | | |
| | | | | | | developmentally | | | |
| | | | | | | down-regulated 5 | | | |
| CPA2 | 0.0157 | 1.33E-03 | 3.506 | 3.416 | 206212_at | carboxy- | single | Yes | No |
| | | | | | | peptidase A2 | | | |
| | | | | | | (pancreatic) | | | |
| PEX16 | 0.0559 | 1.02E-02 | 3.690 | 3.422 | 49878_at | peroxisomal | single | Yes | No |
| | | | | | | biogenesis | | | |
| | | | | | | factor 16 | | | |
| RPL35 | 0.0158 | 1.35E-03 | 3.439 | 3.434 | 200002_at | ribosomal | multiple | No | No |
| | | | | | | protein L35 | | | |
| FACL6 | 0.0188 | 1.79E-03 | 3.228 | 3.436 | 211207_s_at | acyl-CoA | single | Yes | No |
| | | | | | | synthetase | | | |
| | | | | | | long-chain | | | |
| | | | | | | family | | | |
| | | | | | | member 6 | | | |
| FOXO1A | 0.0328 | 4.40E-03 | 3.167 | 3.436 | 202724_s_at | forkhead box | single | Yes | Yes |
| | | | | | | O1A | | | |
| | | | | | | (rhabdomyo- | | | |
| | | | | | | sarcoma) | | | |
| TGFB3 | 0.0237 | 2.67E-03 | 3.511 | 3.440 | 209747_at | transforming | single | Yes | Yes |
| | | | | | | growth factor, | | | |
| | | | | | | beta 3 | | | |
| RPL24 | 0.0682 | 1.38E-02 | 3.055 | 3.445. | 214143_x_at | ribosomal | single | No | No |
| | | | | | | protein L24 | | | |
| HSPC128 | 0.00512 | 1.88E-04 | 3.394 | 3.457 | 218936_s_at | HSPC128 | single | No | No |
| | | | | | | protein | | | |
| PSKH1 | 0.0137 | 1.05E-03 | 3.788 | 3.459 | 213141_at | protein serine | single | No | No |
| | | | | | | kinase H1 | | | |
| RANBP9 | 0.0805 | 1.78E-02 | 3.008 | 3.471 | 202582_s_at | RAN binding | single | Yes | No |
| | | | | | | protein 9 | | | |
| SNAP25 | 0.0337 | 4.59E-03 | 3.119 | 3.476 | 202507_s_at | synaptosomal- | single | Yes | No |
| | | | | | | associated | | | |
| | | | | | | protein, 25kDa | | | |
| FLJ23476 | 0.0126 | 9.08E-04 | 3.680 | 3.501 | 218647_s_at | ischemia/reper | single | No | No |
| | | | | | | fusion | | | |
| | | | | | | inducible | | | |
| | | | | | | protein | | | |
| PHF2 | 0.0669 | 1.33E-02 | 3.458 | 3.502 | 212726_at | PHD finger | single | No | No |
| | | | | | | protein 2 | | | |
| FLJ20331 | 0.00256 | 5.62E-05 | 3.855 | 3.510 | 215063_x_at | hypothetical | single | No | No |
| | | | | | | protein | | | |
| | | | | | | FLJ20331 | | | |
| SMARCA5 | 0.0278 | 3.47E-03 | 3.504 | 3.519 | 213251_at | SWI/SNF | single | Yes | No |
| | | | | | | related, matrix | | | |
| | | | | | | associated, | | | |
| | | | | | | actin | | | |
| | | | | | | dependent | | | |
| | | | | | | regulator of | | | |
| | | | | | | chromatin, | | | |
| | | | | | | subfamily a, | | | |
| | | | | | | member 5 | | | |
| UQCRB | 0.00582 | 2.36E-04 | 3.910 | 3.523 | 209065_at | ubiquinol- | multiple | Yes | No |
| | | | | | | cytochrome c | | | |
| | | | | | | reductase | | | |
| | | | | | | binding | | | |
| | | | | | | protein | | | |
| DKFZp566N034 | 0.00301 | 7.46E-05 | 4.150 | 3.525 | 208238_x_at | | | N/A | N/A |
| TRAPCC2 | 0.0224 | 2.46E-03 | 3.842 | 3.530 | 206853_s_at | | | N/A | N/A |
| SLC35E1 | 0.0152 | 1.28E-03 | 3.961 | 3.538 | 79005_at | solute carrier | single | No | No |
| | | | | | | family 35, | | | |
| | | | | | | member E1 | | | |
| DT1P1A10 | 0.0318 | 4.17E-03 | 4.334 | 3.551 | 213079_at | hypothetical | single | No | No |
| | | | | | | protein | | | |
| | | | | | | DT1P1A10 | | | |
| PRKDC | 0.00631 | 2.70E-04 | 3.401 | 3.553 | 208694_at | protein kinase, | single | Yes | Yes |
| | | | | | | DNA- | | | |
| | | | | | | activated, | | | |
| | | | | | | catalytic | | | |
| | | | | | | polypeptide | | | |
| TTC13 | 0.037 | 5.30E-03 | 3.397 | 3.554 | 219481_at | tetratrico- | single | No | No |
| | | | | | | peptide repeat | | | |
| | | | | | | domain 13 | | | |
| NFRKB | 0.0586 | 1.10E-02 | 3.162 | 3.570 | 213028_at | nuclear factor | single | No | No |
| | | | | | | related to | | | |
| | | | | | | kappa B | | | |
| | | | | | | binding | | | |
| | | | | | | protein | | | |
| B2M | 0.0662 | 1.32E-02 | 3.074 | 3.582 | 201891_s_at | beta-2- | single | Yes | No |
| | | | | | | microglobulin | | | |
| VAMP4 | 0.0241 | 2.77E-03 | 3.512 | 3.591 | 213480_at | vesicle- | single | Yes | No |
| | | | | | | associated | | | |
| | | | | | | membrane | | | |
| | | | | | | protein 4 | | | |
| HSPA8 | 0.0337 | 4.58E-03 | 3.651 | 3.602 | 221891_x_at | heat shock | single | Yes | No |
| | | | | | | 70kDa protein | | | |
| | | | | | | 8 | | | |
| Unknown | 0.00665 | 2.95E-04 | 4.084 | 3.610 | 215557_at | | | N/A | N/A |
| MAP3K7 | 0.032 | 4.23E-03 | 3.601 | 3.616 | 215476_at | | | N/A | N/A |
| 0 | 0.0397 | 6.03E-03 | 3.602 | 3.628 | 212436_at | | | N/A | N/A |
| RARG-1 | 0.00541 | 2.05E-04 | 4.107 | 3.631 | 202882_x_at | nucleolar | multiple | No | No |
| | | | | | | protein 7, | | | |
| | | | | | | 27kDa | | | |
| SSB | 0.00795 | 4.21E-04 | 3.664 | 3.642 | 201139_s_at | Sjogren | single | Yes | Yes |
| | | | | | | syndrome | | | |
| | | | | | | antigen B | | | |
| | | | | | | (autoantigen | | | |
| | | | | | | La) | | | |
| HNRPH1 | 0.0111 | 7.23E-04 | 3.327 | 3.643 | 213619_at | heterogeneous | single | Yes | No |
| | | | | | | nuclear | | | |
| | | | | | | ribonucleo- | | | |
| | | | | | | protein H1 (H) | | | |
| HCDI | 0.0906 | 2.11E-02 | 3.791 | 3.649 | 213398_s_at | chromosome | single | No | No |
| | | | | | | 14 open | | | |
| | | | | | | reading frame | | | |
| | | | | | | 124 | | | |
| COX7A3 | 0.00631 | 2.71E-04 | 4.532 | 3.654 | 217249_x_at | cytochrome c | single | Yes | No |
| | | | | | | oxidase | | | |
| | | | | | | subunit VIIa | | | |
| | | | | | | polypeptide 3 | | | |
| | | | | | | (liver) | | | |
| CDK2 | 0.00404 | 1.25E-04 | 4.295 | 3.677 | 204252_at | cyclin- | single | Yes | Yes |
| | | | | | | dependent | | | |
| | | | | | | kinase 2 | | | |
| ZNF-U69274 | 0.00547 | 2.14E-04 | 4.158 | 3.688 | 204847_at | zinc finger and | single | No | No |
| | | | | | | BTB domain | | | |
| | | | | | | containing 11 | | | |
| ZFP95 | 0.018 | 1.67E-03 | 3.562 | 3.694 | 203730_s_at | zinc finger | single | No | No |
| | | | | | | protein 95 | | | |
| | | | | | | homolog | | | |
| | | | | | | (mouse) | | | |
| Unannotated | 0.00249 | 5.25E-05 | 4.290 | 3.722 | 215628_x_at | | | N/A | N/A |
| PITPNC1 | 0.0956 | 2.28E-02 | 3.059 | 3.752 | 219155_at | phosphatidylin | single | Yes | No |
| | | | | | | ositol transfer | | | |
| | | | | | | protein, | | | |
| | | | | | | cytoplasmic 1 | | | |
| ATP5I | 0.0144 | 1.15E-03 | 3.511 | 3.755 | 209492_x_at | ATP synthase, | single | No | No |
| | | | | | | H+ | | | |
| | | | | | | transporting, | | | |
| | | | | | | mitochondrial | | | |
| | | | | | | F0 complex, | | | |
| | | | | | | subunit e | | | |
| ING1L | 0.0297 | 3.75E-03 | 3.537 | 3.766 | 205981_s_at | inhibitor of | single | Yes | No |
| | | | | | | growth family, | | | |
| | | | | | | member 1-like | | | |
| FLJ34588 | 0.0206 | 2.07E-03 | 3.860 | 3.767 | 212410_at | Smhs2 | single | No | No |
| | | | | | | homolog (rat) | | | |
| FLJ117l2 | 0.0427 | 6.87E-03 | 3.380 | 3.779 | 219056_at | hypothetical | single | No | No |
| | | | | | | protein | | | |
| | | | | | | FLJ11712 | | | |
| PTD004 | 0.0353 | 4.94E-03 | 3.883 | 3.798 | 219293_s_at | hypothetical | single | No | No |
| | | | | | | protein | | | |
| | | | | | | PTD004 | | | |
| MCFD2 | 0.0687 | 1.40E-02 | 3.299 | 3.808 | 212245_at | multiple | single | No | No |
| | | | | | | coagulation | | | |
| | | | | | | factor | | | |
| | | | | | | deficiency 2 | | | |
| HSPA4 | 0.0212 | 2.23E-03 | 4.047 | 3.833 | 208815_x_at | heat shock | single | Yes | No |
| | | | | | | 70kDa protein | | | |
| | | | | | | 4 | | | |
| RPL19 | 0.037 | 5.32E-03 | 3.543 | 3.836 | 200029_at | ribosomal | single | Yes | Yes |
| | | | | | | protein L19 | | | |
| NDUFA6 | 0.00489 | 1.76E-04 | 3.979 | 3.843 | 202001_s_at | NADH | single | Yes | No |
| | | | | | | dehydrogenase | | | |
| | | | | | | (ubiquinone) 1 | | | |
| | | | | | | alpha | | | |
| | | | | | | subcomplex, | | | |
| | | | | | | 6, 14kDa | | | |
| | 0.00126 | 7.93E-06 | 4.337 | 3.850 | 217446_x_at | | | N/A | N/A |
| HNRPD | 0.0375 | 5.51E-03 | 4.357 | 3.860 | 200073_s_at | heterogeneous | multiple | Yes | No |
| | | | | | | nuclear | | | |
| | | | | | | ribonucleo- | | | |
| | | | | | | protein D (AU- | | | |
| | | | | | | rich element | | | |
| | | | | | | RNA binding | | | |
| | | | | | | protein 1, | | | |
| | | | | | | 37kDa) | | | |
| GCSH | 0.00755 | 3.80E-04 | 4.230 | 3.885 | 213129_s_at | glycine | single | Yes | No |
| | | | | | | cleavage | | | |
| | | | | | | system protein | | | |
| | | | | | | H | | | |
| | | | | | | (aminomethyl | | | |
| | | | | | | carrier) | | | |
| BUB3 | 0.0419 | 6.59E-03 | 4.233 | 3.890 | 201457_x_at | BUB3 | multiple | Yes | Yes |
| | | | | | | budding | | | |
| | | | | | | uninhibited by | | | |
| | | | | | | benzimidazoles | | | |
| | | | | | | 3 homolog | | | |
| | | | | | | (yeast) | | | |
| RPL26L1 | 0.00676 | 3.07E-04 | 4.211 | 3.893 | 218830_at | ribosomal | single | No | No |
| | | | | | | protein L26- | | | |
| | | | | | | like 1 | | | |
| GPRC5D | 0.0171 | 1.52E-03 | 4.305 | 3.928 | 221297_at | G protein- | single | No | No |
| | | | | | | coupled | | | |
| | | | | | | receptor, | | | |
| | | | | | | family C, | | | |
| | | | | | | group 5, | | | |
| | | | | | | member D | | | |
| NDUFS5 | 0.0348 | 4.83E-03 | 3.213 | 3.929 | 201757_at | NADH | single | Yes | No |
| | | | | | | dehydrogenase | | | |
| | | | | | | (ubiquinone) | | | |
| | | | | | | Fe-S protein 5, | | | |
| | | | | | | 15kDa | | | |
| | | | | | | (NADH- | | | |
| | | | | | | coenzyme Q | | | |
| | | | | | | reductase) | | | |
| ESRRBL1 | 0.00189 | 3.19E-05 | 4.429 | 3.996 | 218100_s_at | estrogen- | single | Yes | No |
| | | | | | | related | | | |
| | | | | | | receptor beta | | | |
| | | | | | | like 1 | | | |
| LOC144983 | 0.0735 | 1.55E-02 | 3.185 | 3.999 | 216559_x_at | hypothetical | single | No | No |
| | | | | | | protein | | | |
| | | | | | | LOC144983 | | | |
| NDUFB8 | 0.00671 | 3.04E-04 | 4.431 | 4.011 | 201227_s_at | NADH | single | Yes | No |
| | | | | | | dehydrogenase | | | |
| | | | | | | (ubiquinone) 1 | | | |
| | | | | | | beta | | | |
| | | | | | | subcomplex, | | | |
| | | | | | | 8, 19kDa | | | |
| FLJ10996 | 0.0773 | 1.68E-02 | 3.150 | 4.015 | 219774_at | hypothetical | single | No | No |
| | | | | | | protein | | | |
| | | | | | | FLJ10996 | | | |
| RPS15 | 0.0867 | 1.98E-02 | 3.348 | 4.049 | 200819_s_at | ribosomal | single | Yes | Yes |
| | | | | | | protein S15 | | | |
| USP9X | 0.00537 | 2.00E-04 | 4.430 | 4.050 | 201100_s_at | ubiquitin | single | Yes | No |
| | | | | | | specific | | | |
| | | | | | | protease 9, X- | | | |
| | | | | | | linked (fat | | | |
| | | | | | | facets-like, | | | |
| | | | | | | Drosophila) | | | |
| MFN1 | 0.00994 | 5.90E-04 | 4.588 | 4.073 | 207098_s_at | mitofusin 1 | single | Yes | No |
| HNRPDL | 0.0635 | 1.23E-02 | 4.040 | 4.074 | 209067_s_at | heterogeneous | single | Yes | No |
| | | | | | | nuclear | | | |
| | | | | | | ribonucleo- | | | |
| | | | | | | protein D-like | | | |
| ABCE1 | 0.0118 | 8.21E-04 | 3.786 | 4.103 | 201872_s_at | ATP-binding | single | No | No |
| | | | | | | cassette, sub- | | | |
| | | | | | | family E | | | |
| | | | | | | (OABP), | | | |
| | | | | | | member 1 | | | |
| KIAA0036 | 0.00959 | 5.49E-04 | 4.375 | 4.135 | 211707_s_at | IQ | single | No | No |
| | | | | | | calmodulin- | | | |
| | | | | | | binding motif | | | |
| | | | | | | containing 1 | | | |
| UBA2 | 0.0215 | 2.28E-03 | 4.168 | 4.190 | 201177_s_at | SUMO-1 | single | No | No |
| | | | | | | activating | | | |
| | | | | | | enzyme | | | |
| | | | | | | subunit 2 | | | |
| FKSG17 | 0.000732 | 2.00E-06 | 4.514 | 4.213 | 211445_x_at | FKSG17 | single | No | No |
| LEREPO4 | 0.0172 | 1.54E-03 | 4.607 | 4.214 | 201595_s_at | likely ortholog | single | Yes | No |
| | | | | | | of mouse | | | |
| | | | | | | immediate | | | |
| | | | | | | early response, | | | |
| | | | | | | erythropoietin | | | |
| | | | | | | 4 | | | |
| RPL35A | 0.0502 | 8.64E-03 | 3.493 | 4.215 | 213687_s_at | ribosomal | single | No | No |
| | | | | | | protein L35a | | | |
| TRIM44 | 0.0382 | 5.64E-03 | 4.971 | 4.232 | 217760_at | tripartite | single | No | No |
| | | | | | | motif- | | | |
| | | | | | | containing 44 | | | |
| RAD21 | 0.0133 | 9.91E-04 | 4.274 | 4.257 | 200608_s_at | RAD21 | single | Yes | Yes |
| | | | | | | homolog (S. | | | |
| | | | | | | pombe) | | | |
| NDUFB4 | 0.0171 | 1.53E-03 | 5.235 | 4.272 | 218226_s_at | NADH | single | Yes | No |
| | | | | | | dehydrogenase | | | |
| | | | | | | (ubiquinone) 1 | | | |
| | | | | | | beta | | | |
| | | | | | | subcomplex, | | | |
| | | | | | | 4, 15kDa | | | |
| EEF1A1 | 0.071 | 1.47E-02 | 3.476 | 4.296 | 213477_x_at | eukaryotic | single | Yes | Yes |
| | | | | | | translation | | | |
| | | | | | | elongation | | | |
| | | | | | | factor 1 alpha | | | |
| | | | | | | 1 | | | |
| TTC17 | 0.0246 | 2.86E-03 | 3.633 | 4.346 | 218972_at | tetratrico- | single | No | No |
| | | | | | | peptide repeat | | | |
| | | | | | | domain 17 | | | |
| PLEKHH1 | 0.0168 | 1.48E-03 | 3.955 | 4.370 | 64942_at | | | N/A | N/A |
| PSMB1 | 0.0549 | 9.90E-03 | 5.083 | 4.385 | 214288_s_at | proteasome | single | No | No |
| | | | | | | (prosome, | | | |
| | | | | | | macropain) | | | |
| | | | | | | subunit, beta | | | |
| | | | | | | type, 1 | | | |
| RWDD1 | 0.0133 | 9.94E-04 | 3.926 | 4.411 | 219598_s_at | RWD domain | single | No | No |
| | | | | | | containing 1 | | | |
| TAF7 | 0.0635 | 1.24E-02 | 4.126 | 4.421 | 201023_at | TAF7 RNA | single | Yes | No |
| | | | | | | polymerase II, | | | |
| | | | | | | TATA box | | | |
| | | | | | | binding | | | |
| | | | | | | protein (TBP)- | | | |
| | | | | | | associated | | | |
| | | | | | | factor, 55kDa | | | |
| RPL27 | 0.0555 | 1.00E-02 | 3.661 | 4.435 | 200025_s_at | ribosomal | single | No | No |
| | | | | | | protein L27 | | | |
| RPL5 | 0.0114 | 7.59E-04 | 4.900 | 4.532 | 200937_s_at | ribosomal | multiple | No | No |
| | | | | | | protein L5 | | | |
| LOC153561 | 0.00092 | 4.23E-06 | 5.268 | 4.610 | 213089_at | hypothetical | single | No | No |
| | | | | | | protein | | | |
| | | | | | | LOC153561 | | | |
| RPL38 | 0.0856 | 1.95E-02 | 3.262 | 4.643 | 202029_x_at | ribosomal | single | No | No |
| | | | | | | protein L38 | | | |
| RPL36AL | 0.0114 | 7.47E-04 | 4.472 | 4.645 | 201406_at | ribosomal | multiple | No | No |
| | | | | | | protein L36a- | | | |
| | | | | | | like | | | |
| CCNH | 0.0988 | 2.39E-02 | 3.661 | 4.670 | 204093_at | cyclin H | single | Yes | No |
| TMSB10 | 0.0425 | 6.74E-03 | 4.215 | 4.699 | 217733_s_at | thymosin, | single | Yes | No |
| | | | | | | beta 10 | | | |
| RPS25 | 0.0306 | 3.93E-03 | 3.331 | 4.701 | 200091_s_at | ribosomal | single | No | No |
| | | | | | | protein S25 | | | |
| SP100 | 0.0125 | 8.95E-04 | 3.849 | 4.739 | 202863_at | nuclear | single | Yes | No |
| | | | | | | antigen Sp100 | | | |
| VDAC3 | 0.00384 | 1.14E-04 | 5.809 | 4.751 | 208845_at | voltage- | single | Yes | No |
| | | | | | | dependent | | | |
| | | | | | | anion channel | | | |
| | | | | | | 3 | | | |
| H2AFY | 0.00139 | 1.18E-05 | 5.427 | 4.763 | 220375_s_at | H2A histone | single | No | No |
| | | | | | | family, | | | |
| | | | | | | member Y | | | |
| FLJ14668 | 0.011 | 7.10E-04 | 4.890 | 4.774 | 215947_s_at | hypothetical | single | No | No |
| | | | | | | protein | | | |
| | | | | | | FLJ14668 | | | |
| RRN3 | 0.0196 | 1.92E-03 | 3.931 | 4.808 | 216908_x_at | RNA | single | Yes | No |
| | | | | | | polymerase I | | | |
| | | | | | | transcription | | | |
| | | | | | | factor RRN3 | | | |
| MAN1C1 | 0.000899 | 3.93E-06 | 5.500 | 4.877 | 218918_at | mannosidase, | single | Yes | No |
| | | | | | | alpha, class | | | |
| | | | | | | 1C, member 1 | | | |
| HARS | 0.00651 | 2.85E-04 | 5.305 | 4.881 | 202042_at | histidyl-tRNA | single | Yes | No |
| | | | | | | synthetase | | | |
| CDC16 | 0.00384 | 1.14E-04 | 5.179 | 4.904 | 209659_s_at | CDC16 cell | single | Yes | No |
| | | | | | | division cycle | | | |
| | | | | | | 16 homolog | | | |
| | | | | | | (S. cerevisiae) | | | |
| MRCL3 | 0.00482 | 1.70E-04 | 4.825 | 5.009 | 201319_at | myosin | single | Yes | No |
| | | | | | | regulatory | | | |
| | | | | | | light chain | | | |
| | | | | | | MRCL3 | | | |
| SEC63 | 0.00817 | 4.40E-04 | 6.364 | 5.076 | 201916_s_at | SEC63-like | single | Yes | No |
| | | | | | | (S. cerevisiae) | | | |
| RPL31 | 0.03 | 3.81E-03 | 4.290 | 5.084 | 200963_x_at | ribosomal | single | Yes | Yes |
| | | | | | | protein L31 | | | |
| NIFU | 0.0508 | 8.80E-03 | 3.867 | 5.110 | 209075_s_at | iron-sulfur | single | Yes | No |
| | | | | | | cluster | | | |
| | | | | | | assembly | | | |
| | | | | | | enzyme | | | |
| MTMR9 | 0.0186 | 1.76E-03 | 6.285 | 5.141 | 204837_at | myotubularin | single | No | No |
| | | | | | | related protein | | | |
| | | | | | | 9 | | | |
| RPL36A | 0.00129 | 9.75E-06 | 5.312 | 5.196 | 217256_x_at | ribosomal | single | No | No |
| | | | | | | protein L36a | | | |
| | 0.0454 | 7.55E-03 | 3.958 | 5.219 | 200012_x_at | | | N/A | N/A |
| CHCHD7 | 0.042 | 6.64E-03 | 3.531 | 5.286 | 218642_s_at | coiled-coil- | single | No | No |
| | | | | | | helix-coiled- | | | |
| | | | | | | coil-helix | | | |
| | | | | | | domain | | | |
| | | | | | | containing 7 | | | |
| RPS17 | 0.00354 | 9.59E-05 | 5.070 | 5.351 | 212578_x_at | ribosomal | single | No | No |
| | | | | | | protein S17 | | | |
| PCM1 | 0.00216 | 4.26E-05 | 6.574 | 5.370 | 214118_x_at | pericentriolar | single | No | No |
| | | | | | | material 1 | | | |
| RPL34 | 0.0427 | 6.86E-03 | 4.804 | 5.509 | 200026_at | ribosomal | single | No | No |
| | | | | | | protein L34 | | | |
| CBX3 | 0.0211 | 2.20E-03 | 6.761 | 5.622 | 200037_s_at | chromobox | single | No | No |
| | | | | | | homolog 3 | | | |
| | | | | | | (HP1 gamma | | | |
| | | | | | | homolog, | | | |
| | | | | | | Drosophila) | | | |
| MRPS31 | 0.000293 | 1.07E-07 | 5.502 | 5.742 | 212603_at | mitochondrial | single | No | No |
| | | | | | | ribosomal | | | |
| | | | | | | protein S31 | | | |
| RPS3A | 0.0419 | 6.59E-03 | 4.500 | 5.768 | 212391_x_at | ribosomal | multiple | Yes | Yes |
| | | | | | | protein S3A | | | |
| RPS19 | 0.00703 | 3.39E-04 | 5.278 | 5.773 | 202649_x_at | ribosomal | single | Yes | No |
| | | | | | | protein S19 | | | |
| RPS15A | 0.0543 | 9.70E-03 | 3.647 | 5.841 | 200781_s_at | ribosomal | single | No | No |
| | | | | | | protein S15a | | | |
| NTAN1 | 0.00489 | 1.75E-04 | 6.521 | 5.993 | 213062_at | N-terminal | single | Yes | No |
| | | | | | | asparagine | | | |
| | | | | | | amidase | | | |
| GTF3A | 0.02 | 1.98E-03 | 5.494 | 6.029 | 201338_x_at | general | single | Yes | Yes |
| | | | | | | transcription | | | |
| | | | | | | factor IIIA | | | |
| FLJ13213 | 0.0265 | 3.22E-03 | 4.494 | 6.102 | 217828_at | hypothetical | single | No | No |
| | | | | | | protein | | | |
| | | | | | | FLJ13213 | | | |
| RPS7 | 0.0363 | 5.17E-03 | 4.105 | 6.111 | 213941_x_at | ribosomal | single | Yes | Yes |
| | | | | | | protein S7 | | | |
| NAP1L1 | 0.0791 | 1.74E-02 | 5.196 | 6.233 | 212967_x_at | nucleosome | multiple | Yes | No |
| | | | | | | assembly | | | |
| | | | | | | protein 1-like | | | |
| | | | | | | 1 | | | |
| RPS6 | 0.0227 | 2.51E-03 | 5.313 | 6.356 | 209134_s_at | ribosomal | multiple | Yes | Yes |
| | | | | | | protein S6 | | | |
| RPS27 | 0.00161 | 2.12E-05 | 5.908 | 6.361 | 200741_s_at | ribosomal | single | No | No |
| | | | | | | protein S27 | | | |
| | | | | | | (metallopansti | | | |
| | | | | | | mulin 1) | | | |
| DDX5 | 0.0895 | 2.07E-02 | 4.224 | 6.423 | 200033_at | DEAD (Asp- | single | No | No |
| | | | | | | Glu-Ala-Asp) | | | |
| | | | | | | box | | | |
| | | | | | | polypeptide 5 | | | |
| RPLP1 | 0.061 | 1.17E-02 | 4.328 | 6.622 | 200763_s_at | ribosomal | single | Yes | Yes |
| | | | | | | protein, large, | | | |
| | | | | | | P1 | | | |
| RPS24 | 0.0176 | 1.60E-03 | 4.942 | 6.800 | 200061_s_at | ribosomal | single | Yes | Yes |
| | | | | | | protein S24 | | | |
| PTPN2 | 0.00752 | 3.77E-04 | 6.736 | 7.030 | 213136_at | protein | multiple | Yes | Yes |
| | | | | | | tyrosine | | | |
| | | | | | | phosphatase, | | | |
| | | | | | | non-receptor | | | |
| | | | | | | type 2 | | | |
| RPL4 | 0.00294 | 7.14E-05 | 6.268 | 7.111 | 200089_s at | ribosomal | multiple | Yes | Yes |
| | | | | | | protein L4 | | | |
| RPL22 | 0.0111 | 7.15E-04 | 5.862 | 7.320 | 220960_x_at | ribosomal | multiple | Yes | No |
| | | | | | | protein L22 | | | |
| MRPS22 | 0.0116 | 7.92E-04 | 7.774 | 7.370 | 219220_x_at | mitochondrial | single | No | No |
| | | | | | | ribosomal | | | |
| | | | | | | protein S22 | | | |
| GPR153 | 0.0174 | 1.57E-03 | 5.836 | 7.470 | 220725_x_at | | | N/A | N/A |
| MAPRE1 | 0.00298 | 7.30E-05 | 8.747 | 7.718 | 200712_s_at | microtubule- | single | Yes | No |
| | | | | | | associated | | | |
| | | | | | | protein, | | | |
| | | | | | | RP/EB family, | | | |
| | | | | | | member 1 | | | |
| HMGN2 | 0.0735 | 1.56E-02 | 5.160 | 7.778 | 208668_x_at | high-mobility | single | Yes | No |
| | | | | | | group | | | |
| | | | | | | nucleosomal | | | |
| | | | | | | binding | | | |
| | | | | | | domain 2 | | | |
| RPL17 | 0.00283 | 6.65E-05 | 7.454 | 8.038 | 200038_s_at | ribosomal | multiple | No | No |
| | | | | | | protein L17 | | | |
| RPL9 | 0.022 | 2.36E-03 | 5.775 | 8.273 | 200032_s_at | ribosomal | single | Yes | Yes |
| | | | | | | protein L9 | | | |
| FLJ20003 | 0.000336 | 2.04E-07 | 8.503 | 8.278 | 219067_s_at | chromosome | single | No | No |
| | | | | | | 10 open | | | |
| | | | | | | reading frame | | | |
| | | | | | | 86 | | | |
| RPL14 | 0.00211 | 3.94E-05 | 7.439 | 8.807 | 200074_s_at | ribosomal | multiple | Yes | Yes |
| | | | | | | protein L14 | | | |
| RPL6 | 0.00546 | 2.13E-04 | 8.309 | 8.928 | 200034_s_at | ribosomal | single | Yes | Yes |
| | | | | | | protein L6 | | | |
| RPL7 | 0.0159 | 1.37E-03 | 6.500 | 9.048 | 200717_x_at | ribosomal | multiple | Yes | Yes |
| | | | | | | protein L7 | | | |
| FLJ11021 | 0.0189 | 1.79E-03 | 6.351 | 9.554 | 202302_s_at | similar to | single | No | No |
| | | | | | | splicing factor, | | | |
| | | | | | | arginine/serine- | | | |
| | | | | | | rich 4 | | | |
| EIF4G2 | 0.0549 | 9.90E-03 | 8.478 | 10.152 | 200004_at | eukaryotic | single | Yes | Yes |
| | | | | | | translation | | | |
| | | | | | | initiation | | | |
| | | | | | | factor 4 | | | |
| | | | | | | gamma, 2 | | | |
| BTF3 | 0.00687 | 3.17E-04 | 10.155 | 10.364 | 211939_x_at | basic | multiple | No | No |
| | | | | | | transcription | | | |
| | | | | | | factor 3 | | | |
| SFRS14 | 0.00144 | 1.58E-05 | 8.161 | 10.381 | 213505_s_at | splicing factor, | single | No | No |
| | | | | | | arginine/serine- | | | |
| | | | | | | rich 14 | | | |
| SRP14 | 0.0176 | 1.60E-03 | 8.130 | 11.379 | 200007_at | signal | single | Yes | Yes |
| | | | | | | recognition | | | |
| | | | | | | particle 14kDa | | | |
| | | | | | | (homologous | | | |
| | | | | | | Alu RNA | | | |
| | | | | | | binding | | | |
| | | | | | | protein) | | | |
| PTMA | 0.00593 | 2.44E-04 | 10.333 | 11.508 | 200773_x_at | prothymosin, | single | Yes | No |
| | | | | | | alpha (gene | | | |
| | | | | | | sequence 28) | | | |
| RLPS4X | 0.00483 | 1.72E-04 | 9.330 | 13.462 | 216342_x_at | ribosomal | multiple | No | No |
| | | | | | | protein S4, | | | |
| | | | | | | X-linked | | | |

TABLE 25
Association Between Preimmunization Expression Levels of
Genes Involved in Protein Synthesis Machinery and Post-
immunization IgG Response

| | FDR | Odds Ratio | |
|---|---|---|---|
| | association | association | |
| Gene | with IgG | with IgG | |
| Name | response | response | Gene Description |
| MRPS31 | 0.000293 | 5.502 | mitochondrial ribosomal |
| | | | protein S31 |
| RPL7 | 0.000732 | 5.846 | ribosomal protein L7 |
| RPL26 | 0.00121 | 3.342 | ribosomal protein L26 |
| RPL36A | 0.00129 | 5.312 | ribosomal protein L36a |
| RPS27 | 0.00161 | 5.908 | ribosomal rotein S27 |
| | | | (metallopanstimulin 1) |
| RPL14 | 0.00211 | 7.439 | ribosomal protein L14 |
| RPL17 | 0.00283 | 7.454 | ribosomal protein L17 |
| RPL4 | 0.00294 | 6.268 | ribosomal protein L4 |
| RPL7 | 0.00336 | 6.044 | ribosomal protein L7 |
| RPS17 | 0.00354 | 5.070 | ribosomal protein S17 |
| RPL14 | 0.00373 | 5.310 | ribosomal protein L14 |
| RPL17 | 0.00435 | 6.380 | ribosomal protein L17 |
| RPS4X | 0.00483 | 9.330 | ribosomal protein S4, |
| | | | X-linked |
| RPL6 | 0.00546 | 8.309 | ribosomal protein L6 |
| RPS3A | 0.00631 | 3.179 | ribosomal protein S3A |
| RPL17 | 0.00645 | 5.874 | ribosomal protein L17 |
| RPL26L1 | 0.00676 | 4.211 | ribosomal protein L26-like 1 |
| RPS19 | 0.00703 | 5.278 | ribosomal protein S19 |
| RPL35 | 0.00735 | 3.013 | ribosomal protein L35 |
| SSB | 0.00795 | 3.664 | Sjogren syndrome antigen B |
| | | | (autoantigen La) |
| RPL22 | 0.0111 | 5.862 | ribosomal protein L22 |
| RPL36AL | 0.0114 | 4.472 | ribosomal protein L36a-like |
| RPL5 | 0.0114 | 4.900 | ribosomal protein L5 |
| MRPS22 | 0.0116 | 7.774 | mitochondrial ribosomal |
| | | | protein S22 |
| RPL35 | 0.0158 | 3.439 | ribosomal protein L35 |
| RPL7 | 0.0159 | 6.500 | ribosomal protein L7 |
| RPL22 | 0.0174 | 5.543 | ribosomal protein L22 |
| RPS24 | 0.0176 | 4.942 | ribosomal protein S24 |
| RPL36AL | 0.0179 | 4.078 | ribosomal protein L36a-like |
| RPL9 | 0.022 | 5.775 | ribosomal protein L9 |
| RPS4X | 0.0222 | 5.131 | ribosomal protein S4, |
| | | | X-linked |
| RPS6 | 0.0227 | 5.313 | ribosomal protein S6 |
| RPL4 | 0.0229 | 4.112 | ribosomal protein L4 |
| RPL22 | 0.0269 | 5.178 | ribosomal protein L22 |
| RPL31 | 0.03 | 4.290 | ribosomal protein L31 |
| RPS25 | 0.0306 | 3.331 | ribosomal protein S25 |
| FOXO1A | 0.0328 | 3.167 | forkhead box O1A |
| | | | (rhabdomyosarcoma) |
| RPL5 | 0.0354 | 3.356 | ribosomal protein L5 |
| RPS7 | 0.0363 | 4.105 | ribosomal protein S7 |
| RPL19 | 0.037 | 3.543 | ribosomal protein L19 |
| EIF3S1 | 0.0395 | 3.184 | eukaryotic translation |
| | | | initiation factor 3, |
| | | | subunit 1a |
| RPS3A | 0.0419 | 4.500 | ribosomal protein S3A |
| RPL34 | 0.0427 | 4.804 | ribosomal protein L34 |
| RPL4 | 0.0431 | 3.436 | ribosomal protein L4 |
| RPL21 | 0.0454 | 3.958 | ribosomal protein L21 |
| | | | (gene or pseudogene) |
| RPL35A | 0.0502 | 3.493 | ribosomal protein L35a |
| RPS15A | 0.0543 | 3.647 | ribosomal protein S15a |
| EIF4G2 | 0.0549 | 8.478 | eukaryotic translation |
| | | | initiation factor 4 gamma,2 |

TABLE 26
Selection of Genes Associated with IgG Responsiveness

| Ingenuity category | Gene | FDR | OR | Description |
|---|---|---|---|---|
| Protein trafficking | SRP14 | 0.018 | 8.13 | signal recognition particle 14kDa |
| Protein trafficking | MCM3AP | 0.009 | 3.63 | MCM3 minichromosome maintenance |
| | | | | deficient 3 associated protein |
| Protein trafficking | UBL1 | 0.04 | 3.16 | SMT3 suppressor of mif two 3 homolog 1 |
| | | | | (yeast) |
| Protein trafficking | KPNA6 | 0.012 | 0.32 | karyopherin alpha 6 (importin alpha 7) |
| Protein trafficking | ARF3 | 0.005 | 0.32 | ADP-ribosylation factor 3 |
| Protein trafficking | XPO7 | 0.003 | 0.29 | exportin 7 |
| Protein trafficking | KDELR2 | 0.019 | 0.29 | KDEL endoplasmic reticulum protein |
| | | | | retention receptor 2 |
| Protein trafficking | NUP214 | 0.018 | 0.26 | nucleoporin 214kDa |
| Protein synthesis | EIF4G2 | 0.055 | 8.48 | eukaryotic translation initiation factor 4 |
| | | | | gamma, 2 |
| Protein synthesis | RPL6 | 0.005 | 8.31 | ribosomal protein L6 |
| Protein synthesis | RPL4 | 0.003 | 6.27 | ribosomal protein L4 |
| Protein synthesis | RPL7 | 0.003 | 6.04 | ribosomal protein L7 |
| Protein synthesis | RPL9 | 0.022 | 5.77 | ribosomal protein L9 |
| Protein synthesis | RPS6 | 0.0227 | 5.31 | ribosomal protein S6 |
| Protein synthesis | RPL14 | 0.004 | 5.31 | ribosomal protein L14 |
| Protein synthesis | RPS24 | 0.018 | 4.94 | ribosomal protein S24 |
| Protein synthesis | RPLP1 | 0.061 | 4.33 | ribosomal protein, large, P1 |
| Protein synthesis | RPL31 | 0.03 | 4.29 | ribosomal protein L31 |
| Protein synthesis | RPS7 | 0.036 | 4.11 | ribosomal protein S7 |
| Protein synthesis | SSB | 0.008 | 3.66 | Sjogren syndrome antigen B |
| Protein synthesis | RPL19 | 0.037 | 3.54 | ribosomal protein L19 |
| Protein synthesis | EEF1A1 | 0.071 | 3.48 | eukaryotic translation elongation factor 1 |
| | | | | alpha 1 |
| Protein synthesis | RPS15 | 0.087 | 3.35 | ribosomal protein S15 |
| Protein synthesis | RPL26 | 0.001 | 3.34 | ribosomal protein L26 |
| Protein synthesis | EIF3S1 | 0.039 | 3.18 | eukaryotic translation initiation factor 3, |
| | | | | subunit 1 alpha, 35kDa |
| Protein synthesis | FOXO1A | 0.033 | 3.17 | forkhead box O1A (rhabdomyosarcoma) |
| Protein synthesis | TXNRD1 | 0.057 | 0.28 | thioredoxin reductase 1 |
| Protein synthesis | RELA | 0.007 | 0.26 | v-rel reticuloendotheliosis viral oncogene |
| | | | | homolog A, nuclear factor of kappa light |
| | | | | polypeptide gene enhancer in B-cells 3, |
| | | | | p65 (avian) |
| Protein synthesis | PABPC4 | 0.004 | 0.26 | poly(A) binding protein, cytoplasmic 4 |
| | | | | (inducible form) |
| Protein synthesis | EIF4A1 | 0.099 | 0.22 | eukaryotic translation initiation factor 4A, |
| | | | | isoform 1 |
| Protein synthesis | MAP2K3 | 0.0028 | 0.19 | mitogen-activated protein kinase kinase 3 |
| Protein synthesis | MIKNK1 | 0.019 | 0.15 | MAP kinase interacting serine/threonine |
| | | | | kinase 1 |
| Protein synthesis | PTBP1 | 0.002 | 0.11 | polypyrimidine tract binding protein |
| DNA repair, | CDK2 | 0.004 | 4.29 | cyclin-dependent kinase 2 |
| replication, | | | | |
| recombination | | | | |
| DNA repair, | TMPO | 0.009 | 3.99 | thymopoietin |
| replication, | | | | |
| recombination | | | | |
| DNA repair, | PRKDC | 0.006 | 3.40 | protein kinase, DNA-activated, catalytic |
| replication, | | | | polypeptide, role in VDJ recombination |
| recombination | | | | |
| DNA repair, | BUB3 | 0.021 | 3.12 | BUB3 budding uninhibited by |
| replication, | | | | benzimidazoles 3 homolog (yeast) |
| recombination | | | | |
| DNA repair, | XRCC2 | 0.002 | 3.03 | X-ray repair complementing defective |
| replication, | | | | repair, role in VDJ recombination. |
| recombination | | | | |
| DNA repair, | CDKN1A | 0.004 | 0.32 | cyclin-dependent kinase inhibitor 1A |
| replication, | | | | (p21, Cip1) |
| recombination | | | | |
| DNA repair, | PAFAH1B1 | 0.0045 | 0.21 | platelet-activating factor acetylhydrolase |
| replication, | | | | beta subunit (PAF-AH beta) |
| recombination | | | | |

FDR = false discovery rate; OR = odds ratio

TABLE 27
Annotation of IgG-associated Genes.

| | | | | | |
|---|---|---|---|---|---|
| | Gene assigned by Ingenuity to one of | | | +HL,34 | |
| | the following functions: cell-cycle | | | | |
| | (includes DNA synthesis, cell growth | | | | |
| | and proliferation), cell death, cell | | | | |
| | signaling and interaction (includes | | | | |
| | cell signaling and cell-to-cell | | | | |
| | signaling and interaction), immune | | | | |
| | functions (includes immune and | | | Identified | |
| | lymphatic system development and | | | by more | Affymetrix |
| | function and immune response), | FDR IgG | Odds | than one | probeset |
| Gene Name | protein synthesis and trafficking | association | Ratio | probeset | identifier |

| | | | | | |
|---|---|---|---|---|---|
| MRPS31 | Yes | 0.0003 | 5.502 | No | 212603_at |
| FLJ20003 | Not assigned to function by Ingenuity | 0.0003 | 8.503 | No | 219067_s_at |
| PGF | No | 0.0007 | 3.555 | No | 215179_x_a |
| SLC12A9 | Not assigned to function by Ingenuity | 0.0007 | 0.117 | No | 220371_s_at |
| FKSG17 | Not assigned to function by Ingenuity | 0.0007 | 4.514 | No | 211445_x_a |
| MAN1C1 | No | 0.0009 | 5.5 | No | 218918_at |
| LOC153561 | Not assigned to function by Ingenuity | 0.0009 | 5.268 | No | 213089_at |
| POLR1B | No | 0.0011 | 3.59 | No | 220113_x_a |
| MFN2 | No | 0.0012 | 0.205 | No | 201155_s_at |
| RIOK3 | No | 0.0012 | 3.312 | No | 215588_x_a |
| RPL26 | Yes | 0.0012 | 3.342 | No | 222229_x_a |
| RPL36A | Yes | 0.0013 | 5.312 | No | 217256_x_a |
| FLJ10315 | Not assigned to function by Ingenuity | 0.0014 | 0.174 | No | 218770_s_at |
| H2AFY | Not assigned to function by Ingenuity | 0.0014 | 5.427 | No | 220375_s_at |
| MXD4 | No | 0.0014 | 0.254 | No | 212346_s_at |
| EMT | No | 0.0014 | 0.259 | No | 207621_s_at |
| CORO1B | Not assigned to function by Ingenuity | 0.0014 | 0.303 | No | 64486_at |
| SFRS14 | Not assigned to function by Ingenuity | 0.0014 | 8.161 | No | 213505_s_at |
| PLOD | No | 0.0015 | 0.245 | No | 200827_at |
| FLJ11560 | Not assigned to function by Ingenuity | 0.0015 | 0.189 | Yes | 211433_x_a |
| RPS27 | Yes | 0.0016 | 5.908 | No | 200741_s_at |
| XRCC2 | Yes | 0.0017 | 3.033 | No | 207598_x_a |
| GRINA | Not assigned to function by Ingenuity | 0.0017 | 0.281 | No | 212090_at |
| LAMR1 | No | 0.0017 | 3.016 | No | 216806_at |
| DKFZPS64J157 | Not assigned to function by Ingenuity | 0.0018 | 0.158 | No | 217794_at |
| DNASE1L1 | No | 0.0018 | 0.231 | No | 203912_s_at |
| ESRRBL1 | No | 0.0019 | 4.429 | No | 218100_s_at |
| ARPC1B | No | 0.0020 | 0.276 | No | 201954_at |
| PTBP1 | Yes | 0.0020 | 0.112 | Yes | 211270_x_a |
| MAPK7 | No | 0.0021 | 0.255 | No | 35617_at |
| LONP | Not assigned to function by Ingenuity | 0.0021 | 3.226 | No | 221834_at |
| WBSCR5 | No | 0.0021 | 3.921 | No | 211768_at |
| unannotated | Not assigned to function by Ingenuity | 0.0021 | 3.099 | No | 216006_at |
| RPL14 | Yes | 0.0021 | 7.439 | Yes | 200074_s_at |
| PCM1 | Not assigned to function by Ingenuity | 0.0022 | 6.574 | No | 214118_x_a |
| HDGF | No | 0.0024 | 0.137 | No | 216484_x_a |
| unannotated | Not assigned to function by Ingenuity | 0.0025 | 4.29 | No | 215628_x_a |
| IMPDH1 | No | 0.0026 | 0.274 | No | 204169_at |
| FLJ20331 | Not assigned to function by Ingenuity | 0.0026 | 3.855 | No | 215063_x_a |
| PPP2R4 | No | 0.0026 | 0.237 | No | 208874_x_a |
| MAP2K3 | Yes | 0.0028 | 0.195 | No | 215499_at |
| VCP | No | 0.0028 | 0.172 | No | 208648_at |
| GTF2H2 | Not assigned to function by Ingenuity | 0.0028 | 3.033 | No | 221540_x_a |
| PPP2CA | Not assigned to function by Ingenuity | 0.0028 | 3.291 | No | 217713_x_a |
| RPL17 | Yes | 0.0028 | 7.454 | Yes | 200038_s_at |
| GDI1 | No | 0.0029 | 0.283 | No | 201864_at |
| XPO7 | Yes | 0.0029 | 0.298 | No | 212166_at |
| RPL4 | Yes | 0.0029 | 6.268 | Yes | 200089_s_at |
| MAPRE1 | No | 0.0030 | 8.747 | No | 200712_s_at |
| DKFZp566N034 | Not assigned to function by Ingenuity | 0.0030 | 4.15 | No | 208238_x_a |
| COBRA1 | No | 0.0034 | 0.257 | No | 202757_at |
| GSTM3 | No | 0.0035 | 3.641 | No | 202554_s_at |
| RPS17 | Yes | 0.0035 | 5.07 | No | 212578_x_a |
| LASS2 | Not assigned to function by Ingenuity | 0.0036 | 0.259 | No | 222212_s_at |
| NIF3L1BP1 | Not assigned to function by Ingenuity | 0.0036 | 3.506 | No | 218334_at |
| ATP6V0C | No | 0.0036 | 0.215 | No | 36994_at |
| POLR2L | No | 0.0036 | 0.296 | No | 211730_s_at |
| MGC10433 | Not assigned to function by Ingenuity | 0.0036 | 0.192 | No | 205740_s_at |
| C14ORF123 | Not assigned to function by Ingenuity | 0.0037 | 3.718 | No | 218571_s_at |
| ALMS1 | No | 0.0037 | 3.045 | No | 214707_x_a |
| CDC16 | No | 0.0038 | 5.179 | No | 209659_s_at |
| VDAC3 | No | 0.0038 | 5.809 | No | 208845_at |
| ATP6V0A1 | Not assigned to function by Ingenuity | 0.0039 | 0.175 | No | 212383_at |
| CAB45 | Not assigned to function by Ingenuity | 0.0039 | 0.328 | No | 217855_x_a |
| CDKN1A | Yes | 0.0040 | 0.325 | No | 202284_s_at |
| SH3BP2 | No | 0.0040 | 0.207 | No | 209370_s_at |
| CDK2 | Yes | 0.0040 | 4.295 | No | 204252_at |
| FLJ10460 | Not assigned to function by Ingenuity | 0.0043 | 3.085 | No | 220071_x_a |
| PAFAH1B1 | Yes | 0.0045 | 0.212 | No | 200815_s_at |
| BLCAP | Not assigned to function by Ingenuity | 0.0046 | 0.175 | No | 201032_at |
| PABPC4 | Yes | 0.0046 | 0.256 | No | 201064_s_at |
| ARF3 | Yes | 0.0048 | 0.32 | No | 200011_s_at |
| MRCL3 | No | 0.0048 | 4.825 | No | 201319_at |
| LSM5 | Not assigned to function by Ingenuity | 0.0048 | 3.126 | No | 211747_s_at |
| RPS4X | Yes | 0.0048 | 9.33 | Yes | 216342_x_a |
| NDUFA6 | No | 0.0049 | 3.979 | No | 202001_s_at |
| NTAN1 | No | 0.0049 | 6.521 | No | 213062_at |
| RRAGD | Not assigned to function by Ingenuity | 0.0051 | 0.276 | No | 221523_s_at |
| HSPC128 | Not assigned to function by Ingenuity | 0.0051 | 3.394 | No | 218936_s_at |
| USP9X | No | 0.0054 | 4.43 | No | 201100_s_at |
| FLJ10307 | Not assigned to function by Ingenuity | 0.0054 | 0.209 | No | 218753_at |
| RARG-1 | Not assigned to function by Ingenuity | 0.0054 | 4.107 | Yes | 202882_x_a |
| PEPD | Not assigned to function by Ingenuity | 0.0054 | 0.273 | No | 202108_at |
| RBBP6 | No | 0.0054 | 3.065 | No | 212781_at |
| RPL6 | Yes | 0.0055 | 8.309 | No | 200034_s_at |
| ADRM1 | Not assigned to function by Ingenuity | 0.0055 | 0.284 | No | 201281_at |
| ZNF-U69274 | Not assigned to function by Ingenuity | 0.0055 | 4.158 | No | 204847_at |
| CDC40 | No | 0.0056 | 0.177 | No | 203376_at |
| CLIC4 | No | 0.0056 | 0.318 | No | 201560_at |
| UQCRB | No | 0.0058 | 3.91 | Yes | 209065_at |
| PTMA | No | 0.0059 | 10.333 | No | 200773_x_a |
| CLN2 | No | 0.0061 | 0.331 | No | 200742_s_at |
| COX7A3 | No | 0.0063 | 4.532 | No | 217249_x_a |
| PRKDC | Yes | 0.0063 | 3.401 | No | 208694_at |
| SNN | No | 0.0064 | 0.3 | No | 218033_s_at |
| DCTN1 | No | 0.0065 | 0.3 | No | 211780_x_a |
| HARS | No | 0.0065 | 5.305 | No | 202042_at |
| Unknown | Not assigned to function by Ingenuity | 0.0067 | 4.084 | No | 215557_at |
| ACTR1A | No | 0.0067 | 0.171 | No | 200721_s_at |
| NDUFB8 | No | 0.0067 | 4.431 | No | 201227_s_at |
| SNRPB2 | No | 0.0068 | 3.596 | No | 202505_at |
| RPL26L1 | Yes | 0.0068 | 4.211 | No | 218830_at |
| BTF3 | Not assigned to function by Ingenuity | 0.0069 | 10.155 | Yes | 211939_x_a |
| P29 | Not assigned to function by Ingenuity | 0.0070 | 3.659 | No | 202553_s_at |
| RELA | Yes | 0.0070 | 0.26 | No | 201783_s_at |
| GORASP2 | Not assigned to function by Ingenuity | 0.0070 | 0.219 | No | 207812_s_at |
| RPS19 | Yes | 0.0070 | 5.278 | No | 202649_x_a |
| TBCA | Not assigned to function by Ingenuity | 0.0071 | 3.344 | No | 203667_at |
| LOC285148 | Not assigned to function by Ingenuity | 0.0072 | 0.204 | No | 213532_at |
| PTPN2 | Yes | 0.0075 | 6.736 | Yes | 213136_at |
| GCSH | No | 0.0076 | 4.23 | No | 213129_s_at |
| CGI-12 | Not assigned to function by Ingenuity | 0.0076 | 3.378 | No | 219363_s_at |
| SCD4 | Not assigned to function by Ingenuity | 0.0077 | 3.441 | No | 214036_at |
| SSB | yes | 0.0080 | 3.664 | No | 201139_s_at |
| SEC63 | No | 0.0082 | 6.364 | No | 201916_s_at |
| ETHE1 | Not assigned to function by Ingenuity | 0.0087 | 0.289 | No | 204034_at |
| OIP2 | Not assigned to function by Ingenuity | 0.0087 | 3.544 | No | 215136_s_at |
| CENTA1 | No | 0.0089 | 0.255 | No | 90265_at |
| MCM3AP | Yes | 0.0092 | 3.627 | No | 215582_x_a |
| DULLARD | Not assigned to function by Ingenuity | 0.0094 | 0.263 | No | 200035_at |
| KIAA0036 | Not assigned to function by Ingenuity | 0.0096 | 4.375 | No | 211707_s_at |
| MPST | Not assigned to function by Ingenuity | 0.0096 | 0.259 | No | 203524_s_at |
| TMPO | Yes | 0.0098 | 3.992 | No | 209753_s_at |
| MFN1 | No | 0.0099 | 4.588 | No | 207098_s_at |
| LENG4 | No | 0.0101 | 0.302 | No | 205634_x_a |
| FLJ12287 | Not assigned to function by Ingenuity | 0.0101 | 0.332 | No | 219259_at |
| TUBA6 | Not assigned to function by Ingenuity | 0.0104 | 0.301 | Yes | 211750_x_a |
| FURIN | No | 0.0106 | 0.29 | No | 201945_at |
| FLJ14668 | Not assigned to function by Ingenuity | 0.0110 | 4.89 | No | 215947_s_at |
| HNRPH1 | No | 0.0111 | 3.327 | No | 213619_at |
| KIAA0494 | Not assigned to function by Ingenuity | 0.0111 | 0.228 | No | 201776_s_at |
| RPL22 | Yes | 0.0111 | 5.862 | Yes | 220960_x_a |
| FLJ22256 | Not assigned to function by Ingenuity | 0.0113 | 3.503 | No | 220856_x_a |
| CHK | No | 0.0114 | 0.327 | No | 204266_s_at |
| CBARA1 | Not assigned to function by Ingenuity | 0.0114 | 0.192 | No | 216903_s_at |
| WDR13 | Not assigned to function by Ingenuity | 0.0114 | 0.326 | No | 222138_s_at |
| RPL36AL | yes | 0.0114 | 4.472 | Yes | 201406_at |
| RPL5 | yes | 0.0114 | 4.9 | Yes | 200937_s_at |
| KIAA1193 | Not assigned to function by Ingenuity | 0.0115 | 0.251 | No | 4822_s_at |
| K-ALPHA-1 | Not assigned to function by Ingenuity | 0.0116 | 0.225 | Yes | 211058_x_a |
| GBA | Not assigned to function by Ingenuity | 0.0116 | 0.264 | No | 209093_s_at |
| SRF | Yes | 0.0116 | 0.319 | No | 202401_s_at |
| MRPS22 | yes | 0.0116 | 7.774 | No | 219220_x_a |
| KPNA6 | Yes | 0.0117 | 0.32 | No | 212101_at |
| ABCE1 | Not assigned to function by Ingenuity | 0.0118 | 3.786 | No | 201872_s_at |
| PP9099 | Not assigned to function by Ingenuity | 0.0122 | 0.319 | No | 204436_at |
| SP100 | No | 0.0125 | 3.849 | No | 202863_at |
| FLJ23476 | Not assigned to function by Ingenuity | 0.0126 | 3.68 | No | 218647_s_at |
| FTLL1 | Not assigned to function by Ingenuity | 0.0128 | 3.273 | No | 217703_x_a |
| SNX27 | Not assigned to function by Ingenuity | 0.0131 | 0.314 | No | 221498_at |
| C1GALT1 | Not assigned to function by Ingenuity | 0.0132 | 3.612 | No | 219439_at |
| RWDD1 | Not assigned to function by Ingenuity | 0.0133 | 3.926 | No | 219598_s_at |
| RAD21 | Yes | 0.0133 | 4.274 | No | 200608_s_at |
| SEC31L1 | No | 0.0134 | 0.19 | No | 210616_s_at |
| PSKH1 | Not assigned to function by Ingenuity | 0.0137 | 3.788 | No | 213141_at |
| TM6SF1 | Not assigned to function by Ingenuity | 0.0138 | 0.271 | No | 219892_at |
| SGSH | No | 0.0139 | 0.311 | No | 35626_at |
| DAG1 | Yes | 0.0140 | 0.256 | No | 205417_s_at |
| ATP5I | Not assigned to function by Ingenuity | 0.0144 | 3.511 | No | 209492_x_a |
| BAT3 | Not assigned to function by Ingenuity | 0.0147 | 0.326 | No | 201255_x_a |
| NTRK3 | Yes | 0.0148 | 3.298 | No | 217033_x_a |
| GLUD1 | No | 0.0151 | 0.098 | No | 200946_x_a |
| SLC35E1 | Not assigned to function by Ingenuity | 0.0152 | 3.961 | No | 79005_at |
| CPA2 | No | 0.0157 | 3.506 | No | 206212_at |
| RPL35 | yes | 0.0158 | 3.439 | Yes | 200002_at |
| RPL7 | Yes | 0.0159 | 6.5 | Yes | 200717_x_a |
| XPO6 | Not assigned to function by Ingenuity | 0.0161 | 0.237 | No | 211982_x_a |
| MGC16824 | Not assigned to function by Ingenuity | 0.0163 | 0.226 | No | 203173_s_at |
| MGC48332 | Not assigned to function by Ingenuity | 0.0165 | 3.326 | No | 213256_at |
| PLEKHH1 | Not assigned to function by Ingenuity | 0.0168 | 3.955 | No | 64942_at |
| NDUFB4 | No | 0.0171 | 5.235 | No | 218226_s_at |
| GPRC5D | Not assigned to function by Ingenuity | 0.0171 | 4.305 | No | 221297_at |
| LEREPO4 | No | 0.0172 | 4.607 | No | 201595_s_at |
| PXN | No | 0.0174 | 0.296 | No | 201087_at |
| GPR153 | Not assigned to function by Ingenuity | 0.0174 | 5.836 | No | 220725_x_a |
| NUP214 | Yes | 0.0176 | 0.264 | No | 202155_s_at |
| RPS24 | Yes | 0.0176 | 4.942 | No | 200061_s_at |
| SRP14 | Yes | 0.0176 | 8.13 | No | 200007_at |
| FLJ10287 | Not assigned to function by Ingenuity | 0.0177 | 3.262 | No | 219130_at |
| DNAH3 | Not assigned to function by Ingenuity | 0.0180 | 3.282 | No | 209751_s_at |
| ZFP95 | Not assigned to function by Ingenuity | 0.0180 | 3.562 | No | 203730_s_at |
| OS-9 | Not assigned to function by Ingenuity | 0.0184 | 0.329 | No | 200714_x_a |
| MTMR9 | Not assigned to function by Ingenuity | 0.0186 | 6.285 | No | 204837_at |
| NPEPPS | No | 0.0187 | 0.266 | No | 201454_s_at |
| FACL6 | No | 0.0188 | 3.228 | No | 211207_s_at |
| FLJ11021 | Not assigned to function by Ingenuity | 0.0189 | 6.351 | No | 202302_s_at |
| CRKL | No | 0.0192 | 0.18 | No | 212180_at |
| MKNK1 | Yes | 0.0192 | 0.147 | No | 209467_s_at |
| KDELR2 | Yes | 0.0192 | 0.297 | No | 200698_at |
| ZNF505 | Not assigned to function by Ingenuity | 0.0193 | 3.026 | No | 215758_x_a |
| GRK6 | No | 0.0194 | 0.311 | No | 210981_s_at |
| GALNACT-2 | No | 0.0196 | 0.315 | No | 222235_s_at |
| RRN3 | No | 0.0196 | 3.931 | No | 216908_x_a |
| GTF3A | Yes | 0.0200 | 5.494 | No | 201338_x_a |
| TM9SF2 | No | 0.0206 | 0.194 | No | 201078_at |
| FLJ34588 | Not assigned to function by Ingenuity | 0.0206 | 3.86 | No | 212410_at |
| OAZIN | No | 0.0208 | 0.21 | No | 212461_at |
| C20ORF35 | Not assigned to function by Ingenuity | 0.0209 | 0.306 | No | 218094_s_at |
| SMARCD2 | No | 0.0211 | 0.268 | No | 201827_at |
| CBX3 | Not assigned to function by Ingenuity | 0.0211 | 6.761 | No | 200037_s_at |
| HSPA4 | No | 0.0212 | 4.047 | No | 208815_x_a |
| CALM1 | No | 0.0214 | 3.136 | No | 209563_x_a |
| UBA2 | Not assigned to function by Ingenuity | 0.0215 | 4.168 | No | 201177_s_at |
| SLC9A8 | Not assigned to function by Ingenuity | 0.0217 | 0.315 | No | 212947_at |
| GTSE1 | Not assigned to function by Ingenuity | 0.0219 | 3.07 | No | 211040_x_a |
| RPL9 | Yes | 0.0220 | 5.775 | No | 200032_s_at |
| MPP2 | No | 0.0223 | 3.682 | No | 207984_s_at |
| TRAPCC2 | Not assigned to function by Ingenuity | 0.0224 | 3.842 | No | 206853_s_at |
| DSPP | No | 0.0227 | 3.374 | No | 221681_s_at |
| RPS6 | Yes | 0.0227 | 5.313 | Yes | 209134_s_at |
| PLOD3 | No | 0.0236 | 0.277 | No | 202185_at |
| ZNF263 | No | 0.0237 | 3.509 | No | 203707_at |
| TGFB3 | Yes | 0.0237 | 3.511 | No | 209747_at |
| MGC13024 | Not assigned to function by Ingenuity | 0.0238 | 0.273 | No | 221864_at |
| LOCS1257 | Not assigned to function by Ingenuity | 0.0240 | 0.332 | No | 210075_at |
| TCEAL1 | No | 0.0241 | 3.163 | No | 204045_at |
| VAMP4 | No | 0.0241 | 3.512 | No | 213480_at |
| NPL4 | No | 0.0243 | 0.284 | No | 217796_s_at |
| TTC17 | Not assigned to function by Ingenuity | 0.0246 | 3.633 | No | 218972_at |
| EXT2 | No | 0.0261 | 0.198 | No | 202012_s_at |
| CANX | No | 0.0265 | 0.226 | No | 200068_s_at |
| KIAA0121 | Not assigned to function by Ingenuity | 0.0265 | 0.327 | No | 212399_s_at |
| FLJ13213 | Not assigned to function by Ingenuity | 0.0265 | 4.494 | No | 217828_at |
| SGPL1 | No | 0.0270 | 0.191 | Yes | 212321_at |
| SLC30A5 | No | 0.0274 | 3.23 | No | 218989_x_a |
| CAMTA2 | Not assigned to function by Ingenuity | 0.0276 | 0.301 | No | 212948_at |
| GTF2A2 | Not assigned to function by Ingenuity | 0.0277 | 0.277 | No | 202678_at |
| SS18L2 | Not assigned to function by Ingenuity | 0.0277 | 3.151 | No | 218283_at |
| SMARCA5 | No | 0.0278 | 3.504 | No | 213251_at |
| CYCS | No | 0.0281 | 3.257 | No | 208905_at |
| NOL5A | Not assigned to function by Ingenuity | 0.0284 | 3.76 | No | 200874_s_at |
| ING1L | No | 0.0297 | 3.537 | No | 205981_s_at |
| FLJ13910 | Not assigned to function by Ingenuity | 0.0297 | 0.257 | No | 212482_at |
| EIF4A1 | Yes | 0.0299 | 0.221 | No | 211787_s_at |
| RPL31 | Yes | 0.0300 | 4.29 | No | 200963_x_a |
| SOD1 | Yes | 0.0301 | 3.742 | No | 200642_at |
| RPS25 | Yes | 0.0306 | 3.331 | No | 200091_s_at |
| ACTR1B | No | 0.0317 | 0.22 | No | 202135_s_at |
| DT1P1A10 | Not assigned to function by Ingenuity | 0.0318 | 4.334 | No | 213079_at |
| MAP3K7 | Not assigned to function by Ingenuity | 0.0320 | 3.601 | No | 215476_at |
| STOM | Not assigned to function by Ingenuity | 0.0322 | 0.235 | No | 201060_x_a |
| K1AA0676 | Not assigned to function by Ingenuity | 0.0326 | 0.224 | No | 215994_x_a |
| FOXO1A | Yes | 0.0328 | 3.167 | No | 202724_s_at |
| COPS7A | Not assigned to function by Ingenuity | 0.0333 | 0.258 | No | 209029_at |
| SNAP25 | No | 0.0337 | 3.119 | No | 202507_s_at |
| HSPA8 | No | 0.0337 | 3.651 | No | 221891_x_a |
| PLAGL2 | Yes | 0.0343 | 0.269 | No | 202924_s_at |
| NDUFS5 | No | 0.0348 | 3.213 | No | 201757_at |
| PTD004 | Not assigned to function by Ingenuity | 0.0353 | 3.883 | No | 219293_s_at |
| P38IP | Not assigned to function by Ingenuity | 0.0361 | 3.621 | No | 220408_x_a |
| RPS7 | Yes | 0.0363 | 4.105 | No | 213941_x_a |
| CKAP4 | Not assigned to function by Ingenuity | 0.0365 | 0.302 | No | 200998_s_at |
| LOC92482 | Not assigned to function by Ingenuity | 0.0370 | 3.028 | No | 213220_at |
| TTC13 | Not assigned to function by Ingenuity | 0.0370 | 3.397 | No | 219481_at |
| RPL19 | Yes | 0.0370 | 3.543 | No | 200029_at |
| SON | No | 0.0373 | 3.843 | No | 214988_s_at |
| UBE2G1 | Not assigned to function by Ingenuity | 0.0373 | 0.274 | No | 209141_at |
| HNRPD | No | 0.0375 | 4.357 | Yes | 200073_s_at |
| NIP30 | Not assigned to function by Ingenuity | 0.0382 | 3.641 | No | 217896_s_at |
| TRIM44 | Not assigned to function by Ingenuity | 0.0382 | 4.971 | No | 217760_at |
| APG4B | No | 0.0383 | 0.326 | No | 212280_x_a |
| EIF3S1 | Yes | 0.0395 | 3.184 | No | 208264_s_at |
| 0 | Not assigned to function by Ingenuity | 0.0397 | 3.602 | No | 212436_at |
| TLE1 | No | 0.0399 | 3.14 | No | 203221_at |
| TM9SF4 | Not assigned to function by Ingenuity | 0.0399 | 0.332 | No | 212198_s_at |
| UBL1 | Yes | 0.0400 | 3.159 | No | 211069_s_at |
| K1AA0252 | Not assigned to function by Ingenuity | 0.0407 | 3.099 | No | 212302_at |
| DR1 | No | 0.0412 | 0.252 | No | 207654_x_a |
| BUB3 | Yes | 0.0419 | 4.233 | Yes | 201457_x_a |
| RPS3A | Yes | 0.0419 | 4.5 | Yes | 212391_x_a |
| PIGL | No | 0.0420 | 3.247 | No | 205873_at |
| CHCHD7 | Not assigned to function by Ingenuity | 0.0420 | 3.531 | No | 218642_s_at |
| TMSB10 | No | 0.0425 | 4.215 | No | 217733_s_at |
| FLJ11712 | Not assigned to function by Ingenuity | 0.0427 | 3.38 | No | 219056_at |
| RPL34 | Not assigned to function by Ingenuity | 0.0427 | 4.804 | No | 200026_at |
| TCTEL1 | No | 0.0433 | 3.245 | No | 201999_s_at |
| TAX1BP1 | No | 0.0433 | 3.335 | No | 200977_s_at |
| DHX15 | Not assigned to function by Ingenuity | 0.0435 | 3.049 | No | 201385_at |
| TRIM33 | Not assigned to function by Ingenuity | 0.0439 | 0.28 | No | 213184_at |
| MYO9B | No | 0.0442 | 0.284 | No | 214780_s_at |
| RPL21 (or | yes | 0.0454 | 3.958 | No | 200012_x_a |
| RPL21 | | | | | |
| Pseudogene) | | | | | |
| ZNF261 | No | 0.0458 | 3.022 | No | 207559_s_at |
| HK1 | No | 0.0468 | 0.32 | No | 200697_at |
| RAB2L | No | 0.0477 | 0.249 | No | 209110_s_at |
| NDUFA4 | No | 0.0501 | 3.034 | No | 217773_s_at |
| RPL35A | yes | 0.0502 | 3.493 | No | 213687_s_at |
| FLJ23233 | Not assigned to function by Ingenuity | 0.0505 | 3.601 | No | 58367_s_at |
| NIFU | No | 0.0508 | 3.867 | No | 209075_s_at |
| NONO | Not assigned to function by Ingenuity | 0.0513 | 3.745 | No | 210470_x_a |
| LOC57149 | Not assigned to function by Ingenuity | 0.0526 | 3.251 | No | 203897_at |
| AMFR | No | 0.0543 | 0.313 | No | 202204_s_at |
| RPS15A | yes | 0.0543 | 3.647 | No | 200781_s_at |
| PSMB1 | Not assigned to function by Ingenuity | 0.0549 | 5.083 | No | 214288_s_at |
| EIF4G2 | Yes | 0.0549 | 8.478 | No | 200004_at |
| RPL27 | yes | 0.0555 | 3.661 | No | 200025_s_at |
| MGC5508 | Not assigned to function by Ingenuity | 0.0558 | 0.323 | No | 201361_at |
| PEX16 | No | 0.0559 | 3.69 | No | 49878_at |
| TXNRD1 | Yes | 0.0573 | 0.285 | No | 201266_at |
| AKR1C1 | No | 0.0586 | 3.098 | No | 216594_x_a |
| FLJ13725 | Not assigned to function by Ingenuity | 0.0586 | 0.322 | No | 45749_at |
| NFRKB | Not assigned to function by Ingenuity | 0.0586 | 3.162 | No | 213028_at |
| XBP1 | No | 0.0599 | 0.292 | No | 200670_at |
| RPLP1 | Yes | 0.0610 | 4.328 | No | 200763_s_at |
| HNRPDL | No | 0.0635 | 4.04 | No | 209067_s_at |
| TAF7 | No | 0.0635 | 4.126 | No | 201023_at |
| B2M | No | 0.0662 | 3.074 | No | 201891_s_at |
| PHF2 | Not assigned to function by Ingenuity | 0.0669 | 3.458 | No | 212726_at |
| JWA | No | 0.0670 | 0.32 | No | 200760_s_at |
| C6ORF62 | Not assigned to function by Ingenuity | 0.0672 | 3.245 | No | 208809_s_at |
| RPL24 | Not assigned to function by Ingenuity | 0.0682 | 3.055 | No | 214143_x_a |
| C21ORF97 | Not assigned to function by Ingenuity | 0.0686 | 0.286 | No | 218019_s_at |
| MCFD2 | Not assigned to function by Ingenuity | 0.0687 | 3.299 | No | 212245_at |
| EEF1A1 | Yes | 0.0710 | 3.476 | No | 213477_x_a |
| MCAM | No | 0.0733 | 3.745 | No | 211042_x_a |
| HMGN2 | No | 0.0735 | 5.16 | No | 208668_x_a |
| LOC144983 | Not assigned to function by Ingenuity | 0.0735 | 3.185 | No | 216559_x_a |
| NEDD5 | Not assigned to function by Ingenuity | 0.0750 | 3.861 | No | 200015_s_at |
| SH3GLB1 | No | 0.0764 | 0.279 | No | 209090_s_at |
| FLJ10996 | Not assigned to function by Ingenuity | 0.0773 | 3.15 | No | 219774_at |
| TCEB1 | No | 0.0777 | 3.198 | No | 202824_s_at |
| FLJ10521 | Not assigned to function by Ingenuity | 0.0790 | 0.319 | No | 221656_s_at |
| NAP1L1 | No | 0.0791 | 5.196 | Yes | 212967_x_a |
| RANBP9 | No | 0.0805 | 3.008 | No | 202582_s_at |
| SMP1 | Not assigned to function by Ingenuity | 0.0833 | 0.278 | No | 217766_s_at |
| NAB1 | No | 0.0838 | 3.184 | No | 211139_s_at |
| RPL38 | yes | 0.0856 | 3.262 | No | 202029_x_a |
| RPS15 | Yes | 0.0867 | 3.348 | No | 200819_s_at |
| DDX5 | Not assigned to function by Ingenuity | 0.0895 | 4.224 | No | 200033_at |
| HCDI | Not assigned to function by Ingenuity | 0.0906 | 3.791 | No | 213398_s_at |
| PITPNC1 | No | 0.0956 | 3.059 | No | 219155_at |
| SDF2 | No | 0.0965 | 0.227 | No | 203090_at |
| CCNH | No | 0.0988 | 3.661 | No | 204093_at |
| DNAJC8 | Not assigned to function by Ingenuity | 0.0997 | 3.323 | No | 212491_s_at |
| ELK1 | No | 0.0999 | 0.324 | No | 203617_x_a |

TABLE 28

| | Affymetrix | | | | IgG | | | |
|---|---|---|---|---|---|---|---|---|
| | probeset | | Level in IgG | Signal-to- | Odds | FDR | Encephalitis | FDR |
| Rank | identifier | Gene Description | Responders | Noise Score | Ratio | IgG | Odds Ratio | Encephalitis |
| 1 | 202344_at | HSF1 - heat shock | Decreased | 0.82 | 0.383 | 0.00129 | 0.185 | 0.047996 |
| | | transcription factor 1 | | | | | | |
| 2 | 205875_s_at | TREX1 - three prime | Decreased | 0.8 | 0.411 | 0.00159 | 0.318 | 0.06771 |
| | | repair exonuclease 1 | | | | | | |
| 3 | 212907_at | UNK_AI972416- | Decreased | 0.78 | 0.434 | 0.0229 | 0.594 | 0.536404 |
| | | Human hbc647 mRNA | | | | | | |
| | | sequence. | | | | | | |
| 4 | 201574_at | ETF1 - eukaryotic | Decreased | 0.78 | 0.194 | 0.000715 | 0.075 | 0.041668 |
| | | translation termination | | | | | | |
| | | factor 1 | | | | | | |
| 5 | 218037_at | MGC3035 - hypothetical | Decreased | 0.76 | 0.208 | 0.0011 | 0.065 | 0.038226 |
| | | protein MGC3035 | | | | | | |
| 6 | 209215_at | TETRAN - tetracycline | Decreased | 0.76 | 0.399 | 0.00134 | 0.421 | 0.205058 |
| | | transporter-like protein | | | | | | |
| 7 | 201360_at | CST3 - cystatin C | Decreased | 0.74 | 0.481 | 0.00311 | 0.395 | 0.073239 |
| | | (amyloid angiopathy and | | | | | | |
| | | cerebral hemorr | | | | | | |
| 8 | 215706_x_at | ZYX-zyxin | Decreased | 0.74 | 0.547 | 0.00256 | 0.471 | 0.108013 |
| 9 | 201954_at | ARPC1B - actin related | Decreased | 0.73 | 0.276 | 0.00202 | 0.263 | 0.242364 |
| | | protein ⅔ complex, | | | | | | |
| | | subunit 1B, 41k | | | | | | |
| 10 | 221725_at | WASF2 - WAS protein | Decreased | 0.72 | 0.108 | 0.00144 | 0.026 | 0.076275 |
| | | family, member 2 | | | | | | |
| 11 | 201720_s_at | LAPTM5-Lysosomal- | Decreased | 0.69 | 0.212 | 0.00455 | 0.076 | 0.067654 |
| | | associated multispanning | | | | | | |
| | | membrane protei | | | | | | |
| 12 | 217811_at | SELT - selenoprotein T | Decreased | 0.68 | 0.454 | 0.0032 | 0.245 | 0.073377 |
| 13 | 202373_s_at | RAB3-GAP150 - rab3 | Increased | 0.76 | 5.468 | 0.00659 | 70.713 | 0.051166 |
| | | GTPase-activating | | | | | | |
| | | protein, non-catalytic | | | | | | |
| | | subu | | | | | | |
| 14 | 216806_at | UNK_AL136306 - | Increased | 0.73 | 3.016 | 0.00169 | 2.453 | 0.369437 |
| | | Consensus includes | | | | | | |
| | | gb:AL136306 | | | | | | |
| | | /DEF = Human DNA sequ | | | | | | |
| 15 | 213509_x_at | CES2 - carboxylesterase | Increased | 0.68 | 2.777 | 0.00129 | 11.698 | 0.01765 |
| | | 2 (intestine, liver) | | | | | | |
| 16 | 216508_x_at | UNK_AC007277 - | Increased | 0.67 | 2.476 | 0.048 | 7.724 | 0.183538 |
| | | Consensus includes | | | | | | |
| | | gb:AC007277 | | | | | | |
| | | /DEF = Homo sapiens B | | | | | | |
| 17 | 218918_at | MAN1C1 - mannosidase, | Increased | 0.66 | 5.5 | 0.000899 | 9.161 | 0.129904 |
| | | alpha, class 1C, member | | | | | | |
| | | 1 | | | | | | |
| 18 | 212637_s_at | WWP 1 - WW domain- | Increased | 0.66 | 2.226 | 0.0496 | 3.469 | 0.275866 |
| | | containing protein 1 | | | | | | |
| 19 | 215221_at | UNK_AK025064- | Increased | 0.64 | 2.717 | 0.00828 | 2.156 | 0.477634 |
| | | Homo sapiens cDNA: | | | | | | |
| | | FLJ21411 fis, clone | | | | | | |
| | | COL03986. | | | | | | |
| 20 | 202909_at | EPM2AIP1 - EPM2A | Increased | 0.63 | 3.836 | 0.00167 | 8.301 | 0.067315 |
| | | (laforin) interacting | | | | | | |
| | | protein 1 | | | | | | |
| 21 | 204528_s_at | NAP1L1 - nucleosome | Increased | 0.62 | 4.079 | 0.000732 | 2.709 | 0.357819 |
| | | assembly protein 1-like 1 | | | | | | |
| 22 | 203371_s_at | NDUFB3 - NADH | Increased | 0.61 | 4.865 | 0.00985 | 52.955 | 0.053723 |
| | | dehydrogenase | | | | | | |
| | | (ubiquinone) 1 beta | | | | | | |
| | | subcomplex, | | | | | | |
| 23 | 208845_at | UNK_BC002456- | Increased | 0.61 | 5.809 | 0.00384 | 11.618 | 0.159569 |
| | | gb:BC002456.1/ | | | | | | |
| | | DEF = Homo sapiens, | | | | | | |
| | | voltage-dependent | | | | | | |
| 24 | 200685_at | SFRS11 - splicing factor, | Increased | 0.6 | 1.998 | 0.0264 | 1.19 | 0.877872 |
| | | arginine/serine-rich 11 | | | | | | |

TABLE 29

| Patient | Confidence | True | | Correct |
|---|---|---|---|---|
| ID | Score | Class | Classification | Classification |
| 2 | 0.643 | IGG-RESP | IGG-RESP | Yes |
| 4 | 0.715 | IGG-RESP | IGG-RESP | Yes |
| 5 | 0.817 | IGG-RESP | IGG-RESP | Yes |
| 7 | 0.889 | IGG-NON | IGG-NON | Yes |
| 10 | 0.805 | IGG-NON | IGG-NON | Yes |
| 12 | 0.853 | IGG-NON | IGG-RESP | No |
| 14 | 0.744 | IGG-NON | IGG-NON | Yes |
| 15 | 0.047 | IGG-RESP | IGG-RESP | Yes |
| 16 | 0.601 | IGG-RESP | IGG-RESP | Yes |
| 17 | 0.916 | IGG-NON | IGG-NON | Yes |
| 18 | 1.000 | IGG-NON | IGG-NON | Yes |
| 19 | 0.744 | IGG-RESP | IGG-RESP | Yes |
| 22 | 0.520 | IGG-RESP | IGG-RESP | Yes |
| 23 | 0.477 | IGG-NON | IGG-NON | Yes |
| 25 | 1.000 | IGG-NON | IGG-NON | Yes |
| 28 | 0.018 | IGG-RESP | IGG-NON | No |
| 29 | 0.162 | IGG-RESP | IGG-RESP | Yes |
| 31 | 0.379 | IGG-RESP | IGG-NON | No |
| 32 | 1.000 | IGG-RESP | IGG-RESP | Yes |
| 33 | 0.334 | IGG-RESP | IGG-RESP | Yes |
| 34 | 0.844 | IGG-NON | IGG-NON | Yes |
| 36 | 0.556 | IGG-RESP | IGG-RESP | Yes |
| 40 | 0.563 | IGG-RESP | IGG-NON | No |
| 41 | 0.310 | IGG-RESP | IGG-NON | No |
| 43 | 0.602 | IGG-NON | IGG-NON | Yes |
| 44 | 0.835 | IGG-NON | IGG-NON | Yes |
| 48 | 0.756 | IGG-NON | IGG-NON | Yes |
| 52 | 0.911 | IGG-NON | IGG-NON | Yes |
| 53 | 0.002 | IGG-RESP | IGG-RESP | Yes |
| 54 | 0.958 | IGG-NON | IGG-NON | Yes |
| 55 | 0.935 | IGG-NON | IGG-RESP | No |
| 57 | 0.817 | IGG-NON | IGG-NON | Yes |
| 64 | 0.403 | IGG-RESP | IGG-NON | No |
| 66 | 0.062 | IGG-NON | IGG-NON | Yes |
| 68 | 0.822 | IGG-NON | IGG-NON | Yes |
| 69 | 1.000 | IGG-NON | IGG-NON | Yes |
| 70 | 0.621 | IGG-NON | IGG-NON | Yes |
| 71 | 0.868 | IGG-NON | IGG-NON | Yes |
| 252 | 0.703 | IGG-RESP | IGG-NON | No |
| 254 | 0.947 | IGG-NON | IGG-NON | Yes |
| 255 | 0.446 | IGG-RESP | IGG-RESP | Yes |
| 258 | 0.967 | IGG-NON | IGG-NON | Yes |
| 259 | 0.565 | IGG-RESP | IGG-NON | No |
| 260 | 0.172 | IGG-NON | IGG-NON | Yes |
| 262 | 1.000 | IGG-NON | IGG-NON | Yes |
| 263 | 0.747 | IGG-NON | IGG-NON | Yes |
| 266 | 0.739 | IGG-NON | IGG-NON | Yes |
| 269 | 0.971 | IGG-NON | IGG-NON | Yes |
| 271 | 0.038 | IGG-NON | IGG-NON | Yes |
| 274 | 0.479 | IGG-NON | IGG-NON | Yes |
| 277 | 0.885 | IGG-RESP | IGG-RESP | Yes |
| 279 | 1.000 | IGG-NON | IGG-NON | Yes |
| 280 | 0.846 | IGG-NON | IGG-NON | Yes |
| 281 | 0.642 | IGG-NON | IGG-NON | Yes |
| 285 | 1.000 | IGG-NON | IGG-NON | Yes |
| 286 | 0.573 | IGG-NON | IGG-RESP | No |
| 287 | 0.108 | IGG-RESP | IGG-NON | No |
| 289 | 0.833 | IGG-NON | IGG-NON | Yes |
| 290 | 0.553 | IGG-NON | IGG-NON | Yes |
| 291 | 1.000 | IGG-NON | IGG-NON | Yes |
| 293 | 0.713 | IGG-RESP | IGG-RESP | Yes |
| 294 | 0.811 | IGG-NON | IGG-NON | Yes |
| 295 | 0.429 | IGG-NON | IGG-NON | Yes |
| 296 | 0.088 | IGG-RESP | IGG-NON | No |
| 299 | 0.704 | IGG-RESP | IGG-RESP | Yes |
| 300 | 0.041 | IGG-NON | IGG-NON | Yes |
| 301 | 0.634 | IGG-RESP | IGG-RESP | Yes |
| 302 | 0.811 | IGG-NON | IGG-NON | Yes |
| 303 | 0.748 | IGG-NON | IGG-NON | Yes |
| 304 | 0.986 | IGG-NON | IGG-NON | Yes |
| 306 | 0.905 | IGG-RESP | IGG-NON | No |
| 307 | 0.980 | IGG-NON | IGG-NON | Yes |
| 308 | 0.492 | IGG-RESP | IGG-NON | No |
| 314 | 1.000 | IGG-NON | IGG-NON | Yes |
| 315 | 1.000 | IGG-NON | IGG-NON | Yes |
| 316 | 0.942 | IGG-NON | IGG-NON | Yes |
| 319 | 0.721 | IGG-NON | IGG-NON | Yes |
| 503 | 0.907 | IGG-RESP | IGG-RESP | Yes |
| 506 | 0.170 | IGG-NON | IGG-NON | Yes |
| 507 | 0.341 | IGG-NON | IGG-NON | Yes |
| 508 | 0.798 | IGG-RESP | IGG-RESP | Yes |
| 509 | 0.201 | IGG-RESP | IGG-NON | No |
| 514 | 0.987 | IGG-NON | IGG-NON | Yes |
| 515 | 0.667 | IGG-NON | IGG-NON | Yes |
| 516 | 0.942 | IGG-NON | IGG-RESP | No |
| 752 | 0.990 | IGG-RESP | IGG-NON | No |
| 753 | 0.400 | IGG-NON | IGG-NON | Yes |
| 755 | 0.115 | IGG-NON | IGG-RESP | No |
| 756 | 0.892 | IGG-NON | IGG-NON | Yes |
| 757 | 0.613 | IGG-NON | IGG-NON | Yes |
| 758 | 0.552 | IGG-NON | IGG-NON | Yes |
| 760 | 0.712 | IGG-NON | IGG-NON | Yes |
| 762 | 0.574 | IGG-NON | IGG-RESP | No |
| 763 | 0.352 | IGG-NON | IGG-NON | Yes |
| 765 | 0.995 | IGG-NON | IGG-NON | Yes |

TABLE 30

| | | | | IgG | | | |
|---|---|---|---|---|---|---|---|
| | Affymetrix | | Signal-to- | Odds | FDR | Encephalitis | FDR |
| Rank | identifier | Gene Description | Noise Score | Ratio | IgG | Odds Ratio | Encephalitis |
| 1 | 202344_at | HSF1 - heat shock | 0.82 | 0.383 | 0.00129 | 0.185 | 0.047996 |
| | | transcription factor 1 | | | | | |
| 2 | 205875_s_at | TREX 1 - three prime | 0.8 | 0.411 | 0.00159 | 0.318 | 0.06771 |
| | | repair exonuclease 1 | | | | | |
| 3 | 212907_at | UNK_A1972416- | 0.78 | 0.434 | 0.0229 | 0.594 | 0.536404 |
| | | Human hbc647 mRNA | | | | | |
| | | sequence. | | | | | |
| 4 | 202373_s_at | RAB3-GAP150 - rab3 | 0.76 | 5.468 | 0.00659 | 0.713 | 0.051166 |
| | | GTPase-activating | | | | | |
| | | protein, non-catalytic | | | | | |
| | | subu | | | | | |
| 5 | 216806_at | UNK_AL136306- | 0.73 | 3.016 | 0.00169 | 2.453 | 0.369437 |
| | | Consensus includes | | | | | |
| | | gb:AL136306/ | | | | | |
| | | DEF = Human DNA | | | | | |
| | | sequ | | | | | |
| 6 | 213509_x_at | CES2 - | 0.68 | 2.777 | 0.00129 | 11.698 | 0.01765 |
| | | carboxylesterase 2 | | | | | |
| | | (intestine, liver) | | | | | |

TABLE 31

| Patient | Confidence | True | |
|---|---|---|---|
| ID | Score | Class | Classification |
| 2 | 0.694 | IGG-RESP | IGG-RESP |
| 4 | 0.975 | IGG-RESP | IGG-RESP |
| 5 | 1.000 | IGG-RESP | IGG-RESP |
| 7 | 0.577 | IGG-NON | IGG-NON |
| 10 | 1.000 | IGG-NON | IGG-NON |
| 12 | 1.000 | IGG-NON | IGG-RESP |
| 14 | 0.186 | IGG-NON | IGG-NON |
| 15 | 1.000 | IGG-RESP | IGG-RESP |
| 16 | 0.118 | IGG-RESP | IGG-RESP |
| 17 | 0.867 | IGG-NON | IGG-NON |
| 18 | 1.000 | IGG-NON | IGG-NON |
| 19 | 1.000 | IGG-RESP | IGG-RESP |
| 22 | 0.516 | IGG-RESP | IGG-RESP |
| 23 | 1.000 | IGG-NON | IGG-NON |
| 25 | 1.000 | IGG-NON | IGG-NON |
| 28 | 1.000 | IGG-RESP | IGG-NON |
| 29 | 0.045 | IGG-NON | IGG-RESP |
| 31 | 0.257 | IGG-NON | IGG-NON |
| 32 | 1.000 | IGG-RESP | IGG-RESP |
| 33 | 0.297 | IGG-RESP | IGG-RESP |
| 34 | 0.229 | IGG-NON | IGG-NON |
| 36 | 0.858 | IGG-RESP | IGG-RESP |
| 40 | 0.569 | IGG-RESP | IGG-NON |
| 41 | 0.869 | IGG-RESP | IGG-NON |
| 43 | 0.119 | IGG-RESP | IGG-NON |
| 44 | 0.686 | IGG-NON | IGG-NON |
| 48 | 0.241 | IGG-NON | IGG-NON |
| 52 | 0.592 | IGG-NON | IGG-NON |
| 53 | 0.234 | IGG-RESP | IGG-RESP |
| 54 | 1.000 | IGG-NON | IGG-NON |
| 55 | 0.965 | IGG-NON | IGG-RESP |
| 57 | 1.000 | IGG-NON | IGG-NON |
| 64 | 0.176 | IGG-RESP | IGG-NON |
| 66 | 1.000 | IGG-RESP | IGG-NON |
| 68 | 1.000 | IGG-NON | IGG-NON |
| 69 | 1.000 | IGG-NON | IGG-NON |
| 70 | 0.450 | IGG-NON | IGG-NON |
| 71 | 0.593 | IGG-NON | IGG-NON |
| 252 | 0.681 | IGG-RESP | IGG-NON |
| 254 | 1.000 | IGG-NON | IGG-NON |
| 255 | 0.676 | IGG-RESP | IGG-RESP |
| 258 | 1.000 | IGG-NON | IGG-NON |
| 259 | 1.000 | IGG-NON | IGG-NON |
| 260 | 0.272 | IGG-NON | IGG-NON |
| 262 | 1.000 | IGG-NON | IGG-NON |
| 263 | 0.623 | IGG-NON | IGG-NON |
| 266 | 0.973 | IGG-NON | IGG-NON |
| 269 | 1.000 | IGG-NON | IGG-NON |
| 271 | 0.577 | IGG-NON | IGG-NON |
| 274 | 0.665 | IGG-RESP | IGG-NON |
| 277 | 1.000 | IGG-RESP | IGG-RESP |
| 279 | 1.000 | IGG-NON | IGG-NON |
| 280 | 1.000 | IGG-NON | IGG-NON |
| 281 | 0.338 | IGG-NON | IGG-NON |
| 285 | 1.000 | IGG-NON | IGG-NON |
| 286 | 0.036 | IGG-RESP | IGG-RESP |
| 287 | 0.382 | IGG-NON | IGG-NON |
| 289 | 0.156 | IGG-NON | IGG-NON |
| 290 | 0.902 | IGG-NON | IGG-NON |
| 291 | 1.000 | IGG-NON | IGG-NON |
| 293 | 0.042 | IGG-RESP | IGG-RESP |
| 294 | 1.000 | IGG-NON | IGG-NON |
| 295 | 0.437 | IGG-NON | IGG-NON |
| 296 | 0.034 | IGG-NON | IGG-NON |
| 299 | 1.000 | IGG-RESP | IGG-RESP |
| 300 | 0.574 | IGG-RESP | IGG-NON |
| 301 | 0.966 | IGG-RESP | IGG-RESP |
| 302 | 1.000 | IGG-NON | IGG-NON |
| 303 | 0.200 | IGG-NON | IGG-NON |
| 304 | 1.000 | IGG-NON | IGG-NON |
| 306 | 1.000 | IGG-RESP | IGG-NON |
| 307 | 1.000 | IGG-NON | IGG-NON |
| 308 | 1.000 | IGG-RESP | IGG-NON |
| 314 | 1.000 | IGG-NON | IGG-NON |
| 315 | 1.000 | IGG-NON | IGG-NON |
| 316 | 0.996 | IGG-NON | IGG-NON |
| 319 | 0.937 | IGG-NON | IGG-NON |
| 503 | 1.000 | IGG-RESP | IGG-RESP |
| 506 | 0.861 | IGG-RESP | IGG-NON |
| 507 | 0.639 | IGG-NON | IGG-NON |
| 508 | 1.000 | IGG-RESP | IGG-RESP |
| 509 | 0.601 | IGG-RESP | IGG-NON |
| 514 | 1.000 | IGG-NON | IGG-NON |
| 515 | 0.225 | IGG-NON | IGG-NON |
| 516 | 0.766 | IGG-NON | IGG-RESP |
| 752 | 0.908 | IGG-RESP | IGG-NON |
| 753 | 0.244 | IGG-NON | IGG-NON |
| 755 | 0.020 | IGG-NON | IGG-RESP |
| 756 | 1.000 | IGG-NON | IGG-NON |
| 757 | 1.000 | IGG-NON | IGG-NON |
| 758 | 1.000 | IGG-NON | IGG-NON |
| 760 | 0.990 | IGG-NON | IGG-NON |
| 762 | 1.000 | IGG-NON | IGG-RESP |
| 763 | 0.402 | IGG-NON | IGG-NON |
| 765 | 1.000 | IGG-NON | IGG-NON |

TABLE 32
Genes Associated with Meningoencephalitis

| | | Odds | | | |
|---|---|---|---|---|---|
| | | Ratio for | | | |
| | | association | | | |
| | Meningo- | with | | | |
| | encephalitis | meningo- | Unadjusted | | Affymetrix |
| Gene | FDR | encephalitis | p values | Description | identifier |
| STAT1 | 0.004 | 230.416 | 5.10E-07 | signal transducer and | 209969_s_at |
| | | | | activator of transcription | |
| | | | | 1, 91kDa | |
| NHP2L1 | 0.010 | 3136.203 | 2.17E-05 | NHP2 non-histone | 201076_at |
| | | | | chromosome protein 2- | |
| | | | | like 1 (S. cerevisiae) | |
| C10ORF7 | 0.010 | 673.31 | 7.19E-06 | chromosome 10 open | 201725_at |
| | | | | reading frame 7 | |
| FLJ11806 | 0.010 | 651.763 | 1.99E-05 | nuclear protein UKp68 | 213064_at |
| ZW10 | 0.010 | 470.958 | 3.04E-05 | ZW10 homolog, | 204812_at |
| | | | | centromere/kinetochore | |
| | | | | protein (Drosophila) | |
| C12ORF22 | 0.010 | 459.155 | 1.83E-05 | chromosome 12 open | 221260_s_at |
| | | | | reading frame 22 | |
| ICMT | 0.010 | 417.532 | 1.62E-05 | isoprenylcysteine | 201609_x_at |
| | | | | carboxyl | |
| | | | | methyltransferase | |
| RABGAP1 | 0.010 | 303.809 | 1.13E-05 | RAB GTPase activating | 204028_s_at |
| | | | | protein 1 | |
| TRAP240 | 0.010 | 68.675 | 9.52E-06 | thyroid hormone receptor | 201986_at |
| | | | | associated protein 1 | |
| SEC24C | 0.010 | 66.791 | 3.08E-05 | SEC24 related gene | 202361_at |
| | | | | family, member C (S. | |
| | | | | cerevisiae) | |
| BRD2 | 0.010 | 56.318 | 1.06E-05 | bromodomain containing | 208686_s_at |
| | | | | 2 | |
| KPNB1 | 0.010 | 32.282 | 3.83E-05 | karyopherin (importin) | 208975_s_at |
| | | | | beta 1 | |
| GZMB | 0.010 | 31.809 | 3.68E-05 | granzyme B (granzyme | 210164_at |
| | | | | cytotoxic T- | |
| | | | | lymphocyte-associated | |
| | | | | serine esterase 1) | |
| FNBP3 | 0.010 | 13.972 | 3.19E-05 | formin binding protein 3 | 213729_at |
| KLF2 | 0.010 | 0.038 | 3.72E-05 | Kruppel-like factor 2 | 219371_s_at |
| | | | | (lung) | |
| STK17B | 0.010 | 0.025 | 1.38E-05 | serine/threonine kinase | 205214_at |
| | | | | 17b (apoptosis-inducing) | |
| JARID1B | 0.010 | 0.006 | 3.93E-05 | Jumonji, AT rich | 211202_s_at |
| | | | | interactive domain 1B | |
| | | | | (RBP2-like) | |
| MGC21416 | 0.011 | 8.373 | 5.98E-05 | hypothetical protein | 212341_at |
| | | | | MGC21416 | |
| STAT3 | 0.011 | 6.606 | 5.96E-05 | signal transducer and | 208991_at |
| | | | | activator of transcription | |
| | | | | 3 (acute-phase response | |
| | | | | factor) | |
| OSBPL8 | 0.011 | 4.201 | 5.85E-05 | oxysterol binding | 212582_at |
| | | | | protein-like 8 | |
| BTG2 | 0.011 | 0.033 | 5.26E-05 | BTG family, member 2 | 201236_s_at |
| UBE2D3 | 0.011 | 0.002 | 5.40E-05 | ubiquitin-conjugating | 200669_s_at |
| | | | | enzyme E2D 3 (UBC4/5 | |
| | | | | homolog, yeast) | |
| HEAB | 0.011 | 0.001 | 5.41E-05 | ATP/GTP-binding | 204370_at |
| | | | | protein | |
| ATP6V1D | 0.011 | 172.543 | 6.85E-05 | ATPase, H+ transporting, | 208899_x_at |
| | | | | lysosomal 34kDa, V1 | |
| | | | | subunit D | |
| KIF5B | 0.011 | 3.731 | 7.12E-05 | kesin family member | 201991_s_at |
| | | | | 5B | |
| DC8 | 0.012 | 69.508 | 8.01E-05 | DKFZP566O1646 | 209484_s_at |
| | | | | protein | |
| CD84 | 0.013 | 23.97 | 1.01E-04 | CD84 antigen (leukocyte | 205988_at |
| | | | | antigen) | |
| Unknown | 0.013 | 0.015 | 9.59E-05 | no sequence similarity to | 211444_at |
| | | | | any genes or proteins | |
| GLTSCR1 | 0.013 | 0.013 | 9.14E-05 | glioma tumor suppressor | 219445_at |
| | | | | candidate region gene 1 | |
| UGCG | 0.013 | 14.445 | 1.10E-04 | UDP-glucose ceramide | 204881_s_at |
| | | | | glucosyltransferase | |
| SFRS21P | 0.014 | 57.281 | 1.14E-04 | splicing factor, | 206989_s_at |
| | | | | arginine/serine-rich 2, | |
| | | | | interacting protein | |
| MMP24 | 0.014 | 0.022 | 1.16E-04 | matrix metalloproteinase | 78047_s_at |
| | | | | 24 (membrane-inserted) | |
| GCDH | 0.014 | 50.321 | 1.33E-04 | glutaryl-Coenzyme A | 203500_at |
| | | | | dehydrogenase | |
| TNPO3 | 0.014 | 21.713 | 1.32E-04 | transportin 3 | 212318_at |
| MBD4 | 0.014 | 8.79 | 1.28E-04 | methyl-CpG binding | 209579_s_at |
| | | | | domain protein 4 | |
| PABPC1 | 0.014 | 0.006 | 1.29E-04 | poly(A) binding protein, | 215823_x_at |
| | | | | cytoplasmic 1 | |
| VDR | 0.014 | 7.092 | 1.50E-04 | vitamin D (1,25- | 204255_s_at |
| | | | | dihydroxyvitamin D3) | |
| | | | | receptor | |
| H2AFY | 0.015 | 0.016 | 1.62E-04 | H2A histone family, | 207168_s_at |
| | | | | member Y | |
| CBX6 | 0.016 | 34.482 | 1.79E-04 | chromobox homolog 6 | 202047_s_at |
| IL2RA | 0.016 | 11.266 | 1.77E-04 | interleukin 2 receptor, | 211269_s_at |
| | | | | alpha | |
| TTC3 | 0.016 | 5.376 | 1.78E-04 | tetratricopeptide repeat | 208662_s_at |
| | | | | domain 3 | |
| STAT5B | 0.016 | 0.029 | 1.81E-04 | signal transducer and | 212549_at |
| | | | | activator of transcription | |
| | | | | 5B | |
| TRIP13 | 0.016 | 17.331 | 1.88E-04 | thyroid hormone receptor | 204033_at |
| | | | | interactor 13 | |
| FLJ23441 | 0.016 | 17.419 | 1.95E-04 | hypothetical protein | 219217_at |
| | | | | FLJ23441 | |
| STXBP2 | 0.016 | 0.095 | 1.94E-04 | syntaxin binding protein | 209367_at |
| | | | | 2 | |
| LRRFIP1 | 0.016 | 18.564 | 1.99E-04 | leucine rich repeat (in | 201862_s_at |
| | | | | FLII) interacting protein | |
| | | | | 1 | |
| PADI2 | 0.016 | 0.145 | 2.08E-04 | peptidyl arginine | 209791_at |
| | | | | deiminase, type II | |
| HNRPC | 0.016 | 324.673 | 2.15E-04 | heterogeneous nuclear | 214737_x_at |
| | | | | ribonucleoprotein C | |
| | | | | (C1/C2) | |
| PTPRC | 0.017 | 4.891 | 2.29E-04 | protein tyrosine | 212587_s_at |
| | | | | phosphatase, receptor | |
| | | | | type, C | |
| PTDSR | 0.018 | 0.018 | 2.46E-04 | phosphatidylserine | 212723_at |
| | | | | receptor | |
| HUMGT198A | 0.018 | 8.097 | 2.57E-04 | GT198, complete ORF | 205956_x_at |
| TPR | 0.018 | 4.823 | 2.56E-04 | translocated promoter | 201730_s_at |
| | | | | region (to activated MET | |
| | | | | oncogene) | |
| DUT | 0.018 | 40.207 | 2.74E-04 | dUTP pyrophosphatase | 208955_at |
| RAB1A | 0.018 | 0.003 | 2.71E-04 | RAB1A, member RAS | 208724_s_at |
| | | | | oncogene family | |
| HMG2L1 | 0.019 | 5.679 | 2.87E-04 | high-mobility group | 212596_s_at |
| | | | | protein 2-like 1 | |
| RIN3 | 0.019 | 0.105 | 2.92E-04 | Ras and Rab interactor 3 | 60471_at |
| PDCD8 | 0.019 | 119.631 | 3.15E-04 | programmed cell death 8 | 205512_s_at |
| | | | | (apoptosis-inducing | |
| | | | | factor) | |
| GLS | 0.019 | 60.862 | 3.19E-04 | glutaminase | 203159_at |
| CSE1L | 0.019 | 38.753 | 3.13E-04 | CSE1 chromosome | 201112_s_at |
| | | | | segregation 1-like (yeast) | |
| RNMT | 0.019 | 0.050 | 3.15E-04 | RNA (guanine-7-) | 202684_s_at |
| | | | | methyltransferase | |
| TFE3 | 0.019 | 0.041 | 3.18E-04 | transcription factor | 206649_s_at |
| | | | | binding to IGHM | |
| | | | | enhancer 3 | |
| FLJ12788 | 0.020 | 167.936 | 3.23E-04 | hypothetical protein | 218838_s_at |
| | | | | FLJ12788 | |
| MGAT2 | 0.020 | 20.774 | 3.29E-04 | mannosyl (alpha-1,6-)- | 203102_s_at |
| | | | | glycoprotein beta-1,2-N- | |
| | | | | acetylglucosaminyl- | |
| | | | | transferase | |
| CGI-37 | 0.021 | 10.964 | 3.67E-04 | comparative gene | 219031_s_at |
| | | | | identification transcript | |
| | | | | 37 | |
| LUC7A | 0.021 | 7.673 | 3.58E-04 | cisplatin resistance- | 208835_s_at |
| | | | | associated overexpressed | |
| | | | | protein | |
| FBXW7 | 0.021 | 5.619 | 3.66E-04 | F-box and WD-40 | 218751_s_at |
| | | | | domain protein 7 | |
| | | | | (archipelago homolog, | |
| | | | | Drosophila) | |
| DICER1 | 0.021 | 0.073 | 3.62E-04 | Dicer1, Dcr-1 homolog | 216260_at |
| | | | | (Drosophila) | |
| UBCE7IP5 | 0.021 | 0.036 | 3.52E-04 | likely ortholog of mouse | 204598_at |
| | | | | ubiquitin conjugating | |
| | | | | enzyme 7 interacting | |
| | | | | protein 5 | |
| C21ORF80 | 0.021 | 0.032 | 3.62E-04 | protein O- | 209578_s_at |
| | | | | fucosyltransferase 2 | |
| TXNL2 | 0.021 | 152.265 | 3.83E-04 | thioredoxin-like 2 | 209080_x_at |
| PRKRA | 0.022 | 0.027 | 3.98E-04 | protein kinase, | 209139_s_at |
| | | | | interferon-inducible | |
| | | | | double stranded RNA | |
| | | | | dependent activator | |
| BARD1 | 0.022 | 11.776 | 4.11E-04 | BRCA1 associated RING | 205345_at |
| | | | | domain 1 | |
| SH3BP5 | 0.022 | 11.205 | 4.16E-04 | SH3-domain binding | 201810_s_at |
| | | | | protein 5 (BTK- | |
| | | | | associated) | |
| OBRGRP | 0.022 | 4.025 | 4.13E-04 | leptin receptor gene- | 202378_s_at |
| | | | | related protein | |
| C1ORF33 | 0.023 | 12.564 | 4.43E-04 | chromosome 1 open | 220688_s_at |
| | | | | reading frame 33 | |
| M96 | 0.023 | 9.28 | 4.39E-04 | likely ortholog of mouse | 203346_s_at |
| | | | | metal response element | |
| | | | | binding transcription | |
| | | | | factor 2 | |
| Unknown | 0.023 | 0.109 | 4.44E-04 | Unknown containing a | 214801_at |
| | | | | LAP1C protein domain | |
| IPO4 | 0.023 | 29.56 | 4.62E-04 | importin 4 | 218305_at |
| DNCL1 | 0.023 | 6.81 | 4.56E-04 | dynein, cytoplasmic, | 200703_at |
| | | | | light polypeptide 1 | |
| BAZ1A | 0.023 | 6.808 | 4.63E-04 | bromodomain adjacent to | 217985_s_at |
| | | | | zinc finger domain, 1A | |
| NALP1 | 0.023 | 0.133 | 4.53E-04 | NACHT, leucine rich | 218380_at |
| | | | | repeat and PYD | |
| | | | | containing 1 | |
| GNAS | 0.023 | 0.071 | 4.59E-04 | GNAS complex locus | 200780_x_at |
| TH1L | 0.024 | 13.185 | 4.76E-04 | TH1-like (Drosophila) | 220607_x_at |
| IRS2 | 0.024 | 0.060 | 4.80E-04 | insulin receptor substrate | 209185_s_at |
| | | | | 2 | |
| LTF | 0.025 | 0.325 | 5.08E-04 | lactotransferrin | 202018_s_at |
| MIRAB13 | 0.026 | 0.109 | 5.40E-04 | molecule interacting with | 221779_at |
| | | | | Rab13 | |
| BATF | 0.026 | 9.718 | 5.45E-04 | basic leucine zipper | 205965_at |
| | | | | transcription factor, | |
| | | | | ATF-like | |
| FLN29 | 0.026 | 176.965 | 5.51E-04 | FLN29 gene product | 35254_at |
| HAX1 | 0.026 | 34.12 | 5.59E-04 | HS1 binding protein | 201145_at |
| MYO1B | 0.026 | 18.41 | 5.61E-04 | myosin IB | 212365_at |
| SLC5A3 | 0.026 | 4.832 | 5.56E-04 | solute carrier family 5 | 213164_at |
| | | | | (inositol transporters), | |
| | | | | member 3 | |
| PADI4 | 0.026 | 0.108 | 5.62E-04 | peptidyl arginine | 220001_at |
| | | | | deiminase, type IV | |
| STK10 | 0.026 | 0.052 | 5.72E-04 | serine/threonine kinase | 40420_at |
| | | | | 10 | |
| RAB2 | 0.027 | 0.002 | 5.96E-04 | RAB2, member RAS | 208734_x_at |
| | | | | oncogene family | |
| BPI | 0.027 | 0.219 | 6.23E-04 | bactericidal/permeability- | 205557_at |
| | | | | increasing protein | |
| DEFA4 | 0.027 | 0.196 | 6.31E-04 | defensin, alpha 4, | 207269_at |
| | | | | corticostatin | |
| KPNA6 | 0.028 | 34.224 | 6.49E-04 | karyopherin alpha 6 | 212103_at |
| | | | | (importin alpha 7) | |
| C19ORF10 | 0.028 | 45.058 | 6.57E-04 | chromosome 19 open | 221739_at |
| | | | | reading frame 10 | |
| DKFZPS64G2022 | 0.028 | 11.966 | 6.66E-04 | DKFZP564G2022 | 212202_s_at |
| | | | | protein | |
| SNRK | 0.028 | 0.043 | 6.63E-04 | SNF-1 related kinase | 209481_at |
| GBP1 | 0.028 | 5.53 | 6.70E-04 | guanylate binding protein | 202269_x_at |
| | | | | 1, interferon-inducible, | |
| | | | | 67kDa | |
| ZFP36 | 0.029 | 0.108 | 7.02E-04 | zinc finger protein 36, | 201531_at |
| | | | | C3H type, homolog | |
| | | | | (mouse) | |
| ZNF238 | 0.029 | 0.120 | 7.15E-04 | zinc finger protein 238 | 212774_at |
| SIPA1 | 0.029 | 0.053 | 7.17E-04 | signal-induced | 204164_at |
| | | | | proliferation-associated | |
| | | | | gene 1 | |
| CXCL10 | 0.029 | 7.825 | 7.34E-04 | chemokine (C-X-C | 204533_at |
| | | | | motif) ligand 10 | |
| RRM2 | 0.029 | 5.394 | 7.24E-04 | ribonucleotide reductase | 209773_s_at |
| | | | | M2 polypeptide | |
| RAB31 | 0.029 | 3.04 | 7.52E-04 | RAB31, member RAS | 217762_s_at |
| | | | | oncogene family | |
| USP36 | 0.029 | 0.071 | 7.53E-04 | ubiquitin specific | 220370_s_at |
| | | | | protease 36 | |
| PTP4A1 | 0.029 | 0.034 | 7.54E-04 | protein tyrosine | 200732_s_at |
| | | | | phosphatase type IVA, | |
| | | | | member 1 | |
| DPCK | 0.029 | 156.071 | 7.58E-04 | Coenzyme A synthase | 201913_s_at |
| ALDOC | 0.029 | 11.591 | 7.75E-04 | aldolase C, fructose- | 202022_at |
| | | | | bisphosphate | |
| PXMP3 | 0.030 | 39.115 | 8.14E-04 | peroxisomal membrane | 210296_s_at |
| | | | | protein 3, 35kDa | |
| | | | | (Zellweger syndrome) | |
| ZFP36L1 | 0.030 | 0.036 | 8.11E-04 | zinc finger protein 36, | 211962_s_at |
| | | | | C3H type-like 1 | |
| CYLN2 | 0.030 | 0.060 | 8.26E-04 | cytoplasmic linker 2 | 211031_s_at |
| STAU | 0.031 | 0.078 | 8.49E-04 | staufen, RNA binding | 213037_x_at |
| | | | | protein (Drosophila) | |
| PHF1 | 0.031 | 0.130 | 8.60E-04 | PHD finger protein 1 | 202928_s_at |
| HN1 | 0.031 | 18.055 | 8.74E-04 | hematological and | 217755_at |
| | | | | neurological expressed 1 | |
| STOML2 | 0.031 | 6.512 | 8.78E-04 | stomatin (EPB72)-like 2 | 215416_s_at |
| ARID3B | 0.031 | 0.149 | 8.77E-04 | AT rich interactive | 218964_at |
| | | | | domain 3B (BRIGHT- | |
| | | | | like) | |
| IL19 | 0.031 | 8.869 | 8.93E-04 | interleukin 19 | 220745_at |
| WSX1 | 0.032 | 46.587 | 9.17E-04 | interleukin 27 receptor, | 205926_at |
| | | | | alpha | |
| NFE2L1 | 0.032 | 33.502 | 9.06E-04 | nuclear factor (erythroid- | 200759_x_at |
| | | | | derived 2)-like 1 | |
| TDE1 | 0.032 | 17.535 | 9.38E-04 | tumor differentially | 211769_x_at |
| | | | | expressed 1 | |
| NALP2 | 0.032 | 16.21 | 9.48E-04 | NACHT, leucine rich | 221690_s_at |
| | | | | repeat and PYD | |
| | | | | containing 2 | |
| POLA | 0.032 | 14.919 | 8.99E-04 | polymerase (DNA | 204835_at |
| | | | | directed), alpha | |
| CKLFSF6 | 0.032 | 13.746 | 9.50E-04 | chemokine-like factor | 217947_at |
| | | | | super family 6 | |
| SSH1 | 0.032 | 11.182 | 9.13E-04 | slingshot homolog 1 | 221753_at |
| | | | | (Drosophila) | |
| MINK | 0.032 | 0.145 | 9.49E-04 | misshapen/NIK-related | 214246_x_at |
| | | | | kinase | |
| DKFZP434H132 | 0.032 | 0.143 | 9.22E-04 | DKFZP434H132 protein | 215087_at |
| JM5 | 0.032 | 0.114 | 9.56E-04 | WD repeat domain, X- | 209216_at |
| | | | | linked 1 | |
| FLJ13479 | 0.032 | 0.010 | 9.37E-04 | hypothetical protein | 219047_s_at |
| | | | | FLJ13479 | |
| MKI67 | 0.032 | 69.144 | 1.01E-03 | antigen identified by | 212021_s_at |
| | | | | monoclonal antibody Ki- | |
| | | | | 67 | |
| RBX1 | 0.032 | 27.734 | 1.01E-03 | ring-box 1 | 218117_{—at} |
| TIMM13 | 0.032 | 22.616 | 1.00E-03 | translocase of inner | 218188_s_at |
| | | | | mitochondrial membrane | |
| | | | | 13 homolog (yeast) | |
| ECHDC1 | 0.032 | 16.161 | 1.01E-03 | enoyl Coenzyme A | 219974_x_at |
| | | | | hydratase domain | |
| | | | | containing 1 | |
| KIAA0930 | 0.032 | 14.228 | 1.01E-03 | chromosome 22 open | 212421_at |
| | | | | reading frame 9 | |
| HEG | 0.032 | 6.044 | 1.02E-03 | HEG homolog | 212822_at |
| MASK | 0.032 | 5.562 | 1.01E-03 | ankyrin repeat and KH | 208772_at |
| | | | | domain containing 1 | |
| JUNB | 0.032 | 0.108 | 1.02E-03 | jun B proto-oncogene | 201473_at |
| C9ORF28 | 0.032 | 0.037 | 1.01E-03 | chromosome 9 open | 52975_at |
| | | | | reading frame 28 | |
| RLF | 0.032 | 0.028 | 1.01E-03 | rearranged L-myc fusion | 204243_at |
| | | | | sequence | |
| AB026190 | 0.033 | 12.367 | 1.06E-03 | Kelch motif containing | 204177_s_at |
| | | | | protein | |
| GTF2H5 | 0.033 | 8.729 | 1.09E-03 | GTF2H5, general | 213357_at |
| | | | | transcription factor IIH, | |
| | | | | polypeptide 5 | |
| RBMS1 | 0.033 | 5.153 | 1.09E-03 | RNA binding motif, | 209868_s_at |
| | | | | single stranded | |
| | | | | interacting protein 1 | |
| ENIGMA | 0.033 | 0.081 | 1.09E-03 | PDZ and LIM domain 7 | 203370_s_at |
| | | | | (enigma) | |
| MIR | 0.033 | 0.128 | 1.10E-03 | c-mir, cellular modulator | 221824_s_at |
| | | | | of immune recognition | |
| SRRM2 | 0.033 | 5.461 | 1.11E-03 | serine/arginine repetitive | 208610_s_at |
| | | | | matrix 2 | |
| SRR | 0.033 | 15.068 | 1.12E-03 | serine racemase | 219205_at |
| MCL1 | 0.033 | 0.058 | 1.12E-03 | myeloid cell leukemia | 200797_s_at |
| | | | | sequence 1 (BCL2- | |
| | | | | related) | |
| FACL5 | 0.034 | 89.075 | 1.17E-03 | acyl-CoA synthetase | 218322_s_at |
| | | | | long-chain family | |
| | | | | member 5 | |
| CPSF1 | 0.034 | 0.209 | 1.16E-03 | cleavage and | 33132_at |
| | | | | polyadenylation specific | |
| | | | | factor 1, 160kDa | |
| AK2 | 0.034 | 17.668 | 1.19E-03 | adenylate kinase 2 | 212175_s_at |
| PTTG11P | 0.034 | 0.004 | 1.19E-03 | pituitary tumor- | 200677_at |
| | | | | transforming 1 | |
| | | | | interacting protein | |
| GTPBP1 | 0.034 | 0.032 | 1.19E-03 | GTP binding protein 1 | 219357_at |
| UNG | 0.035 | 10.732 | 1.24E-03 | uracil-DNA glycosylase | 202330_s_at |
| RPS28 | 0.035 | 0.215 | 1.23E-03 | ribosomal protein S28 | 216380_x_at |
| PAX5 | 0.035 | 8.402 | 1.24E-03 | paired box gene 5 (B-cell | 221969_at |
| | | | | lineage specific | |
| | | | | activator) | |
| PSMD8 | 0.035 | 11.013 | 1.29E-03 | proteasome (prosome, | 200820_at |
| | | | | macropain) 26S subunit, | |
| | | | | non-ATPase, 8 | |
| NUDT1 | 0.035 | 10.67 | 1.29E-03 | nudix (nucleoside | 204766_s_at |
| | | | | diphosphate linked | |
| | | | | moiety X)-type motif 1 | |
| SLC25A12 | 0.035 | 52.625 | 1.30E-03 | solute carrier family 25 | 203339_at |
| | | | | (mitochondrial carrier, | |
| | | | | Aralar), member 12 | |
| C1ORF24 | 0.036 | 12.539 | 1.31E-03 | chromosome 1 open | 217966_s_at |
| | | | | reading frame 24 | |
| HTATIP2 | 0.036 | 15.356 | 1.32E-03 | HIV-1 Tat interactive | 207180_s_at |
| | | | | protein 2, 30kDa | |
| SRPK2 | 0.036 | 3.184 | 1.34E-03 | SFRS protein kinase 2 | 203181_x_at |
| PRKAR1A | 0.036 | 16.407 | 1.34E-03 | protein kinase, cAMP- | 200604_s_at |
| | | | | dependent, regulatory, | |
| | | | | type I, alpha (tissue | |
| | | | | specific extinguisher 1) | |
| CD80 | 0.036 | 26.52 | 1.36E-03 | CD80 antigen (CD28 | 207176_s_at |
| | | | | antigen ligand 1, B7-1 | |
| | | | | antigen) | |
| MGC3248 | 0.036 | 20.329 | 1.37E-03 | dynactin 4 | 209231_s_at |
| UBXD2 | 0.036 | 6.211 | 1.39E-03 | UBX domain containing | 212007_at |
| | | | | 2 | |
| GALNT1 | 0.036 | 36.544 | 1.40E-03 | UDP-N-acetyl-alpha-D- | 201723_s_at |
| | | | | galactosamine:polypeptide | |
| | | | | N-acetylgalactosaminyl- | |
| | | | | transferase 1 (GalNAc-T1) | |
| STX18 | 0.036 | 23.897 | 1.43E-03 | syntaxin 18 | 218763_at |
| PDCD11 | 0.036 | 15.892 | 1.41E-03 | programmed cell death | 212424_at |
| | | | | 11 | |
| ISGF3G | 0.036 | 7.836 | 1.42E-03 | interferon-stimulated | 203882_at |
| | | | | transcription factor 3, | |
| | | | | gamma 48kDa | |
| RAB7 | 0.036 | 0.083 | 1.42E-03 | RAB7, member RAS | 211960_s_at |
| | | | | oncogene family | |
| CDC42 | 0.036 | 0.051 | 1.42E-03 | cell division cycle 42 | 210232_at |
| | | | | (GTP binding protein, | |
| | | | | 25kDa) | |
| NFATC1 | 0.036 | 80.225 | 1.55E-03 | nuclear factor of | 210162_s_at |
| | | | | activated T-cells, | |
| | | | | cytoplasmic, calcineurin- | |
| | | | | dependent 1 | |
| PSMD1 | 0.036 | 19.918 | 1.51E-03 | proteasome (prosome, | 201198_s_at |
| | | | | macropain) 26S subunit, | |
| | | | | non-ATPase, 1 | |
| COL4A3BP | 0.036 | 17.703 | 1.55E-03 | collagen, type IV, alpha | 219625_s_at |
| | | | | 3 (Goodpasture antigen) | |
| | | | | binding protein | |
| NR3C1 | 0.036 | 11.05 | 1.55E-03 | nuclear receptor | 201865_x_at |
| | | | | subfamily 3, group C, | |
| | | | | member 1 | |
| | | | | (glucocorticoid receptor) | |
| SEC63 | 0.036 | 7.604 | 1.54E-03 | SEC63-like (S. | 201914_s_at |
| | | | | cerevisiae) | |
| PSMD11 | 0.036 | 5.523 | 1.46E-03 | proteasome (prosome, | 208777_s_at |
| | | | | macropain) 26S subunit, | |
| | | | | non-ATPase, 11 | |
| H2AV | 0.036 | 0.268 | 1.57E-03 | H2A histone family, | 212206_s_at |
| | | | | member V | |
| CABIN1 | 0.036 | 0.162 | 1.55E-03 | calcineurin binding | 37652_at |
| | | | | protein 1 | |
| NET1 | 0.036 | 0.146 | 1.53E-03 | neuroepithelial cell | 201830_s_at |
| | | | | transforming gene 1 | |
| NFIL3 | 0.036 | 0.116 | 1.44E-03 | nuclear factor, | 203574_at |
| | | | | interleukin 3 regulated | |
| MOAP1 | 0.036 | 0.115 | 1.47E-03 | modulator of apoptosis 1 | 212508_at |
| SKP1A | 0.036 | 0.113 | 1.47E-03 | S-phase kinase- | 200719_at |
| | | | | associated protein 1A | |
| | | | | (p19A) | |
| FLJ11127 | 0.036 | 0.069 | 1.53E-03 | hypothetical protein | 219694_at |
| | | | | FLJ11127 | |
| G1P3 | 0.036 | 0.069 | 1.58E-03 | interferon, alpha- | 204415_at |
| | | | | inducible protein (clone | |
| | | | | IFI-6-16) | |
| BNIP3L | 0.036 | 0.044 | 1.55E-03 | BCL2/adenovirus E1B | 221478_at |
| | | | | 19kDa interacting protein | |
| | | | | 3-like | |
| C6ORF82 | 0.036 | 0.041 | 1.50E-03 | chromosome 6 open | 221488_s_at |
| | | | | reading frame 82 | |
| XTP2 | 0.037 | 4.327 | 1.62E-03 | HBxAg transactivated | 214055_x_at |
| | | | | protein 2 | |
| MBNL3 | 0.037 | 0.058 | 1.62E-03 | muscleblind-like 3 | 219814_at |
| | | | | (Drosophila) | |
| PDHB | 0.037 | 33.983 | 1.63E-03 | pyruvate dehydrogenase | 208911_s_at |
| | | | | (lipoamide) beta | |
| CKS1B | 0.038 | 16.085 | 1.71E-03 | CDC28 protein kinase | 201897_s_at |
| | | | | regulatory subunit 1B | |
| GALNS | 0.038 | 0.227 | 1.71E-03 | galactosamine (N- | 206335_at |
| | | | | acetyl)-6-sulfate | |
| | | | | sulfatase (Morquio | |
| | | | | syndrome, | |
| | | | | mucopolysaccharidosis | |
| | | | | type IVA) | |
| USP12 | 0.038 | 48.047 | 1.72E-03 | USP12, ubiquitin | 213327_s_at |
| | | | | specific protease 12 | |
| EIF5 | 0.038 | 8.566 | 1.73E-03 | eukaryotic translation | 208290_s_at |
| | | | | initiation factor 5 | |
| KIAA0650 | 0.038 | 0.146 | 1.73E-03 | KIAA0650 protein | 212577_at |
| UQCRFS1 | 0.038 | 0.060 | 1.74E-03 | ubiquinol-cytochrome c | 208909_at |
| | | | | reductase, Rieske iron- | |
| | | | | sulfur polypeptide 1 | |
| ACO1 | 0.038 | 49.485 | 1.78E-03 | aconitase 1, soluble | 207071_s_at |
| MRPL13 | 0.038 | 9.48 | 1.77E-03 | mitochondrial ribosomal | 218049_s_at |
| | | | | protein L13 | |
| SCGF | 0.038 | 0.120 | 1.75E-03 | stem cell growth factor; | 211709_s_at |
| | | | | lymphocyte secreted C- | |
| | | | | type lectin | |
| CHC1L | 0.038 | 0.084 | 1.79E-03 | chromosome | 204759_at |
| | | | | condensation 1-like | |
| TRIAD3 | 0.039 | 29.384 | 1.80E-03 | TRIAD3 protein | 218426_s_at |
| RFP | 0.039 | 35.742 | 1.87E-03 | ret finger protein | 212116_at |
| PSMD13 | 0.039 | 16.384 | 1.84E-03 | proteasome (prosome, | 201233_at |
| | | | | macropain) 26S subunit, | |
| | | | | non-ATPase, 13 | |
| ACOX1 | 0.039 | 15.909 | 1.86E-03 | acyl-Coenzyme A | 209600_s_at |
| | | | | oxidase 1, palmitoyl | |
| ITGAV | 0.039 | 12.837 | 1.82E-03 | integrin, alpha V | 202351_at |
| | | | | (vitronectin receptor, | |
| | | | | alpha polypeptide, | |
| | | | | antigen CD51) | |
| SEC23B | 0.039 | 11.687 | 1.83E-03 | Sec23 homolog B (S. | 201583_s_at |
| | | | | cerevisiae) | |
| RPA3 | 0.039 | 10.718 | 1.84E-03 | replication protein A3, | 209507_at |
| | | | | 14kDa | |
| KLF7 | 0.039 | 7.918 | 1.83E-03 | Kruppel-like factor 7 | 204334_at |
| | | | | (ubiquitous) | |
| AGTPBP1 | 0.039 | 0.099 | 1.87E-03 | ATP/GTP binding | 204500_s_at |
| | | | | protein 1 | |
| CGI-127 | 0.039 | 0.039 | 1.86E-03 | yippee protein | 217783_s_at |
| KIAA0892 | 0.039 | 0.071 | 1.90E-03 | KIAA0892 | 212505_s_at |
| APLP2 | 0.039 | 0.155 | 1.92E-03 | amyloid beta (A4) | 208248_x_at |
| | | | | precursor-like protein 2 | |
| IL7R | 0.039 | 3.182 | 1.94E-03 | interleukin 7 receptor | 205798_at |
| SR140 | 0.039 | 0.144 | 1.95E-03 | U2-associated SR140 | 212058_at |
| | | | | protein | |
| HMGCL | 0.040 | 11.109 | 1.99E-03 | 3-hydroxymethyl-3- | 202772_at |
| | | | | methylglutaryl- | |
| | | | | Coenzyme A lyase | |
| | | | | (hydroxymethylglutarica | |
| | | | | ciduria) | |
| TDP1 | 0.040 | 10.611 | 1.98E-03 | tyrosyl-DNA | 219715_s_at |
| | | | | phosphodiesterase 1 | |
| VDAC3 | 0.040 | 7.789 | 1.97E-03 | voltage-dependent anion | 208846_s_at |
| | | | | channel 3 | |
| HIPK1 | 0.040 | 0.025 | 2.01E-03 | homeodomain interacting | 212291_at |
| | | | | protein kinase 1 | |
| FLJ14639 | 0.040 | 38.716 | 2.03E-03 | nuclear factor of | 212809_at |
| | | | | activated T-cells, | |
| | | | | cytoplasmic, calcineurin- | |
| | | | | dependent 2 interacting | |
| | | | | protein | |
| CGI-01 | 0.040 | 9.62 | 2.04E-03 | CGI-01 protein | 212405_s_at |
| FLJ11078 | 0.040 | 0.094 | 2.02E-03 | hypothetical protein | 219354_{—at} |
| | | | | FLJ11078 | |
| CGI-128 | 0.041 | 54.238 | 2.10E-03 | CGI-128 protein | 218074_at |
| IL9 | 0.041 | 9.187 | 2.12E-03 | interleukin 9 | 208193_at |
| NUP43 | 0.041 | 6.165 | 2.13E-03 | nucleoporin 43kDa | 219007_at |
| CCNL1 | 0.041 | 0.153 | 2.12E-03 | cyclin L1 | 220046_s_at |
| GORASP2 | 0.041 | 0.100 | 2.13E-03 | golgi reassembly | 208843_s_at |
| | | | | stacking protein 2, | |
| | | | | 5kDa | |
| AP162 | 0.041 | 0.069 | 2.15E-03 | pleckstrin homology | 212717_at |
| | | | | domain containing, | |
| | | | | family M (with RUN | |
| | | | | domain) member 1 | |
| PLSCR3 | 0.041 | 0.029 | 2.16E-03 | phospholipid scramblase | 56197_at |
| | | | | 3 | |
| MGLL | 0.042 | 14.896 | 2.21E-03 | monoglyceride lipase | 211026_s_at |
| NCOA3 | 0.042 | 13.528 | 2.23E-03 | nuclear receptor | 207700_s_at |
| | | | | coactivator 3 | |
| RNUT1 | 0.042 | 11.552 | 2.22E-03 | RNA, U transporter 1 | 207438_s_at |
| ALEX3 | 0.042 | 5.508 | 2.21E-03 | armadillo repeat | 217858_s_at |
| | | | | containing, X-linked 3 | |
| TNFSF10 | 0.042 | 4.806 | 2.24E-03 | tumor necrosis factor | 202688_at |
| | | | | (ligand) superfamily, | |
| | | | | member 10 | |
| PPP6C | 0.042 | 0.045 | 2.24E-03 | protein phosphatase 6, | 203529_at |
| | | | | catalytic subunit | |
| CENPC1 | 0.042 | 0.106 | 2.25E-03 | centromere protein C 1 | 204739_at |
| NR1D1 | 0.042 | 0.197 | 2.25E-03 | nuclear receptor | 204760_s_at |
| | | | | subfamily 1, group D, | |
| | | | | member 1 | |
| MTMR2 | 0.042 | 11.89 | 2.28E-03 | myotubularin related | 203211_s_at |
| | | | | protein 2 | |
| FDPS | 0.042 | 11.053 | 2.27E-03 | farnesyl diphosphate | 201275_at |
| | | | | synthase (farnesyl | |
| | | | | pyrophosphate | |
| | | | | synthetase, | |
| | | | | dimethylallyltrans- | |
| | | | | transferase, | |
| | | | | geranyltranstransferase) | |
| FLJ12439 | 0.042 | 6.764 | 2.27E-03 | hypothetical protein | 219420_s_at |
| | | | | FLJ12439 | |
| TFEB | 0.042 | 0.138 | 2.27E-03 | transcription factor EB | 50221_at |
| Unknown | 0.042 | 0.315 | 2.31E-03 | no sequence similanty to | 222315_at |
| | | | | other genes or proteins | |
| KIAA1332 | 0.042 | 0.061 | 2.31E-03 | F-box protein 42 | 221813_at |
| C14ORF159 | 0.042 | 0.132 | 2.32E-03 | chromosome 14 open | 218298_s_at |
| | | | | reading frame 159 | |
| PSME2 | 0.042 | 10.064 | 2.34E-03 | proteasome (prosome, | 201762_s_at |
| | | | | macropain) activator | |
| | | | | subunit 2 (PA28 beta) | |
| MPHOSPH6 | 0.043 | 10.656 | 2.38E-03 | M-phase phosphoprotein | 203740_at |
| | | | | 6 | |
| YWHAB | 0.043 | 10.596 | 2.40E-03 | tyrosine 3- | 217717_s_at |
| | | | | monooxygenase/tryptophan | |
| | | | | 5-monooxygenase | |
| | | | | activation protein, beta | |
| | | | | polypeptide | |
| MCM7 | 0.043 | 7.75 | 2.40E-03 | MCM7 minichromosome | 208795_s_at |
| | | | | maintenance deficient 7 | |
| | | | | (S. cerevisiae) | |
| PSMD2 | 0.043 | 334.893 | 2.43E-03 | proteasome (prosome, | 200830_at |
| | | | | macropain) 26S subunit, | |
| | | | | non-ATPase, 2 | |
| AMPD2 | 0.043 | 0.122 | 2.45E-03 | adenosine | 212360_at |
| | | | | monophosphate | |
| | | | | deaminase 2 (isoform L) | |
| CCNE1 | 0.044 | 6.88 | 2.48E-03 | cyclin E1 | 213523_at |
| MMP7 | 0.044 | 6.512 | 2.48E-03 | matrix metalloproteinase | 204259_at |
| | | | | 7 (matrilysin, uterine) | |
| GTF2H1 | 0.044 | 12.954 | 2.51E-03 | general transcription | 202453_s_at |
| | | | | factor IIH, polypeptide 1, | |
| | | | | 62kDa | |
| FNBP1 | 0.044 | 5.151 | 2.52E-03 | formin binding protein 1 | 213940_s_at |
| UBD | 0.044 | 7.847 | 2.54E-03 | ubiquitin D | 205890_s_at |
| FLJ38984 | 0.045 | 19.598 | 2.57E-03 | hypothetical protein | 212791_at |
| | | | | FLJ38984 | |
| TLE4 | 0.045 | 0.108 | 2.58E-03 | transducin-like enhancer | 204872_at |
| | | | | of split 4 (E(sp1) | |
| | | | | homolog, Drosophila) | |
| ITM2B | 0.045 | 0.032 | 2.60E-03 | integral membrane | 217732_s_at |
| | | | | protein 2B | |
| HSD17B7 | 0.045 | 14.99 | 2.62E-03 | hydroxysteroid (17-beta) | 220081_x_at |
| | | | | dehydrogenase 7 | |
| KIAA1115 | 0.047 | 33.455 | 2.74E-03 | KIAA1115 | 209229_s_at |
| COAS1 | 0.047 | 3.854 | 2.74E-03 | chomosome one | 214693_x_at |
| | | | | amplified sequence 1 | |
| | | | | cyclophilin | |
| XRCC5 | 0.047 | 17.167 | 2.77E-03 | X-ray repair | 208643_s_at |
| | | | | complementing defective | |
| | | | | repair in Chinese hamster | |
| | | | | cells 5 (double-strand- | |
| | | | | break rejoining; Ku | |
| | | | | autoantigen, 80kDa) | |
| STMN1 | 0.047 | 11.125 | 2.76E-03 | stathmin 1/oncoprotein | 200783_s_at |
| | | | | 18 | |
| CTLA4 | 0.047 | 8.016 | 2.77E-03 | cytotoxic T-lymphocyte- | 221331_x_at |
| | | | | associated protein 4 | |
| STAG2 | 0.047 | 6.595 | 2.78E-03 | stromal antigen 2 | 207983_s_at |
| KIAA0404 | 0.047 | 0.144 | 2.78E-03 | KIAA0404 protein | 213300_at |
| SF3B4 | 0.047 | 0.180 | 2.80E-03 | splicing factor 3b, | 209044_x_at |
| | | | | subunit 4, 49kDa | |
| CXCL9 | 0.047 | 6.108 | 2.81E-03 | chemokine (C-X-C | 203915_at |
| | | | | motif) ligand 9 | |
| ITGAX | 0.047 | 0.032 | 2.85E-03 | integrin, alpha X (antigen | 210184_at |
| | | | | CD11C (p150), alpha | |
| | | | | polypeptide) | |
| FLJ14888 | 0.048 | 25.043 | 2.86E-03 | hypothetical protein | 213031_s_at |
| | | | | FLJ14888 | |
| FLJ10803 | 0.048 | 31.56 | 2.90E-03 | hypothetical protein | 209445_x_at |
| | | | | FLJ10803 | |
| OSBPL9 | 0.048 | 288.036 | 2.91E-03 | oxysterol binding | 218047_at |
| | | | | protein-like 9 | |
| PTEN | 0.048 | 0.107 | 2.91E-03 | phosphatase and tensin | 204054_at |
| | | | | homolog (mutated in | |
| | | | | multiple advanced | |
| | | | | cancers 1) | |
| EFHD2 | 0.048 | 0.128 | 2.93E-03 | EF hand domain | 217992_s_at |
| | | | | containing 2 | |
| PPIH | 0.048 | 29.937 | 2.99E-03 | peptidyl prolyl isomerase | 204228_at |
| | | | | H (cyclophilin H) | |
| NKTR | 0.048 | 4.902 | 3.00E-03 | natural killer-tumor | 202379_s_at |
| | | | | recognition sequence | |
| BAZ2A | 0.048 | 4.766 | 2.99E-03 | bromodomain adjacent to | 201353_s_at |
| | | | | zinc finger domain, 2A | |
| DOCK2 | 0.048 | 0.100 | 2.96E-03 | dedicator of cytokinesis | 213160_at |
| | | | | 2 | |
| FGR | 0.048 | 0.088 | 3.02E-03 | Gardner-Rasheed feline | 208438_s_at |
| | | | | sarcoma viral (v-fgr) | |
| | | | | oncogene homolog | |
| ZCCHC2 | 0.048 | 0.080 | 2.98E-03 | zinc finger, CCHC | 219062_s_at |
| | | | | domain containing 2 | |
| QKI | 0.049 | 29.983 | 3.12E-03 | quaking homolog, KH | 212263_at |
| | | | | domain RNA binding | |
| | | | | (mouse) | |
| SUCLA2 | 0.049 | 10.996 | 3.14E-03 | succinate-CoA ligase, | 202930_s_at |
| | | | | ADP-forming, beta | |
| | | | | subunit | |
| MATR3 | 0.049 | 0.124 | 3.11E-03 | matrin3 | 200626_s_at |
| GABR1 | 0.049 | 0.117 | 3.09E-03 | gamma-aminobutyric | 203146_s_at |
| | | | | acid (GABA) B receptor, | |
| | | | | 1 | |
| SPN | 0.049 | 0.102 | 3.14E-03 | sialophorin (gpL115, | 206057_x_at |
| | | | | leukosialin, CD43) | |
| KIAA1536 | 0.049 | 0.073 | 3.10E-03 | KIAA1536 protein | 209002_s_at |
| PABPC3 | 0.049 | 0.038 | 3.06E-03 | poly(A) binding protein, | 215157_x_at |
| | | | | cytoplasmic 3 | |
| C3ORF4 | 0.049 | 5.543 | 3.17E-03 | chromosome 3 open | 208925_at |
| | | | | reading frame 4 | |
| CYLD | 0.049 | 0.161 | 3.18E-03 | cylindromatosis (turban | 60084_at |
| | | | | tumor syndrome) | |
| FLJ21347 | 0.049 | 0.098 | 3.18E-03 | hypothetical protein | 218164_at |
| | | | | FLJ21347 | |
| FBS1 | 0.049 | 0.031 | 3.17E-03 | fibrosin 1 | 218255_s_at |
| AIM2 | 0.049 | 9.506 | 3.20E-03 | absent in melanoma 2 | 206513_at |
| PTX1 | 0.049 | 9.051 | 3.20E-03 | PTX1 protein | 218135_at |
| CLN5 | 0.049 | 11.634 | 3.27E-03 | ceroid-lipofuscinosis, | 204084_s_at |
| | | | | neuronal 5 | |
| EPRS | 0.049 | 9.17 | 3.28E-03 | glutamyl-prolyl-tRNA | 200842_s_at |
| | | | | synthetase | |
| LRDD | 0.049 | 0.219 | 3.27E-03 | leucine-rich repeats and | 219019_at |
| | | | | death domain containing | |
| LOC283537 | 0.049 | 0.094 | 3.28E-03 | hypothetical protein | 214719_at |
| | | | | LOC283537 | |
| PEX3 | 0.050 | 9.809 | 3.31E-03 | peroxisomal biogenesis | 203972_s_at |
| | | | | factor 3 | |
| NCOA2 | 0.050 | 0.220 | 3.34E-03 | nuclear receptor | 212867_at |
| | | | | coactivator 2 | |
| ARHQ | 0.050 | 41.236 | 3.38E-03 | ras homolog gene family, | 212119_at |
| | | | | member Q | |
| PFKM | 0.050 | 18.355 | 3.36E-03 | phosphofructokinase, | 210976_s_at |
| | | | | muscle | |
| BHC80 | 0.050 | 0.124 | 3.37E-03 | BRAF35/HDAC2 | 203278_s_at |
| | | | | complex (80 kDa) | |
| CD2BP2 | 0.050 | 59.36 | 3.45E-03 | CD2 antigen | 202256_at |
| | | | | (cytoplasmic tail) | |
| | | | | binding protein 2 | |
| WARS | 0.050 | 23.882 | 3.48E-03 | tryptophanyl-tRNA | 200628_s_at |
| | | | | synthetase | |
| FXC1 | 0.050 | 13.071 | 3.50E-03 | fracture callus 1 homolog | 217981_s_at |
| | | | | (rat) | |
| TSTA3 | 0.050 | 6.918 | 3.49E-03 | tissue specific | 201644_at |
| | | | | transplantation antigen | |
| | | | | P35B | |
| ESPL1 | 0.050 | 6.537 | 3.54E-03 | extra spindle poles like 1 | 204817_at |
| | | | | (S. cerevisiae) | |
| PWP1 | 0.050 | 4.459 | 3.54E-03 | nuclear phosphoprotein | 201606_s_at |
| | | | | similar to S. cerevisiae | |
| | | | | PWP1 | |
| KRAS2 | 0.050 | 3.71 | 3.54E-03 | v-Ki-ras2 Kirsten rat | 214352_s_at |
| | | | | sarcoma 2 viral oncogene | |
| | | | | homolog | |
| ZNF408 | 0.050 | 0.239 | 3.53E-03 | zinc finger protein 408 | 219224_x_at |
| TCF7L2 | 0.050 | 0.223 | 3.51E-03 | transcription factor 7-like | 216035_x_at |
| | | | | 2 (T-cell specific, HMG- | |
| | | | | box) | |
| RGS2 | 0.050 | 0.176 | 3.51E-03 | regulator of G-protein | 202388_at |
| | | | | signalling 2, 24kDa | |
| PLEKHF2 | 0.050 | 0.154 | 3.54E-03 | pleckstrin homology | 218640_s_at |
| | | | | domain containing, | |
| | | | | family F (with FYVE | |
| | | | | domain) member 2 | |
| EDG6 | 0.050 | 0.144 | 3.51E-03 | endothelial | 206437_at |
| | | | | differentiation, G- | |
| | | | | protein-coupled receptor 6 | |
| KIAA1076 | 0.050 | 0.117 | 3.55E-03 | KIAA1076 protein | 213153_at |
| DRE1 | 0.050 | 0.113 | 3.41E-03 | DRE1 protein | 221985_at |
| C14ORF32 | 0.050 | 0.097 | 3.52E-03 | chromosome 14 open | 212643_at |
| | | | | reading frame 32 | |
| MAP3K71P2 | 0.050 | 0.079 | 3.39E-03 | mitogen-activated | 212184_s_at |
| | | | | protein kinase kinase | |
| | | | | kinase 7 interacting | |
| | | | | protein 2 | |
| ARL4 | 0.050 | 0.063 | 3.44E-03 | ADP-ribosylation factor- | 205020_s_at |
| | | | | like 4A | |
| RPA2 | 0.050 | 17.449 | 3.57E-03 | replication protein A2, | 201756_at |
| | | | | 32kDa | |
| NUP50 | 0.051 | 13.853 | 3.61E-03 | nucleoporin 50kDa | 218294_s_at |
| KIAA0555 | 0.051 | 7.853 | 3.61E-03 | KIAA0555 gene product | 205888_s_at |
| GAS7 | 0.051 | 0.091 | 3.60E-03 | growth arrest-specific 7 | 202192_s_at |
| SSFA2 | 0.051 | 0.036 | 3.62E-03 | sperm specific antigen 2 | 202506_at |
| GMEB2 | 0.051 | 0.097 | 3.68E-03 | glucocorticoid | 44146_at |
| | | | | modulatory element | |
| | | | | binding protein 2 | |
| PIR51 | 0.051 | 9.238 | 3.70E-03 | RAD51-interacting | 204146_at |
| | | | | protein | |
| C9ORF83 | 0.051 | 8.68 | 3.71E-03 | chromosome 9 open | 218085_at |
| | | | | reading frame 83 | |
| PRO1843 | 0.051 | 0.126 | 3.73E-03 | hypothetical protein | 219599_at |
| | | | | PRO1843 | |
| VEGF | 0.052 | 0.124 | 3.78E-03 | vascular endothelial | 212171_x_at |
| | | | | growth factor | |
| DNM1L | 0.052 | 16.425 | 3.80E-03 | dynamin 1-like | 203105_s_at |
| RERE | 0.052 | 0.093 | 3.82E-03 | arginine-glutamic acid | 200940_s_at |
| | | | | dipeptide (RE) repeats | |
| ARID1A | 0.052 | 11.487 | 3.83E-03 | AT rich interactive | 212152_x_at |
| | | | | domain 1A (SWI- like) | |
| FLJ10815 | 0.052 | 9.617 | 3.83E-03 | hypothetical protein | 56821_at |
| | | | | FLJ10815 | |
| PSMA4 | 0.052 | 51.574 | 3.87E-03 | proteasome (prosome, | 203396_at |
| | | | | macropain) subunit, | |
| | | | | alpha type, 4 | |
| GNL1 | 0.052 | 19.339 | 3.87E-03 | guanine nucleotide | 203307_at |
| | | | | binding protein-like 1 | |
| CIAO1 | 0.052 | 17.811 | 3.87E-03 | WD40 protein Ciao1 | 203536_s_at |
| MNT | 0.052 | 0.113 | 3.86E-03 | MAX binding protein | 204206_at |
| CXCL5 | 0.052 | 4.558 | 3.88E-03 | chemokine (C-X-C | 214974_x_at |
| | | | | motif) ligand 5 | |
| FLJ32731 | 0.052 | 0.180 | 3.90E-03 | hypothetical protein | 218017_s_at |
| | | | | FLJ32731 | |
| MYCBP | 0.053 | 23.309 | 3.99E-03 | c-myc binding protein | 203359_s_at |
| KIAA0102 | 0.053 | 9.997 | 3.93E-03 | KIAA0102 gene product | 201240_s_at |
| PROSC | 0.053 | 5.622 | 3.97E-03 | proline synthetase co- | 209385_s_at |
| | | | | transcribed homolog | |
| | | | | (bacterial) | |
| LYL1 | 0.053 | 0.235 | 3.97E-03 | lymphoblastic leukemia | 210044_s_at |
| | | | | derived sequence 1 | |
| DUSP10 | 0.053 | 0.099 | 3.97E-03 | dual specificity | 221563_at |
| | | | | phosphatase 10 | |
| MKRN1 | 0.053 | 0.095 | 3.98E-03 | makorin, ring finger | 209845_at |
| | | | | protein, 1 | |
| Unknown | 0.053 | 0.081 | 3.99E-03 | gene of unknown | 65588_at |
| | | | | function | |
| Unknown | 0.053 | 0.271 | 4.01E-03 | Unknown | 211996_s_at |
| CKS2 | 0.053 | 5.629 | 4.02E-03 | CDC28 protein kinase | 204170_s_at |
| | | | | regulatory subunit 2 | |
| KMO | 0.053 | 5.843 | 4.05E-03 | kynurenine 3- | 211138_s_at |
| | | | | monooxygenase | |
| | | | | (kyrnurenine 3- | |
| | | | | hydroxylase) | |
| SGK | 0.053 | 0.161 | 4.04E-03 | serum/glucocorticoid | 201739_at |
| | | | | regulated kinase | |
| C20ORF104 | 0.053 | 0.091 | 4.05E-03 | chromosome 20 open | 209422_at |
| | | | | reading frame 104 | |
| ARS2 | 0.053 | 0.028 | 4.04E-03 | arsenate resistance | 201680_x_at |
| | | | | protein ARS2 | |
| ZNF259 | 0.053 | 26.34 | 4.12E-03 | zinc finger protein 259 | 200054_at |
| SERP1 | 0.054 | 0.022 | 4.16E-03 | stress-associated | 200971_s_at |
| | | | | endoplasmic reticulum | |
| | | | | protein 1 | |
| GC20 | 0.054 | 0.016 | 4.16E-03 | translation factor sui1 | 201738_at |
| | | | | homolog | |
| TRAPPC3 | 0.054 | 11.773 | 4.18E-03 | trafficking protein | 203511_s_at |
| | | | | particle complex 3 | |
| MSF | 0.054 | 0.222 | 4.19E-03 | MLL septin-like fusion | 208657_s_at |
| CDC40 | 0.054 | 0.074 | 4.18E-03 | cell division cycle 40 | 203377_s_at |
| | | | | homolog (yeast) | |
| PPP3CA | 0.054 | 5.417 | 4.21E-03 | protein phosphatase 3 | 202425_x_at |
| | | | | (formerly 2B), catalytic | |
| | | | | subunit, alpha isoform | |
| | | | | (calcineurin A alpha) | |
| FLJ14753 | 0.054 | 0.021 | 4.22E-03 | hypothetical protein | 211185_s_at |
| | | | | FLJ14753 | |
| PELI1 | 0.054 | 0.175 | 4.24E-03 | pellino homolog 1 | 218319_at |
| | | | | (Drosophila) | |
| PRKCSH | 0.054 | 0.064 | 4.24E-03 | protein kinase C | 214080_x_at |
| | | | | substrate 80K-H | |
| SPINT2 | 0.054 | 0.115 | 4.29E-03 | serine protease inhibitor, | 210715_s_at |
| | | | | Kunitz type, 2 | |
| PSARL | 0.055 | 50.956 | 4.33E-03 | presenilin associated, | 218271_s_at |
| | | | | rhomboid-like | |
| HT007 | 0.056 | 10.945 | 4.40E-03 | uncharacterized | 221622_s_at |
| | | | | hypothalamus protein | |
| | | | | HT007 | |
| RAD51C | 0.056 | 5.167 | 4.44E-03 | RAD51 homolog C (S. | 209849_s_at |
| | | | | cerevisiae) | |
| TRIP-BR2 | 0.056 | 3.547 | 4.46E-03 | SERTA domain | 202656_s_at |
| | | | | containing 2 | |
| TRA1 | 0.056 | 12.843 | 4.49E-03 | tumor rejection antigen | 200598_s_at |
| | | | | (gp96) 1 | |
| DKFZP586D0919 | 0.056 | 25.287 | 4.52E-03 | hepatocellularcarcinoma- | 213861_s_at |
| | | | | associated antigen | |
| | | | | HCA557a | |
| CIC | 0.056 | 0.300 | 4.52E-03 | capicua homolog | 212784_at |
| | | | | (Drosophila) | |
| PIK3CA | 0.056 | 0.075 | 4.51E-03 | phosphoinositide-3- | 204369_at |
| | | | | kinase, catalytic, alpha | |
| | | | | polypeptide | |
| HSPC051 | 0.057 | 12.156 | 4.53E-03 | ubiquinol-cytochrome c | 218190_s_at |
| | | | | reductase complex (7.2 | |
| | | | | kD) | |
| ELAVL1 | 0.057 | 4.903 | 4.57E-03 | ELAV (embryonic lethal, | 201726_at |
| | | | | abnormal vision, | |
| | | | | Drosophila)-like 1 (Hu | |
| | | | | antigen R) | |
| NADSYN1 | 0.057 | 0.182 | 4.56E-03 | NAD synthetase 1 | 218840_s_at |
| CCL22 | 0.057 | 3.61 | 4.58E-03 | chemokine (C-C motif) | 207861_at |
| | | | | ligand 22 | |
| CCNB2 | 0.057 | 12.529 | 4.62E-03 | cyclin B2 | 202705_at |
| C20ORF67 | 0.057 | 0.176 | 4.61E-03 | chromosome 20 open | 222044_at |
| | | | | reading frame 67 | |
| LOC51064 | 0.057 | 0.128 | 4.64E-03 | glutathione S-transferase | 217751_at |
| | | | | kappa 1 | |
| POLR2K | 0.057 | 7.759 | 4.70E-03 | polymerase (RNA) II | 202635_s_at |
| | | | | (DNA directed) | |
| | | | | polypeptide K, 7.0kDa | |
| LRP8 | 0.057 | 7.185 | 4.71E-03 | low density lipoprotein | 205282_at |
| | | | | receptor-related protein | |
| | | | | 8, apolipoprotein e | |
| | | | | receptor | |
| FLJ20080 | 0.057 | 9.512 | 4.73E-03 | aftiphilin protein | 217939_s_at |
| ACADM | 0.058 | 6.254 | 4.82E-03 | acyl-Coenzyme A | 202502_at |
| | | | | dehydrogenase, C-4 to C- | |
| | | | | 12 straight chain | |
| JUND | 0.058 | 0.059 | 4.82E-03 | jun D proto-oncogene | 203752_s_at |
| FLJ20534 | 0.058 | 12.83 | 4.86E-03 | hypothetical protein | 218646_at |
| | | | | FLJ20534 | |
| TOB1 | 0.058 | 0.172 | 4.87E-03 | transducer of ERBB2, 1 | 202704_at |
| ACTG1 | 0.058 | 0.006 | 4.88E-03 | actin, gamma 1 | 212988_x_at |
| FLJ10534 | 0.059 | 7.735 | 4.92E-03 | hypothetical protein | 221987_s_at |
| | | | | FLJ10534 | |
| CTPS | 0.059 | 5.567 | 4.93E-03 | CTP synthase | 202613_at |
| TCP1 | 0.059 | 21.895 | 5.00E-03 | t-complex 1 | 208778_s_at |
| D1S155E | 0.059 | 0.073 | 5.00E-03 | NRAS-related gene | 219939_s_at |
| TIMELESS | 0.059 | 6.145 | 5.02E-03 | timeless homolog | 203046_s_at |
| | | | | (Drosophila) | |
| NCOR1 | 0.059 | 59.364 | 5.05E-03 | nuclear receptor co- | 200854_at |
| | | | | repressor 1 | |
| DDEF1 | 0.059 | 7.693 | 5.07E-03 | development and | 221039_s_at |
| | | | | differentiation enhancing | |
| | | | | factor 1 | |
| UBE2L3 | 0.059 | 7.274 | 5.14E-03 | ubiquitin-conjugating | 200684_s_at |
| | | | | enzyme E2L 3 | |
| C9ORF40 | 0.059 | 7.261 | 5.11E-03 | chromosome 9 open | 218904_s_at |
| | | | | reading frame 40 | |
| PHF3 | 0.059 | 0.081 | 5.14E-03 | PHD finger protein 3 | 217952_x_at |
| DKFZP564D0478 | 0.059 | 0.049 | 5.14E-03 | hypothetical protein | 52078_at |
| | | | | DKFZp564D0478 | |
| CSK | 0.059 | 0.040 | 5.11E-03 | c-src tyrosine kinase | 202329_at |
| FBXL12 | 0.059 | 0.008 | 5.11E-03 | F-box and leucine-rich | 220127_s_at |
| | | | | repeat protein 12 | |
| CSNK2A1 | 0.059 | 9.961 | 5.17E-03 | casein kinase 2, alpha 1 | 212075_s_at |
| | | | | polypeptide | |
| KIAA0483 | 0.059 | 22.767 | 5.18E-03 | F-box protein 28 | 202272_s_at |
| NEDD8 | 0.060 | 5.847 | 5.21E-03 | neural precursor cell | 201840_at |
| | | | | expressed, | |
| | | | | developmentally down- | |
| | | | | regulated 8 | |
| TNFRSF9 | 0.060 | 3.452 | 5.20E-03 | tumor necrosis factor | 207536_s_at |
| | | | | receptor superfamily, | |
| | | | | member 9 | |
| KIAA0738 | 0.060 | 7.398 | 5.25E-03 | KIAA0738 gene product | 204403_x_at |
| ZNF161 | 0.060 | 0.086 | 5.24E-03 | zinc finger protein 161 | 202171_at |
| SIAT9 | 0.060 | 0.018 | 5.29E-03 | sialyltransferase 9 (CMP- | 203217_s_at |
| | | | | NeuAc: lactosylceramide | |
| | | | | alpha-2,3- | |
| | | | | sialyltransferase; GM3 | |
| | | | | synthase) | |
| MADH7 | 0.060 | 0.113 | 5.35E-03 | SMAD, mothers against | 204790_at |
| | | | | DPP homolog 7 | |
| | | | | (Drosophila) | |
| USP3 | 0.060 | 0.051 | 5.36E-03 | ubiquitin specific | 221654_s_at |
| | | | | protease 3 | |
| KHDRBS1 | 0.060 | 7.898 | 5.39E-03 | KH domain containing, | 201488_x_at |
| | | | | RNA binding, signal | |
| | | | | transduction associated 1 | |
| C5ORF6 | 0.061 | 0.047 | 5.42E-03 | chromosome 5 open | 218023_s_at |
| | | | | reading frame 6 | |
| GLG1 | 0.061 | 6.776 | 5.48E-03 | golgi apparatus protein 1 | 207966_s_at |
| TCF8 | 0.061 | 0.300 | 5.47E-03 | transcription factor 8 | 208078_s_at |
| | | | | (represses interleukin 2 | |
| | | | | expression) | |
| RBAF600 | 0.061 | 0.011 | 5.50E-03 | retinoblastoma- | 211950_at |
| | | | | associated factor 600 | |
| SLC35D2 | 0.061 | 21.354 | 5.54E-03 | solute carrier family 35, | 213082_s_at |
| | | | | member D2 | |
| PIGA | 0.061 | 0.156 | 5.55E-03 | phosphatidylinositol | 205281_s_at |
| | | | | glycan, class A | |
| | | | | (paroxysmal nocturnal | |
| | | | | hemoglobinuria) | |
| DUSP3 | 0.061 | 9.995 | 5.57E-03 | dual specificity | 201536_at |
| | | | | phosphatase 3 (vaccinia | |
| | | | | virus phosphatase VH1- | |
| | | | | related) | |
| DSCR1 | 0.061 | 26.47 | 5.59E-03 | Down syndrome critical | 208370_s_at |
| | | | | region gene 1 | |
| CGI-51 | 0.062 | 26.286 | 5.74E-03 | CGI-51 protein | 201570_at |
| TIP120A | 0.062 | 14.465 | 5.68E-03 | TBP-interacting protein | 208839_s_at |
| MAC30 | 0.062 | 9.549 | 5.71E-03 | hypothetical protein | 212282_at |
| | | | | MAC30 | |
| PTMA | 0.062 | 9.39 | 5.73E-03 | prothymosin, alpha (gene | 216384_x_at |
| | | | | sequence 28) | |
| WDR12 | 0.062 | 6.497 | 5.69E-03 | WD repeat domain 12 | 218512_at |
| POLE2 | 0.062 | 5.826 | 5.65E-03 | polymerase (DNA | 205909_at |
| | | | | directed), epsilon 2 (p59 | |
| | | | | subunit) | |
| NRG1 | 0.062 | 0.308 | 5.70E-03 | neuregulin 1 | 206343_s_at |
| SLC22A18 | 0.062 | 0.139 | 5.66E-03 | solute carrier family 22 | 204981_at |
| | | | | (organic cation | |
| | | | | transporter), member 18 | |
| VAMP2 | 0.062 | 0.092 | 5.71E-03 | vesicle-associated | 214792_x_at |
| | | | | membrane protein 2 | |
| | | | | (synaptobrevin 2) | |
| Unknown | 0.062 | 0.069 | 5.71E-03 | no sequence similarity to | 213215_at |
| | | | | any genes or proteins | |
| TRAF6 | 0.062 | 0.049 | 5.76E-03 | TNF receptor-associated | 205558_at |
| | | | | factor 6 | |
| EV12B | 0.062 | 0.109 | 5.81E-03 | ecotropic viral | 211742_s_at |
| | | | | integration site 2B | |
| TIEG2 | 0.062 | 0.231 | 5.84E-03 | TGFB inducible early | 218486_at |
| | | | | growth response 2 | |
| COPS5 | 0.062 | 8.168 | 5.89E-03 | COP9 constitutive | 201652_at |
| | | | | photomorphogenic | |
| | | | | homolog subunit 5 | |
| | | | | (Arabidopsis) | |
| RNF139 | 0.062 | 0.143 | 5.92E-03 | ring finger protein 139 | 209510_at |
| PCMT1 | 0.063 | 8.822 | 6.03E-03 | protein-L-isoaspartate | 205202_at |
| | | | | (D-aspartate) O- | |
| | | | | methyltransferase | |
| MPO | 0.063 | 0.146 | 6.03E-03 | myeloperoxidase | 203949_at |
| KCNK4 | 0.063 | 6.788 | 6.07E-03 | potassium channel, | 219883_at |
| | | | | subfamily K, member 4 | |
| SSA2 | 0.063 | 5.063 | 6.09E-03 | Sjogren syndrome | 210438_x_at |
| | | | | antigen A2 (60kDa, | |
| | | | | ribonucleoprotein | |
| | | | | autoantigen SS-A/Ro) | |
| Unknown | 0.064 | 4.635 | 6.15E-03 | no sequence similarity to | 217586_x_at |
| | | | | any genes or proteins | |
| DSIPI | 0.064 | 0.237 | 6.17E-03 | delta sleep inducing | 208763_s_at |
| | | | | peptide, immunoreactor | |
| KIAA0683 | 0.064 | 13.161 | 6.26E-03 | KIAA0683 gene product | 34260_at |
| POLD3 | 0.064 | 8.246 | 6.27E-03 | polymerase (DNA- | 212836_at |
| | | | | directed), delta 3, | |
| | | | | accessory subunit | |
| EEF2 | 0.064 | 0.180 | 6.27E-03 | eukaryotic translation | 204102_s_at |
| | | | | elongation factor 2 | |
| KIAA1002 | 0.064 | 0.133 | 6.25E-03 | KIAA1002 protein | 203831_at |
| NEIL1 | 0.064 | 0.090 | 6.26E-03 | nei endonuclease VIII- | 219396_s_at |
| | | | | like 1 (E. coli) | |
| FLJ10099 | 0.065 | 25.454 | 6.34E-03 | hypothetical protein | 218008_at |
| | | | | FLJ10099 | |
| KIAA0582 | 0.065 | 6.047 | 6.34E-03 | KIAA0582 | 212677_s_at |
| HADHSC | 0.065 | 0.155 | 6.39E-03 | L-3-hydroxyacyl- | 201034_at |
| | | | | Coenzyme A | |
| | | | | dehydrogenase, short | |
| | | | | chain | |
| C2ORF6 | 0.065 | 8.647 | 6.40E-03 | MOB1, Mps One Binder | 201298_s_at |
| | | | | kinase activator-like 1B | |
| | | | | (yeast) | |
| VIPR1 | 0.065 | 0.154 | 6.41E-03 | vasoactive intestinal | 205019_s_at |
| | | | | peptide receptor 1 | |
| SLC4A1AP | 0.065 | 36.646 | 6.42E-03 | solute carrier family 4 | 218682_s_at |
| | | | | (anion exchanger), | |
| | | | | member 1, adaptor | |
| | | | | protein | |
| ARL7 | 0.065 | 0.187 | 6.44E-03 | ADP-ribosylation factor- | 202207_at |
| | | | | like 7 | |
| DXYS155E | 0.065 | 0.032 | 6.44E-03 | DNA segment on | 203624_at |
| | | | | chromosome X and Y | |
| | | | | (unique) 155 expressed | |
| | | | | sequence | |
| BIRC2 | 0.065 | 0.118 | 6.45E-03 | baculoviral IAP repeat- | 202076_at |
| | | | | containing 2 | |
| STAT5A | 0.065 | 56.762 | 6.55E-03 | signal transducer and | 203010_at |
| | | | | activator of transcription | |
| | | | | 5A | |
| PHB | 0.065 | 8.89 | 6.56E-03 | prohibitin | 200659_s_at |
| MYLK | 0.065 | 3.958 | 6.55E-03 | myosin, light polypeptide | 202555_s_at |
| | | | | kinase | |
| ATP5L | 0.065 | 0.096 | 6.57E-03 | ATP synthase, H+ | 210453_x_at |
| | | | | transporting, | |
| | | | | mitochondrial F0 | |
| | | | | complex, subunit g | |
| KEAP1 | 0.066 | 7.403 | 6.58E-03 | kelch-like ECH- | 202417_at |
| | | | | associated protein 1 | |
| CAMKK2 | 0.066 | 3.454 | 6.63E-03 | calcium/calmodulin- | 213812_s_at |
| | | | | dependent protein kinase | |
| | | | | kinase 2, beta | |
| PRKCN | 0.066 | 0.189 | 6.62E-03 | protein kinase C, nu | 218236_s_at |
| MARK4 | 0.066 | 0.196 | 6.65E-03 | MAP/microtubule | 55065_at |
| | | | | affinity-regulating kinase 4 | |
| CDK4 | 0.066 | 5.678 | 6.69E-03 | cyclin-dependent kinase 4 | 202246_s_at |
| PAICS | 0.066 | 5.533 | 6.71E-03 | phosphoribosylaminoimi | 201013_s_at |
| | | | | dazole carboxylase, | |
| | | | | phosphoribosylaminoimi | |
| | | | | dazole | |
| | | | | succinocarboxamide | |
| | | | | synthetase | |
| CGI-90 | 0.066 | 9.14 | 6.77E-03 | CGI-90 protein | 218549_s_at |
| TNIP3 | 0.066 | 5.962 | 6.76E-03 | TNFAIP3 interacting | 220655_at |
| | | | | protein 3 | |
| NFKB1 | 0.066 | 5.354 | 6.75E-03 | nuclear factor of kappa | 209239_at |
| | | | | light polypeptide gene | |
| | | | | enhancer in B-cells 1 | |
| | | | | (p105) | |
| C1ORF9 | 0.066 | 0.058 | 6.77E-03 | chromosome 1 open | 203429_s_at |
| | | | | reading frame 9 | |
| POP5 | 0.067 | 8.456 | 6.83E-03 | processing of precursor | 204839_at |
| | | | | 5, ribonuclease P/MRP | |
| | | | | subunit (S. cerevisiae) | |
| ILI2B | 0.067 | 4.425 | 6.83E-03 | interleukin 12B (natural | 207901_at |
| | | | | killer cell stimulatory | |
| | | | | factor 2, cytotoxic | |
| | | | | lymphocyte maturation | |
| | | | | factor 2, p40) | |
| RUNX1 | 0.067 | 0.037 | 6.82E-03 | runt-related transcription | 208129_x_at |
| | | | | factor 1 (acute myeloid | |
| | | | | leukemia 1; aml1 | |
| | | | | oncogene) | |
| C20ORF121 | 0.067 | 0.125 | 6.84E-03 | chromosome 20 open | 221472_at |
| | | | | reading frame 121 | |
| EIF2B2 | 0.067 | 28.683 | 6.91E-03 | eukaryotic translation | 202461_at |
| | | | | initiation factor 2B, | |
| | | | | subunit 2 beta, 39kDa | |
| MGC4825 | 0.067 | 14.636 | 6.92E-03 | hypothetical protein | 221620_s_at |
| | | | | MGC4825 | |
| ILF3 | 0.067 | 11.625 | 6.86E-03 | interleukin enhancer | 217805_at |
| | | | | binding factor 3, 90kDa | |
| MRPL19 | 0.067 | 7.652 | 6.91E-03 | mitochondrial ribosomal | 203465_at |
| | | | | protein L19 | |
| KIAA0982 | 0.067 | 0.170 | 6.86E-03 | WD repeat domain 37 | 211383_s_at |
| CCR1 | 0.067 | 4.928 | 6.97E-03 | chemokine (C-C motif) | 205099_s_at |
| | | | | receptor 1 | |
| TNF | 0.067 | 3.471 | 6.96E-03 | tumor necrosis factor | 207113_s_at |
| | | | | (TNF superfamily, | |
| | | | | member 2) | |
| LYRIC | 0.067 | 0.074 | 6.95E-03 | LYRIC/3D3 | 212248_at |
| FLJ21603 | 0.067 | 0.052 | 6.98E-03 | zinc finger protein 552 | 219741_x_at |
| PRKAG1 | 0.067 | 8.572 | 7.02E-03 | protein kinase, AMP- | 201805_at |
| | | | | activated, gamma 1 non- | |
| | | | | catalytic subunit | |
| NISCH | 0.067 | 0.054 | 7.00E-03 | nischarin | 201591_s_at |
| COX7C | 0.067 | 0.116 | 7.13E-03 | cytochrome c oxidase | 201134_x_at |
| | | | | subunit VIIc | |
| BRD3 | 0.067 | 0.076 | 7.13E-03 | BRD3, bromodomain | 212547_at |
| | | | | containing 3 | |
| SRF72 | 0.068 | 3.342 | 7.17E-03 | signal recognition | 208095_s_at |
| | | | | particle 72kDa | |
| CA12 | 0.068 | 4.9 | 7.23E-03 | carbonic anhydrase XII | 203963_at |
| KLF3 | 0.068 | 0.147 | 7.26E-03 | Kruppel-like factor 3 | 219657_s_at |
| | | | | (basic) | |
| FLJ20546 | 0.068 | 9.408 | 7.35E-03 | interphase cyctoplasmic | 219122_s_at |
| | | | | foci protein 45 | |
| BLM | 0.068 | 13.128 | 7.35E-03 | Bloom syndrome | 205733_at |
| PLAC8 | 0.069 | 0.161 | 7.39E-03 | placenta-specific 8 | 219014_at |
| KIAA1012 | 0.069 | 32.462 | 7.42E-03 | KIAA1012 | 207305_s_at |
| SRPK1 | 0.069 | 7.399 | 7.45E-03 | SFRS protein kinase 1 | 202200_s_at |
| CLECSF12 | 0.069 | 0.260 | 7.47E-03 | C-type (calcium | 221698_s_at |
| | | | | dependent, carbohydrate- | |
| | | | | recognition domain) | |
| | | | | lectin, superfamily | |
| | | | | member 12 | |
| COPS8 | 0.069 | 60.228 | 7.51E-03 | COP9 constitutive | 202142_at |
| | | | | photomorphogenic | |
| | | | | homolog subunit 8 | |
| | | | | (Arabidopsis) | |
| MGC11256 | 0.069 | 11.319 | 7.51E-03 | hypothetical protein | 218358_at |
| | | | | MGC11256 | |
| MRPS11 | 0.069 | 10.289 | 7.51E-03 | mitochondrial ribosomal | 211595_s_at |
| | | | | protein S11 | |
| SLC2A14 | 0.069 | 0.219 | 7.52E-03 | solute carrier family 2 | 216236_s_at |
| | | | | (facilitated glucose | |
| | | | | transporter), member 14 | |
| CUTL1 | 0.069 | 0.106 | 7.53E-03 | cut-like 1, CCAAT | 202367_at |
| | | | | displacement protein | |
| | | | | (Drosophila) | |
| PAFAH1B1 | 0.069 | 18.233 | 7.55E-03 | platelet-activating factor | 200816_s_at |
| | | | | acetylhydrolase, isoform | |
| | | | | Ib, alpha subunit 45kDa | |
| AKAP13 | 0.069 | 8.326 | 7.57E-03 | A kinase (PRKA) anchor | 209534_x_at |
| | | | | protein 13 | |
| HIST1H4C | 0.071 | 4.545 | 7.77E-03 | histone 1, H4c | 205967_at |
| PSME4 | 0.071 | 0.102 | 7.90E-03 | proteasome (prosome, | 212219_at |
| | | | | macropain) activator | |
| | | | | subunit 4 | |
| KIAA0152 | 0.072 | 23.99 | 7.98E-03 | KIAA0152 gene product | 200617_at |
| CINP | 0.072 | 18.638 | 7.94E-03 | cyclin-dependent kinase | 218267_at |
| | | | | 2-interacting protein | |
| EIF5B | 0.072 | 5.498 | 7.95E-03 | eukaryotic translation | 201027_s_at |
| | | | | initiation factor 5B | |
| G0S2 | 0.072 | 0.266 | 7.97E-03 | putative lymphocyte | 213524_s_at |
| | | | | G0/G1 switch gene | |
| SENP3 | 0.072 | 15.871 | 8.02E-03 | SUMO1/sentrin/SMT3 | 203871_at |
| | | | | specific protease 3 | |
| HNRPR | 0.072 | 10.584 | 8.12E-03 | heterogeneous nuclear | 208765_s_at |
| | | | | ribonucleoprotein R | |
| FN5 | 0.072 | 10.552 | 8.11E-03 | FN5 protein | 219806_s_at |
| ATP6V1C1 | 0.072 | 6.714 | 8.11E-03 | ATPase, H+ transporting, | 202874_s_at |
| | | | | lysosomal 42kDa, V1 | |
| | | | | subunit C, isoform 1 | |
| COL18A1 | 0.072 | 0.057 | 8.11E-03 | collagen, type XVIII, | 209081_s_at |
| | | | | alpha 1 | |
| EEF1E1 | 0.072 | 9.813 | 8.14E-03 | eukaryotic translation | 204905_s_at |
| | | | | elongation factor 1 | |
| | | | | epsilon 1 | |
| TANK | 0.072 | 6.588 | 8.15E-03 | TRAF family member- | 207616_s_at |
| | | | | associated NFKB | |
| | | | | activator | |
| IFIT5 | 0.073 | 0.128 | 8.23E-03 | interferon-induced | 203595_s_at |
| | | | | protein with | |
| | | | | tetratricopeptide repeats 5 | |
| TUBA3 | 0.073 | 0.124 | 8.25E-03 | tubulin, alpha 3 | 209118_s_at |
| UBE2J1 | 0.074 | 10.265 | 8.35E-03 | ubiquitin-conjugating | 217823_s_at |
| | | | | enzyme E2, J1 (UBC6 | |
| | | | | homolog, yeast) | |
| PER1 | 0.074 | 0.118 | 8.35E-03 | period homolog 1 | 36829_at |
| | | | | (Drosophila) | |
| DGCR14 | 0.074 | 0.150 | 8.36E-03 | DiGeorge syndrome | 32032_at |
| | | | | critical region gene 14 | |
| CGI-49 | 0.074 | 15.54 | 8.39E-03 | CGI-49 protein | 201825_s_at |
| CEACAM8 | 0.074 | 0.244 | 8.43E-03 | carcmoembryonic | 206676_at |
| | | | | antigen-related cell | |
| | | | | adhesion molecule 8 | |
| GNAI2 | 0.074 | 0.178 | 8.43E-03 | guanine nucleotide | 201040_at |
| | | | | binding protein (G | |
| | | | | protein), alpha inhibiting | |
| | | | | activity polypeptide 2 | |
| ACAT1 | 0.074 | 13.587 | 8.50E-03 | acetyl-Coenzyme A | 205412_at |
| | | | | acetyltransferase 1 | |
| | | | | (acetoacetyl Coenzyme | |
| | | | | A thiolase) | |
| GOT1 | 0.074 | 6.694 | 8.50E-03 | glutamic-oxaloacetic | 208813_at |
| | | | | transaminase 1, soluble | |
| | | | | (aspartate | |
| | | | | aminotransferase 1) | |
| SMG1 | 0.074 | 0.096 | 8.48E-03 | PI-3-kinase-related | 208118_x_at |
| | | | | kinase SMG-1 | |
| 13CDNA73 | 0.074 | 0.086 | 8.50E-03 | hypothetical protein | 204072_s_at |
| | | | | CG003 | |
| TUBB | 0.074 | 0.250 | 8.55E-03 | tubulin, beta polypeptide | 204141_at |
| CHD4 | 0.075 | 12.6 | 8.60E-03 | chromodomain helicase | 201183_s_at |
| | | | | DNA binding protein 4 | |
| RGS10 | 0.075 | 4.866 | 8.70E-03 | regulator of G-protein | 214000_s_at |
| | | | | signalling 10 | |
| CAMP | 0.075 | 0.253 | 8.74E-03 | cathelicidin antimicrobial | 210244_at |
| | | | | peptide | |
| APOM | 0.075 | 0.107 | 8.78E-03 | apolipoprotein M | 214910_s_at |
| FLJ21868 | 0.076 | 0.096 | 8.80E-03 | transducer of regulated | 218648_at |
| | | | | cAMP response element- | |
| | | | | binding protein (CREB) 3 | |
| MCM10 | 0.076 | 4.325 | 8.83E-03 | MCM10 | 220651_s_at |
| | | | | minichromosome | |
| | | | | maintenance deficient 10 | |
| | | | | (S. cerevisiae) | |
| C11ORF2 | 0.076 | 0.215 | 8.86E-03 | chromosome 11 open | 217969_at |
| | | | | reading frame2 | |
| Unknown | 0.076 | 0.089 | 8.91E-03 | gene of unknown | 217625_x_at |
| | | | | function | |
| MPZL1 | 0.076 | 26.093 | 8.96E-03 | myelin protein zero-like 1 | 201874_at |
| MRPL48 | 0.077 | 15.923 | 9.04E-03 | mitochondrial ribosomal | 218281_at |
| | | | | protein L48 | |
| SET | 0.077 | 10.103 | 9.11E-03 | SET translocation | 210231_x_at |
| | | | | (myeloid leukemia- | |
| | | | | associated) | |
| C16ORF35 | 0.077 | 0.221 | 9.11E-03 | chromosome 16 open | 214273_x_at |
| | | | | reading frame 35 | |
| RARA | 0.077 | 0.123 | 9.07E-03 | retinoic acid receptor, | 203749_s_at |
| | | | | alpha | |
| T1 | 0.077 | 0.084 | 9.11E-03 | Tularik gene 1 | 56829_at |
| NCBP1 | 0.077 | 6.422 | 9.14E-03 | nuclear cap binding | 209520_s_at |
| | | | | protein subunit 1, 80kDa | |
| INHBA | 0.077 | 3.484 | 9.19E-03 | inhibin, beta A (activin | 210511_s_at |
| | | | | A, activin AB alpha | |
| | | | | polypeptide) | |
| NINJ2 | 0.077 | 0.217 | 9.19E-03 | ninjurin 2 | 219594_at |
| NCOA1 | 0.077 | 0.124 | 9.15E-03 | nuclear receptor | 290105_at |
| | | | | coactivator 1 | |
| JMJD1 | 0.077 | 0.113 | 9.18E-03 | jumonji domain | 212689_s_at |
| | | | | containing 1A | |
| UVRAG | 0.077 | 0.043 | 9.17E-03 | UV radiation resistance | 203241_at |
| | | | | associated gene | |
| CXCL13 | 0.077 | 4.537 | 9.22E-03 | chemokine (C-X-C | 205242_at |
| | | | | motif) ligand 13 (B-cell | |
| | | | | chemoattractant) | |
| CYB5 | 0.078 | 7.271 | 9.28E-03 | cytochrome b-5 | 209366_x_at |
| KLRD1 | 0.079 | 13.813 | 9.47E-03 | killer cell lectin-like | 207796_x_at |
| | | | | receptor subfamily D, | |
| | | | | member 1 | |
| APG12L | 0.079 | 10.273 | 9.48E-03 | APG12 autophagy 12- | 213026_at |
| | | | | like (S. cerevisiae) | |
| PLEKHB2 | 0.079 | 0.035 | 9.48E-03 | pleckstrin homology | 201410_at |
| | | | | domain containing, | |
| | | | | family B (evectins) | |
| | | | | member 2 | |
| TNPO1 | 0.079 | 16.302 | 9.50E-03 | transportin 1 | 207657_x_at |
| PDPK1 | 0.079 | 6.544 | 9.55E-03 | 3-phosphoinositide | 32029_at |
| | | | | dependent protein | |
| | | | | kinase-1 | |
| SLCO3A1 | 0.079 | 0.139 | 9.54E-03 | solute carrier organic | 210542_s_at |
| | | | | anion transporter family, | |
| | | | | member 3A1 | |
| YT521 | 0.079 | 0.097 | 9.52E-03 | splicing factor YT521-B | 212455_at |
| FOSL2 | 0.079 | 0.091 | 9.52E-03 | FOS-like antigen 2 | 218881_s_at |
| NDUFB8 | 0.079 | 0.060 | 9.58E-03 | NADH dehydrogenase | 214241_at |
| | | | | (ubiquinone) 1 beta | |
| | | | | subcomplex, 8, 19kDa | |
| TRIM44 | 0.079 | 6.37 | 9.59E-03 | tripartite motif- | 217759_at |
| | | | | containing 44 | |
| UPS4 | 0.079 | 23.919 | 9.68E-03 | ubiquitin specific | 202682_s_at |
| | | | | protease 4 (proto- | |
| | | | | oncogene) | |
| SEC61A1 | 0.079 | 14.769 | 9.69E-03 | Sec61 alpha 1 subunit (S. | 217716_s_at |
| | | | | cerevisiae) | |
| SF3B1 | 0.079 | 0.048 | 9.69E-03 | splicing factor 3b, | 201071_x_at |
| | | | | subunit 1, 155kDa | |
| HA-1 | 0.080 | 0.193 | 9.73E-03 | minor histocompatibility | 212873_at |
| | | | | antigen HA-1 | |
| SMARCD3 | 0.080 | 0.202 | 9.78E-03 | SWI/SNF related, matrix | 204099_at |
| | | | | associated, actin | |
| | | | | dependent regulator of | |
| | | | | chromatin, subfamily d, | |
| | | | | member 3 | |
| AP3D1 | 0.080 | 79.171 | 9.81E-03 | adaptor-related protein | 206592_s_at |
| | | | | complex 3, delta 1 | |
| | | | | subunit | |
| EEG1 | 0.080 | 6.217 | 9.81E-03 | solute carrier family 43, | 213113_s_at |
| | | | | member 3 | |
| SIGIRR | 0.080 | 0.267 | 9.80E-03 | single Ig IL-1R-related | 218921_at |
| | | | | molecule | |
| Unknown | 0.080 | 0.157 | 9.83E-03 | gene of unknown | 221988_at |
| | | | | function | |
| S100A10 | 0.080 | 0.077 | 9.85E-03 | S100 calcium binding | 200872_at |
| | | | | protein A10 (annexin II | |
| | | | | ligand, calpactin I, light | |
| | | | | polypeptide (p11)) | |
| KIAA0553 | 0.080 | 4.734 | 9.87E-03 | KIAA0553 protein | 212487_at |
| PTMAP7 | 0.081 | 4.827 | 9.99E-03 | prothymosin, alpha | 208549_x_at |
| | | | | pseudogene 7 | |
| FLJ12671 | 0.081 | 11.68 | 1.00E-02 | hypothetical protein | 208114_s_at |
| | | | | FLJ12671 | |
| MELK | 0.081 | 4.422 | 1.00E-02 | maternal embryonic | 204825_at |
| | | | | leucine zipper kinase | |
| ELMO2 | 0.081 | 0.052 | 1.00E-02 | engulfment and cell | 55692_at |
| | | | | motility 2 (ced-12 | |
| | | | | homolog, C. elegans) | |
| DDX41 | 0.081 | 10.888 | 1.01E-02 | DEAD (Asp-Glu-Ala- | 217840_at |
| | | | | Asp) box polypeptide 41 | |
| MGC39821 | 0.081 | 14.096 | 1.01E-02 | hypothetical protein | 216126_at |
| | | | | MGC39821 | |
| IMMT | 0.081 | 24.233 | 1.02E-02 | inner membrane protein, | 200955_at |
| | | | | mitochondrial (mitofilin) | |
| ASNA1 | 0.081 | 8.267 | 1.01E-02 | arsA arsenite transporter, | 202024_at |
| | | | | ATP-binding, homolog 1 | |
| | | | | (bacterial) | |
| TM7SF1 | 0.081 | 7.167 | 1.01E-02 | transmembrane 7 | 204137_at |
| | | | | superfamily member 1 | |
| | | | | (upregulated in kidney) | |
| CDC2 | 0.081 | 5.354 | 1.02E-02 | cell division cycle 2, G1 | 203213_at |
| | | | | to S and G2 to M | |
| G3BP2 | 0.081 | 0.061 | 1.02E-02 | Ras-GTPase activating | 208840_s_at |
| | | | | protein SH3 domain- | |
| | | | | binding protein 2 | |
| KIAA0143 | 0.081 | 24.981 | 1.02E-02 | KIAA0143 protein | 212150_at |
| CSNK1A1 | 0.081 | 33.586 | 1.02E-02 | caseinkinase 1, alpha 1 | 213086_s_at |
| LOC203069 | 0.081 | 0.050 | 1.02E-02 | hypothetical protein | 35156_at |
| | | | | LOC203069 | |
| MRPL28 | 0.081 | 15.604 | 1.02E-02 | mitochondrial ribosomal | 204599_s_at |
| | | | | protein L28 | |
| FLJ20989 | 0.081 | 9.325 | 1.03E-02 | hypothetical protein | 218187_s_at |
| | | | | FLJ20989 | |
| SLC18A2 | 0.081 | 0.148 | 1.03E-02 | solute carrier family 18 | 213549_at |
| | | | | (vesicular monoamine), | |
| | | | | member 2 | |
| KIAA0399 | 0.081 | 0.142 | 1.03E-02 | zinc finger, ZZ-type with | 212601_at |
| | | | | EF hand domain 1 | |
| MTCP1 | 0.081 | 0.114 | 1.03E-02 | mature T-cell | 210212_x_at |
| | | | | proliferation 1 | |
| POT1 | 0.081 | 12.322 | 1.03E-02 | protection of telomeres 1 | 204354_at |
| CUL4A | 0.082 | 15.206 | 1.03E-02 | cullin 4A | 201423_s_at |
| LAPTM4B | 0.082 | 4.808 | 1.04E-02 | lysosomal associated | 214039_s_at |
| | | | | protein transmembrane 4 | |
| | | | | beta | |
| TYMS | 0.082 | 3.794 | 1.04E-02 | thymidylate synthetase | 202589_at |
| ERCC1 | 0.082 | 0.089 | 1.04E-02 | excision repair cross- | 203719_at |
| | | | | complementing rodent | |
| | | | | repair deficiency, | |
| | | | | complementation group 1 | |
| | | | | (includes overlapping | |
| | | | | antisense sequence) | |
| PSMC3 | 0.082 | 3.801 | 1.04E-02 | proteasome (prosome, | 201267_s_at |
| | | | | macropain) 26S subunit, | |
| | | | | ATPase, 3 | |
| PIAS1 | 0.082 | 5.994 | 1.05E-02 | protein inhibitor of | |
| | | | | activated STAT, 1 | |
| CDYL | 0.083 | 14.787 | 1.07E-02 | chromodomain protein, | 203098_at |
| | | | | Y-like | |
| ACAT2 | 0.083 | 6.322 | 1.07E-02 | acetyl-Coenzyme A | 209608_s_at |
| | | | | acetyltransferase 2 | |
| | | | | (acetoacetyl Coenzyme | |
| | | | | A thiolase) | |
| RBBP8 | 0.083 | 13.511 | 1.07E-02 | retinoblastoma binding | 203344_s_at |
| | | | | protein 8 | |
| ATF1 | 0.083 | 13.031 | 1.07E-02 | activating transcription | 222103_at |
| | | | | factor 1 | |
| CBX1 | 0.083 | 7.07 | 1.08E-02 | chromobox homolog 1 | 201518_at |
| | | | | (HP1 beta homolog | |
| | | | | Drosophila) | |
| CLK1 | 0.084 | 0.090 | 1.08E-02 | CDC-like kinase 1 | 210346_s_at |
| PBF | 0.084 | 0.218 | 1.08E-02 | zinc finger protein 395 | 218149_s_at |
| SLC2A4RG | 0.084 | 0.080 | 1.09E-02 | SLC2A4 regulator | 218494_s_at |
| KAI1 | 0.084 | 6.32 | 1.09E-02 | kangai 1 (suppression of | 203904_x_at |
| | | | | tumorigenicity 6, | |
| | | | | prostate; CD82 antigen | |
| | | | | (R2 leukocyte antigen, | |
| | | | | antigen detected by | |
| | | | | monoclonal and antibody | |
| | | | | IA4)) | |
| EP300 | 0.084 | 10.703 | 1.10E-02 | E1A binding protein | 213579_s_at |
| | | | | p300 | |
| DNAJB6 | 0.085 | 4.765 | 1.11E-02 | DnaJ (Hsp40) homolog, | 209015_s_at |
| | | | | subfamily B, member 6 | |
| UGDH | 0.085 | 5.91 | 1.12E-02 | UDP-glucose | 203343_at |
| | | | | dehydrogenase | |
| C20ORF9 | 0.085 | 56.405 | 1.12E-02 | chromosome 20 open | 218709_s_at |
| | | | | reading frame 9 | |
| NIT2 | 0.086 | 7.547 | 1.13E-02 | Nit protein 2 | 218557_at |
| RBM5 | 0.086 | 13.578 | 1.13E-02 | RNA binding motif | 201394_s_at |
| | | | | protein 5 | |
| HNRPH3 | 0.086 | 0.088 | 1.14E-02 | heterogeneous nuclear | 210110_x_at |
| | | | | ribonucleoprotein H3 | |
| | | | | (2H9) | |
| DHX40 | 0.087 | 0.073 | 1.15E-02 | DEAH (Asp-Glu-Ala- | 218277_s_at |
| | | | | His) box polypeptide 40 | |
| PSMB9 | 0.087 | 8.276 | 1.15E-02 | proteasome (prosome, | 204279_at |
| | | | | macropain) subunit, beta | |
| | | | | type, 9 (large | |
| | | | | multifunctional protease 2) | |
| MSCP | 0.087 | 7.256 | 1.15E-02 | MSCP, mitochondrial | 221155_x_at |
| | | | | solute carrier protein | |
| C10ORF22 | 0.087 | 6.428 | 1.16E-02 | chromosome 10 open | 212500_at |
| | | | | reading frame 22 | |
| UROD | 0.087 | 13.864 | 1.16E-02 | uroporphyrinogen | 208971_at |
| | | | | decarboxylase | |
| MTSS1 | 0.087 | 16.897 | 1.16E-02 | metastasis suppressor 1 | 203037_s_at |
| GAB2 | 0.088 | 0.115 | 1.17E-02 | GRB2-associated | 203853_s_at |
| | | | | binding protein 2 | |
| FLJ11856 | 0.089 | 15.457 | 1.19E-02 | putative G-protein | 218151_x_at |
| | | | | coupled receptor | |
| | | | | GPCR41 | |
| SNX1 | 0.089 | 0.246 | 1.19E-02 | sorting nexin 1 | 213364_s_at |
| CGI-94 | 0.089 | 12.952 | 1.19E-02 | comparative gene | 218235_s_at |
| | | | | identification transcript | |
| | | | | 94 | |
| ANAPC2 | 0.089 | 0.250 | 1.19E-02 | anaphase promoting | 218555_at |
| | | | | complex subunit 2 | |
| PPBP | 0.090 | 7.479 | 1.21E-02 | pro-platelet basic protein | 214146_s_at |
| | | | | (chemokine (C-X-C | |
| | | | | motif) ligand 7) | |
| LOC51315 | 0.090 | 6.733 | 1.21E-02 | hypothetical protein | 218303_x_at |
| | | | | LOC51315 | |
| ARHGEF2 | 0.090 | 9.18 | 1.21E-02 | rho/rac guanine | 207629_s_at |
| | | | | nucleotide exchange | |
| | | | | factor (GEF) 2 | |
| ANKRD10 | 0.090 | 0.121 | 1.22E-02 | ankyrin repeat domain 10 | 218093_s_at |
| NUDC | 0.091 | 10.798 | 1.23E-02 | nuclear distribution gene | 201173_x_at |
| | | | | C homolog (A. nidulans) | |
| PFKP | 0.091 | 6.294 | 1.24E-02 | phosphofructokinase, | 201037_at |
| | | | | platelet | |
| NDUFAF1 | 0.091 | 5.219 | 1.24E-02 | NADH dehydrogenase | 204125_at |
| | | | | (ubiquinone) 1 alpha | |
| | | | | subcomplex, assembly | |
| | | | | factor 1 | |
| STX7 | 0.091 | 4.414 | 1.24E-02 | STX7, syntaxin 7 | 212632_at |
| SLAMF8 | 0.091 | 3.465 | 1.24E-02 | SLAM family member 8 | 219386_s_at |
| CPSF6 | 0.091 | 0.146 | 1.24E-02 | cleavage and | 202469_s_at |
| | | | | polyadenylation specific | |
| | | | | factor 6, 68kDa | |
| LSM2 | 0.091 | 8.984 | 1.25E-02 | LSM2 homolog, U6 | 209449_at |
| | | | | small nuclear RNA | |
| | | | | associated (S. cerevisiae) | |
| LAP1B | 0.091 | 0.054 | 1.25E-02 | lamina-associated | 212408_at |
| | | | | polypeptide 1B | |
| RASA4 | 0.091 | 0.189 | 1.25E-02 | RAS p21 protein | 212707_s_at |
| | | | | activator 4 | |
| POGK | 0.091 | 6.766 | 1.26E-02 | pogo transposable | 218229_s_at |
| | | | | element with KRAB | |
| | | | | domain | |
| SSR1 | 0.092 | 0.122 | 1.26E-02 | signal sequence receptor, | 200891_s_at |
| | | | | alpha (translocon- | |
| | | | | associated protein alpha) | |
| ATP1B1 | 0.092 | 5.102 | 1.27E-02 | ATPase, Na+/K+ | 201242_s_at |
| | | | | transporting, beta 1 | |
| | | | | polypeptide | |
| PA2G4 | 0.092 | 7.067 | 1.27E-02 | proliferation-associated | 208676_s_at |
| | | | | 2G4, 38kDa | |
| USF2 | 0.092 | 0.244 | 1.27E-02 | upstream transcription | 202152_x_at |
| | | | | factor 2, c-fos interacting | |
| CASP3 | 0.092 | 14.85 | 1.27E-02 | caspase 3, apoptosis- | 202763_at |
| | | | | related cysteine protease | |
| PPIB | 0.092 | 5.353 | 1.28E-02 | peptidylprolyl isomerase | 200968_s_at |
| | | | | B (cyclophilin B) | |
| SP2 | 0.093 | 0.135 | 1.29E-02 | Sp2 transcription factor | 204367_at |
| GRP58 | 0.093 | 15.372 | 1.29E-02 | glucose regulated | 208612_at |
| | | | | protein, 58kDa | |
| KIAA0863 | 0.093 | 0.097 | 1.29E-02 | KIAA0863 protein | 203322_at |
| CD24 | 0.094 | 0.031 | 1.31E-02 | CD24 antigen (small cell | 266_s_at |
| | | | | lung carcinoma cluster 4 | |
| | | | | antigen) | |
| TCFL1 | 0.094 | 19.522 | 1.31E-02 | transcription factor-like 1 | 202261_at |
| MCM4 | 0.094 | 5.113 | 1.31E-02 | MCM4 minichromosome | 222037_at |
| | | | | maintenance deficient 4 | |
| | | | | (S. cerevisiae) | |
| SULT1A1 | 0.094 | 0.047 | 1.31E-02 | sulfotransferase family, | 211385_x_at |
| | | | | cytosolic, 1A, phenol- | |
| | | | | preferring, member 1 | |
| FARSLA | 0.094 | 4.776 | 1.32E-02 | phenylalanine-tRNA | 216602_s_at |
| | | | | synthetase-like, alpha | |
| | | | | subunit | |
| PCF11 | 0.094 | 0.197 | 1.32E-02 | pre-mRNA cleavage | 203378_at |
| | | | | complex II protein Pcfl 1 | |
| TNS | 0.094 | 0.064 | 1.32E-02 | tensin | 221747_at |
| HBP1 | 0.094 | 0.062 | 1.32E-02 | HMG-box transcription | 209102_s_at |
| | | | | factor 1 | |
| ILVBL | 0.094 | 31.351 | 1.33E-02 | ilvB (bacterial | 210624_s_at |
| | | | | acetolactate synthase)- | |
| | | | | like | |
| ZNF24 | 0.094 | 24.761 | 1.33E-02 | zinc finger protein 24 | 212534_at |
| | | | | (KOX 17) | |
| DYRK1A | 0.094 | 0.134 | 1.33E-02 | dual-specificity tyrosine- | 209033_s_at |
| | | | | (Y)-phosphorylation | |
| | | | | regulated kinase 1A | |
| GGA1 | 0.094 | 0.088 | 1.34E-02 | golgi associated, gamma | 45572_s_at |
| | | | | adaptin ear containing, | |
| | | | | ARF binding protein 1 | |
| WDR26 | 0.094 | 0.037 | 1.33E-02 | WD repeat domain 26 | 218107_at |
| HNRPAB | 0.094 | 7.05 | 1.34E-02 | heterogeneous nuclear | 201277_s_at |
| | | | | ribonucleoprotein A/B | |
| NOLC1 | 0.094 | 5.681 | 1.34E-02 | nucleolar and coiled- | 205895_s_at |
| | | | | body phosphoprotein 1 | |
| ASH2L | 0.094 | 23.135 | 1.34E-02 | ash2 (absent, small, or | 209517_s_at |
| | | | | homeotic)-like | |
| | | | | (Drosophila) | |
| PPM1F | 0.094 | 0.140 | 1.34E-02 | protein phosphatase 1F | 37384_at |
| | | | | (PP2C domain | |
| | | | | containing) | |
| SASH1 | 0.095 | 8.308 | 1.36E-02 | SAM and SH3 domain | 41644_at |
| | | | | containing 1 | |
| RPL13 | 0.095 | 0.167 | 1.36E-02 | ribosomal protein L13 | 214351_x_at |
| RPS2 | 0.095 | 3.043 | 1.37E-02 | DNA replication | 221521_s_at |
| | | | | complex GINS protein | |
| | | | | PSF2 | |
| TMEM14A | 0.095 | 6.614 | 1.37E-02 | transmembrane protein 14A | 218477_at |
| FLJ35827 | 0.095 | 0.121 | 1.37E-02 | hypothetical protein | 212969_x_at |
| | | | | FLJ35827 | |
| REPIN1 | 0.096 | 0.114 | 1.38E-02 | replication initiator 1 | 219041_s_at |
| EI24 | 0.096 | 14.543 | 1.39E-02 | etoposide induced 2.4 | 208289_s_at |
| | | | | mRNA | |
| MRPS7 | 0.096 | 9.557 | 1.39E-02 | mitochondrial ribosomal | 217932_at |
| | | | | protein S7 | |
| TRIM8 | 0.096 | 0.173 | 1.39E-02 | tripartite motif- | 221012_s_at |
| | | | | containing 8 | |
| ERP70 | 0.096 | 28.582 | 1.39E-02 | protein disulfide | 208658_at |
| | | | | isomerase related protein | |
| | | | | (calcium-binding protein, | |
| | | | | intestinal-related) | |
| GABPB2 | 0.096 | 6.265 | 1.39E-02 | GA binding protein | 204618_s_at |
| | | | | transcription factor, beta | |
| | | | | subunit 2, 47kDa | |
| KIAA0763 | 0.096 | 0.150 | 1.40E-02 | KIAA0763 gene product | 203906_at |
| NGX6 | 0.096 | 0.125 | 1.40E-02 | chromosome 9 open | 207839_s_at |
| | | | | reading frame 127 | |
| CREBL2 | 0.097 | 15.292 | 1.41E-02 | cAMP responsive | 201989_s_at |
| | | | | element binding protein- | |
| | | | | like 2 | |
| NEK7 | 0.097 | 9.293 | 1.41E-02 | NIMA (never in mitosis | 212530_at |
| | | | | gene a)-related kinase 7 | |
| CAMLG | 0.097 | 0.165 | 1.41E-02 | calcium modulating | 203538_at |
| | | | | ligand | |
| INSIG2 | 0.097 | 9.42 | 1.42E-02 | insulin induced gene 2 | 209566_at |
| CCT7 | 0.097 | 4.279 | 1.42E-02 | chaperonin containing | 200812_at |
| | | | | TCP1, subunit 7 (eta) | |
| CCNI | 0.097 | 0.063 | 1.43E-02 | cyclin 1 | |
| DAPP1 | 0.098 | 7.907 | 1.44E-02 | dual adaptor of | 219290_x_at |
| | | | | phosphotyrosine and 3- | |
| | | | | phosphoinositides | |
| RBM8A | 0.098 | 4.954 | 1.45E-02 | RNA binding motif | 217857_s_at |
| | | | | protein 8A | |
| USP21 | 0.098 | 0.207 | 1.45E-02 | ubiquitin specific | 218367_x_at |
| | | | | protease 21 | |
| PRKCI | 0.098 | 6.662 | 1.45E-02 | protein kinase C, iota | 209678_s_at |
| WBP11 | 0.098 | 0.042 | 1.45E-02 | WW domain binding | 217822_at |
| | | | | protein 11 | |
| CCRL2 | 0.098 | 4.788 | 1.46E-02 | chemokine (C-C motif) | 211434_s_at |
| | | | | receptor-like 2 | |
| MRPL12 | 0.098 | 7.889 | 1.46E-02 | mitochondrial ribosomal | 203931_s_at |
| | | | | protein L12 | |
| PSMB5 | 0.099 | 5.428 | 1.47E-02 | proteasome (prosome, | 208799_at |
| | | | | macropain) subunit, beta | |
| | | | | type, 5 | |
| EBI3 | 0.099 | 3.985 | 1.47E-02 | Epstein-Barr virus | 219424_at |
| | | | | induced gene 3 | |
| BCAP31 | 0.099 | 9.401 | 1.48E-02 | B-cell receptor- | 200837_at |
| | | | | associated protein 31 | |
| ZNF297B | 0.099 | 6.866 | 1.48E-02 | zinc finger protein 297B | 204181_s_at |
| KNS2 | 0.099 | 5.27 | 1.49E-02 | kinesin 2 60/70kDa | 212878_s_at |
| SRD5A1 | 0.100 | 0.212 | 1.49E-02 | steroid-5-alpha- | 204675_at |
| | | | | reductase, alpha | |
| | | | | polypeptide 1 (3-oxo-5 | |
| | | | | alpha-steroid delta 4- | |
| | | | | dehydrogenase alpha 1) | |
| MSH2 | 0.100 | 6.541 | 1.50E-02 | mutS homolog 2, colon | 209421_at |
| | | | | cancer, nonpolyposis | |
| | | | | type 1 (E. coli) | |
| EIF2B1 | 0.100 | 20.633 | 1.50E-02 | eukaryotic translation | 201632_at |
| | | | | initiation factor 2B, | |
| | | | | subunit 1 alpha, 26kDa | |
| ID3 | 0.100 | 0.052 | 1.50E-02 | inhibitor of DNA binding | 207826_s_at |
| | | | | 3, dominant negative | |
| | | | | helix-loop-helix protein | |
| IRAK1BP1 | 0.100 | 0.191 | 1.51E-02 | interleukin-1 receptor- | 213074_at |
| | | | | associated kinase 1 | |
| | | | | binding protein 1 | |

TABLE 33
Annotation of Genes Associated with Meningoencephalitis

| | | | Ingenuity assignment to one of the | |
|---|---|---|---|---|
| | | | following functions: cell-cycle | |
| | | | (includes DNA synthesis, cell | |
| | | | growth and proliferation), cell | |
| | | | death, cell signaling and | |
| | | | interaction (includes cell signaling | |
| | | | and cell-to-cell signaling and | |
| | | | interaction), immune functions | |
| | | | (includes immune and lymphatic | |
| | Odds Ratio for | | system development and function | Identified by |
| | association with | FDR association | and immune response), protein | more than one |
| Gene | meningoencephalitis | meningoencephalitis | synthesis and trafficking | probeset |
| STAT1 | 230.416 | 0.004 | Yes | Yes |
| NHP2L1 | 3136.203 | 0.010 | Yes | No |
| C10ORF7 | 673.31 | 0.010 | Yes | No |
| ZW10 | 470.958 | 0.010 | Yes | No |
| ICMT | 417.532 | 0.010 | Yes | No |
| RABGAP1 | 303.809 | 0.010 | Yes | No |
| BRD2 | 56.318 | 0.010 | Yes | Yes |
| KPNB1 | 32.282 | 0.010 | Yes | Yes |
| GZMB | 31.809 | 0.010 | Yes | No |
| KLF2 | 0.038 | 0.010 | Yes | No |
| STK17B | 0.025 | 0.010 | Yes | No |
| FLJ11806 | 651.763 | 0.010 | Not assigned to function by | Yes |
| | | | Ingenuity | |
| C12ORF22 | 459.155 | 0.010 | Not assigned to function by | No |
| | | | Ingenuity | |
| SEC24C | 66.791 | 0.010 | Not assigned to function by | No |
| | | | Ingenuity | |
| FNBP3 | 13.972 | 0.010 | Not assigned to function by | No |
| | | | Ingenuity | |
| JARID1B | 0.006 | 0.010 | Not assigned to function by | Yes |
| | | | Ingenuity | |
| TRAP240 | 68.675 | 0.010 | No | No |
| STAT3 | 6.606 | 0.011 | Yes | No |
| BTG2 | 0.033 | 0.011 | Yes | No |
| MGC21416 | 8.373 | 0.011 | Not assigned to function by | Yes |
| | | | Ingenuity | |
| OSBPL8 | 4.201 | 0.011 | Not assigned to function by | No |
| | | | Ingenuity | |
| HEAB | 0.001 | 0.011 | Not assigned to function by | No |
| | | | Ingenuity | |
| UBE2D3 | 0.002 | 0.011 | No | Yes |
| ATP6V1D | 172.543 | 0.011 | Not assigned to function by | Yes |
| | | | Ingenuity | |
| KIF5B | 3.731 | 0.011 | No | No |
| DC8 | 69.508 | 0.012 | Not assigned to function by | No |
| | | | Ingenuity | |
| GLTSCR1 | 0.013 | 0.013 | Not assigned to function by | No |
| | | | Ingenuity | |
| CD84 | 23.97 | 0.013 | No | No |
| UGCG | 14.445 | 0.013 | Yes | No |
| SFRS2IP | 57.281 | 0.014 | Not assigned to function by | Yes |
| | | | Ingenuity | |
| MMP24 | 0.022 | 0.014 | No | No |
| MBD4 | 8.79 | 0.014 | Yes | No |
| TNPO3 | 21.713 | 0.014 | Not assigned to function by | No |
| | | | Ingenuity | |
| GCDH | 50.321 | 0.014 | No | No |
| PABPC1 | 0.006 | 0.014 | No | Yes |
| VDR | 7.092 | 0.014 | Yes | No |
| H2AFY | 0.016 | 0.015 | Not assigned to function by | No |
| | | | Ingenuity | |
| IL2RA | 11.266 | 0.016 | Yes | No |
| STAT5B | 0.029 | 0.016 | Yes | No |
| CBX6 | 34.482 | 0.016 | Not assigned to function by | No |
| | | | Ingenuity | |
| TTC3 | 5.376 | 0.016 | No | No |
| TRIP13 | 17.331 | 0.016 | No | No |
| FLJ23441 | 17.419 | 0.016 | No | No |
| STXBP2 | 0.095 | 0.016 | No | No |
| LRRFIP1 | 18.564 | 0.016 | No | Yes |
| PADI2 | 0.145 | 0.016 | Not assigned to function by | No |
| | | | Ingenuity | |
| HNRPC | 324.673 | 0.016 | Yes | No |
| PTPRC | 4.891 | 0.017 | Yes | No |
| PTDSR | 0.018 | 0.018 | Yes | No |
| TPR | 4.823 | 0.018 | Yes | No |
| HUMGT198A | 8.097 | 0.018 | No | No |
| DUT | 40.207 | 0.018 | Yes | Yes |
| RAB1A | 0.003 | 0.018 | Yes | No |
| HMG2L1 | 5.679 | 0.019 | Not assigned to function by | No |
| | | | Ingenuity | |
| RIN3 | 0.105 | 0.019 | Not assigned to function by | No |
| | | | Ingenuity | |
| PDCD8 | 119.631 | 0.019 | Yes | No |
| CSE1L | 38.753 | 0.019 | Yes | No |
| RNMT | 0.050 | 0.019 | Yes | No |
| TFE3 | 0.041 | 0.019 | Yes | No |
| GLS | 60.862 | 0.019 | No | No |
| FLJ12788 | 167.936 | 0.020 | Not assigned to function by | No |
| | | | Ingenuity | |
| MGAT2 | 20.774 | 0.020 | No | No |
| CGI-37 | 10.964 | 0.021 | Not assigned to function by | No |
| | | | Ingenuity | |
| C21ORF80 | 0.032 | 0.021 | Not assigned to function by | No |
| | | | Ingenuity | |
| LUC7A | 7.673 | 0.021 | No | No |
| FBXW7 | 5.619 | 0.021 | No | No |
| DICER1 | 0.073 | 0.021 | No | No |
| UBCE71P5 | 0.036 | 0.021 | No | No |
| TXNL2 | 152.265 | 0.021 | Not assigned to function by | No |
| | | | Ingenuity | |
| PRKRA | 0.027 | 0.022 | Yes | No |
| BARD1 | 11.776 | 0.022 | Yes | No |
| SH3BP5 | 11.205 | 0.022 | Yes | No |
| OBRGRP | 4.025 | 0.022 | Not assigned to function by | No |
| | | | Ingenuity | |
| C1ORF33 | 12.564 | 0.023 | Not assigned to function by | No |
| | | | Ingenuity | |
| M96 | 9.28 | 0.023 | Not assigned to function by | Yes |
| | | | Ingenuity | |
| DNCL1 | 6.81 | 0.023 | Yes | No |
| BAZ1A | 6.808 | 0.023 | Yes | No |
| NALP1 | 0.133 | 0.023 | Yes | No |
| GNAS | 0.071 | 0.023 | Yes | Yes |
| IPO4 | 29.56 | 0.023 | No | No |
| TH1L | 13.185 | 0.024 | Not assigned to function by | No |
| | | | Ingenuity | |
| IRS2 | 0.060 | 0.024 | Yes | No |
| LTF | 0.325 | 0.025 | Yes | No |
| MIRAB13 | 0.109 | 0.026 | Not assigned to function by | No |
| | | | Ingenuity | |
| BATF | 9.718 | 0.026 | Yes | No |
| FLN29 | 176.965 | 0.026 | Not assigned to function by | No |
| | | | Ingenuity | |
| HAX1 | 34.12 | 0.026 | No | No |
| MYO1B | 18.41 | 0.026 | No | No |
| SLC5A3 | 4.832 | 0.026 | No | No |
| PADI4 | 0.108 | 0.026 | No | No |
| STK10 | 0.052 | 0.026 | Not assigned to function by | No |
| | | | Ingenuity | |
| RAB2 | 0.002 | 0.027 | Yes | No |
| BPI | 0.219 | 0.027 | Yes | No |
| DEFA4 | 0.196 | 0.027 | Not assigned to function by | No |
| | | | Ingenuity | |
| KPNA6 | 34.224 | 0.028 | Yes | No |
| C19ORF10 | 45.058 | 0.028 | Yes | No |
| DKFZP564G2022 | 11.966 | 0.028 | Not assigned to function by | No |
| | | | Ingenuity | |
| SNRK | 0.043 | 0.028 | Not assigned to function by | No |
| | | | Ingenuity | |
| GBP1 | 5.53 | 0.028 | Yes | Yes |
| ZFP36 | 0.108 | 0.029 | Yes | No |
| SIPA1 | 0.053 | 0.029 | Yes | No |
| ZNF238 | 0.120 | 0.029 | No | No |
| CXCL10 | 7.825 | 0.029 | Yes | No |
| RRM2 | 5.394 | 0.029 | No | Yes |
| RAB31 | 3.04 | 0.029 | Not assigned to function by | No |
| | | | Ingenuity | |
| USP36 | 0.071 | 0.029 | Not assigned to function by | No |
| | | | Ingenuity | |
| PTP4A1 | 0.034 | 0.029 | No | No |
| DPCK | 156.071 | 0.029 | No | No |
| ALDOC | 11.591 | 0.029 | No | No |
| ZFP36L1 | 0.036 | 0.030 | Yes | No |
| PXMP3 | 39.115 | 0.030 | No | No |
| CYLN2 | 0.060 | 0.030 | Not assigned to function by | No |
| | | | Ingenuity | |
| STAU | 0.078 | 0.031 | No | Yes |
| PHF1 | 0.130 | 0.031 | Not assigned to function by | No |
| | | | Ingenuity | |
| HN1 | 18.055 | 0.031 | Not assigned to function by | No |
| | | | Ingenuity | |
| STOML2 | 6.512 | 0.031 | Not assigned to function by | No |
| | | | Ingenuity | |
| ARID3B | 0.149 | 0.031 | Not assigned to function by | No |
| | | | Ingenuity | |
| IL19 | 8.869 | 0.031 | Yes | No |
| WSX1 | 46.587 | 0.032 | Yes | No |
| NFE2L1 | 33.502 | 0.032 | Yes | No |
| TDE1 | 17.535 | 0.032 | Yes | No |
| POLA | 14.919 | 0.032 | Yes | No |
| NALP2 | 16.21 | 0.032 | Not assigned to function by | No |
| | | | Ingenuity | |
| CKLFSF6 | 13.746 | 0.032 | Not assigned to function by | No |
| | | | Ingenuity | |
| SSH1 | 11.182 | 0.032 | Not assigned to function by | No |
| | | | Ingenuity | |
| DKFZP434H132 | 0.143 | 0.032 | Not assigned to function by | No |
| | | | Ingenuity | |
| JM5 | 0.114 | 0.032 | Not assigned to function by | No |
| | | | Ingenuity | |
| FLJ13479 | 0.010 | 0.032 | Not assigned to function by | No |
| | | | Ingenuity | |
| MINK | 0.145 | 0.032 | No | No |
| MK167 | 69.144 | 0.032 | Yes | No |
| TIMM13 | 22.616 | 0.032 | Yes | No |
| JUNB | 0.108 | 0.032 | Yes | No |
| RBX1 | 27.734 | 0.032 | Not assigned to function by | No |
| | | | Ingenuity | |
| ECHDC1 | 16.161 | 0.032 | Not assigned to function by | No |
| | | | Ingenuity | |
| KIAA0930 | 14.228 | 0.032 | Not assigned to function by | No |
| | | | Ingenuity | |
| HEG | 6.044 | 0.032 | Not assigned to function by | No |
| | | | Ingenuity | |
| MASK | 5.562 | 0.032 | Not assigned to function by | No |
| | | | Ingenuity | |
| C9ORF28 | 0.037 | 0.032 | Not assigned to function by | No |
| | | | Ingenuity | |
| RLF | 0.028 | 0.032 | Not assigned to function by | No |
| | | | Ingenuity | |
| AB026190 | 12.367 | 0.033 | No | No |
| GTF2H5 | 8.729 | 0.033 | Not assigned to function by | No |
| | | | Ingenuity | |
| RBMS1 | 5.153 | 0.033 | Not assigned to function by | Yes |
| | | | Ingenuity | |
| ENIGMA | 0.081 | 0.033 | No | No |
| MIR | 0.128 | 0.033 | No | No |
| SRRM2 | 5.461 | 0.033 | Not assigned to function by | No |
| | | | Ingenuity | |
| MCL1 | 0.058 | 0.033 | Yes | Yes |
| SRR | 15.068 | 0.033 | No | No |
| FACL5 | 89.075 | 0.034 | Yes | No |
| CPSF1 | 0.209 | 0.034 | No | No |
| PTTG1IP | 0.004 | 0.034 | Yes | No |
| AK2 | 17.668 | 0.034 | No | No |
| GTPBP1 | 0.032 | 0.034 | Yes | No |
| UNG | 10.732 | 0.035 | Yes | No |
| RPS28 | 0.215 | 0.035 | Yes | No |
| PAX5 | 8.402 | 0.035 | Yes | No |
| PSMD8 | 11.013 | 0.035 | Not assigned to function by | No |
| | | | Ingenuity | |
| NUDT1 | 10.67 | 0.035 | No | No |
| SLC25A12 | 52.625 | 0.035 | No | No |
| C1ORF24 | 12.539 | 0.036 | Not assigned to function by | No |
| | | | Ingenuity | |
| HTATIP2 | 15.356 | 0.036 | Yes | No |
| SRPK2 | 3.184 | 0.036 | Not assigned to function by | No |
| | | | Ingenuity | |
| PRKAR1A | 16.407 | 0.036 | Yes | No |
| CD80 | 26.52 | 0.036 | Yes | No |
| MGC3248 | 20.329 | 0.036 | Not assigned to function by | No |
| | | | Ingenuity | |
| UBXD2 | 6.211 | 0.036 | Not assigned to function by | No |
| | | | Ingenuity | |
| PDCD11 | 15.892 | 0.036 | Yes | No |
| ISGF3G | 7.836 | 0.036 | Yes | No |
| RAB7 | 0.083 | 0.036 | Yes | No |
| CDC42 | 0.051 | 0.036 | Yes | Yes |
| GALNT1 | 36.544 | 0.036 | No | No |
| STX18 | 23.897 | 0.036 | No | No |
| NFATC1 | 80.225 | 0.036 | Yes | No |
| NR3C1 | 11.05 | 0.036 | Yes | Yes |
| CABIN1 | 0.162 | 0.036 | Yes | No |
| NET1 | 0.146 | 0.036 | Yes | No |
| NFIL3 | 0.116 | 0.036 | Yes | No |
| MOAP1 | 0.115 | 0.036 | Yes | No |
| SKP1A | 0.113 | 0.036 | Yes | No |
| G1P3 | 0.069 | 0.036 | Yes | No |
| BNIP3L | 0.044 | 0.036 | Yes | No |
| PSMD1 | 19.918 | 0.036 | Not assigned to function by | No |
| | | | Ingenuity | |
| PSMD11 | 5.523 | 0.036 | Not assigned to function by | No |
| | | | Ingenuity | |
| H2AV | 0.268 | 0.036 | Not assigned to function by | No |
| | | | Ingenuity | |
| FLJ11127 | 0.069 | 0.036 | Not assigned to function by | No |
| | | | Ingenuity | |
| C6ORF82 | 0.041 | 0.036 | Not assigned to function by | No |
| | | | Ingenuity | |
| COL4A3BP | 17.703 | 0.036 | No | No |
| SEC63 | 7.604 | 0.036 | No | No |
| XTP2 | 4.327 | 0.037 | Not assigned to function by | Yes |
| | | | Ingenuity | |
| MBNL3 | 0.058 | 0.037 | No | No |
| PDHB | 33.983 | 0.037 | No | No |
| CKS1B | 16.085 | 0.038 | Yes | No |
| GALNS | 0.227 | 0.038 | Not assigned to function by | No |
| | | | Ingenuity | |
| EIF5 | 8.566 | 0.038 | Yes | Yes |
| USP12 | 48.047 | 0.038 | Not assigned to function by | No |
| | | | Ingenuity | |
| KIAA0650 | 0.146 | 0.038 | Not assigned to function by | Yes |
| | | | Ingenuity | |
| UQCRFS1 | 0.060 | 0.038 | No | No |
| ACO1 | 49.485 | 0.038 | Yes | No |
| MRPL13 | 9.48 | 0.038 | Yes | No |
| SCGF | 0.120 | 0.038 | Yes | No |
| CHC1L | 0.084 | 0.038 | Not assigned to function by | No |
| | | | Ingenuity | |
| TRIAD3 | 29.384 | 0.039 | Yes | No |
| RFP | 35.742 | 0.039 | Yes | Yes |
| ITGAV | 12.837 | 0.039 | Yes | No |
| RPA3 | 10.718 | 0.039 | Yes | No |
| PSMD13 | 16.384 | 0.039 | Not assigned to function by | Yes |
| | | | Ingenuity | |
| AGTPBP1 | 0.099 | 0.039 | Not assigned to function by | No |
| | | | Ingenuity | |
| CGI-127 | 0.039 | 0.039 | Not assigned to function by | No |
| | | | Ingenuity | |
| ACOX1 | 15.909 | 0.039 | No | No |
| SEC23B | 11.687 | 0.039 | No | No |
| KIF7 | 7.918 | 0.039 | No | No |
| KIAA0892 | 0.071 | 0.039 | Not assigned to function by | No |
| | | | Ingenuity | |
| APLP2 | 0.155 | 0.039 | No | Yes |
| IL7R | 3.182 | 0.039 | Yes | No |
| SR140 | 0.144 | 0.039 | Not assigned to function by | No |
| | | | Ingenuity | |
| TDP1 | 10.611 | 0.040 | Not assigned to function by | No |
| | | | Ingenuity | |
| HMGCL | 11.109 | 0.040 | No | No |
| VDAC3 | 7.789 | 0.040 | No | No |
| HIPK1 | 0.025 | 0.040 | Yes | No |
| CGI-01 | 9.62 | 0.040 | Not assigned to function by | No |
| | | | Ingenuity | |
| FLJ11078 | 0.094 | 0.040 | Not assigned to function by | No |
| | | | Ingenuity | |
| FLJ14639 | 38.716 | 0.040 | No | No |
| CGI-128 | 54.238 | 0.041 | Not assigned to function by | No |
| | | | Ingenuity | |
| IL9 | 9.187 | 0.041 | Yes | No |
| CCNL1 | 0.153 | 0.041 | Not assigned to function by | No |
| | | | Ingenuity | |
| GORASP2 | 0.100 | 0.041 | Not assigned to function by | No |
| | | | Ingenuity | |
| NUP43 | 6.165 | 0.041 | No | No |
| AP162 | 0.069 | 0.041 | Not assigned to function by | No |
| | | | Ingenuity | |
| PLSCR3 | 0.029 | 0.041 | Not assigned to function by | No |
| | | | Ingenuity | |
| NCOA3 | 13.528 | 0.042 | Yes | No |
| TNFSF10 | 4.806 | 0.042 | Yes | No |
| PPP6C | 0.045 | 0.042 | Yes | No |
| RNUT1 | 11.552 | 0.042 | Not assigned to function by | No |
| | | | Ingenuity | |
| ALEX3 | 5.508 | 0.042 | Not assigned to function by | No |
| | | | Ingenuity | |
| MGLL | 14.896 | 0.042 | No | No |
| CENPC1 | 0.106 | 0.042 | Yes | No |
| NR1D1 | 0.197 | 0.042 | Yes | No |
| FLJ12439 | 6.764 | 0.042 | Not assigned to function by | No |
| | | | Ingenuity | |
| MTMR2 | 11.89 | 0.042 | No | No |
| FDPS | 11.053 | 0.042 | No | No |
| TFEB | 0.138 | 0.042 | No | No |
| KIAA1332 | 0.061 | 0.042 | Not assigned to function by | No |
| | | | Ingenuity | |
| C14ORF159 | 0.132 | 0.042 | Not assigned to function by | No |
| | | | Ingenuity | |
| PSME2 | 10.064 | 0.042 | Yes | No |
| MPHOSPH6 | 10.656 | 0.043 | Yes | No |
| YWHAB | 10.596 | 0.043 | Yes | No |
| MCM7 | 7.75 | 0.043 | Yes | No |
| PSMD2 | 334.893 | 0.043 | Not assigned to function by | No |
| | | | Ingenuity | |
| AMPD2 | 0.122 | 0.043 | No | No |
| CCNE1 | 6.88 | 0.044 | Yes | No |
| MMP7 | 6.512 | 0.044 | Yes | No |
| GTF2H1 | 12.954 | 0.044 | Yes | No |
| FNBP1 | 5.151 | 0.044 | No | No |
| UBD | 7.847 | 0.044 | Yes | No |
| FLJ38984 | 19.598 | 0.045 | Not assigned to function by | No |
| | | | Ingenuity | |
| TLE4 | 0.108 | 0.045 | No | No |
| ITM2B | 0.032 | 0.045 | Yes | No |
| HSD17B7 | 14.99 | 0.045 | No | No |
| KIAA1115 | 33.455 | 0.047 | Not assigned to function by | No |
| | | | Ingenuity | |
| COAS1 | 3.854 | 0.047 | Not assigned to function by | No |
| | | | Ingenuity | |
| XRCC5 | 17.167 | 0.047 | Yes | No |
| STMN1 | 11.125 | 0.047 | Yes | No |
| CTLA4 | 8.016 | 0.047 | Yes | No |
| STAG2 | 6.595 | 0.047 | Not assigned to function by | No |
| | | | Ingenuity | |
| KIAA0404 | 0.144 | 0.047 | Not assigned to function by | No |
| | | | Ingenuity | |
| SF3B4 | 0.180 | 0.047 | No | No |
| CXCL9 | 6.108 | 0.047 | Yes | No |
| ITGAX | 0.032 | 0.047 | No | No |
| FLJ14888 | 25.043 | 0.048 | Not assigned to function by | No |
| | | | Ingenuity | |
| FLJ10803 | 31.56 | 0.048 | Not assigned to function by | No |
| | | | Ingenuity | |
| PTEN | 0.107 | 0.048 | Yes | No |
| OSBPL9 | 288.036 | 0.048 | Not assigned to function by | No |
| | | | Ingenuity | |
| EFHD2 | 0.128 | 0.048 | Not assigned to function by | No |
| | | | Ingenuity | |
| PPIH | 29.937 | 0.048 | Yes | No |
| DOCK2 | 0.100 | 0.048 | Yes | No |
| FGR | 0.088 | 0.048 | Yes | No |
| NKTR | 4.902 | 0.048 | Not assigned to function by | No |
| | | | Ingenuity | |
| ZCCHC2 | 0.080 | 0.048 | Not assigned to function by | No |
| | | | Ingenuity | |
| BAZ2A | 4.766 | 0.048 | No | Yes |
| QKI | 29.983 | 0.049 | Yes | No |
| SPN | 0.102 | 0.049 | Yes | No |
| MATR3 | 0.124 | 0.049 | Not assigned to function by | No |
| | | | Ingenuity | |
| KIAA1536 | 0.073 | 0.049 | Not assigned to function by | No |
| | | | Ingenuity | |
| PABPC3 | 0.038 | 0.049 | Not assigned to function by | No |
| | | | Ingenuity | |
| SUCLA2 | 10.996 | 0.049 | No | No |
| GABBR1 | 0.117 | 0.049 | No | No |
| FBS1 | 0.031 | 0.049 | Yes | No |
| C3ORF4 | 5.543 | 0.049 | Not assigned to function by | No |
| | | | Ingenuity | |
| CYLD | 0.161 | 0.049 | Not assigned to function by | Yes |
| | | | Ingenuity | |
| FLJ21347 | 0.098 | 0.049 | Not assigned to function by | No |
| | | | Ingenuity | |
| AIM2 | 9.506 | 0.049 | Yes | No |
| PTX1 | 9.051 | 0.049 | Not assigned to function by | No |
| | | | Ingenuity | |
| LRDD | 0.219 | 0.049 | Yes | No |
| LOC283537 | 0.094 | 0.049 | Not assigned to function by | No |
| | | | Ingenuity | |
| CLN5 | 11.634 | 0.049 | No | No |
| EPRS | 9.17 | 0.049 | No | No |
| PEX3 | 9.809 | 0.050 | No | No |
| NCOA2 | 0.220 | 0.050 | No | No |
| BHC80 | 0.124 | 0.050 | Not assigned to function by | No |
| | | | Ingenuity | |
| ARHQ | 41.236 | 0.050 | No | No |
| PFKM | 18.355 | 0.050 | No | No |
| WARS | 23.882 | 0.050 | Yes | Yes |
| ESPL1 | 6.537 | 0.050 | Yes | No |
| KRAS2 | 3.71 | 0.050 | Yes | No |
| RGS2 | 0.176 | 0.050 | Yes | No |
| EDG6 | 0.144 | 0.050 | Yes | No |
| MAP3K71P2 | 0.079 | 0.050 | Yes | No |
| CD2BP2 | 59.36 | 0.050 | Not assigned to function by | No |
| | | | Ingenuity | |
| ZNF408 | 0.239 | 0.050 | Not assigned to function by | No |
| | | | Ingenuity | |
| PLEKHF2 | 0.154 | 0.050 | Not assigned to function by | No |
| | | | Ingenuity | |
| KIAA1076 | 0.117 | 0.050 | Not assigned to function by | No |
| | | | Ingenuity | |
| DRE1 | 0.113 | 0.050 | Not assigned to function by | No |
| | | | Ingenuity | |
| C14ORF32 | 0.097 | 0.050 | Not assigned to function by | No |
| | | | Ingenuity | |
| FXC1 | 13.071 | 0.050 | No | No |
| TSTA3 | 6.918 | 0.050 | No | No |
| PWP1 | 4.459 | 0.050 | No | No |
| TCF7L2 | 0.223 | 0.050 | No | No |
| ARL4 | 0.063 | 0.050 | No | No |
| RPA2 | 17.449 | 0.050 | Yes | No |
| GAS7 | 0.091 | 0.051 | Yes | No |
| KIAA0555 | 7.853 | 0.051 | Not assigned to function by | No |
| | | | Ingenuity | |
| SSFA2 | 0.036 | 0.051 | Not assigned to function by | No |
| | | | Ingenuity | |
| NUP50 | 13.853 | 0.051 | No | No |
| GMEB2 | 0.097 | 0.051 | No | No |
| PIR51 | 9.238 | 0.051 | Yes | No |
| C9ORF83 | 8.68 | 0.051 | Not assigned to function by | No |
| | | | Ingenuity | |
| PRO1843 | 0.126 | 0.051 | Not assigned to function by | No |
| | | | Ingenuity | |
| VEGF | 0.124 | 0.052 | Yes | Yes |
| RERE | 0.093 | 0.052 | Yes | No |
| DNM1L | 16.425 | 0.052 | No | No |
| ARID1A | 11.487 | 0.052 | Yes | No |
| FLJ10815 | 9.617 | 0.052 | Not assigned to function by | No |
| | | | Ingenuity | |
| CIAO1 | 17.811 | 0.052 | Yes | No |
| MNT | 0.113 | 0.052 | Yes | No |
| PSMA4 | 51.574 | 0.052 | Not assigned to function by | No |
| | | | Ingenuity | |
| GNL1 | 19.339 | 0.052 | No | No |
| CXCL5 | 4.558 | 0.052 | Yes | No |
| FLJ32731 | 0.180 | 0.052 | Not assigned to function by | No |
| | | | Ingenuity | |
| DUSP10 | 0.099 | 0.053 | Yes | No |
| KIAA0102 | 9.997 | 0.053 | Not assigned to function by | No |
| | | | Ingenuity | |
| PROSC | 5.622 | 0.053 | Not assigned to function by | No |
| | | | Ingenuity | |
| LYL1 | 0.235 | 0.053 | Not assigned to function by | No |
| | | | Ingenuity | |
| MKRN1 | 0.095 | 0.053 | Not assigned to function by | No |
| | | | Ingenuity | |
| MYCBP | 23.309 | 0.053 | No | No |
| CKS2 | 5.629 | 0.053 | Yes | No |
| SGK | 0.161 | 0.053 | Yes | No |
| C20ORF104 | 0.091 | 0.053 | Not assigned to function by | No |
| | | | Ingenuity | |
| KMO | 5.843 | 0.053 | No | No |
| ARS2 | 0.028 | 0.053 | No | Yes |
| ZNF259 | 26.34 | 0.053 | Yes | No |
| GC20 | 0.016 | 0.054 | Yes | No |
| SERP1 | 0.022 | 0.054 | No | No |
| MSF | 0.222 | 0.054 | Yes | No |
| TRAPPC3 | 11.773 | 0.054 | Not assigned to function by | No |
| | | | Ingenuity | |
| CDC40 | 0.074 | 0.054 | No | No |
| PPP3CA | 5.417 | 0.054 | Yes | No |
| FLJ14753 | 0.021 | 0.054 | Not assigned to function by | No |
| | | | Ingenuity | |
| PELI1 | 0.175 | 0.054 | Not assigned to function by | No |
| | | | Ingenuity | |
| PRKCSH | 0.064 | 0.054 | No | No |
| SPINT2 | 0.115 | 0.054 | No | No |
| PSARL | 50.956 | 0.055 | Not assigned to function by | No |
| | | | Ingenuity | |
| HT007 | 10.945 | 0.056 | Not assigned to function by | No |
| | | | Ingenuity | |
| RAD51C | 5.167 | 0.056 | Yes | No |
| TRIP-BR2 | 3.547 | 0.056 | Not assigned to function by | No |
| | | | Ingenuity | |
| TRA1 | 12.843 | 0.056 | Yes | No |
| PIK3CA | 0.075 | 0.056 | Yes | No |
| DKFZP586D0919 | 25.287 | 0.056 | Not assigned to function by | No |
| | | | Ingenuity | |
| CIC | 0.300 | 0.056 | Not assigned to function by | No |
| | | | Ingenuity | |
| HSPC051 | 12.156 | 0.057 | No | No |
| NADSYN1 | 0.182 | 0.057 | Not assigned to function by | No |
| | | | Ingenuity | |
| ELAVL1 | 4.903 | 0.057 | No | No |
| CCL22 | 3.61 | 0.057 | Yes | No |
| C20ORF67 | 0.176 | 0.057 | Not assigned to function by | No |
| | | | Ingenuity | |
| CCNB2 | 12.529 | 0.057 | No | No |
| LOC51064 | 0.128 | 0.057 | No | No |
| POLR2K | 7.759 | 0.057 | No | No |
| LRP8 | 7.185 | 0.057 | No | No |
| FLJ20080 | 9.512 | 0.057 | Not assigned to function by | No |
| | | | Ingenuity | |
| JUND | 0.059 | 0.058 | Yes | No |
| ACADM | 6.254 | 0.058 | No | No |
| FLJ20534 | 12.83 | 0.058 | Not assigned to function by | No |
| | | | Ingenuity | |
| TOB1 | 0.172 | 0.058 | Yes | No |
| ACTG1 | 0.006 | 0.058 | No | Yes |
| FLJ10534 | 7.735 | 0.059 | Not assigned to function by | Yes |
| | | | Ingenuity | |
| CTPS | 5.567 | 0.059 | No | No |
| TCP1 | 21.895 | 0.059 | No | No |
| D1S155E | 0.073 | 0.059 | No | No |
| TIMELESS | 6.145 | 0.059 | Yes | No |
| NCOR1 | 59.364 | 0.059 | No | No |
| CSK | 0.040 | 0.059 | Yes | No |
| C9ORF40 | 7.261 | 0.059 | Not assigned to function by | No |
| | | | Ingenuity | |
| PHF3 | 0.081 | 0.059 | Not assigned to function by | No |
| | | | Ingenuity | |
| DKFZP564D0478 | 0.049 | 0.059 | Not assigned to function by | No |
| | | | Ingenuity | |
| DDEF1 | 7.693 | 0.059 | No | No |
| UBE2L3 | 7.274 | 0.059 | No | No |
| FBXL12 | 0.008 | 0.059 | No | No |
| CSNK2A1 | 9.961 | 0.059 | Yes | No |
| KIAA0483 | 22.767 | 0.059 | Not assigned to function by | No |
| | | | Ingenuity | |
| TNFRSF9 | 3.452 | 0.060 | Yes | No |
| NEDD8 | 5.847 | 0.060 | No | No |
| ZNF161 | 0.086 | 0.060 | Yes | No |
| KIAA0738 | 7.398 | 0.060 | Not assigned to function by | No |
| | | | Ingenuity | |
| SIAT9 | 0.018 | 0.060 | No | No |
| MADH7 | 0.113 | 0.060 | Yes | No |
| USP3 | 0.051 | 0.060 | No | No |
| KHDRBS1 | 7.898 | 0.060 | Yes | No |
| C5ORF6 | 0.047 | 0.061 | Not assigned to function by | No |
| | | | Ingenuity | |
| TCF8 | 0.300 | 0.061 | Yes | No |
| GLG1 | 6.776 | 0.061 | Not assigned to function by | No |
| | | | Ingenuity | |
| RBAF600 | 0.011 | 0.061 | Not assigned to function by | No |
| | | | Ingenuity | |
| SLC35D2 | 21.354 | 0.061 | Not assigned to function by | No |
| | | | Ingenuity | |
| PIGA | 0.156 | 0.061 | Yes | No |
| DUSP3 | 9.995 | 0.061 | No | No |
| DSCR1 | 26.47 | 0.061 | Yes | No |
| PTMA | 9.39 | 0.062 | Yes | Yes |
| POLE2 | 5.826 | 0.062 | Yes | No |
| NRG1 | 0.308 | 0.062 | Yes | No |
| TRAF6 | 0.049 | 0.062 | Yes | No |
| CGI-51 | 26.286 | 0.062 | Not assigned to function by | No |
| | | | Ingenuity | |
| TIP120A | 14.465 | 0.062 | No | No |
| MAC30 | 9.549 | 0.062 | No | No |
| WDR12 | 6.497 | 0.062 | No | No |
| SLC22A18 | 0.139 | 0.062 | No | No |
| VAMP2 | 0.092 | 0.062 | No | Yes |
| EVI2B | 0.109 | 0.062 | Not assigned to function by | No |
| | | | Ingenuity | |
| TIEG2 | 0.231 | 0.062 | Yes | No |
| COPS5 | 8.168 | 0.062 | Yes | No |
| RNF139 | 0.143 | 0.062 | No | No |
| MPO | 0.146 | 0.063 | Yes | No |
| PCMT1 | 8.822 | 0.063 | No | No |
| KCNK4 | 6.788 | 0.063 | No | No |
| SSA2 | 5.063 | 0.063 | No | No |
| Unknown | 4.635 | 0.064 | Not assigned to function by | No |
| | | | Ingenuity | |
| Unknown | 0.315 | 0.064 | Not assigned to function by | No |
| | | | Ingenuity | |
| Unknown | 0.271 | 0.064 | Not assigned to function by | No |
| | | | Ingenuity | |
| Unknown | 0.157 | 0.064 | Not assigned to function by | No |
| | | | Ingenuity | |
| Unknown | 0.109 | 0.064 | Not assigned to function by | No |
| | | | Ingenuity | |
| Unknown | 0.089 | 0.064 | Not assigned to function by | No |
| | | | Ingenuity | |
| Unknown | 0.081 | 0.064 | Not assigned to function by | No |
| | | | Ingenuity | |
| Unknown | 0.069 | 0.064 | Not assigned to function by | No |
| | | | Ingenuity | |
| Unknown | 0.015 | 0.064 | Not assigned to function by | No |
| | | | Ingenuity | |
| DSIPI | 0.237 | 0.064 | Yes | No |
| POLD3 | 8.246 | 0.064 | Yes | No |
| EEF2 | 0.180 | 0.064 | Yes | No |
| NEIL1 | 0.090 | 0.064 | Yes | No |
| KIAA0683 | 13.161 | 0.064 | Not assigned to function by | No |
| | | | Ingenuity | |
| KIAA1002 | 0.133 | 0.064 | Not assigned to function by | No |
| | | | Ingenuity | |
| FLJ10099 | 25.454 | 0.064 | Not assigned to function by | No |
| | | | Ingenuity | |
| KIAA0582 | 6.047 | 0.065 | Not assigned to function by | No |
| | | | Ingenuity | |
| HADHSC | 0.155 | 0.065 | No | No |
| VIPR1 | 0.154 | 0.065 | Yes | No |
| C2ORF6 | 8.647 | 0.065 | Not assigned to function by | No |
| | | | Ingenuity | |
| SLC4A1AP | 36.646 | 0.065 | Not assigned to function by | No |
| | | | Ingenuity | |
| ARL7 | 0.187 | 0.065 | Not assigned to function by | Yes |
| | | | Ingenuity | |
| DXYS155E | 0.032 | 0.065 | Not assigned to function by | No |
| | | | Ingenuity | |
| BIRC2 | 0.118 | 0.065 | Yes | No |
| STAT5A | 56.762 | 0.065 | Yes | No |
| PHB | 8.89 | 0.065 | Yes | No |
| MYLK | 3.958 | 0.065 | Yes | No |
| ATP5L | 0.096 | 0.065 | Not assigned to function by | Yes |
| | | | Ingenuity | |
| KEAP1 | 7.403 | 0.066 | Not assigned to function by | No |
| | | | Ingenuity | |
| CAMKK2 | 3.454 | 0.066 | No | No |
| PRKCN | 0.189 | 0.066 | No | No |
| MARK4 | 0.196 | 0.066 | Not assigned to function by | No |
| | | | Ingenuity | |
| CDK4 | 5.678 | 0.066 | Yes | No |
| PAICS | 5.533 | 0.066 | No | Yes |
| NFKB1 | 5.354 | 0.066 | Yes | No |
| CGI-90 | 9.14 | 0.066 | Not assigned to function by | No |
| | | | Ingenuity | |
| TNIP3 | 5.962 | 0.066 | Not assigned to function by | No |
| | | | Ingenuity | |
| C1ORF9 | 0.058 | 0.066 | Not assigned to function by | No |
| | | | Ingenuity | |
| IL12B | 4.425 | 0.067 | Yes | No |
| RUNX1 | 0.037 | 0.067 | Yes | No |
| POP5 | 8.456 | 0.067 | No | No |
| C20ORF121 | 0.125 | 0.067 | Not assigned to function by | No |
| | | | Ingenuity | |
| EIF2B2 | 28.683 | 0.067 | Yes | No |
| MGC4825 | 14.636 | 0.067 | Not assigned to function by | No |
| | | | Ingenuity | |
| MRPL19 | 7.652 | 0.067 | Not assigned to function by | No |
| | | | Ingenuity | |
| KIAA0982 | 0.170 | 0.067 | Not assigned to function by | No |
| | | | Ingenuity | |
| ILF3 | 11.625 | 0.067 | No | No |
| CCR1 | 4.928 | 0.067 | Yes | No |
| TNF | 3.471 | 0.067 | Yes | No |
| LYRIC | 0.074 | 0.067 | Not assigned to function by | Yes |
| | | | Ingenuity | |
| FLJ21603 | 0.052 | 0.067 | Not assigned to function by | No |
| | | | Ingenuity | |
| PRKAG1 | 8.572 | 0.067 | No | No |
| NISCH | 0.054 | 0.067 | No | No |
| BRD3 | 0.076 | 0.067 | Not assigned to function by | No |
| | | | Ingenuity | |
| COX7C | 0.116 | 0.067 | No | Yes |
| SRP72 | 3.342 | 0.068 | Not assigned to function by | No |
| | | | Ingenuity | |
| CA12 | 4.9 | 0.068 | No | No |
| KLF3 | 0.147 | 0.068 | No | No |
| FLJ20546 | 9.408 | 0.068 | Not assigned to function by | No |
| | | | Ingenuity | |
| BLM | 13.128 | 0.068 | Yes | No |
| PLAC8 | 0.161 | 0.069 | Not assigned to function by | No |
| | | | Ingenuity | |
| KIAA1012 | 32.462 | 0.069 | Not assigned to function by | No |
| | | | Ingenuity | |
| SRPK1 | 7.399 | 0.069 | No | No |
| CLECSF12 | 0.260 | 0.069 | Yes | No |
| COPS8 | 60.228 | 0.069 | Not assigned to function by | No |
| | | | Ingenuity | |
| MGC11256 | 11.319 | 0.069 | Not assigned to function by | No |
| | | | Ingenuity | |
| MRPS11 | 10.289 | 0.069 | Not assigned to function by | No |
| | | | Ingenuity | |
| SLC2A14 | 0.219 | 0.069 | Not assigned to function by | No |
| | | | Ingenuity | |
| CUTL1 | 0.106 | 0.069 | No | No |
| PAFAH1B1 | 18.233 | 0.069 | Yes | No |
| AKAP13 | 8.326 | 0.069 | Yes | Yes |
| HIST1H4C | 4.545 | 0.071 | Not assigned to function by | No |
| | | | Ingenuity | |
| PSME4 | 0.102 | 0.071 | Not assigned to function by | No |
| | | | Ingenuity | |
| EIF5B | 5.498 | 0.072 | Yes | No |
| KIAA0152 | 23.99 | 0.072 | Not assigned to function by | No |
| | | | Ingenuity | |
| CINP | 18.638 | 0.072 | Not assigned to function by | No |
| | | | Ingenuity | |
| G0S2 | 0.266 | 0.072 | Not assigned to function by | No |
| | | | Ingenuity | |
| SENP3 | 15.871 | 0.072 | No | No |
| COL18A1 | 0.057 | 0.072 | Yes | No |
| FN5 | 10.552 | 0.072 | Not assigned to function by | No |
| | | | Ingenuity | |
| HNRPR | 10.584 | 0.072 | No | No |
| ATP6V1C1 | 6.714 | 0.072 | No | Yes |
| EEF1E1 | 9.813 | 0.072 | Not assigned to function by | No |
| | | | Ingenuity | |
| TANK | 6.588 | 0.072 | No | No |
| IFIT5 | 0.128 | 0.073 | Not assigned to function by | No |
| | | | Ingenuity | |
| TUBA3 | 0.124 | 0.073 | Not assigned to function by | No |
| | | | Ingenuity | |
| UBE2J1 | 10.265 | 0.074 | Not assigned to function by | No |
| | | | Ingenuity | |
| PER1 | 0.118 | 0.074 | No | No |
| DGCR14 | 0.150 | 0.074 | Not assigned to function by | No |
| | | | Ingenuity | |
| CGI-49 | 15.54 | 0.074 | Not assigned to function by | No |
| | | | Ingenuity | |
| CEACAM8 | 0.244 | 0.074 | Yes | No |
| GNAI2 | 0.178 | 0.074 | Yes | No |
| 13CDNA73 | 0.086 | 0.074 | Not assigned to function by | No |
| | | | Ingenuity | |
| ACAT1 | 13.587 | 0.074 | No | No |
| GOT1 | 6.694 | 0.074 | No | No |
| SMG1 | 0.096 | 0.074 | No | No |
| TUBB | 0.250 | 0.074 | Not assigned to function by | No |
| | | | Ingenuity | |
| CHD4 | 12.6 | 0.075 | No | No |
| RGS10 | 4.866 | 0.075 | No | No |
| CAMP | 0.253 | 0.075 | Yes | No |
| APOM | 0.107 | 0.075 | No | No |
| FLJ21868 | 0.096 | 0.076 | Not assigned to function by | No |
| | | | Ingenuity | |
| MCM10 | 4.325 | 0.076 | Not assigned to function by | No |
| | | | Ingenuity | |
| C11ORF2 | 0.215 | 0.076 | Not assigned to function by | No |
| | | | Ingenuity | |
| MPZL1 | 26.093 | 0.076 | No | No |
| MRPL48 | 15.923 | 0.077 | Not assigned to function by | No |
| | | | Ingenuity | |
| SET | 10.103 | 0.077 | Yes | No |
| RARA | 0.123 | 0.077 | Yes | No |
| C16ORF35 | 0.221 | 0.077 | Not assigned to function by | No |
| | | | Ingenuity | |
| T1 | 0.084 | 0.077 | Not assigned to function by | No |
| | | | Ingenuity | |
| INHBA | 3.484 | 0.077 | Yes | No |
| NCOA1 | 0.124 | 0.077 | Yes | No |
| JMJD1 | 0.113 | 0.077 | Not assigned to function by | No |
| | | | Ingenuity | |
| UVRAG | 0.043 | 0.077 | Not assigned to function by | No |
| | | | Ingenuity | |
| NCBP1 | 6.422 | 0.077 | No | No |
| NINJ2 | 0.217 | 0.077 | No | No |
| CXCL13 | 4.537 | 0.077 | Yes | No |
| CYB5 | 7.271 | 0.078 | Yes | Yes |
| KLRD1 | 13.813 | 0.079 | Yes | No |
| PLEKHB2 | 0.035 | 0.079 | Not assigned to function by | No |
| | | | Ingenuity | |
| APG12L | 10.273 | 0.079 | No | No |
| TNPO1 | 16.302 | 0.079 | Yes | No |
| PDPK1 | 6.544 | 0.079 | Yes | No |
| FOSL2 | 0.091 | 0.079 | Yes | No |
| SLCO3A1 | 0.139 | 0.079 | Not assigned to function by | No |
| | | | Ingenuity | |
| YT521 | 0.097 | 0.079 | No | No |
| NDUFB8 | 0.060 | 0.079 | No | No |
| TRIM44 | 6.37 | 0.079 | Not assigned to function by | No |
| | | | Ingenuity | |
| USP4 | 23.919 | 0.079 | Not assigned to function by | No |
| | | | Ingenuity | |
| SEC61A1 | 14.769 | 0.079 | Not assigned to function by | No |
| | | | Ingenuity | |
| SF3B1 | 0.048 | 0.079 | Not assigned to function by | Yes |
| | | | Ingenuity | |
| HA-1 | 0.193 | 0.080 | Not assigned to function by | No |
| | | | Ingenuity | |
| SMARCD3 | 0.202 | 0.080 | No | No |
| AP3D1 | 79.171 | 0.080 | Yes | No |
| EG1 | 6.217 | 0.080 | Not assigned to function by | No |
| | | | Ingenuity | |
| SIGIRR | 0.267 | 0.080 | Not assigned to function by | No |
| | | | Ingenuity | |
| S100A10 | 0.077 | 0.080 | Yes | No |
| KIAA0553 | 4.734 | 0.080 | Not assigned to function by | No |
| | | | Ingenuity | |
| PTMAP7 | 4.827 | 0.081 | Not assigned to function by | No |
| | | | Ingenuity | |
| FLJ12671 | 11.68 | 0.081 | Not assigned to function by | No |
| | | | Ingenuity | |
| MELK | 4.422 | 0.081 | Not assigned to function by | No |
| | | | Ingenuity | |
| ELMO2 | 0.052 | 0.081 | Not assigned to function by | No |
| | | | Ingenuity | |
| DDX41 | 10.888 | 0.081 | Yes | No |
| MGC39821 | 14.096 | 0.081 | No | No |
| CDC2 | 5.354 | 0.081 | Yes | No |
| IMMT | 24.233 | 0.081 | Not assigned to function by | No |
| | | | Ingenuity | |
| TM7SF1 | 7.167 | 0.081 | Not assigned to function by | No |
| | | | Ingenuity | |
| G3BP2 | 0.061 | 0.081 | Not assigned to function by | No |
| | | | Ingenuity | |
| ASNA1 | 8.267 | 0.081 | No | No |
| KIAA0143 | 24.981 | 0.081 | Not assigned to function by | No |
| | | | Ingenuity | |
| CSNK1A1 | 33.586 | 0.081 | No | Yes |
| LOC203069 | 0.050 | 0.081 | Not assigned to function by | No |
| | | | Ingenuity | |
| MRPL28 | 15.604 | 0.081 | Not assigned to function by | No |
| | | | Ingenuity | |
| SLC18A2 | 0.148 | 0.081 | Yes | No |
| MTCP1 | 0.114 | 0.081 | Yes | No |
| FLJ20989 | 9.325 | 0.081 | Not assigned to function by | No |
| | | | Ingenuity | |
| KIAA0399 | 0.142 | 0.081 | Not assigned to function by | Yes |
| | | | Ingenuity | |
| POT1 | 12.322 | 0.081 | Not assigned to function by | No |
| | | | Ingenuity | |
| CUL4A | 15.206 | 0.082 | Not assigned to function by | No |
| | | | Ingenuity | |
| LAPTM4B | 4.808 | 0.082 | Not assigned to function by | No |
| | | | Ingenuity | |
| TYMS | 3.794 | 0.082 | Yes | No |
| ERCC1 | 0.089 | 0.082 | Yes | No |
| PSMC3 | 3.801 | 0.082 | Yes | No |
| PIAS1 | 5.994 | 0.082 | Yes | No |
| CDYL | 14.787 | 0.083 | No | No |
| ACAT2 | 6.322 | 0.083 | No | No |
| RBBP8 | 13.511 | 0.083 | Not assigned to function by | No |
| | | | Ingenuity | |
| ATF1 | 13.031 | 0.083 | Yes | No |
| CBX1 | 7.07 | 0.083 | No | No |
| CLK1 | 0.090 | 0.084 | Yes | Yes |
| PBF | 0.218 | 0.084 | Not assigned to function by | No |
| | | | Ingenuity | |
| SLC2A4RG | 0.080 | 0.084 | No | No |
| KAI1 | 6.32 | 0.084 | Yes | No |
| EP300 | 10.703 | 0.084 | Yes | No |
| DNAJB6 | 4.765 | 0.085 | Yes | No |
| UGDH | 5.91 | 0.085 | No | No |
| C20ORF9 | 56.405 | 0.085 | Not assigned to function by | No |
| | | | Ingenuity | |
| NIT2 | 7.547 | 0.086 | Not assigned to function by | No |
| | | | Ingenuity | |
| RBM5 | 13.578 | 0.086 | Yes | No |
| HNRPH3 | 0.088 | 0.086 | No | No |
| DHX40 | 0.073 | 0.087 | Not assigned to function by | No |
| | | | Ingenuity | |
| MSCP | 7.256 | 0.087 | Not assigned to function by | No |
| | | | Ingenuity | |
| PSMB9 | 8.276 | 0.087 | No | No |
| C10ORF22 | 6.428 | 0.087 | Not assigned to function by | No |
| | | | Ingenuity | |
| UROD | 13.864 | 0.087 | Not assigned to function by | No |
| | | | Ingenuity | |
| MTSS1 | 16.897 | 0.087 | No | No |
| GAB2 | 0.115 | 0.088 | Yes | No |
| FLJ11856 | 15.457 | 0.089 | Not assigned to function by | No |
| | | | Ingenuity | |
| SNX1 | 0.246 | 0.089 | No | No |
| CGI-94 | 12.952 | 0.089 | Not assigned to function by | No |
| | | | Ingenuity | |
| ANAPC2 | 0.250 | 0.089 | Yes | No |
| PPBP | 7.479 | 0.090 | Yes | No |
| LOC51315 | 6.733 | 0.090 | Not assigned to function by | No |
| | | | Ingenuity | |
| ARHGEF2 | 9.18 | 0.090 | Yes | No |
| ANKRD10 | 0.121 | 0.090 | Not assigned to function by | No |
| | | | Ingenuity | |
| NUDC | 10.798 | 0.091 | Yes | No |
| STX7 | 4.414 | 0.091 | Yes | No |
| NDUFAF1 | 5.219 | 0.091 | Not assigned to function by | No |
| | | | Ingenuity | |
| SLAMF8 | 3.465 | 0.091 | Not assigned to function by | No |
| | | | Ingenuity | |
| PFKP | 6.294 | 0.091 | No | No |
| CPSF6 | 0.146 | 0.091 | No | No |
| LSM2 | 8.984 | 0.091 | Not assigned to function by | No |
| | | | Ingenuity | |
| LAP1B | 0.054 | 0.091 | Not assigned to function by | No |
| | | | Ingenuity | |
| RASA4 | 0.189 | 0.091 | Not assigned to function by | No |
| | | | Ingenuity | |
| POGK | 6.766 | 0.091 | Not assigned to function by | No |
| | | | Ingenuity | |
| SSR1 | 0.122 | 0.092 | Yes | No |
| ATP1B1 | 5.102 | 0.092 | No | No |
| PA2G4 | 7.067 | 0.092 | Yes | No |
| USF2 | 0.244 | 0.092 | Yes | No |
| CASP3 | 14.85 | 0.092 | Yes | No |
| PPIB | 5.353 | 0.092 | Yes | No |
| SP2 | 0.135 | 0.093 | Not assigned to function by | No |
| | | | Ingenuity | |
| GRP58 | 15.372 | 0.093 | Yes | No |
| KIAA0863 | 0.097 | 0.093 | Not assigned to function by | No |
| | | | Ingenuity | |
| CD24 | 0.031 | 0.094 | Yes | No |
| TCFL1 | 19.522 | 0.094 | No | No |
| MCM4 | 5.113 | 0.094 | Yes | No |
| SULT1A1 | 0.047 | 0.094 | No | No |
| FARSLA | 4.776 | 0.094 | Not assigned to function by | No |
| | | | Ingenuity | |
| PCF11 | 0.197 | 0.094 | Not assigned to function by | No |
| | | | Ingenuity | |
| TNS | 0.064 | 0.094 | No | Yes |
| HBP1 | 0.062 | 0.094 | No | No |
| GGA1 | 0.088 | 0.094 | Yes | No |
| ILVBL | 31.351 | 0.094 | Not assigned to function by | No |
| | | | Ingenuity | |
| WDR26 | 0.037 | 0.094 | Not assigned to function by | No |
| | | | Ingenuity | |
| ZNF24 | 24.761 | 0.094 | No | No |
| DYRK1A | 0.134 | 0.094 | No | No |
| NOLC1 | 5.681 | 0.094 | Yes | No |
| HNRPAB | 7.05 | 0.094 | No | No |
| PPM1F | 0.140 | 0.094 | Yes | No |
| ASH2L | 23.135 | 0.094 | Not assigned to function by | No |
| | | | Ingenuity | |
| SASH1 | 8.308 | 0.095 | Not assigned to function by | No |
| | | | Ingenuity | |
| RPL13 | 0.167 | 0.095 | Not assigned to function by | No |
| | | | Ingenuity | |
| PFS2 | 3.043 | 0.095 | Not assigned to function by | No |
| | | | Ingenuity | |
| TMEM14A | 6.614 | 0.095 | Not assigned to function by | No |
| | | | Ingenuity | |
| FLJ35827 | 0.121 | 0.095 | Not assigned to function by | No |
| | | | Ingenuity | |
| REPIN1 | 0.114 | 0.096 | Yes | No |
| EI24 | 14.543 | 0.096 | Yes | No |
| MRPS7 | 9.557 | 0.096 | Not assigned to function by | No |
| | | | Ingenuity | |
| TRIM8 | 0.173 | 0.096 | Not assigned to function by | No |
| | | | Ingenuity | |
| GABPB2 | 6.265 | 0.096 | Yes | No |
| ERP70 | 28.582 | 0.096 | No | No |
| KIAA0763 | 0.150 | 0.096 | No | No |
| NGX6 | 0.125 | 0.096 | Not assigned to function by | No |
| | | | Ingenuity | |
| CREBL2 | 15.292 | 0.097 | No | No |
| NEK7 | 9.293 | 0.097 | Not assigned to function by | No |
| | | | Ingenuity | |
| CAMLG | 0.165 | 0.097 | No | No |
| CCT7 | 4.279 | 0.097 | Yes | No |
| INSIG2 | 9.42 | 0.097 | No | No |
| CCNI | 0.063 | 0.097 | No | Yes |
| DAPP1 | 7.907 | 0.098 | No | No |
| USP21 | 0.207 | 0.098 | Yes | No |
| RBM8A | 4.954 | 0.098 | No | No |
| PRKCI | 6.662 | 0.098 | Yes | No |
| WBP11 | 0.042 | 0.098 | Not assigned to function by | No |
| | | | Ingenuity | |
| CCRL2 | 4.788 | 0.098 | Not assigned to function by | No |
| | | | Ingenuity | |
| MRPL12 | 7.889 | 0.098 | Yes | No |
| PSMB5 | 5.428 | 0.099 | Not assigned to function by | No |
| | | | Ingenuity | |
| EBI3 | 3.985 | 0.099 | Not assigned to function by | No |
| | | | Ingenuity | |
| BCAP31 | 9.401 | 0.099 | Yes | No |
| ZNF297B | 6.866 | 0.099 | Not assigned to function by | No |
| | | | Ingenuity | |
| KNS2 | 5.27 | 0.099 | No | No |
| SRD5A1 | 0.212 | 0.100 | No | No |
| MSH2 | 6.541 | 0.100 | Yes | No |
| ID3 | 0.052 | 0.100 | Yes | No |
| EIF2B1 | 20.633 | 0.100 | No | No |
| IRAK1BP1 | 0.191 | 0.100 | No | No |

TABLE 34
Meningoencephalitis-associated Genes Connected to Cell Death

| | Odds Ratio for | | | |
|---|---|---|---|---|
| | association with | Meningoencephalitis | | Affymetrix |
| Gene name | meningoencephalitis | FDR | Pathway associations | identifier |
| HNRPC | 324.673 | 0.016 | Cell Death pathways:IgM | 214737_x_at |
| STAT1 | 230.416 | 0.004 | Cell Death pathways:TNF | 209969_s_at |
| | | | superfamily, TCR, p53 | |
| PDCD8 | 119.631 | 0.019 | Cell Death pathways | 205512_s_at |
| NFATC1 | 80.225 | 0.036 | Cell Death pathways | 210162_s_at |
| STAT5A | 56.762 | 0.065 | TNF superfamily | 203010_at |
| BRD2 | 56.318 | 0.010 | Cell Death pathways:cell | 208686_s_at |
| | | | cycle | |
| IL27RA | 46.587 | 0.032 | Cell Death pathways | 205926_at |
| DUT | 40.207 | 0.018 | Cell Death pathways:IGM | 208955_at |
| CSE1L | 38.753 | 0.019 | Cell Death pathways:TNF | 201112_s_at |
| | | | superfamily | |
| GZMB | 31.809 | 0.010 | Cell Death pathways:target | 210164_at |
| | | | cell killing | |
| QKI | 29.983 | 0.049 | Cell Death pathways | 212263_at |
| TRIAD3 | 29.384 | 0.039 | Cell Death pathways:TNF | 218426_s_at |
| | | | superfamily | |
| CD80 | 26.520 | 0.036 | Cell Death pathways:TCR | 207176_s_at |
| | | | costimulation | |
| DSCR1 | 26.470 | 0.061 | Cell Death pathways | 208370_s_at |
| PAFAH1B1 | 18.233 | 0.069 | Cell Death pathways | 200816_s_at |
| TDE1 | 17.535 | 0.032 | Cell Death pathways | 211769_x_at |
| XRCC5 | 17.167 | 0.047 | Cell Death pathways | 208643_s_at |
| PRKAR1A | 16.407 | 0.036 | Cell Death pathways | 200604_s_at |
| PDCD11 | 15.892 | 0.036 | Cell Death pathways:TNF | 212424_at |
| | | | superfamily | |
| GRP58 | 15.372 | 0.093 | Cell Death pathways | 208612_at |
| HTATIP2 | 15.356 | 0.036 | Cell Death pathways | 207180_s_at |
| CASP3 | 14.850 | 0.092 | Cell Death pathways:TNF | 202763_at |
| EI24 | 14.543 | 0.096 | Cell Death pathways | 208289_s_at |
| UGCG | 14.445 | 0.013 | Cell Death pathways | 204881_s_at |
| KLRD1 | 13.813 | 0.079 | Cell Death pathways | 207796_x_at |
| RBM5 | 13.578 | 0.086 | Cell Death pathways:TNF | 201394_s_at |
| | | | superfamily | |
| NCOA3 | 13.528 | 0.042 | Cell Death pathways | 207700_s_at |
| BLM | 13.128 | 0.068 | Cell Death pathways:p53 | 205733_at |
| ATF1 | 13.031 | 0.083 | Cell Death | 222103_at |
| | | | pathways:apoptosis | |
| TRA1 | 12.843 | 0.056 | Cell Death pathways | 200598_s_at |
| ITGAV | 12.837 | 0.039 | Cell Death pathways | 202351_at |
| BARD1 | 11.776 | 0.022 | Cell Death pathways:p53 | 205345_at |
| IL2RA | 11.266 | 0.016 | Cell Death pathways | 211269_s_at |
| SH3BP5 | 11.205 | 0.022 | Cell Death pathways | 201810_s_at |
| STMN1 | 11.125 | 0.047 | Cell Death pathways | 200783_s_at |
| NR3C1 | 11.050 | 0.036 | Cell Death pathways | 201865_x_at |
| DDX41 | 10.888 | 0.081 | Cell Death pathways | 217840_at |
| UNG | 10.732 | 0.035 | Cell Death pathways | 202330_s_at |
| EP300 | 10.703 | 0.084 | Cell Death pathways | 213579_s_at |
| CSNK2A1 | 9.961 | 0.059 | Cell Death pathways | 212075_s_at |
| AIM2 | 9.506 | 0.049 | Cell Death pathways:Cell | 206513_at |
| | | | death, cell proliferation | |
| BCAP31 | 9.401 | 0.099 | Cell Death pathways | 200837_at |
| PTMA | 9.390 | 0.062 | Cell Death pathways | 216384_x_at |
| IL9 | 9.187 | 0.041 | Cell Death pathways | 208193_at |
| PHB | 8.890 | 0.065 | Cell Death pathways | 200659_s_at |
| IL19 | 8.869 | 0.031 | Cell Death pathways:TNF | 220745_at |
| | | | superfamily | |
| MBD4 | 8.790 | 0.014 | Cell Death pathways | 209579_s_at |
| PAX5 | 8.402 | 0.035 | Cell Death pathways | 221969_at |
| CTLA4 | 8.016 | 0.047 | Cell Death pathways:TCR | 221331_x_at |
| | | | costimulation | |
| KHDRBS1 | 7.898 | 0.060 | Cell Death pathways | 201488_x_at |
| UBD | 7.847 | 0.044 | Cell Death pathways | 205890_s_at |
| PPBP | 7.479 | 0.090 | Cell Death pathways | 214146_s_at |
| CYB5 | 7.271 | 0.078 | Cell Death pathways | 209366_x_at |
| VDR | 7.092 | 0.014 | Cell Death pathways | 204255_s_at |
| CCNE1 | 6.880 | 0.044 | Cell Death pathways:p53 | 213523_at |
| DNCL1 | 6.810 | 0.023 | Cell Death pathways | 200703_at |
| PRKCI | 6.662 | 0.098 | Cell Death pathways:TNF | 209678_s_at |
| | | | superfamily, TGF | |
| | | | superfamily | |
| STAT3 | 6.606 | 0.011 | Cell Death pathways:TNF | 208991_at |
| | | | superfamily | |
| PDPK1 | 6.544 | 0.079 | Cell Death pathways | 32029_at |
| MSH2 | 6.541 | 0.100 | Cell Death pathways | 209421_at |
| ESPL1 | 6.537 | 0.050 | Cell Death pathways | 204817_at |
| MMP7 | 6.512 | 0.044 | Cell Death pathways:TNF | 204259_at |
| | | | superfamily | |
| KAI1 | 6.320 | 0.084 | Cell Death pathways | 203904_x_at |
| GABPB2 | 6.265 | 0.096 | Cell Death pathways | 204618_s_at |
| PIAS1 | 5.994 | 0.082 | Cell Death pathways:TNF | 217864_s_at |
| | | | superfamily, TCR, p53 | |
| CDK4 | 5.678 | 0.066 | Cell Death pathways:p53 | 202246_s_at |
| RRM2 | 5.394 | 0.029 | Cell Death pathways | 209773_s_at |
| NFKB1 | 5.354 | 0.066 | Cell Death pathways:TNF | 209239_at |
| | | | superfamily | |
| CDC2 | 5.354 | 0.081 | Cell Death pathways:p53 | 203213_at |
| RAD51C | 5.167 | 0.056 | Cell Death pathways | 209849_s_at |
| CCR1 | 4.928 | 0.067 | Cell Death pathways | 205099_s_at |
| PTPRC | 4.891 | 0.017 | Cell Death pathways | 212587_s_at |
| TNFSF10 | 4.806 | 0.042 | Cell Death pathways:TNF | 202688_at |
| | | | superfamily | |
| DNAJB6 | 4.765 | 0.085 | Cell Death pathways | 209015_s_at |
| IL12B | 4.425 | 0.067 | Cell Death pathways:TGFb, | 207901_at |
| | | | TNF | |
| MYLK | 3.958 | 0.065 | Cell Death pathways | 202555_s_at |
| TYMS | 3.794 | 0.082 | Cell Death pathways | 202589_at |
| KRAS2 | 3.710 | 0.050 | Cell Death pathways:TNF | 214352_s_at |
| | | | superfamily | |
| INHBA | 3.484 | 0.077 | Cell Death | 210511_s_at |
| | | | pathways:INHBA:TGF | |
| | | | superfamily check | |
| TNF | 3.471 | 0.067 | Cell Death pathways:TNF | 207113_s_at |
| | | | superfamily | |
| TNFRSF9 | 3.452 | 0.060 | Cell Death pathways:TNF | 207536_s_at |
| | | | superfamily | |
| IL7R | 3.182 | 0.039 | Cell Death pathways | 205798_at |
| NRG1 | 0.308 | 0.062 | Cell Death pathways | 206343_s_at |
| DSIPI | 0.237 | 0.064 | Cell Death pathways | 208763_s_at |
| NCOA2 | 0.220 | 0.050 | Cell Death pathways | 212867_at |
| LRDD | 0.219 | 0.049 | Cell Death pathways:TNF | 219019_at |
| | | | superfamily, p53 | |
| BPI | 0.219 | 0.027 | Cell Death pathways | 205557_at |
| NR1D1 | 0.197 | 0.042 | Cell Death pathways | 204760_s_at |
| CABIN1 | 0.162 | 0.036 | Cell Death pathways:TCR | 37652_at |
| SGK | 0.161 | 0.053 | Cell Death pathways | 201739_at |
| VIPR1 | 0.154 | 0.065 | Cell Death pathways | 205019_s_at |
| SLC18A2 | 0.148 | 0.081 | Cell Death pathways | 213549_at |
| MPO | 0.146 | 0.063 | Cell Death pathways | 203949_at |
| PPM1F | 0.140 | 0.094 | Cell Death pathways | 37384_at |
| NALP1 | 0.133 | 0.023 | Cell Death pathways | 218380_at |
| VEGF | 0.124 | 0.052 | Cell Death pathways | 212171_x_at |
| NCOA1 | 0.124 | 0.077 | Cell Death pathways | 209105_at |
| RARA | 0.123 | 0.077 | Cell Death pathways:TCR | 203749_s_at |
| SCGF | 0.120 | 0.038 | Cell Death pathways | 211709_s_at |
| BIRC2 | 0.118 | 0.065 | Cell Death | 202076_at |
| | | | pathways:apoptosis | |
| NFIL3 | 0.116 | 0.036 | Cell Death pathways:p53 | 203574_at |
| MOAP1 | 0.115 | 0.036 | Cell Death pathways | 212508_at |
| SMAD7 | 0.113 | 0.060 | Cell Death pathways:TGFb | 204790_at |
| ZFP36 | 0.108 | 0.029 | Cell Death pathways:TNF | 201531_at |
| | | | superfamily | |
| JUNB | 0.108 | 0.032 | Cell Death pathways | 201473_at |
| PTEN | 0.107 | 0.048 | Cell Death pathways:TNF | 204054_at |
| | | | superfamily, p53 | |
| SPN | 0.102 | 0.049 | Cell Death pathways | 206057_x_at |
| GORASP2 | 0.100 | 0.041 | Cell Death pathways | 208843_s_at |
| DUSP10 | 0.099 | 0.053 | Cell Death pathways:p53 | 221563_at |
| SMG1 | 0.096 | 0.074 | Cell Death pathways:p53 | 208118_x_at |
| FOSL2 | 0.091 | 0.079 | Cell Death pathways | 218881_s_at |
| ERCC1 | 0.089 | 0.082 | Cell Death pathways | 203719_at |
| FGR | 0.088 | 0.048 | Cell Death pathways | 208438_s_at |
| MAP3K7IP2 | 0.079 | 0.050 | Cell Death pathways | 212184_s_at |
| PIK3CA | 0.075 | 0.056 | Cell Death pathways:TNF | 204369_at |
| | | | superfamily, TGF | |
| | | | superfamily | |
| GNAS | 0.071 | 0.023 | Cell Death pathways | 200780_x_at |
| IRS2 | 0.060 | 0.024 | Cell Death pathways | 209185_s_at |
| JUND | 0.059 | 0.058 | Cell Death pathways:TGF | 203752_s_at |
| | | | superfamily | |
| MCL1 | 0.058 | 0.033 | Cell Death pathways | 200797_s_at |
| COL18A1 | 0.057 | 0.072 | Cell Death pathways | 209081_s_at |
| ID3 | 0.052 | 0.100 | Cell Death pathways:cell | 207826_s_at |
| | | | cycle, apoptosis | |
| CDC42 | 0.051 | 0.036 | Cell Death pathways:p53 | 210232_at |
| TRAF6 | 0.049 | 0.062 | Cell Death pathways:TNF | 205558_at |
| | | | superfamily | |
| BNIP3L | 0.044 | 0.036 | Cell Death | 221478_at |
| | | | pathways:apoptosis | |
| KLF2 | 0.038 | 0.010 | Cell Death pathways | 219371_s_at |
| RUNX1 | 0.037 | 0.067 | Cell Death pathways | 208129_x_at |
| BTG2 | 0.033 | 0.011 | Cell Death pathways | 201236_s_at |
| ITM2B | 0.032 | 0.045 | Cell Death pathways | 217732_s_at |
| CD24 | 0.031 | 0.094 | Cell Death pathways | 266_s_at |
| STAT5B | 0.029 | 0.016 | :TNF superfamily | 212549_at |
| PRKRA | 0.027 | 0.022 | Cell Death pathways:TNF | 209139_s_at |
| | | | superfamily | |
| STK17B | 0.025 | 0.010 | Cell Death | 205214_at |
| | | | pathways:apoptosis | |
| HIPK1 | 0.025 | 0.040 | Cell Death pathways | 212291_at |
| PTDSR | 0.018 | 0.018 | Cell Death pathways | 212723_at |

TABLE 35
Selection of Genes Associated with Risk of
Meningoencephalitis and Cell Death

| Cell Death | | | Odds | |
|---|---|---|---|---|
| pathways | Gene | FDR | Ratio | Description |
| IgM | HNRPC | 0.016 | 324.673 | heterogeneous nuclear ribonucleoprotein C |
| | | | | (C1/C2) |
| IgM | DUT | 0.018 | 40.207 | dUTP pyrophosphatase |
| P53 | BARD1 | 0.022 | 11.776 | BRCA1 associated RING domain 1 |
| P53 | CDC42 | 0.036 | 0.051 | cell division cycle 42 (GTP binding protein, |
| | | | | 25 kDa) |
| P53 | NFIL3 | 0.036 | 0.116 | nuclear factor, interleukin 3 regulated |
| P53 | CCNE1 | 0.044 | 6.880 | cyclin E1 |
| P53 | DUSP10 | 0.053 | 0.099 | dual specificity phosphatase 10 |
| P53 | CDK4 | 0.066 | 5.678 | cyclin-dependent kinase 4 |
| P53 | BLM | 0.068 | 13.128 | Bloom syndrome |
| P53 | SMG1 | 0.074 | 0.096 | PI-3-kinase-related kinase SMG-1 |
| P53 | CDC2 | 0.081 | 5.354 | cell division cycle 2, G1 to S and G2 to M |
| target cell | GZMB | 0.010 | 31.809 | granzyme B (cytotoxic T-lymphocyte- |
| killing | | | | associated serine esterase 1) |
| TCR | CABIN1 | 0.036 | 0.162 | calcineurin binding protein 1 |
| TCR | CD80 | 0.036 | 26.520 | CD80 antigen (CD28 antigen ligand 1, B7-1 |
| costimulation | | | | antigen) |
| TCR | CTLA4 | 0.047 | 8.016 | cytotoxic T-lymphocyte-associated protein 4 |
| costimulation | | | | |
| TGF | SMAD7 | 0.060 | 0.113 | SMAD7 |
| TGF | INHBA | 0.077 | 3.484 | inhibin, beta A (activin A, activin AB alpha |
| | | | | polypeptide) |
| TGF | JUND | 0.058 | 0.059 | jun D proto-oncogene |
| TNF | CASP3 | 0.092 | 14.850 | caspase 3, apoptosis-related cysteine |
| | | | | protease |
| TNF | STAT5B, | 0.016 | 0.029 | signal transducer and activator of |
| | 3′UTR | | | transcription 5,3″UTR |
| TNF | CSE1L | 0.019 | 38.753 | CSE1 chromosome segregation 1-like |
| | | | | (yeast) |
| TNF | PRKRA | 0.022 | 0.027 | protein kinase, interferon-inducible double |
| | | | | stranded RNA dependent |
| TNF | ZFP36 | 0.029 | 0.108 | zinc finger protein 36, C3H type, homolog |
| | | | | (mouse) |
| TNF | IL19 | 0.031 | 8.869 | interleukin 19 |
| TNF | PDCD11 | 0.036 | 15.892 | programmed cell death 11 |
| TNF | TRIAD3 | 0.039 | 29.384 | TRIAD3 protein |
| TNF | TNESF10 | 0.042 | 4.806 | tumor necrosis factor (ligand) superfamily, |
| | | | | member 10 |
| TNF | MMP7 | 0.044 | 6.512 | matrix metalloproteinase 7 (matrilysin, |
| | | | | uterine) |
| TNF | KRAS2 | 0.050 | 3.710 | v-Ki-ras2 Kirsten rat sarcoma 2 viral |
| | | | | oncogene homolog |
| TNF | TNFRSF9 | 0.060 | 3.452 | tumor necrosis factor receptor superfamily, |
| | | | | member 9 |
| TNF | TRAF6 | 0.062 | 0.049 | TNF receptor-associated factor 6 |
| TNF | STAT5A | 0.065 | 56.762 | signal transducer and activator of |
| | | | | transcription 5A |
| TNF | NFKB1 | 0.066 | 5.354 | NFKB1 (p105) |
| TNF | TNF | 0.067 | 3.471 | tumor necrosis factor (TNF superfamily, |
| | | | | member 2) |
| TNF | RBM5 | 0.086 | 13.578 | RNA binding motif protein 5 |
| TNF, p53 | PTEN | 0.048 | 0.107 | phosphatase and tensin homolog |
| TNF, p53 | LRDD | 0.049 | 0.219 | leucine-rich repeats and death domain |
| | | | | containing |
| TNF, p53, TCR | STAT1 | 0.004 | 230.416 | signal transducer and activator of |
| | | | | transcription 1, 91 kDa |
| TNF, p53, TCR | PIAS1 | 0.082 | 5.994 | protein inhibitor of activated STAT, 1 |
| TNF, p53, TGF | STAT3 | 0.011 | 6.606 | signal transducer and activator of |
| | | | | transcription 3 |
| TNF, TCR | RARA | 0.077 | 0.123 | retinoic acid receptor, alpha |
| TNF, TGF | PIK3CA | 0.056 | 0.075 | phosphoinositide-3-kinase, catalytic, alpha |
| | | | | polypeptide |
| TNF, TGF | IL12B | 0.067 | 4.425 | interleukin 12B |
| TNF, TGF | PRKCI | 0.098 | 6.662 | protein kinase C, iota |

TABLE 36
Optimal Classifier of Meningoencephalitis Patients Selected by GeneCluster

| | Permutation-based | Permutation-based | Odds Ratio |
|---|---|---|---|
| | p value < 0.01 in | p value < 0.001 in | (logistic |
| Gene | GeneCluster | GeneCluster | regression) |
| STAT3 | Yes | Yes | 6.61 |
| BRD2 - bromodomain | Yes | Yes | 56.32 |
| containing 2 | | | |
| KIF5B - kinesin family | Yes | Yes | 3.73 |
| member 5B | | | |
| LRRFIP1 - leucine rich repeat | Yes | Yes | 18.56 |
| (in FLII) interacting | | | |
| RAB2 - member RAS | Yes | No | 0.002 |
| oncogene family | | | |
| ZNF408 - zinc finger protein | Yes | Yes | 0.239 |
| 408 | | | |
| BTG2 - BTG family, member 2 | Yes | Yes | 0.033 |
| Stat5B 3′UTR | Yes | Yes | 0.029 |

TABLE 37

| | | | | | | | | Resampling |
|---|---|---|---|---|---|---|---|---|
| | | | | | | | Sum | FDR |
| | Affymetrix | Gene | | Affymetrix | Gene | | (absolute | estimate |
| Pair | identifier | symbol | Description | identifier | symbol | Description | log-odds) | (q.value) |
| 1 | 213064_at | FLJ11806 | nuclear protein UKp68 | 221718_s_at | AKAP13 | A kinase (PRKA) anchor protein | 16272922 | <0.0007 |
| | | | | | | 13 | | |
| 2 | 213064_at | FLJ11806 | nuclear protein UKp68 | 211962_s_at | ZFP36L1 | zinc finger protein 36, C3H type- | 910473 | <0.0007 |
| | | | | | | like 1 | | |
| 3 | 201730_s_at | TPR | translocated promoter | 209969_s_at | STAT1 | signal transducer and activator of | 799523 | <0.0007 |
| | | | region (to activated | | | transcription 1, 91 kDa | | |
| | | | MET oncogene) | | | | | |
| 4 | 212152_x_at | ARID1A | AT rich interactive | 209969_s_at | STAT1 | signal transducer and activator of | 743094 | <0.0007 |
| | | | domain 1A (SWI-like) | | | transcription 1, 91 kDa | | |
| 5 | 213064_at | FLJ11806 | nuclear protein UKp68 | 221753_at | SSH1 | slingshot homolog 1 (Drosophila) | 615906 | <0.0007 |
| 6 | 211960_s_at | RAB7 | RAB7, member RAS | 209969_s_at | STAT1 | signal transducer and activator of | 595073 | <0.0007 |
| | | | oncogene family | | | transcription 1, 91 kDa | | |
| 7 | 213064_at | FLJ11806 | nuclear protein UKp68 | 202469_s_at | CPSF6 | cleavage and polyadenylation | 519469 | <0.0007 |
| | | | | | | specific factor 6, 68 kDa | | |
| 8 | 213064_at | FLJ11806 | nuclear protein UKp68 | 210110_x_at | HNRPH3 | heterogeneous nuclear | 454540 | <0.0007 |
| | | | | | | ribonucleoprotein H3 (2H9) | | |
| 9 | 208657_s_at | MSF | MLL septin-like fusion | 209969_s_at | STAT1 | signal transducer and activator of | 409646 | <0.0007 |
| | | | | | | transcription 1, 91 kDa | | |
| 10 | 213064_at | FLJ11806 | nuclear protein UKp68 | 205281_s_at | PIGA | phosphatidylinositol glycan, class | 358825 | <0.0007 |
| | | | | | | A (paroxysmal nocturnal | | |
| | | | | | | hemoglobinuria) | | |
| 11 | 221753_at | SSH1 | slingshot homolog 1 | 209969_s_at | STAT1 | signal transducer and activator of | 325766 | <0.0007 |
| | | | (Drosophila) | | | transcription 1, 91 kDa | | |
| 12 | 211960_s_at | RAB7 | RAB7, member RAS | 213064_at | FLJ11806 | nuclear protein UKp68 | 307504 | <0.0007 |
| | | | oncogene family | | | | | |
| 13 | 202270_at | GBP1 | guanylate binding | 215823_x_at | PABPC1 | poly(A) binding protein, cyto- | 284704 | <0.0007 |
| | | | protein 1, interferon- | | | plasmic 1 | | |
| | | | inducible, 67 kDa | | | | | |
| 14 | 209969_s_at | STAT1 | signal transducer and | 201394_s_at | RBM5 | RNA binding motif protein 5 | 281277 | <0.0007 |
| | | | activator of transcrip- | | | | | |
| | | | tion 1, 91 kDa | | | | | |
| 15 | 203159_at | GLS | glutaminase | 209969_s_at | STAT1 | signal transducer and activator of | 270315 | <0.0007 |
| | | | | | | transcription 1, 91 kDa | | |
| 16 | 202256_at | CD2BP2 | CD2 antigen (cyto- | 209969_s_at | STAT1 | signal transducer and activator of | 257425 | <0.0007 |
| | | | plasmic tail) binding | | | transcription 1, 91 kDa | | |
| | | | protein 2 | | | | | |
| 17 | 209484_s_at | DC8 | DKFZP566O1646 | 202256_at | CD2BP2 | CD2 antigen (cytoplasmic tail) | 240944 | <0.0007 |
| | | | protein | | | binding protein 2 | | |
| 18 | 214911_s_at | BRD2 | bromodomain contain- | 209969_s_at | STAT1 | signal transducer and activator of | 239410 | <0.0007 |
| | | | ing 2 | | | transcription 1, 91 kDa | | |
| 19 | 205988_at | CD84 | CD84 antigen (leuko- | 209969_s_at | STAT1 | signal transducer and activator of | 215312 | <0.0007 |
| | | | cyte antigen) | | | transcription 1, 91 kDa | | |
| 20 | 200626_s_at | MATR3 | matrin 3 | 213064_at | FLJ11806 | nuclear protein UKp68 | 197228 | <0.0007 |

What is claimed is:

1. A method for developing a genomically guided therapeutic product for treating Alzheimer's disease (AD), the method comprising the step of compiling pharmacogenomic information to associate a unique gene expression pattern of a patient sample with a particular clinical response to a treatment for AD.

2. The method of claim 1, wherein the step of compiling comprises the following steps:
(1) procuring at least one patient sample from a patient of a first population of patients and at least one patient sample from a patient of a second population of patients, wherein the first population consists of one or more patients who developed the particular clinical response to the treatment for AD and wherein the second population consists of one or more patients who did not develop the particular response to the treatment for AD;
(2) acquiring a gene expression pattern from each procured patient sample; and
(3) determining whether at least most of the patient samples procured from the first population have a unique gene expression pattern not found in at least most of the patient samples procured from the second population,
wherein a determination that at least most of the patient samples procured from the first population have a unique gene expression pattern not found in at least most of the patient samples procured from the second population results in associating the unique gene expression pattern with the particular clinical response to the treatment for AD.

3. The method of claim 2, wherein the particular clinical response is an adverse clinical response.

4. The method of claim 3, wherein the second population of one or more patients who did not develop the adverse clinical response to the treatment also developed a favorable clinical response.

5. The method of claim 4, further comprising the step of excluding patients from the first population of patients who also developed a favorable clinical response to the treatment for AD.

6. The method of claim 4, further comprising, after the step of procuring and before the step of acquiring, the step of culturing the procured patient samples.

7. The method of claim 6, wherein the patient samples are peripheral blood mononuclear cells.

8. The method of claim 7, wherein the gene expression pattern is selected from the group consisting of protein gene expression patterns and RNA gene expression patterns.

9. The method of claim 1, wherein the treatment for AD comprises administering AN1792, and wherein the step of compiling comprises defining one or more gene expression patterns associated with the development of inflammation after administration of AN1792.

10. The method of claim 3, wherein the treatment for AD comprises administering AN1792.

11. The method of claim 10, wherein the adverse clinical response is inflammation.

12. The method of claim 11, wherein inflammation is selected from the group consisting of encephalitis, meningoencephalitis, vasculitis, cellulitis, and nephritis.

13. A gene expression pattern, wherein the gene expression pattern is associated with a particular clinical response to administration of AN1792.

14. The gene expression pattern of claim 13, wherein the gene expression pattern comprises a panel of genes.

15. The gene expression pattern of claim 14, wherein the panel of genes comprises one or more genes selected from the group consisting of the genes listed in Tables 10, the genes listed in Table 11, the genes listed in Table 12, the genes listed in Table 18, the genes listed in Table 24, the genes listed in Table 25, the genes listed in Table 26, the genes listed in Table 27, the genes listed in Table 28, the genes listed in Table 29, the genes listed in Table 30, the genes listed in Table 31, the genes listed in Table 32, the genes listed in Table 33, the genes listed in Table 34, the genes listed in Table 35, and the genes listed in Table 36.

16. The gene expression pattern of claim 14, wherein the panel of genes comprises the genes listed in Table 36.

17. The gene expression pattern of claim 14, wherein the panel of genes comprises a pair of genes.

18. The gene expression pattern of claim 17, wherein the panel of genes comprises a pair of genes selected from the pairs of genes listed in Table 37.

19. The gene expression pattern of claim 13, wherein the particular clinical response is an adverse clinical response.

20. The gene expression pattern of claim 19, wherein the adverse clinical response is inflammation.

21. The gene expression pattern of claim 20, wherein the gene expression pattern is selected from the group consisting of protein gene expression patterns and RNA gene expression patterns.

22. A method for treating AD comprising:
(1) predicting that a candidate patient will not have an adverse clinical response to a treatment for AD; and
(2) administering the treatment for AD to the candidate patient.

23. The method of claim 22, wherein the step of predicting comprises determining that the candidate patient does not have a gene expression pattern associated with an adverse clinical response to the treatment for AD.

24. The method of claim 22, wherein the step of predicting comprises the following steps:
(1) procuring a test sample from the candidate patient; and
(2) determining whether the test sample from the candidate patient has a test gene expression pattern that is substantially similar to a reference gene expression pattern associated with an adverse clinical response,
wherein if it is determined that the test sample does not have a test gene expression pattern that is substantially similar to the reference gene expression pattern, it may be predicted that the candidate patient will not develop the adverse clinical response.

25. The method of claim 24, wherein the step of procuring a test sample from the candidate patient comprises the following steps:
(1) collecting a blood sample from the patient;
(2) isolating blood cells from the sample;
(3) purifying total RNA from the cells, thereby producing an RNA sample; and
(4) assaying RNA expression levels from the RNA sample to obtain a test gene expression pattern.

26. The method of claim 24, wherein the treatment for AD comprises administering AN1792.

27. The method of claim 26, wherein the adverse clinical response is inflammation.

28. The method of claim 27, wherein inflammation is selected from the group consisting of encephalitis, meningoencephalitis, vasculitis, cellulitis, and nephritis.

29. The method of claim 28, wherein the reference gene expression pattern associated with the adverse clinical response comprises an expression pattern of one or more genes selected from the group consisting of the genes listed in Table 32, the genes listed in Table 33, the genes listed in Table 34, the genes listed in Table 35, the genes listed in Table 36, and the genes listed in Table 37.

30. The method of claim 28, further comprising after the step of isolating and before the step of purifying, the step of culturing the cells with AN1792.

31. The method of claim 30, wherein the reference gene expression pattern associated with the adverse clinical response comprises an expression pattern of one or more genes selected from the group consisting of the genes listed in Table 10, the genes listed in Table 11, and the genes listed in Table 12.
